(19) United States
(12) Patent Application Publication
(10) Pub. No.: US 2021/0330611 A1
(43) Pub. Date: Oct. 28, 2021

(54) THERAPY FOR NEUROLOGICAL DISEASES/DISORDERS

(71) Applicants: Katerina Akassoglou, San Francisco, CA (US); Anke Meyer-Franke, Menlo Park, CA (US); Alex Pico, San Francisco, CA (US); Kean-Hooi Ang, San Francisco, CA (US); Michelle Rose Arkin, San Francisco, CA (US)

(72) Inventors: Katerina Akassoglou, San Francisco, CA (US); Anke Meyer-Franke, Menlo Park, CA (US); Alex Pico, San Francisco, CA (US); Kean-Hooi Ang, San Francisco, CA (US); Michelle Rose Arkin, San Francisco, CA (US)

(21) Appl. No.: 17/229,394

(22) Filed: Apr. 13, 2021

Related U.S. Application Data
(60) Provisional application No. 63/009,212, filed on Apr. 13, 2020.

(51) Int. Cl. A61K 31/137; A61K 31/365; A61K 45/06; A61K 31/5415; A61K 31/7048; A61K 31/7024; A61K 31/475; A61K 31/165

(52) U.S. Cl. CPC A61K 31/137; A61K 31/365; A61K 45/06; A61K 31/165; A61K 31/7048; A61K 31/7024; A61K 31/475; A61K 31/5415

(57) ABSTRACT

Provided herein are compositions and methods to treat or prevent neurodegeneration in a mammal, inhibit microglial activation in the CNS of a mammal, promote survival of CNS neurons in a mammal, prevent or reduce the rate of demyelination and/or neuronal injury in a mammal, promote remyelination in a mammal and/or treat or prevent or decrease oxidative stress in a mammal.

### CLAIM OF PRIORITY

This application claims the benefit of priority of U.S. Provisional Patent Application No. 63/009,212, filed on Apr. 13, 2020, the benefit of priority of which is claimed hereby, and which is incorporated by reference herein in its entirety.

### GOVERNMENT SUPPORT

This invention was made with government support under Grant No. R35 NS097976 awarded by the National Institutes of Health. The government has certain rights in this invention.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Neurodegeneration is the progressive loss of structure and/or function of neurons, which may lead to the death of the affected neurons. Neurodegenerative diseases include Alzheimer's disease, Parkinson's disease, amyotrophic lateral sclerosis, Huntington's disease and multiple sclerosis. Although these diseases have different etiologies and symptoms, they all result in progressive degeneration and/or death of neuron cells. Despite their differences, these diseases also display similarities that can relate these diseases on a cellular or molecular level. Such similarities offer therapeutic advances using modalities common to each of these diseases.

Clinical management of neurodegenerative remains a significant challenge in medicine, however, as they do not address the cellular or molecular basis of the disease. Although some degree of axonal remyelination by oligodendrocytes takes place early during the course of MS, the ability to repair the CNS eventually fails, leading to irreversible tissue injury and an increase in disease-related disabilities.

Currently approved therapies for CNS demyelinating diseases, such as multiple sclerosis (MS), are primarily immunomodulatory, and typically do not have direct effects on CNS repair. Similarly, drugs for other neurodegenerative diseases such as Alzheimer's disease and Parkinson's disease do not address the neuronal death and loss of function, but rather ameliorate associated symptoms.

Thus, there is a need for additional therapies that prevent and/or ameliorate neurodegeneration. The present invention meets such need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides methods and compositions for treating a CNS disease, disorder or injury. The present invention also provides methods and compositions for preserving or protecting neural structure and/or function in a subject in need thereof, such as in a mammalian subject by administering one or more agents and/or compositions described herein to the subject.

One embodiment provides a method of treating or preventing neurodegeneration in a mammal, such as a human, comprising administering to the mammal in need thereof an effective amount of a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

In a related aspect, the invention features a method of preventing progression of a CNS disorder in a subject in need of treatment. The method comprises administering to the subject a composition comprising a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof in an amount sufficient to thereby arrest the CNS disorder and prevent further neuronal injury and/or death. In certain embodiments, said treatment may result in reduction of one or more symptoms associated with the disease. In some embodiments, the treatment results in reducing, retarding or preventing a relapse, or the worsening of progression of the disease in the subject.

Some embodiments provide for methods and compositions for preventing or ameliorating demyelination in a subject, such as mammalian subject, by administering one or more compositions that comprise a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

Other embodiments provide methods and compositions for enhancing myelination and/or re-myelination in a mammalian subject, such as a human subject, by administering one or more compositions that comprise a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

Further embodiments provide methods and compositions for decreasing neurodegeneration associated with plaque formation (e.g., amyloid plaque formation) in a mammalian subject, such as a human subject.

In some embodiments, the methods and compositions of the invention are used for decreasing neurodegeneration in a patient with multiple sclerosis.

In some embodiments, the methods and compositions of the invention are used for decreasing neurodegeneration in a patient with Alzheimer's disease.

Other embodiments provide methods and compositions for decreasing neurodegeneration in a mammalian subject with a genetic predisposition for Alzheimer's disease. Examples of such genetic predisposition include mutations m the amyloid precursor protein (APP) gene, Presenilin 1 (PSEN1) and Presenilin 2 (PSEN2) genes, and ApoE4.

One embodiment provides a method of treating or preventing or decreasing oxidative stress in a mammal comprising administering to the mammal in need thereof an effective amount of a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

Another embodiment provides a method for inhibiting microglial activation in the CNS of a mammal with a disease, disorder, or injury involving demyelination, dysmyelination, or neurodegeneration, comprising administering to the mammal an effective amount of a composition comprising a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

One embodiment provides a method of preventing demyelination and neuronal injury in a mammal in need thereof, the method comprising administering to the mammal a therapeutically effective amount of a pharmaceutical composition comprising a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof so as to prevent an increase in demyelination and injury of CNS neurons in said mammal.

Another embodiment provides a method for promoting survival of CNS neurons in a mammal, comprising administering to a mammal in need thereof an effective amount of a composition comprising a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof so as to decrease neuronal injury/promote survival of neurons in the CNS of said mammal.

One embodiment provides a method of inhibiting microglial activation in CNS neurons comprising contacting CNS neurons with a composition comprising a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

Another embodiment provides a method for promoting remyelination in a mammal comprising administering to the mammal an effective amount of a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

One embodiment provides a method for reducing the rate of demyelination and neuronal injury in a mammal comprising administering to the mammal an effective amount of a modulator of calcium signaling, a modulator of microtubule dynamics, a modulator of calcium signaling, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

One embodiment provides a method to treat or prevent neurodegeneration in a mammal comparing administering to said mammal an effect amount of one or more of the small molecules provided in Table 1 and/or Table 2, provided that the small molecule is not acivicin.

Another embodiment provides a pharmaceutical composition comprising at least one modulator of calcium signaling, at least one modulator of microtubule dynamics, at least one modulator of calcium signaling, at least one modulator of chemokine signaling, at least one modulator of DNA replication, at least one modulator of dopamine receptor signaling, at least one modulator of cAMP signaling, at least one modulator of glucocorticoid-receptor signaling, at least one modulator of purine nucleotide biosynthesis, at least one modulator of neurotransmitter transport or a combination thereof, in an amount effective to treat or prevent neurodegeneration, and a pharmaceutically acceptable carrier, diluent, or excipient.

One embodiment provides a pharmaceutical composition with selectivity to inhibit fibrin-induced microglia activation, but not LPS-induced activation comprising at least one modulator of microtubules, at least one modulator of glucocorticoids, at least one modulator of progesterone, at least one modulator of bacterial growth, at least one modulator of β2-adrenoceptor signaling, at least one modulator of parasite growth, at least one modulator of alpha 2-adrenergic receptors, at least one modulator of alpha 1-adrenergic receptor, at least one modulator of 5-HT4 receptors, at least one modulator of HMG-CoA reductase, at least one modulator that decreases free radicals, at least one modulator that decreases prostaglandins, at least one modulator that regulates mineralocorticoid receptor signaling or a combination thereof, in an amount effective to treat or prevent neurodegeneration, and a pharmaceutically acceptable carrier, diluent, or excipient.

In one embodiment, the modulator of calcium signaling is a calcium channel blocker, a vasodilator or an adrenoreceptor agonist. In another embodiment, calcium channel blocker comprises fendiline. In one embodiment, the vasodilator comprises nylidrin. In one embodiment, the modulator of microtubule dynamics is an inhibitor of microtubule assembly. In one embodiment, the inhibitor of microtubule assembly comprises vinblastine (vinblastine sulfate), colchicine and/or podofilox. In another embodiment, the modulator of chemokine signaling comprises tannic acid. In one embodiment, the modulator of DNA replication is an inhibitor of DNA topoisomerase II, such as teniposide. In one embodiment, the modulator of dopamine receptor signaling is blocker of dopamine receptors, such as prochlorperazine or thioridazine. In another embodiment, the modulator of cAMP signaling comprises nylidrin, prochlorperazine or thioridazine. In one embodiment, the modulator of glucocorticoid-receptor signaling comprises betamethasone, dexamethasone, dexamethasone acetate, dexamethasone sodium phosphate, fludrocortisone acetate, fluocinolone acetonide, fluocinonide, fluorometholone, flurandrenolide, hydrocortisone, hydrocortisone acetate, hydrocortisone butyrate, hydrocortisone hemisuccinate, hydrocortisone sodium phosphate, methylprednisolone, prednisolone or triamcinolone diacetate. In another embodiment, the modulator of purine nucleotide biosynthesis is an inhibitor of inosine-5′-monophosphate dehydrogenase (IMPDH), such as mycophenolic acid (MPA). In one embodiment, the modulator of neurotransmitter transport is an inhibitor of norepinephrine reuptake, such as maprotiline.

In another embodiment, said mammal has been diagnosed with a disease, disorder, or injury involving demyelination, dysmyelination, or neurodegeneration. In one embodiment, said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillain-Barre syndrome, Marie-Charcot-Tooth disease and Bell's palsy.

One embodiment also includes pharmaceutical compositions and kits that contain one or more agent that can be used to inhibit degeneration of a neuron or a portion thereof, as described herein. The pharmaceutical compositions and kits can optionally include one or more pharmaceutically acceptable excipients.

Another embodiment features a packaged composition (e.g., a packaged pharmaceutical composition) that includes at least one agent disclosed herein that is labeled and/or contains instructions for use of said agent for treating a CNS disorder and/or oxidative stress. The agent can be in a form suitable for any route of administration, e.g., oral administration, peripheral administration, intrathecal administration, etc. One or more active agents can be included in the packaged pharmaceutical composition.

These aspects and other features and advantages of the invention are described below in more detail. Those skilled in the art will recognize or be able to ascertain using no more than routine experimentation, many equivalents to the specific embodiments of the invention described herein. Such equivalents are intended to be encompassed by the following claims.

### BRIEF DESCRIPTION OF THE DRAWINGS AND APPENDICES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-G. Single cell oxidative stress transcriptome of CNS innate immunity. a, Workflow for Tox-seq in EAE and HTS of primary microglia followed by in vivo validation and drug target network overlays. b, UMAP plot of single CD11b⁺ROS⁻ and CD11b⁺ROS⁺ cells identified by unsupervised clustering analysis (n=8,701 cells from spinal cord of 2 healthy and 2 EAE mice). The gray outlines demarcate individual clusters as shown in (f). Color scheme is based on Tox-seq label, which is sample type (healthy or EAE) and ROS expression (CD11b+ROS− and CD11b+ROS+). Clusters are further grouped by cell type identified by gene signatures shown in (g). Cells from EAE mice were collected at onset of disease (clinical score 1.0). c, Fraction of cells in each cluster colored based on Tox-seq label (key). d, Percent of CD11b⁺ROS⁻ and CD11b⁺ROS⁺ cells from EAE mice. e, Single cell expression heat map of genes from GO term “Reactive oxygen species metabolic process” (P=1.38×10-19, Benjamini-Hochberg test). Genes are grouped by gene function and related biological process (BP) terms. f, UMAP plot of 8,701 CD11 b⁺ cells with clusters numerically labeled (key). Selected top three DEGs per cluster are shown. g, Dot plot of gene makers across all CD11 b⁺ clusters. The dendrogram above dot plot depicts unsupervised hierarchical clustering.

FIGS. 2A-E. Overlay of CNS innate immune cell clusters with oxidative stress core signature genes. a, UMAP plots of unsupervised subclustering analysis of microglia (n=3,033 cells from 2 healthy and 2 EAE mice) or monocyte/macrophages (n=5,240 cells from 2 EAE mice) showing clusters numerically labeled. b, Ridge plot of Cx3cr 1 gene expression levels across all microglia and monocyte/macrophage clusters. c, Heat maps of top significant GO terms for each single cell cluster (P<0.05, Benjamini-Hochberg test). d, UMAP plots of microglia or monocyte/macrophages clusters showing single-cell gene expression overlays for Cybb and H2-Ab/(top), Cybb and Cyba (middle) or Cd74 and H2-Ab1 (bottom). Cells highly co-expressing both gene marker pairs are depicted as double⁺ cells in yellow (right key). Clusters from healthy and EAE mice are shown (bottom key). e, Heat map depicting comparison of Tox-seq microglia subclusters in EAE with signatures of microglia from human active/chronic MS lesions (16). Average gene expression is depicted as scaled z-score expression (key).

FIGS. 3A-I. Validation of the oxidative stress core signature in EAE. a-f, Violin plots of log expression levels of Clec4e (a), F10 (encodes coagulation factor X) (b), Cybb (encodes the NADPH oxidase subunit gp91-phox) (c), H2-Ab1 (encodes MHC II) (d),Il1b (e) and P2ry12 (f) across microglia and monocyte/macrophages subclustered single-cell populations. Dots depict single cells. g, Confocal microscopy of a white matter (WM) lesion from Cx3cr1^{GFP/+}Ccr2^{RFP/+} mice with MOG₃₅₋₅₅-induced EAE, showing CX3CR1⁺ cells (green), CCR2⁺ cells (red), and immunoreactivity for the oxidative stress marker NADPH oxidase subunit gp91-phox (blue) and the antigen presentation marker MHC II (white). Data are representative of n=4 mice; tissues were collected at onset of EAE (clinical score 1.0). Yellow arrows, gp91-phox⁺MHC microglia; orange arrows, gp91-phox⁺MHC II⁻ microglia; pink arrowhead, gp91-phox⁺MHC⁺ monocyte/macrophages; green arrowhead, gp91-phox⁻MHC⁺ monocyte/macrophages; grey arrowhead, gp91-phox⁺MHC II⁻ monocyte/macrophages. h, i, Confocal microscopy from spinal cords of mice as in g, showing CX3CR1⁺ cells (green), CCR2⁺ cells (red), and immunoreactivity for coagulation factor X (blue) (h), or CLEC4E (blue) (i). Data are representative of n=4 mice; tissues were collected at onset of EAE (clinical score 1.0). Arrows and arrowheads denote microglia and monocyte/macrophages, respectively. Scale bar, 50 μm (g-h).

FIGS. 4A-B. Bulk Tox-seq and co-expression gene network analysis of ROS⁺ microglia in EAE. a, Heat map of GO analysis from bulk RNA-seq of ROS⁺ and MHC microglia and infiltrating monocyte/macrophages compared to respective baseline (ROS⁻,MHC II⁻) cells from EAE mice. Key indicates scaled z-score expression. Data are from n=3 biological replicates per group. b, Co-expression gene networks from bulk RNA-seq of ROS⁺ microglia. Darker interaction lines represent greater evidence for co-expression. Key indicates log2 fold change expression and is relative to MHC microglia; red border indicates significance of P<0.05 (Benjamini-Hochberg test).

FIGS. 5A-C. Microglia HTS and oxidative stress gene network with Tox-seq data overlay. a, Microscopy of image-based assay to detect activated primary microglia. Insets, higher magnification. Representative of >100 experiments. Scale bars, 100 μm. b, A scatter plot of HTS screening. Normalized percent inhibition of coated fibrin and LPS stimuli by 1907 clinical drugs and bioactive molecules. Molecules that caused cell death or less than 50% inhibition were classified as toxic (gray circles) or inactive (white circles), respectively. Hits were defined as molecules that exhibited greater than 50% inhibition of either fibrin or LPS activation (light red circles). Acivicin [(2S)-2-amino-2-[(5S)-3-chloro-4,5-dihydro-1,2-oxazol-5-yl] acetic acid] is indicated in (bright red circle highlighted). Scatter-plot quadrants (key) (Q1; position of molecules with LPS inhibition, Q2; position of molecules with LPS and fibrin inhibition, Q3; position of molecules with fibrin inhibition, Q4; position of inactive molecules). c, Oxidative stress gene network visualizes the cellular transport, biosynthesis, redox cycle, glutathionylation and extracellular recycling of glutathione (GSH). Drugs are represented as ovals, gene and protein targets as rectangles (key); split-box view represents cell specific gene expression changes in microglia (left) or monocytes (right); blue shading, genes downregulated in MHC II⁺ cells (fold changes log2<−1); Red shading, genes upregulated in ROS⁺ cells (fold changes log2>1); the red border indicates significance of P<0.05 (two-tailed moderated t-test).

FIGS. 6A-F. GGT inhibition in innate immune cells. a, Normalized dose response of acivicin on fibrin-induced (black circles) or LPS-induced (blue circles) microglia activation without cell toxicity <20 μM (red circles) (key). Data are from one experiment performed in triplicates (mean±s.d.). b, Quantification of ROS production (assessed via DHE) in mouse BMDMs (left) and human macrophages (right) unstimulated or stimulated for 24 h with fibrin in the presence of acivicin or GGsTop (grid plot). Data are from n=3 independent experiments performed in quadruplicate (mouse) and sextuplicate (human) (mean±s.e.m.). A.U., arbitrary units. *** P<0.001, **** P<0.0001. (one-way ANOVA with Tukey's multiple comparisons test). c, GGT activity in BMDMs left unstimulated or stimulated with fibrin after acivicin or vehicle control treatment (key). Data are from n=3 independent experiments performed in duplicate (mean±s.e.m.). * P<0.05 (two-way analysis of variance (ANOVA) with Sidak's multiple comparisons test). d, Quantification of glutathione (GSH) RealThiol probe (F405/F488 ratio) by real time confocal imaging of live BMDMs. The fluorescence ratio F405/F488 of RealThiol probe reflects the ratio of RealThiol probe bound to intracellular GSH over unbound intracellular probe and is proportional to the GSH concentration. F405/F488 was expressed as percentage of that of the untreated cells (control). Data are from n=7 independent experiments (mean±s.e.m.). n.s., not significant. * P<0.05 (two-tailed paired t-test with Benjamini-Hochberg test). e, Quantification of ROS production (assessed via DHE) in BMDMs isolated from Ggt1^{+/+} or Ggt1^{dwg/dwg} mice (key) left unstimulated or stimulated for 24 h with fibrin. Data are from n=4 mice per group performed in quintuplicate (mean±s.e.m.). A.U., arbitrary units. **P<0.01 (two-way ANOVA with Sidak's multiple-comparisons test). f, qRT-PCR analysis of Nos2, Cxcl10, Cc15 and Il1b expression in BMDMs isolated from Ggt1^{+/+}and Ggt1^{dwg/dwg} mice (key) stimulated with fibrin. Data are from n=5 mice per group (mean±s.e.m.). ** P<0.01 (two-tailed Mann-Whitney test). Each circle symbol represents an individual experiment (a-d) or mouse (e, f).

FIGS. 7A-J. Acivicin suppresses neuroinflammation and neurodegeneration in EAE. a, Confocal microscopy of spinal cord sections from unimmunized healthy mice and MOG₃₅₋₅₅-induced EAE mice at peak disease, immunostained for GGT1 (red). Nuclei are stained with 4′,6-diamidino-2-phenylindole (DAPI, blue). WM, white matter; Scale bars, 100 μm. Image quantification of GGT1 immunoreactivity is shown. Data are representative of n=5 mice per group (mean±s.e.m) ** P <0.01 (two-tailed Mann-Whitney test). b, Representative flow cytometry contour plots (left) showing GGT1 expression in CD11b⁺CD45^{low}Ly6C⁻ (microglia) and CD11b⁺CD45^{high}Ly6C^{high} (infiltrating monocyte/macrophages) cells from spinal cord of mice with MOG₃₅₋₅₅-induced EAE or unimmunized healthy controls. GGT1 expression is depicted as percent of gated population ±s.d. Quantification (bottom) of GGT1⁺ microglia and monocyte/macrophages are shown. Cells were analyzed at peak of EAE (clinical score 3.0). Data are from n=4 mice (healthy) and n=5 mice (EAE) (mean±s.e.m.). * P<0.05, ** P<0.01 (one-way ANOVA with Tukey's multiple comparisons test). c, Clinical scores for relapsing EAE of SJL/J mice immunized (upward arrow) with PLP₁₃₉₋₁₅₁, followed by therapeutic injection of acivicin or saline (key) every day starting at the peak of the initial paralytic episode (dotted vertical line). Data are from n=7 mice (EAE+acivicin) and n=6 mice (EAE+saline) (mean±s.e.m.). * P<0.05, ** P<0.01 (two-tailed permutation test). d, Clinical scores of adoptive transfer EAE induced by TH1 cells (upward arrow) after daily prophylactic administration of acivicin or saline (key) starting day 0. Data are from n=10 mice (EAE+acivicin) and n=7 mice (EAE+saline) (mean±s.e.m.). * P<0.05, ** P<0.01, *** P<0.001 (two-tailed permutation test). e, GGT activity in spinal cords from healthy or MOG₃₅ 55-EAE mice treated with acivicin or saline (grid plot). Data are from n=5 mice (control), n=6 mice (EAE+saline), and n=5 mice (EAE+acivicin), each performed in technical duplicates (mean±s.e.m.). **** P<0.0001 (one-way ANOVA with Tukey's multiple comparisons test). f, Quantification of 4-HNE immunostaining in spinal cords from mice with peak MOG₃₅₋₅₅-EAE disease after prophylactic injection with acivicin or saline. Data are from n=8 mice (saline) and n=7 mice (acivicin) (mean±s.e.m.). *** P<0.001 by two-tailed Mann-Whitney test. g, Quantification of serum protein carbonyl levels in MOG₃₅₋₅₅ EAE mice after prophylactic injection with acivicin or saline. Data are from n=9 mice (Healthy, control), n=7 mice (EAE+saline), and n=7 mice (EAE+acivicin) (mean±s.e.m.). *** P<0.001, **** P<0.0001 (one-way ANOVA with Tukey's multiple comparisons test). h, qRT-PCR analysis of gene expression from spinal cord extracts of unimmunized healthy controls and MOG₃₅₋₅₅-EAE mice at peak disease, treated with saline or acivicin. Data are from n=5 mice (unimmunized healthy control), n=7 mice (EAE+saline), or n=7 mice (EAE+acivicin) (mean±s.e.m.). * P<0.05 (one-way ANOVA with Bonferroni multiple comparisons test). i, Microscopy (left) of spinal cord sections from mice with MOG₃₅₋₅₅-induced EAE treated with acivicin or saline, showing SMI-32 immunoreactivity (top; indicative of axonal damage) and myelin basic protein (MBP) immunoreactivity (bottom; indicative of myelin); dashed lines demarcate demyelinated white matter of the spinal cord column. Scale bars, 25 μm (top), 80 μm (bottom). Image quantification of SMI-32 and MBP immunoreactivity is shown (right). Data are from n=8 mice (EAE+saline) and n=7 mice (EAE+acivicin) (mean±s.e.m.). * P<0.05, *** P<0.001, two-tailed Mann-Whitney test. j, Microscopy (left) of spinal cord sections from Cx3cr1^{GFP/+}Ccr2^{RFP/+} mice with peak MOG₃₅₋₅₅-EAE treated with acivicin or saline, showing CX3CR1⁺ (GFP, green) and CCR2⁺ (RFP, red) cells. Image quantification (right) of CX3CR1⁺ (top) and CCR2⁺ cells (bottom) is shown. Scale bars, 100 μm. Data are from n=8 mice (EAE+saline) and n=7 mice (EAE+acivicin) (mean±s.e.m.). ** P<0.01, *** P<0.001 (two-tailed Mann-Whitney test). Each circle symbol (a, b, e-j) represents an individual mouse.

FIGS. 8A-D. Acivicin suppresses demyelination, axonal damage and oxidative stress in chronic EAE. a, Schematic of chronic EAE of NOD mice immunized with MOG₃₅₋₅₅, followed by therapeutic injection of acivicin or saline every day starting at day 80 until day 113 of the chronic phase. b, Clinical scores for chronic EAE of NOD mice immunized (upward arrow) with MOG₃₅₋₅₅, followed by therapeutic injection of acivicin or saline (key) every day starting at day 80 of the chronic phase (dotted vertical line). Data are from n=8 mice (EAE+acivicin) and n=9 mice (EAE+saline) (mean±s.e.m.). * P<0.05 (two-tailed permutation test). c, Microscopy (left) of spinal cord sections from mice with chronic MOG₃₅₋₅₅ EAE treated with acivicin or saline every day starting at day 80 until day 113 labeled for MBP; right, quantification of area devoid of MBP immunoreactivity. Dashed lines (left) demarcate demyelinated white matter of the spinal cord column. Scale bar, 300 μm. Data are from n=7 mice per group (mean±s.e.m). ** P<0.01 by two-tailed Mann-Whitney test. d, Microscopy (left) of spinal cord sections from mice with chronic MOG₃₅₋₅₅ EAE treated with acivicin or saline every day starting at day 80 until day 113 labeled for SMI-32 immunoreactivity (top), iNOS (middle), or NADPH oxidase subunit gp91-phox (bottom); right, quantification of area with SMI-32 immunoreactivity, area with iNOS immunoreactivity, or area with gp91-phox immunoreactivity in white matter. Scale bar, 100 μm. Data are from n=7 mice per group (mean±s.e.m). ** P<0.01, *** P<0.001 by two-tailed Mann-Whitney test. Each circle symbol represents an individual mouse.

FIGS. 9A-H. Validation of single cell oxidative stress transcriptome of CNS innate immunity. a, Flow cytometry plots of live CD11b⁺ROS⁻ and live CD11b⁺ROS⁺ cells from spinal cord of individual healthy and EAE mice. Percent of cell population is shown inside gate. b, Representative histogram plots (top) and quantification of mean fluorescence intensity (MFI, bottom) of ROS production (assessed via DCFDA) in CD11 b⁺ cells from healthy and EAE mice. Data are from n=4 mice per group (a, b) (mean±s.d.). *P<0.05 (two-tailed Mann-Whitney test). c, Violin plots of number (n) Gene (nGene) and unique molecular identifiers (nUMI) post normalization and shown for each biological replicate used for scRNA-seq data analysis. Data are from n=2 mice (Healthy) and n=2 mice (EAE). d, Correlation gene expression scatter plot between healthy (left) or EAE (right) biological replicates from scRNA-seq dataset (n=2 healthy and 2 EAE mice). The average gene expression is shown for cluster 1 and cluster 6 with Pearson correlation=0.98 and 0.99, respectively. e, UMAP plots (left) of each cells sample type and summarized in table (right). f, Violin plots of log expression levels of potential astrocytic (Gfap), neuronal (Tubb3 and Rbfox3), oligodendrocyte (Mog and Plp1) contaminates in each cluster population identified in (g). Genes shown were not differentially expressed. Actb is shown as a reference gene. g, UMAP plots of all 14 populations identified by unsupervised clustering analysis (top) and pseudo colored based on cell type (bottom, key). Representative core gene signatures are shown for each cell type. DEGs in cluster 2 are immediate early response genes (e.g. fun, Fos, Egrl) and likely due to artifact as recently reported (54). h, Heat map of the top 5 differentially expressed genes per single cell cluster as shown in (g). The clusters are ordered by unsupervised hierarchical clustering. Key indicates scaled z-score expression.

FIGS. 10A-F. Subcluster analysis of CNS innate immune populations and comparison with canonical myeloid gene signatures. a, Heat map of oxidative stress subclusted populations from Tox-seq analyzed for gene markers for select microglia (green text) (19,20), monocyte/macrophage (orange text) (20,26,27), and CNS-associated macrophage (black text) (20). Key indicates average gene expression (log scale). Asterisk indicates genes previously validated by cell-fate mapping (20). b, Violin plots from Tox-seq clusters Mg5 and Mp1 showing single-cell gene expression levels of highly conserved microglia core genes (28). c, Heat maps of the top 5 differentially DEGs across microglia (top) or monocytes/macrophages (bottom) single cell subclusters. Key indicates scaled z-score gene expression. d, UMAP plots of monocytes/macrophages subclusted and colored based on FACS-purified EAE CD11b⁺ROS+ cells (red dots; DCFDA expressing cells), high expressing NAPDH oxidase (Cybb⁺×Cyba⁺) cells (green dots) and overlay between the two populations (yellow dots). The percent colocalization between the number of FACS-purified EAE CD11b⁺ROS⁺ and high expressing NAPDH oxidase cells is shown in key. e, Violin plots of log expression levels of Sparc across all microglia (Mg) and monocyte/macrophage (Mp) clusters from scRNA-seq. Dots depict individual cells. f, Confocal microscopy quantification related to FIG. 3g, and presented as percent of CX3CR1⁺ and CCR2^{rfp+} cells colocalized with gp91-phox and MHCII in spinal cord sections from Cx3cr1^{gfp/+}:Ccr2^{rfp+} mice with MOG₃₅₋₅₅-induced EAE. Data are representative of n=4 mice per group (mean±s.e.m.).

FIGS. 11A-G. Bulk RNA-seq of ROS+ and MHC+CNS innate immune cells. a, Representative flow cytometry plots of CD45^{low}CD11 b⁺ (microglia) and CD45^{high}CD11b⁺ (monocytes) cells sorted based on MHCII and ROS production (assessed via DCFDA) from EAE spinal cord. b, Cell percentages of sorted microglia and monocytes populations. Data are from n=3 independent experiments with 5 spinal cord EAE tissues pooled per experiment. c, d, Quantification of MFI of ROS production (assessed via DCFDA) (c) and MHCII (d) expression in CD45lowCD11b+ and CD45highCD11 b cells. Data are from n=3 experiments (mean±s.e.m.) ** P=0.0011 (unpaired t-test). e, Gene expression (FPKM) profiles of select microglia and monocyte/macrophage markers across each sorted population. Base, MHCII⁻ROS⁻ cells. f, Heat map of bulk RNA-seq cluster analysis from CD45lowCD11b+ cells. Six gene clusters (C1-6) were annotated with functions for selected genes based on expression levels. g, Heat map of bulk RNAseq cluster analysis from CD45highCD11b+ cells. Four gene clusters (C1-4) were annotated with functions for selected genes based on expression levels. Key indicates normalized z-score expression.

FIGS. 12A-E. Effects of acivicin on microglia, macrophage and whole blood cell numbers. a, Representative images showing morphology and density of primary microglia treated with vehicle or acivicin for 48 h stained with nuclei marker Hoechst or CellMask Red Whole Cell Stain. Quantification of the total number of microglia. Data are representative of one experiment with technical replicates (open symbols). b, Representative confocal images showing morphology and density of control- and acivicin-treated BMDMs treated for 24 h. Scale bar, 100 μm. Insets depict digitally zoomed in areas of regions of interest. Quantification of the total number of BMDMs in the field of view. Data are from n=7 independent experiment (mean±s.e.m.). ns, not significant (by two-tailed unpaired Student's t-test). c, Representative flow cytometric plots of the gating strategy for BMDMs treated with acivicin for 24 h or left untreated. Cell viability of single CD45⁺ cells was assessed by Sytox blue live/dead nuclei stain. Quantification of percent cell viability of CD45⁺ BMDMs treated with acivicin or left untreated. Data are from 2 independent experiments with n=4 mice per group (mean±s.d.). d, Quantification of complete blood count analysis of white blood cells (WBCs), red blood cells (RBC), platelets (PLT) (left) and composition of WBCs (right) of blood samples from healthy mice treated with saline or acivicin for 10 days. Data are from n=8 mice (saline) or n=7 mice (acivicin) (mean+s.e.m). d, Representative flow cytometric plots of CD45 and CD11 b expressing immune cells from the spinal cord of healthy mice treated with acivicin or saline every day for 14 days. Quantification of total microglia cell numbers in healthy mice treated with acivicin or saline. Data are from n=3 mice per group (mean±s.e.m.). ns, not significant (by two-tailed Mann-Whitney test).

FIGS. 13A-C. Pharmacologic or genetic inhibition of GGT in macrophages. a, b, GGT activity measured by fluorescent probe gGlu in fibrin stimulated BMDMs in the absence and presence of acivicin (a) or GGsTop (b) (8.3, 2.8, 0.93, 0.31, 0.10, 0.03, or 0.01 μM; wedge). Data are from n=4 independent experiments performed in duplicate (mean±s.e.m). * P<0.05, ** P<0.01, *** P<0.001, **** P<0.0001 (one-way ANOVA with Dunnett's multiple comparison test). c, qRT-PCR analysis of Ccl5, Cxcl3, Cxcl10 and Il1b expression in BMDMs isolated from Ggt1+/+ and Ggt1^{dwg/dwg} mice left unstimulated or stimulated with LPS for 6 hr (key). Data are from n=4 mice per group (mean±s.e.m). *P<0.05, **** P<0.0001 (two-way ANOVA with Tukey's multiple comparison test).

FIGS. 14A-I. Effects of acivicin in EAE, antigen-presenting cell function and GGT1 expression. a, Clinical scores of MOG35-55 EAE mice after daily prophylactic administration of acivicin or saline (key) starting day 0. Data are from n=14 mice (acivicin), n=15 mice (saline) (mean±s.e.m). * P<0.05, ** P<0.01, *** P<0.00 (two-tailed permutation test). b, Clinical scores for MOG₃₅₋₅₅-induced EAE (upward arrow), followed by prophylactic injection of GGsTop (5 mg/kg, i.p.) or saline (key) every day beginning on day 0. Data are from n=13 mice (MOG₃₅₋₅₅+saline) or n=12 mice (MOG₃₅₋₅₅ GGsTop) (mean±s.e.m). * P<0.05, ** P<0.01, *** P<0.001 (two-tailed permutation test). c-f, Flow cytometry analysis of splenocytes from MOG₃₅₋₅₅-EAE mice treated with saline or acivicin for 10 days and assessed for IL-17⁺ or IFN-γ⁺ CD4⁺ Tcells (c, d,), effector memory (TEM), central memory (TCM), and naive CD4⁺ T cells (E), or CD25⁺FoxP3⁺ Treg cells (f). Data are from n=5 mice per group (mean±s.e.m.). **** P<0.0001 (two-way ANOVA with Tukey's multiple comparison test) (a, b); ** P<0.01 (two-tailed Mann-Whitney test) (c, d). g, h, Flow cytometry analysis of BrdU⁺ 2D2 T cells activated with LPS primed BMDMs (pulsed with MOG₃₅₋₅₅ peptide) or anti-CD3 and anti-CD28 in the presence or absence of acivicin (5, 0.5, or 0.05 μM; wedge). Data are from n=3 independent experiments performed in duplicate (mean+s.e.m.). * P<0.05, ** P<0.01, **** P<0.001, n.s., not significant (one-way ANOVA with Tukey's multiple comparison test). i, Confocal microscopy of spinal cord sections from NOD mice with chronic MOG₃₅₋₅₅ EAE, treated with acivicin or saline every day starting at day 80 until day 113, showing GGT1 and CD11 b immunoreactivity. Quantification of GGT1 immunoreactivity in white matter of spinal cord. Data are from n=7 mice (EAE+acivicin) and n=7 mice (EAE+saline) (mean±s.e.m). ** P<0.01 by two-tailed Mann-Whitney test. Each symbol represents an individual mouse (c-f, i) or an individual experiment (g, h).

FIGS. 15A-C. Effects of acivicin in LPS-induced neurodegeneration. a, GGT activity in substantia nigra (SN) extracts 12 h following PBS or LPS injection in mice pretreated with acivicin or saline for 5 d. Data are from n=6 mice (control, PBS, or LPS+saline) or n=8 mice (LPS+acivicin) (mean±s.e.m.). ** P<0.01, *** P<0.001 (two-way ANOVA with Tukey's multiple comparison test). b, c, Microscopy images of brain sections spanning SN 7 d following PBS or LPS injection in mice pretreated with acivicin or saline, tissues were stained for tyrosinehydroxylase (TH) (b) or Iba-1 (c). Scale bars, 300 μm (b, c). Quantification of TH (b) and Iba-1 (c) immunoreactivity in SN is shown (right). Data are from n=6 mice (saline) and n=5 mice (acivicin) (b); n=6 mice (saline+PBS or saline+LPS) and n=5 mice (acivicin+PBS or acivicin+LPS) (c) (mean±s.e.m.). * P<0.05 (two-tailed Mann-Whitney test) (b); ** P<0.01 (two-way ANOVA with Tukey's multiple comparison test) (c). Each symbol (a-c) represents an individual mouse.

### DETAILED DESCRIPTION OF THE INVENTION

The practice of the methods and compositions described herein may employ, unless otherwise indicated, conventional techniques of pharmaceutical chemistry, drug formulation techniques, dosage regimes, and biochemistry, all of which are within the skill of those who practice in the art. Such conventional techniques include the use of combinations of therapeutic regimes including but not limited to the methods described herein; technologies for formulations of adjunct therapies used in combination with known, conventional therapies and/or new therapies for the treatment of neurodegeneration, delivery methods that are useful for the compositions of the invention, and the like. Specific illustrations of suitable techniques can be had by reference to the examples herein.

Oxidative stress is a central part of innate-immune induced neurodegeneration. However, the transcriptomic landscape of the central nervous system (CNS) innate immune cells contributing to oxidative stress is unknown, and therapies to target their neurotoxic functions are not widely available.

Provided herein is the oxidative stress innate immune cell atlas in neuroinflammatory disease and disclosure of the discovery of new druggable pathways. Transcriptional profiling of oxidative stress-producing CNS innate immune cells (Tox-seq) identified a core oxidative stress gene signature coupled to coagulation and glutathione pathway genes shared between a microglia cluster and infiltrating macrophages. Tox-seq followed by a microglia high-throughput screen (HTS) and oxidative stress gene network analysis, identified several candidates including the glutathione regulator acivicin with potent therapeutic effects decreasing oxidative stress and axonal damage in chronic and relapsing models of multiple sclerosis (MS). Thus, oxidative stress transcriptomics identified neurotoxic CNS innate immune populations and can enable the discovery of selective neuroprotective strategies.

In the following description, numerous specific details are set forth to provide a more thorough understanding of the present invention. However, it will be apparent to one of skill in the art that the present invention may be practiced without one or more of these specific details. In other instances, features and procedures well known/available to those skilled in the art have not been described in order to avoid obscuring the invention.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

A “CNS disorder” can be any disease, disorder or injury associated with the toxicity of a population of cells within the CNS. In one example, the CNS disorder is associated with a pathological process such as neurodegeneration, demyelination, dysmyelination, axonal injury, and/or dysfunction or death of an oligodendrocyte or a neuronal cell, or loss of neuronal synapsis/connectivity. In other examples, the CNS disorder is a disease associated with plaque formation, e.g., amyloid plaque formation. CNS disorders include neurodegenerative disorders that affect the brain or spinal cord of a mammal. In certain embodiments, the CNS disorder has one or more inflammatory components.

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease (AD), diffuse Lewy body type of Alzheimer's disease, Parkinson's disease, Down syndrome, progressive multiple sclerosis (MS), dementia, mild cognitive impairment (MCI), amyotrophic lateral sclerosis (ALS), traumatic brain injuries, ischemia, stroke, cerebral ischemic brain damage, ischemic or hemorrhaging stroke, multi-infarct dementia, hereditary cerebral hemorrhage with amyloidosis of the Dutch-type, cerebral amyloid angiopathy (including single and recurrent lobar hemorrhages), neurodegeneration induced by viral infection (e.g. AIDS, encephalopathies) and other degenerative dementias, including dementias of mixed vascular and degenerative origin, dementia associated with Parkinson's disease, dementia associated with progressive supranuclear palsy and dementia associated with cortical basal degeneration, epilepsy, seizures, and Huntington's disease.

As used herein, a disease, disorder or condition is “treated” if at least one pathophysiological measurement associated with the disease is decreased and/or progression of a pathophysiological process is reversed, halted or reduced. For example, a disease, disorder or condition can be “treated” if the number of plaques present in the CNS of a patient with a neurodegenerative disease is reduced, remains constant, or the creation of new plaques is slowed by the administration of an agent. In another example, a disease, disorder or condition can be “treated” if one or more symptoms of the disease or disorder is reduced, alleviated, terminated, slowed, or prevented. Measurement of one or more exemplary clinical hallmarks and/or symptoms of a disease can be used to aid in determining the disease status in an individual and the treatment of one or more symptoms associated therewith.

The term “administering” as used herein refers to administering to a subject and/or contacting a neuron or portion thereof with an inhibitor as described herein. This includes administration of the inhibitor to a subject in which the neuron is present, as well as introducing the inhibitor into a medium in which a neuron is cultured. Administration “in combination with” one or more further agents includes concurrent and consecutive administration, in any order.

The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment according to the invention include cerebellar granule neurons, dorsal root ganglion neurons, and cortical neurons.

The terms “mammal” and “mammalian subject” as used herein refers to any animal classified as a mammal, including humans, higher non-human primates, rodents, and domestic and farm animals, such as cows, horses, dogs, and cats. In some embodiments of the invention, the mammal is a human.

The term “pharmaceutical composition” refers to a formulation containing the disclosed compounds in a form suitable for administration to a subject. In one embodiment, the pharmaceutical composition is in bulk or in unit dosage form. The unit dosage form is any of a variety of forms, including, for example, a tablet, capsule, or a vial. The quantity of active ingredient in a unit dose of composition is an effective amount and is varied according to the particular treatment involved.

The phrase “therapeutically effective amount” or “effective amount” used in reference to an agent of the invention is an art-recognized term. In certain embodiments, the term refers to an amount of an agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

“Inhibitors,” “activators,” and “modulators” are used to refer to activating, inhibitory, or modulating (increase, inhibit, decrease or activate expression or activity as compared to control (an untreated or healthy subject/mammal) molecules. Inhibitors are compounds that, e.g., bind to, partially or totally block activity, decrease, prevent, delay activation, inactivate, desensitize, or down regulate the activity or expression. “Activators” are compounds that increase, open, activate, facilitate, enhance activation, sensitize, agonize, or up regulate activity, e.g., agonists.

In certain embodiments, a therapeutically effective amount of an agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent. In certain embodiments, a therapeutically effective amount is the amount effective to induce endogenous oligodendrocyte precursor cell differentiation and/or maturation, thereby promoting myelination in the subject's central nervous system.

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

### GENERAL DESCRIPTION OF THE INVENTION

Discovery of small molecule compounds that inhibit innate immune activation: To identify druggable pathways shared by both microglia and macrophages, function-selective transcriptomics was combined with drug network analysis of pathways identified through a small molecule screen in primary microglia. A high-content, high-throughput screen (HTS) was developed to discover new small molecule inhibitors that suppress microglia activation. Primary microglia were stimulated by the innate immune activators fibrin or lipopolysaccharide (LPS). 1,907 clinical drugs and bioactive compounds were screened using increased cell size (≥800 μm²) as a marker of activation and decreased size (<150 μm²) as a marker of toxicity. 128 compounds were identified that inhibited fibrin-induced microglia activation by ≥50% without toxicity (≤3% cell death) (Table 1). A total of 31 compounds, of which 27 of them inhibited fibrin- and/or LPS-induced microglia activation, with known molecular targets and potential clinical relevance for neurological diseases (Table 2) were selected to generate a “microglia drug-target network” containing ten subnetworks targeting different pathways: chemokine signaling, GGT, calcium signaling, purine nucleotide biosynthesis, cAMP signaling, neurotransmitter transport, DNA replication, dopamine-receptor signaling, microtubule dynamics, multidrug resistance, and glucocorticoid-receptor signaling.

Agents/Compounds

Agents of the invention include, but are not limited to: Modulators of calcium signaling include, but are not limited to, a calcium channel blocker, a vasodilator, β2 adrenoreceptor agonist, fendiline (fendiline hydrochloride) and/or nylidrin (buphenine, nylidrin hydrochloride).

Modulators of microtubule dynamics include, but are not limited to, inhibitors of microtubule assembly, vinblastine (vinblastine sulfate), colchicine and/or podofilox.

Modulators of chemokine signaling include, but are not limited to, tannic acid.

Modulators of DNA replication include, but are not limited to, teniposide (an inhibitor or DNA topoisomerase II).

Modulators of beta2-adrenoreceptor signaling, alpha 2-adrenergic receptor signaling, and alpha 1 adrenergic receptor signaling include, but are not limited to, Ritodrine Hydrochloride, Levonordefrin, Salmeterol, Xylazine Hydrochloride, Idazoxan Hydrochloride, Naftopidil Dihydrochloride

Modulators of progesterone include, but are not limited to Medroxyprogesterone Acetate, Melengestrol Acetate.

Modulators of dopamine receptor signaling include, but are not limited to, prochlorperazine (prochlorperazine dimaleate) (blocker of dopamine receptors) and/or thioridazine (thioridazine hydrochloride)(antagonist of dopamine; blocks dopamine receptors).

Modulators of cAMP signaling include, but are not limited to, nylidrin (buphenine, nylidrin hydrochloride), prochlorperazine (prochlorperazine dimaleate) and/or thioridazine (thioridazine hydrochloride).

Modulators of glucocorticoid-receptor signaling (including glucocorticoid receptor (NR3C1)) include, but are not limited to, betamethasone, dexamethasone, dexamethasone acetate, dexamethasone sodium phosphate, fludrocortisone acetate, fluocinolone acetonide, fluocinonide, fluorometholone, flurandrenolide, hydrocortisone, hydrocortisone acetate, hydrocortisone butyrate, hydrocortisone hemisuccinate, hydrocortisone sodium phosphate, methylprednisolone, prednisolone, and/or triamcinolone diacetate.

Modulators of purine nucleotide biosynthesis include, but are not limited to, inhibitors of inosine-5′-monophosphate dehydrogenase (IMPDH) and/or mycophenolic acid (MPA; mycophenolate).

Modulators of neurotransmitter transport include, but are not limited to, an inhibitor of norepinephrine reuptake and/or maprotiline (maprotiline hydrochloride).

Administration

Pharmaceutical formulations of the agents described herein are prepared by combining the agent having the desired degree of purity with optional physiologically acceptable carriers, excipients, or stabilizers (see, e.g., Remington's Pharmaceutical Sciences (18th edition), ed. A. Gennaro, 1990, Mack Publishing Co., Easton, Pa.). Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counter-ions such as sodium; and/or nonionic surfactants such as Tween, Pluronics, or PEG.

Agents to be used for in vivo administration can be sterile, which can be achieved by filtration through sterile filtration membranes, prior to or following lyophilization and reconstitution. Therapeutic compositions may be placed into a container having a sterile access port, for example, an intravenous solution bag or vial.

Agents described herein can be optionally combined with or administered in concert with each other or other agents known to be useful in the treatment of the relevant disease or condition.

Thus, in the treatment of demyelinating diseases, the agents can be administered in combination with injectable compositions including interferon beta 1a inhibitors or interferon beta 1b inhibitors, glatiramer acetate, and daclizumab; oral medications such as teriflunomide, fingolimod, and dimethyl fumarate; or infused medications such as alemtuzumab, mitoxantrone, or natalizumab.

In the treatment of Alzheimer's disease, agents can be administered with acetylcholinesterase inhibitors (e.g., donepezil, galantamine, and rivastigmine) and/or NMDA receptor antagonists (e.g., memantine).

In the treatment of ALS, for example, agents can be administered in combination with Riluzole (Rilutek), minocycline, insulin-like growth factor 1 (IGF-1), and/or methylcobalamin.

In another example, in the treatment of Parkinson's disease, agents can be administered with L-dopa, dopamine agonists (e.g., bromocriptine, pergolide, pramipexole, ropinirole, cabergoline, apomorphine, and lisuride), dopa decarboxylase inhibitors (e.g., levodopa, benserazide, and carbidopa), and/or MAO-B inhibitors (e.g., selegiline and rasagiline).

The combination therapies can involve concurrent or sequential administration, by the same or different routes, as determined to be appropriate by those of skill in the art. The invention also includes pharmaceutical compositions and kits.

The route of administration of the agents is selected in accordance with known methods, e.g., injection or infusion by intravenous, intraperitoneal, intracerebral, intramuscular, intraocular, intraarterial or intralesional routes, topical administration, or by sustained release systems as described below.

For intracerebral use, the agents can be administered continuously by infusion into the fluid reservoirs of the CNS, although bolus injection may be acceptable. The agents can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the agents can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et al., Mov. Disord. 2:143, 1987.

Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped compounds, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy.

A therapeutically effective amount of an agent will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from, for example, about 1 μg/kg to up to 100 mg/kg or more (e.g., about 1 μg/kg to 1 mg/kg, about 1 μg/kg to about 5 mg/kg, about 1 mg/kg to 10 mg/kg, about 5 mg/kg to about 200 mg/kg, about 50 mg/kg to about 150 mg/mg, about 100 mg/kg to about 500 mg/kg, about 100 mg/kg to about 400 mg/kg, and about 200 mg/kg to about 400 mg/kg), depending on the factors mentioned above. Typically, the clinician will administer an active inhibitor until a dosage is reached that results in improvement in or, optimally, elimination of, one or more symptoms of the treated disease or condition. The progress of this therapy is easily monitored by conventional assays. One or more agent provided herein may be administered together or at different times (e.g., one agent is administered prior to the administration of a second agent). One or more agent may be administered to a subject using different techniques (e.g., one agent may be administered orally, while a second agent is administered via intramuscular injection or intranasally). One or more agent may be administered such that the one or more agent has a pharmacologic effect in a subject at the same time. Alternatively, one or more agent may be administered, such that the pharmacological activity of the first administered agent is expired prior the administration of one or more secondarily administered agents.

One skilled in the art, upon reading the present specification, will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The present invention also provides a therapeutic kit containing materials useful for the treatment or prevention of the disorders and conditions described above is provided. The therapeutic kit may include a container and a label or package insert on or associated with the container. Suitable containers include, for example, bottles, vials, syringes, etc. The containers may be formed from a variety of materials such as glass or plastic. The container holds a pharmaceutical composition that is by itself or when combined with another agent effective for treating or preventing the condition and may have a sterile access port (e.g., an intravenous solution bag or a vial having a stopper pierceable by a hypodermic injection needle). At least one active agent in the pharmaceutical composition is one of the agents described herein above. The label or package insert indicates that the composition is used for treating the condition of choice. Moreover, the kit may include (a) a first container with a pharmaceutical composition contained therein, wherein the composition includes an agent described herein; and (b) a second container with a pharmaceutical composition contained therein, wherein the composition includes a different agent. The therapeutic kit in this embodiment of the invention may further include a package insert indicating that the compositions can be used to treat a particular condition. Alternatively, or additionally, the therapeutic kit may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Assessment of Treatment

In some embodiments, the successful treatment of a subject with an agent described herein is determined by at least about a 10%-100% decrease in one or more symptoms of a CNS disorder. Examples of such symptoms include, but are not limited to, slowness of movement, loss of balance, depression, decreased cognitive function, short-term memory loss, long-term memory loss, confusion, changes in personality, language difficulties, loss of sensory perception, sensitivity to touch, numbness in extremities, tremors, ataxia, muscle weakness, muscle paralysis, muscle cramps, muscle spasms, significant changes in eating habits, excessive fear or worry, insomnia, delusions, hallucinations, fatigue, back pain, chest pain, digestive problems, headache, rapid heart rate, dizziness, and visual changes.

For example, clinical signs of MS are routinely classified and standardized, e.g., using an EDSS rating system based on neurological examination and long-distance ambulation. As used herein, the “Expanded Disability Status Scale” or “EDSS” is intended to have its customary meaning in the medical practice. EDSS is a rating system that is frequently used for classifying and standardizing MS. The accepted scores range from 0 (normal) to 10 (death due to MS). Typically, patients having an EDSS score of about 4-6 will have moderate disability (e.g., limited ability to walk), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability impairing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

In certain embodiments, the evaluation of disease progression includes a measure of upper extremity function (e.g., a 9HP assessment). Alternatively, or in combination, disease progression includes a measure of lower extremity function. Alternatively, or in combination, disease progression includes a measure of ambulatory function, e.g., short distance ambulatory function (e.g., T25FW). Alternatively, or in combination, disease progression includes a measure of ambulatory function, e.g., longer distance ambulatory function (e.g., a 6-minute walk test). In one embodiment, the disease progression includes a measure of ambulatory function other than EDSS ambulatory function. In one embodiment, disease progression includes a measure of upper extremity function e.g., a 9HP assessment, and a measure of ambulatory function, e.g., short distance ambulatory function (e.g., T25FW). In one embodiment, disease progression includes a measure of upper extremity function (e.g., a 9HP assessment) and a measure of lower extremity function. In one embodiment, disease progression includes a measure of upper extremity function (e.g., a 9HP assessment), a measure of lower extremity function, and a measure of ambulatory function, e.g., short distance ambulatory function (e.g., T25FW) and/or longer distance ambulatory function (e.g., a 6-minute timed walk test (e.g., 6MWT)). In one embodiment, one, two or the combination of the T25FW, 6MWT and 9HP assessments can be used to acquire a disease progression value. The measure of ambulatory function (e.g., short distance ambulatory function (e.g., T25FW) or longer distance ambulatory function (e.g., a timed (e.g., 6-minute) walk test (e.g., 6MWT)) and/or measure of upper extremity function (e.g., a 9HP assessment) can further be used in combination with the EDSS to evaluate MS, e.g., progressive forms of MS.

Alzheimer's disease (AD) is a neurodegenerative disorder that results in the loss of cortical neurons, especially in the associative neocortex and hippocampus which in aim leads to slow and progressive loss of cognitive functions, ultimately leading to dementia and death. Major hallmarks of the disease are aggregation and deposition of misfolded proteins such as aggregated beta-amyloid peptide as extracellular senile or neuritic ‘plaques’, and hyperphosphorylated tau protein as intracellular neurofibrillary tangles.

Genetic predispositions for AD are divided into two forms: early-onset familial AD (<60 years), and late-onset sporadic AD (>60 years). Rare, disease causing mutations in Amyloid precursor protein (APP), Presenilin 1 (PSEN1), and Presenilin 2 (PSEN2) genes are known to result in early-onset familial AD while, APOE (allele 4) is the single most important risk factor for late-onset AD. In specific embodiments, the methods of the invention are used to treat subjects with a genetic predisposition for wither early onset familial AD or late-onset sporadic AD.

Although Alzheimer's disease develops differently for every individual, there are many common symptoms. In the early stages, the most common symptom is difficulty in remembering recent events. As the disease advances, symptoms can include confusion, irritability, aggression, mood swings, trouble with language, and long-term memory loss.

Clinical Decision Support Systems (CDSS) comprising computer hardware, software, and/or systems can be used to determine a diagnosis for a patient who has certain symptoms associated with Alzheimer's disease. CDSS often include at least three component parts: a knowledge basis, an inference engine, and a communication mechanism. The knowledge base may comprise compiled information about symptoms, pharmaceuticals, and other medical information. The inference engine may comprise formulas, algorithms, etc. for combining information in the knowledge base with actual patient data. The communication mechanism may be ways to input patient data and to output helpful information based on the knowledge base and inference engine. For example, information may be inputted by a physician using a computer keyboard or tablet and displayed to the physician on a computer monitor or portable device.

In certain aspects, the assessment of treatment includes radiological assessment, e.g., single photon emission computed tomography (SPECT), Positron Emission Tomography (PET), Magnetic Resonance Imaging (MRI) and scintigraphy. For example, multiple sclerosis can be assessed using radiologic assessment of CNS plaques, e.g. by MRI. In another example, AD plaque load can be assessed, e.g., using Aβ-PET.

The assessment of treatment according to the present invention may also be performed using scanning database systems and methods such as those described in US Appln. No. 20150039346.

### EXAMPLE

The following example is put forth so as to provide those of ordinary skill in the art with a complete disclosure and description of how to make and use the present invention, and is not intended to limit the scope of what the inventors regard as their invention, nor is the example intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

Transcriptional Profiling and Therapeutic Targeting of Oxidative Stress in Neuroinflammation

### INTRODUCTION

Oxidative injury is a pathologic feature linked to neurodegeneration, myelin damage and disease progression in MS and other neurodegenerative diseases (1-6). Oxidative stress mediated by reactive oxygen species (ROS) release from CNS innate immune cells promotes neurodegeneration and demyelination (1,3,7-10). Innate immune-mediated oxidative injury has been proposed as a critical process underlying the progression of MS from the relapsing phenotype to relentless neurodegeneration (11,12). In progressive MS, neurodegeneration is associated with robust microglia activation and oxidative stress (12,13). However, the mechanisms in innate immune cells that trigger oxidative injury in neuroinflammation remain poorly understood. Single-cell technology has led to an appreciation of the heterogeneity of CNS innate immune responses with distinct gene profiles between microglia and CNS infiltrating macrophages in MS, Alzheimer's disease (AD), and related animal models (14-21). However, the functional transcriptomic landscape of oxidative stress inducing innate immune cells is unknown. Furthermore, the discovery of drugs capable of selectively suppressing innate immune-driven neurodegeneration has been hindered by the lack of molecular understanding of the neurotoxic functions of CNS innate immune cells.

Here, the innate immune cell atlas of oxidative stress in neuroinflammatory disease and the discovery of new therapeutic targets is reported. To functionally dissect the oxidative stress signature of CNS innate immunity at the single-cell level, a Toxic-RNA-seq (Toxseq) was developed to transcriptionally profile ROS+innate immune cells. A core oxidative stress signature shared among a microglia cluster and subsets of infiltrating myeloid cells in mice were identified, as well as microglia from MS lesions. Tox-seq followed by microglia HTS of a library of 1,907 clinical drugs and bioactive compounds and oxidative stress gene network analysis identified glutathione transferase activity and the compound acivicin, which inhibits the degradation of the antioxidant glutathione by targeting γ-glutamyl transferase (GGT). Therapeutic administration of acivicin reversed clinical signs, decreased oxidative stress, and protected from neurodegeneration in chronic EAE, even when administered eighty days after disease onset. Thus, these studies determine the transcriptomic landscape of oxidative stress in CNS innate immunity and provide druggable pathways for therapeutic targeting of neurotoxic innate immune populations.

Materials and Methods

Animals SJL/J, NOD, C57BL/6, and Ggt1^{dwg/dwg} 38 mice were purchased from The Jackson Laboratory, and Sprague-Dawley rat Po litters were purchased from Charles River Laboratories. Ccr2^{RFP/RFP} mice (56) on C57BL/6 background (provided by I. F. Charo, Gladstone Institutes) were crossed with Cx3cr1^{GFP/GFP} mice (57) to generate Cx3cr1^{GFP/+} Ccr2^{RFP/+} mice. Mice were housed under IACUC guidelines in a temperature and humidity-controlled facility with 12 h light-12 h dark cycle and ad libitum feeding. All animal protocols were approved by the Committee of Animal Research at the University of California, San Francisco, and were in accordance with the National Institutes of Health guidelines.

EAE. Active EAE was induced in 8- to 10-week-old female SJL/J mice, C57BL/6 mice, and Cx3cr1^{GFP/+} Ccr2^{RFP/+} mice by subcutaneous immunization with 50 μg MOG₃₅₋₅₅ peptide (MEVGWYRSPFSRVVHLYRNGK; Auspep) or 100 μg PLP139-151 peptide (HSLGKWLGHPDKF; Auspep) in complete Freund's Adjuvant (Sigma-Aldrich) supplemented with 400 μg of heat-inactivated Mycobacterium tuberculosis H37Ra (Difco Laboratories). At day 0 and 2 after immunization, mice were given intraperitoneal injection of 200 ng (C57BL/6) or 75 ng (SJL/J) pertussis toxin (Sigma-Aldrich). For chronic NOD EAE model, 10- to 12-week-old NOD mice were immunized with 150 μg MOG₃₅₋₅₅ peptide, followed by administration of 200 ng pertussis toxin on days 0 and 2 as described 43. For adoptive transfer of EAE in SJL/J mice, donor SJL/J mice were immunized as described above, and on day 10 post immunization, cells were isolated from the draining lymph nodes and spleen. Lymphocytes were re-stimulated with 20 μg/ml PLP₁₃₉₋₁₅₁ and 10 ng/ml IL-12 (eBioscience) for 4 d, 3×10⁷ restimulated cells were transferred to healthy SJL/J recipient mice as described (9). For prophylactic treatment, mice were each administered with 5 mg/kg acivicin (Santa Cruz Biotechnology, dissolved at 1 mg/ml in saline), 5 mg/kg GGsTop (Tocris) or saline daily from day 0. For therapeutic treatment, acivicin or saline was injected daily starting at the peak of the initial paralytic episode or at day 80 during the chronic phase of EAE in NOD mice. Mice were randomly assigned to treatment groups, scored and drug treated in a blinded manner Mice that did not develop symptoms of EAE were not excluded from the analysis. Experimental groups were unblinded to treatment assignment at the end of the experiments. Mice were observed daily, and clinical scores were assigned as follows by observers blinded to treatment: 0, no symptoms; 1, loss of tail tone; 2, ataxia; 3, hindlimb paralysis; 4 hindlimb and forelimb paralysis; 5, moribund. EAE onset was defined by weight loss (>1 gram) and first day of symptoms (score >0).

Cell isolation from spinal cord. Mice were perfused with ice-cold phosphate buffered saline (PBS) and spinal cord tissue was flushed out from the spinal column with PBS. Spinal cords were incubated in HBSS with 0.2% collagenase, type 3 (Worthington) gently shaking for 30 min at 37° C. under trypsin-free, mild dissociation conditions. Tissue were mechanically dissociated and passed through a 40 μm cell strainer (Falcon). Single-cell suspension was collected in 5 ml RPMI-1640 medium without phenol red supplemented with 25 mM HEPES, 1% Penicillin/Streptavidin and 10% heat-inactivated fetal bovine serum (FBS; Thermo Fisher Scientific) and myelin was depleted following the myelin removal beads II manufactures guidelines (Miltenyi Biotec). Myelin-depleted cell suspensions were processed for flow cytometry, bulk RNA-seq or scRNA-seq as described below. For bulk RNA-seq, myelin depleted (myelin removal beads II, Miltenyi Biotec) cell suspensions from spinal cords of five mice per experiment were pooled. For scRNAseq, single cell suspensions from individual animals were processed independently.

ROS labeling for Tox-seq. Following surface antigen flow cytometry staining, live cells isolated from spinal cord were stained for intracellular ROS in vitro using membrane permeable, fluorescent reagent 2′,7′-dichlorofluorescein diacetate (DCFDA; Abcam) as described (22). DCFDA recognizes ROS and is also a redox indicator probe that responds to cellular oxidant stress including reactive nitrogen species and elevated iron. For Bulk RNA-seq, cells were incubated with 10 μM DCFDA in PBS supplemented with 2% FBS, 2 mM EDTA (Gibco) for 30 min at 37° C., and directly analyzed by flow cytometry. For scRNA-seq, to minimize cell activation and cell death cells were incubated with 10 μM DCFDA in PBS supplemented with 2% FBS without EDTA at 4° C. for 30 min prior to cell sorting.

Fluorescence-activated cell sorting analysis for Tox-seq. For the scRNA-seq experiment, myelin-depleted cell suspensions were treated for 5 min at 4° C. with Fc-block in BSA staining buffer (BD) and then incubated for 30 min at 4° C. with CD11 b APC-Cy7 (M1/70) antibodies. Cells were washed with stain buffer once and then in vitro ROS labeling (DCFDA) was performed as described above. Cells were incubated for 5 min at 4° C. with 1 μM sytox blue live/dead stain and then cells were sorted using FACSAria II. After centrifugation at 300 g and 4° C. for 10 min, cell pellets were resuspended in cold PBS supplemented with 2% FBS at 333 cells/μl and immediately processed for scRNA-seq as described below.

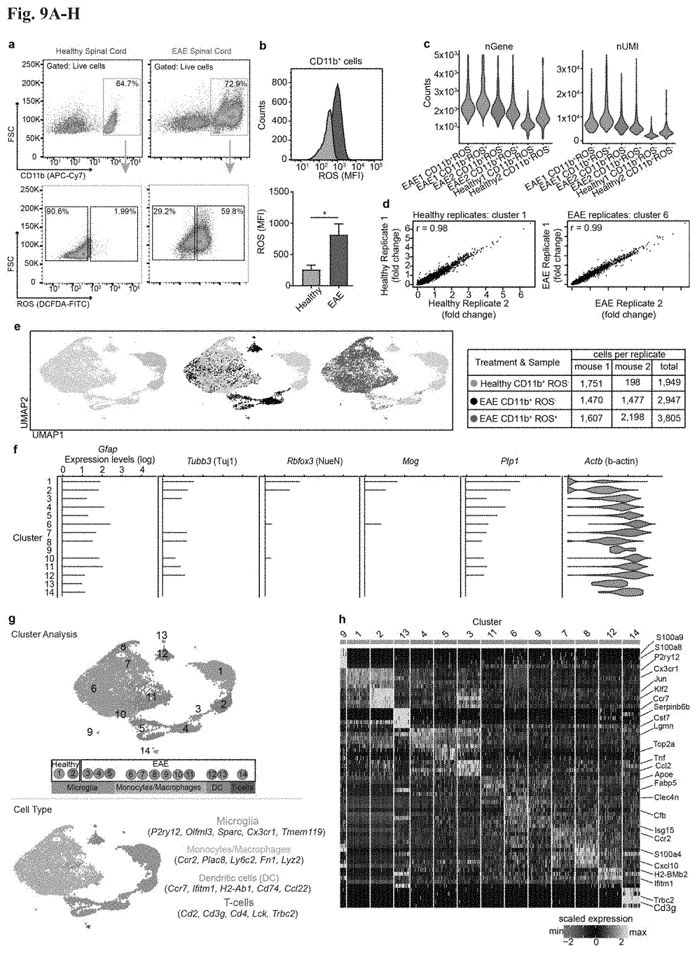

Droplet-based scRNA-seq. For Tox-seq, 30 μL of each live sytox blue ⁻CD11b⁺ROS⁻ and live sytox blue ⁻CD11b⁺ROS⁺ sorted cell population (233 cells/μl) from healthy and EAE spinal cords (disease score 1.0) were run on the 10× Genomics Chromium platform, and libraries were prepared following manufactures instructions for the Chromium Single Cell 3′ v2 Reagent Kit (FIG. 9A, B). Balanced library pools were run across four lanes of an Illumnia Hiseq4000 with a targeted sequencing depth of 100,000 reads per cell. Reads from each sample were aligned to the mm 10 reference assembly using the 10× Genomics Cell Ranger Count version 2.0.1, and then each sample was combined into one dataset using the 10× Genomics Cell Ranger Aggr package. The Cellranger data summary analysis revealed 9,079 barcoded cells with 1,917 median genes per cell, 5,906 median unique molecular identifiers (UMI) counts per cell, and 135,639 post-normalization mean reads per cell. An average of 90% sequencing saturation was achieved per sample.

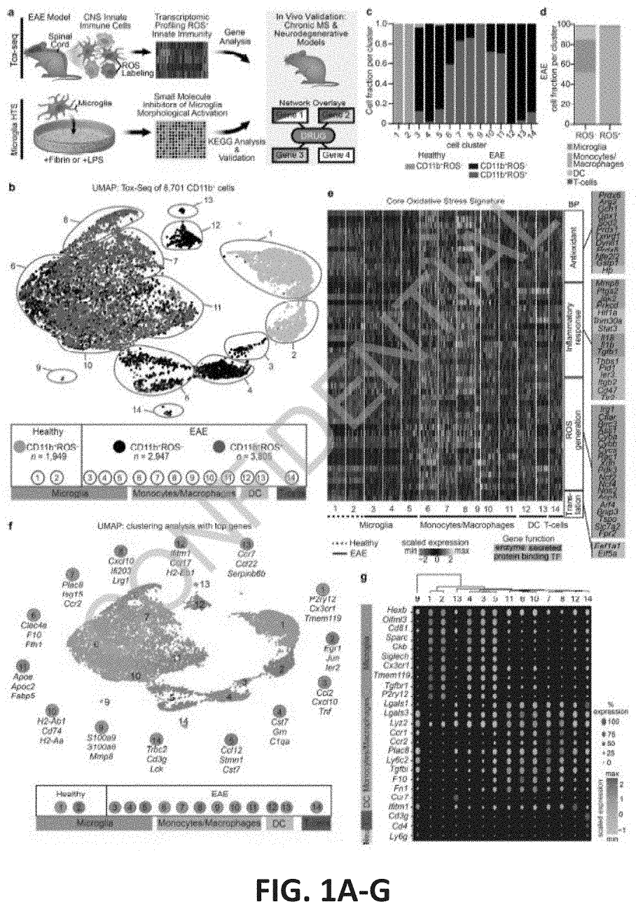

Unbiased graph-based clustering analysis of scRNA-seq data. The R toolkit Seurat (23) was used for quality control processing, graph-based clustering, visualizations, and differential gene expression analyses of scRNA-seq data and performed in R version 3.4.2. Cellranger Aggr aggregated dataset of 9,079 cells were filtered through quality control parameters that included parameters to keep cells with 200-5,000 nFeature_RNA per cell, and eliminate unhealthy cells with >5% and >25% mitochondrial and ribosomal genes, respectively. The percent of mitochondrial (percent.mito) and ribosomal (percent.ribo) genes were regressed out. All remaining variable genes were used for downstream analyses, including immediate early response genes induced by cell isolation procedure 24. Following QC, 17,814 genes across 8,701 single cells were subjected to downstream analyses following Seurat version 3 default parameters unless otherwise stated (Supp FIG. 1c, d). To determine statistically significant principal components (PC), JackStraw was performed with num.replicate=100. For clustering analysis, FindNeighbors and FindClusters were used with the first 20 significant principle components determined by Jackstraw analysis and a resolution=0.8, respectively. For subclustering analysis, monocyte/macrophage or microglia clusters in FIG. 1h, were separately re-clustered by running through the Seurat pipeline to determine new cell clusters as described above. FindClusters was implemented at a resolution=0.4 for monocyte/macrophages or resolution=0.3 for microglia subcluster analysis. Differentially expressed genes (DEGs) for each cluster was determined by FindAllMarkers with parameters set at min.pct, 0.25; log2fc.threshold, 0.25 using wilcox statistical test. Genes that met the above criteria with adjusted p-value <0.05 (Benjamini-Hochberg correction) were used for downstream functional enrichment analysis. GO enrichment analysis of DEGs was performed in DAVID (58) and Metascape bioinformatic resources. For GO analysis all DEGs with log2 fold change >0.25 and adjusted p-value <0.05 were used to generate GO terms via Metascape analysis using default settings.

Bulk RNA-seq and data analysis. For bulk RNA-seq experiment, surface stained cells were resuspended in 100 μl PBS with 1% FBS for cell sorting using FACSAria II (BD Bioscience). CompBeads Compensation Particles (BD Biosciences) individually stained with each of the fluorescently labeled antibodies were used for color compensation. In each experiment, the maximal cell number of sorted microglia and macrophages was assessed based on CD11b (M1/70) and CD45 (30-F11) signal intensities. The sum of sorted cells from three independent experiments (n=15 mice total) was 240,606 microglia and 183,530 macrophages. ROS⁺ (DCFDA) cells were gated based on FITC signals (cut-off 2×10³), and MHC II⁺ cells gated based on PE-Cy7 signal (cut-off 3×10³) compared to the unstained cells. Microglia and macrophage subpopulations were classified into MHC II⁺ ROS⁺, MHC II⁻ ROS⁻(baseline), MHC II⁻ ROS⁺ and MHC II⁺ROS⁻. In each sorting experiment, eight distinct cell populations were collected separately on ice. After centrifugation at 300 g and 4° C. for 6 min, cell pellets were stored at −80° C. in 150 μl RLT buffer (Qiagen) adding 1% 2-mercaptoethanol (Gibco) prior to RNA isolation for bulk RNA-seq.

Total RNA was isolated using the RNeasy Plus Micro Kit (Qiagen) according to the manufacturer's instructions. cDNA was generated from full-length RNA using the NuGEN Ovation RNA-seq V2 kit, and then sheared by the Covaris S2 Sonicator to yield uniform size fragments. The NuGen Ovation Ultralow V2 kit was used to add adapters, barcoding and amplification. Libraries were purified using Agencourt XP magnetic beads and quantified by KAPA qPCR (Illumina) Four libraries per lane were pooled for a single end (SE 50 bp) run on the Illumnia HiSeq 4000 platform. Input sequences were provided in FASTQ format. Trimming of known adapters and low-quality regions of reads was performed using Fastq-mcf59. Sequence quality control was assessed using the program FastQC and RSeQC. Reads were aligned to the mm9 mouse genome assembly using Tophat 2.0.13, and the number of reads mapping to each gene were counted using “featureCounts”, part of the Subread suite. Differential analysis was performed using edgeR60 Bioconductor package. The data set was filtered by including all genes which had at least in two replicates a CPM (counts per million) between 0.5 and 5000. The remaining genes were normalized using calcNormFactors (TMM) (“weighted trimmed mean of M-values”) in edgeR 61. Calculation of P-values was performed in edgeR for the differential expression between samples. The built-in R function “p.adjust” was used to calculate the FDR (false discovery rate) for each P value using the Benjamini-Hochberg method. To identify differentially expressed genes that were overrepresented in existing annotated genes, the data was analyzed with GO Elite (62). Clustering was performed first by doing k-means clustering with 100 clusters on log transform expression levels then by Hierarchical Ordered Partitioning and Collapsing Hybrid (HOPACH) (63) on the k-means clusters to prune highly similar clusters.

Co-expression clusters from enriched GO terms for bulk RNA dataset. Functional enrichment analysis of a data set with 2,145 differentially expressed genes in ROS⁺ versus MHC II⁺ microglia was performed in Cytoscape (32) using BiNGO plugin and GO annotations downloaded on 8 Dec. 2016. Filtering for GO term results with <2000 genes, 592 Biological Process terms with a corrected p-value <0.05 were found. GO term enrichment was also performed on the exclusive subsets of ROS-expressed genes (1,613) and MHC II-expressed genes (924), resulting in 58 and 783 filtered Biological Process terms, respectively. From these GO term results, 6 were selected that represented processes of interest, maximum specificity (i.e., low total gene membership) and exclusive significance (i.e., non-overlapping terms). Interaction networks were constructed for each set of differentially expressed genes associated with these 6 terms using GeneMANIA (64). Default co-expression and physical interaction sources were selected from the network construction; the option to add a number of related genes was set to zero; all other settings were default values. The interaction data from GeneMANIA was imported into Cytoscape for visualization and data overlay. Co-expression clusters were defined as the largest connected set of either up- or down-regulated genes and extracted as subnetworks.

Preparation of fibrin plates for HTS. Fibrin-coated 384-well plates were prepared as follows: Using a EL406 liquid handler (BioTek), columns 1-22 received 30 μL of 2 U/mL Thrombin (Sigma-Aldrich) in a buffer of 20 mM HEPES, pH 7.4, and 14 mM CaCl2, followed by 30 μL of 12 μg/mL human plasminogen-free fibrinogen (EMD Milipore) in 20 mM HEPES, pH 7.4 for a final concentration of 6 μg/mL of fibrinogen. After incubation for 90 min in 37° C. to allow fibrin formation, plates were dried overnight in a 37° C. incubator equipped with fans to circulate the air.

Image-based HTS of small molecule inhibitors of microglia activation. Primary rat microglia were isolated and cultured in the presence of heat-inactivated Performance Plus FBS (Thermo Fisher Scientific) as described (18,65). FBS in microglia culture was used to obtain a sufficiently high yield of microglia that was required for the HTS of 2,000 compounds. Since microglia activation can be influenced by the culture conditions (18,65), FBS was batch-tested with three quality control criteria: high cell yield, no effect on morphologic activation at baseline, and response to LPS induced morphologic activation by at least 50%. Microglia HTS was performed in 384-well PDL coated plates (Greiner). To screen for compounds that inhibited fibrin-activated microglia, 50 μL of DMEM containing 10% FBS was added to the fibrin-coated plate using an EL406 liquid dispenser-aspirator (Biotek). A library of 1,907 clinical drugs and bioactive compounds, compiled by the Small Molecule Discovery Center at the University of California, San Francisco, was screened at 10 μM. 100 nL of each compound (10 mM stock solution in DMSO) was added using a Biomek FXP automated laboratory workstation (Beckman Coulter) outfitted with a 50 nL pintool (V&P Scientific). Columns 1-2 contained DMSO-treated control cells (defining maximal activation/0% inhibition); columns 3-22 contained test compounds, and columns 23-24 contained unstimulated control cells (defining minimal activation/100% inhibition). 3,000 microglia cells were added to each well in 50 μL of DMEM containing 10% FBS, giving a final compound concentration of 10 μM in 0.1% DMSO. Assay plates were incubated at 37° C., 5% CO₂ for 48 h. Using the EL406 liquid dispenser-aspirator, cells were then fixed with 4% paraformaldehyde solution, permeabilized with 0.1% Triton-X100, and stained with 0.5 μg/mL CellMask Red (Thermo Fisher Scientific) and 2 μg/mL Hoechst nuclear dye (Thermo Fisher Scientific) with PBS washes between steps. The plates were stored in PBS for readout by imaging.

To screen for inhibitors of LPS (Sigma-Aldrich)-activated microglia, PDL-precoated 384-well plates (Greiner) were used. 50 μL of microglia cell suspension (3,000 cells per well) were added to each well with a WellMate multi-channel liquid dispenser (Thermo Fisher Scientific). 100 nL of each test compound (10 mM stock solutions in DMSO) was then added to columns 3-22 using the Biomek FXP and 50 nL pintool. One hour after the addition of compounds, 50 μL of a 1 ng/mL LPS solution was added to columns 1-22 using the EL406 liquid dispenser-aspirator, yielding an assay concentration of 0.5 ng/mL LPS in 100 μL assay volume. Columns 1-2 contained stimulated cells treated with DMSO (defining maximal activation/0% inhibition) and columns 22-24 contained unstimulated cells (defining minimal activation/100% inhibition). Assay plates were incubated at 37° C., 5% CO₂ for 48 h. Cells were then fixed, permeabilized, stained and washed as described for the fibrin assay (9).

To measure microglial activation, assay plates were imaged using an INCell Analyzer 2000 automated fluorescent microscope (GE Healthcare) equipped with a 10× objective and excitation/emission filter pairs of 350 nm/455 nm (Hoechst stain) and 579 nm/624 nm (CellMask Red). Images were analyzed with the INCell Developer Toolbox feature extraction software (GE Healthcare). Cell nuclei stained with Hoechst dye were segmented using a “nuclear” segmentation method, with a minimum target area of 30 μm2 and sensitivity of 75%. Exclusion criteria for cell segmentation was set to intensity <120 units, or area >1000 μm2 The CellMask Red-stained cell bodies were segmented using an “intensity” segmentation method with a set threshold between 200-4095 intensity units. The borders of adjacent contacting cells were resolved using the “clump breaking” post-processing segmentation method that utilized discrete nuclei as seeds. Only cells containing a nucleus within the cell body area were analyzed. Activated microglia were defined as cells with a size ≥800 μm², whereas cell size <150 μm² was classified as dead cells. The number of activated microglia was divided by the total number of cells in the well to yield a fraction of activation. This activated fraction was normalized to the stimulated/untreated wells (columns 1-2) and unstimulated wells (columns 23-24) to determine the percentage of inhibition of microglial activation. Similarly, the percentage of dead cells was calculated by comparing the fraction of dead cells in a well to the stimulated and unstimulated controls. For each assay plate, Z-prime values were calculated as described (9). The average Z′ value was 0.5 for the six fibrin screening plates and 0.63 for the six LPS screening plates.

Compounds that were toxic to fewer than 3% of microglial cells and inhibited activation by >50% in either LPS or fibrin-treated plates were considered active. The 50% inhibition cutoff value was set as 3 standard deviation from the mean value of untreated control. Active compounds were reconfirmed in dose-response assays conducted in triplicates of 10 concentrations, with 3-fold serial dilutions ranging from 0.001 μM-20 μM. IC₅₀ values were estimated from normalized % inhibition values, using the four-parameter non-linear regression analysis (Graphpad Prism).

Oxidative stress pathway modeling. A novel oxidative stress pathway was constructed based on compiling information from the literature and existing pathway diagrams, that included glutathione metabolism, redox, biosynthesis, uptake, breakdown, glutathionylation and acivicin inhibition. The gamma-glutamyl cycle was simplified for clarity. PathVisio (33) was used to construct the pathway model, which was deposited at WikiPathways (WP4466) (66) and then imported into Cytoscape for RNA-seq data overlay.

Immunohistochemistry. For immunohistochemical analysis, spinal cords were processed as Described (9,29,30,67,68). Antibodies used were as follows: mouse anti-gp91 (CYBB, 1:200; 53, BD Biosciences), mouse anti-iNOS (1:500, 610329, BD Biosciences), rabbit anti-Iba-1 (1:1000; 019-19741, Wako), rat anti-MHC II (1:300; M5/114.15.2, Thermo Fisher Scientific), rabbit coagulation factor X (F10; NBP1-33320, NOVUS), mouse anti-CLEC4E (1:700; AT16E3, abcam), mouse anti-neurofilament H non-phosphorylated (1:100; SMI-32, BioLegend), mouse anti-myelin basic protein (1:100; SMI-99, BioLegend), rabbit polyclonal anti-GGT1 (1:100; SAB2701966, Sigma), or goat polyclonal anti-4HNE (1:200; ab46544, Abcam) and Alexa 647, 488, 405 (1:500; Jackson ImmunoResearch) the Vector-Red and Vector-Blue alkaline phosphatase substrate kit (Vector Labs) for detection. Sections were stained with DAPI (1:1000, Thermo Fisher Scientific) for 3 min at room temperature. For mouse primary antibodies, the Mouse on Mouse (M.O.M.) kit (Vector Labs) was used according to the manufacturer's protocol. Analysis was based on an established method of neuropathology for sampling multiple spinal cord sections and comparing similar anatomical regions as described (68-70). Images were acquired with an Axioplan II epifluorescence microscope (Zeiss) equipped with Plan-Neofluar objectives (10×0.3 NA, 20×0.5 NA, or 40×0.75 NA) or all-in-one BZ-X700 fluorescence microscope (Keyence), Fluoview FV 1000 (Olympus) confocal microscope and Fluoview software v3.1b with Olymus 40× and 0.8 NA water-immersion lens as described (9), or Aperio Versa scanner (Leica) with Aperio Imagescope 12.4 and 1.25×, 10×, and 20× lenses. Images of similar anatomical locations were quantified using NIH ImageJ (version 1.50) by observers blinded to experimental conditions.

Flow cytometry. Cells were incubated with anti-mouse CD16/CD32 antibody (2.4G2) diluted in PBS with 2% FBS, and 2 mM EDTA at 4° C. for 15 min to block Fc receptor binding. Cells were stained with fluorescent-conjugated antibodies: CD3 (17A2), CD4 (GK1.5), CD8 (53-6.7), CD62L (MEL-14), CD44 (1M7), CD25 (3C7), CD11b (M1/70), CD45 (30-F11), and MHC II (M5/114.15.2). For surface labeling of GGT1, unconjugated mouse monoclonal anti-GGT1 (1:100; ab55138, Abcam) was incubated with surface markers for 1 hr at 4° C. Then FITC conjugated species-specific secondary antibody (1:100) was added for 1 hr at 4° C. For intracellular staining of IFN-γ and IL-17A, T cells were stimulated for 4 hr with Cell Activation Cocktail (BioLegend). Cells were then fixed with Cytofix/Cytoperm solution (BD Bioscience) and stained with antibodies to IL-17A (TC11-18H10.1), or IFN-γ (XMG1.2). Foxp3 (FJK-16S) staining was performed according to the manufacturer's protocol (eBioscience). Cells were analyzed by flow cytometry on an LSR II (BD Biosciences) with FlowJo software (Tree Star Inc.). Antibodies were purchased from BioLegend, BD Biosciences, or eBioscience.

Bone marrow—derived macrophage cultures. Bone marrow—derived macrophages (BMDMs) were prepared as described (67). In brief, bone-marrow cells were isolated from tibia and femur of C57BL/6 mice, Ggt1^{+/+} mice or Ggt1^{dwg/dwg} mice and cultured in RPMI-1640 medium supplemented with 10% FBS, 1% penicillin-streptomycin (Corning), and 10 ng/ml murine M-CSF (14-8983-80, Thermo Fisher Scientific). On days 6-7, adherent BMDMs were harvested by adding PBS with 5 mM EDTA to the plates and used for assays.

Human macrophage cultures. Human peripheral blood mononuclear cells (PBMCs) were purchased from AllCells, LLC (Alameda, Calif.). To differentiate PBMC into monocyte-derived macrophages, 2×10⁶ PBMC/mL were plated in RPMI-1640 media supplemented with 10% FBS, 1% penicillin-streptomycin (Corning), and 50 ng/ml human M-CSF (300-25, Peprotech) in tissue-culture treated dishes (Corning). After 24 h, non-adherent cells were removed, and adherent cells were cultivated for 7-8 additional days at 37° C. in 5% CO₂ to promote their full differentiation into macrophages. Oxidant detection with DHE. Intracellular concentrations of ROS were measured using dihydroethidium (DHE) as described (9). Cultured BMDMs or human macrophages were incubated in medium containing 5 μM DHE (Invitrogen) for 30 min Cells were plated on 96-well, black μ-clear-bottom microtiter plates (Greiner Bio-One) pre-coated with 25 μg/ml fibrin. For GGT inhibition, BMDMs or human macrophages were pre-incubated with 5 μM acivicin or GGsTop for 1 h, and the cells were plated on fibrin-coated 96-well plates as described above. PBS was used as vehicle control. Cells were then incubated for 24-48 h and fixed with 4% PFA for 10 min. The DHE fluorescence was detected at an excitation/emission wavelength of 518 nm/605 nm using a SpectraMax M5 microplate reader (Molecular Devices) with SoftMax Pro 5.2 software (Phoenix Technologies Ltd.).

Real-time imaging of glutathione. The real-time GSH dynamics in living BMDMs was determined with the fluorescent GSH probe, RealThiol RT-AM as described (37). Cells were plated on 2-well chamber slide (Nunc) pre-coated with 25 μg/ml fibrin. Cells were stimulated with fibrin with or without 5 μM acivicin or GGsTop. The GSH synthesis inhibitor, buthionine sulfoximine (BSO, Sigma-Aldrich) was used as positive control for reducing intracellular GSH levels at 100 μM. After 24 h incubation, cells were loaded with 1 μM RT-AM probe and were incubated for 5 min before imaging. The cells were kept at 37° C. during the entire experiment. Following incubation, fluorescence emissions after sequential excitation at 405 nm and 488 nm was acquired using sequential confocal laser scanning microscopy (Olympus FV1000; Olympus, Tokyo, Japan) with a 10× objective and 2× optical magnification. For each independent experiment, laser power and detector settings were set using the control/untreated cells incubated with RT-AM alone as reference. All settings were kept constant throughout the experiment. The ratio bound intracellular RT-AM:unbound intracellular RT-AM for each treatment condition was calculated by subtracting the 488-nm fluorescence signal from the 405 nm fluorescence signal from 30-50 cells/treatment for each independent experiment. The ratio calculated for each treatment was expressed as percentage from that of the untreated cells (control).

Blood sampling and hematological analysis. Hematological analysis was carried out for mice treated with saline or acivicin for 10 days. Blood samples were collected from the heart of each anesthetized mouse via cardiac puncture. The complete blood cell counts were measured with an automated blood count analyzer (Hemavet, Drew Scientific Inc).

Protein carbonyl content assay. Blood was isolated from EAE mice via terminal cardiac puncture, and serum levels of protein carbonylation were measured with OxiSelect™ Protein Carbonyl ELISA (Cell Biolabs) according to the manufacturer's protocol. The absorbance at 450 nm was measured using a SpectraMax M5 microplate reader (Molecular Devices) with SoftMax Pro 5.2 software (Phoenix Technologies Ltd.).

Real time qPCR. Total RNA was isolated from fibrin- or LPS-stimulated BMDMs (Ggt1^{dwg/dwg} or Ggt1^{+/+} mice) with the RNAeasy Mini kit (Qiagen) according to the manufacturer's instructions. cDNA was prepared with High Capacity cDNA Reverse Transcription Kit (Applied Biosystems). Real-time qPCR analysis was performed on spinal cord tissues prepared from MOG₃₅₋₅₅ EAE mice. SYBR green-based qPCR was performed using murine primers to Cxcl10, Nos2, Cxcl3, Ccl5, Il1b, or Il12b. Results were analyzed with the Opticon 2 Software and the comparative CT method. Gene expression was normalized to Gapdh and presented as fold change relative to control.

GGT activity assay. GGT activity was measured using the MaxDiscovery gamma-Glutamyl Transferase (GGT) Enzymatic Assay Kit (Bioo Scientific) according to the manufacturer's protocol. BMDMs were pre-incubated for 1 h with 10 μM GGT inhibitor acivicin (Santa Cruz Biotechnology) before plating cells on 25 μg/ml fibrin-coated 6-well culture plates that were prepared as described (67). For in vivo GGT activity assay, spinal cord tissues from the onset of MOG35-55 EAE or LPS-injected substantia nigra area at 12 h were prepared. Tissues were homogenized in 0.1 M Tris-HCl and centrifuged at 13,000 g for 30 min at 4° C. The supernatant was collected and subsequently assessed for GGT activity. The absorbance at 405 nm was detected with a SpectraMax M5 microplate reader (Molecular Devices) with SoftMax Pro 5.2 software (Phoenix Technologies Ltd.). The GGT activity in IU/1 was calculated following the manufacturer's protocol by multiplying the average increase in absorbance at 405 nm over 10 min GGT activity was also measured using a fluorescent probe, ProteoGREEN-gGlu (Goryo Chemical, Hokkaido, Japan), according to the manufacturer's protocol. Cultured BMDMs were plated on 96-well, black μ-clear-bottom microtiter plates (Greiner Bio-One) pre-coated with 25 μg/ml fibrin. BMDMs were incubated with ProteoGREEN-gGlu with acivicin or GGsTop (diluted at threefold concentrations from 0.01 μM to 8.3 μM). The fluorescence intensity (excitation/emission filter pairs of 488 nm/520 nm) was measured using a SpectraMax M5 microplate reader (Molecular Devices) with SoftMax Pro 5.2 software (Phoenix Technologies).

Stereotactic LPS injection and drug treatment. Mice were anaesthetized with isoflurane and placed in a stereotactic apparatus (David Kopf Instruments). LPS (Sigma-Aldrich) was dissolved in endotoxin-free distilled water (HyClone) and was diluted to 1 μg/ml with PBS. LPS (2 μl of 1 μg/ml) or PBS was slowly injected (0.3 μl/min) using a 10-μl Hamilton syringe attached to a 33-G needle into the substantia nigra at coordinates (anteroposterior, 3.0 mm; mediolateral, 1.3 mm; dorsoventral, 4.7 mm from the bregma, according to Paxinos and Watson) Animals received acivicin (5 mg/kg) or saline intraperitoneally beginning 5 days before LPS injection and for 7 days after LPS injection. After 12 h of the LPS injection, the substantia nigra area was immediately collected on ice and kept at −80° C. for later analysis of GGT activity. After 7 d of the LPS injection, mice were perfused with 4% PFA, and the brains were post-fixed in 4% PFA overnight at 4° C. Immunohistochemistry in coronal brain sections (30 μm) were performed using antibodies to tyrosine hydroxylase (TH, 1:2000; P40101, Pel Freez) and Iba-1 (1:1000; 019-19741, Wako). Images were acquired with all-in-one BZ-X700 fluorescence microscope. Images were quantified using NIH ImageJ (version 1.50) by blinded observers.

T-cell proliferation assay. For antigen-specific T cell proliferation, näive CD4+ T cells were magnetically sorted from the TCR-transgenic 2D2 mice using naïve CD4+ T cell isolation kits (Miltenyi Biotec). BMDMs were incubated with 5 μM acivicin, primed with MOG₃₅₋₅₅ peptide (10 μg/ml) and then stimulated with LPS (10 ng/ml) for 24 h. CD4 T cells were cultured with MOG₃₅₋₅₅ peptide primed BMDMs for 3 days and BrdU incorporation was assessed as described previously (67). For non-antigen-specific T cell proliferation, naïve CD4⁺ T cells were treated with acivicin and stimulated with mouse T-activator CD3/CD28 Dynabeads (Thermo Fisher Scientific) for 3 d. Cells were then fixed, permeabilized, and stained with FITC-conjugated anti-BrdU (BrdU Flow Kits, BD Biosciences).

Statistical analyses. Statistical analyses were performed with GraphPad Prism (Version 7). Data are presented as mean±s.e.m. No statistical methods were used to predetermine sample size, but sample sizes are similar to those reported previously. Statistical significance was determined with two-sided unpaired student's t-test, or non-parametric, two-sided Mann-Whitney test, or a oneway or two-way ANOVA analysis of variance followed by Bonferroni or Tukey's post-test (multiple comparisons). Mice were age and sex-matched and were randomly assigned to experimental groups. The assignment of EAE scores, histopathological analysis and quantification were done in a blinded manner. The statistical significance of the changes in the mean clinical score for each day of the EAE experiment was estimated using permutation tests (9). The corresponding P values were estimated using 1000 permutations. In each permutation, mice were randomly permuted.

Results

Single cell oxidative stress transcriptome of CNS innate immunity: To functionally profile the oxidative stress transcriptome of CNS innate immunity and identify neuroprotective drugs, a strategy for single-cell RNA-seq (scRNA-seq) transcriptional profiling of ROS+ CNS innate immune cells was developed and termed Tox-seq and performed microglia HTS of a small molecule library, followed by network analysis (FIG. 1a). To obtain ROS+ innate immune cells, all live cells isolated from spinal cord were stained for intracellular ROS ex vivo using 2′,7′-dichlorofluorescein diacetate (DCFDA) and the innate immune cell fraction was collected by CD11b+ fluorescence-activated cell sorting (FACS). DCFDA is a membrane permeable fluorescent redox indicator probe that detects cellular oxidant stress including ROS, reactive nitrogen species and elevated iron (22). For Tox-seq, the transcriptomes of 8,701 CD11b+ cells labelled for ROS production via scRNA-seq from the spinal cords of healthy and EAE mice at disease onset were analyzed (FIG. 9A-E). Using unsupervised clustering analysis (23) overlaid with the functional ROS characterization, transcriptionally distinct CD11b+ROS+ and CD11b+ROS− populations were identified as visualized by uniform manifold approximation and projection (UMAP) (FIG. 1b and FIG. 9E). The majority of clusters with at least 50% ROS+ cells were infiltrating monocyte/macrophage (Mp; clusters Mp6 to Mp 11), while microglia (Mg) clusters 3 and 5 (Mg3 and Mg5) were at least 15% ROS+ cells (FIG. 1b-d). ROS+ cells were not identified in the healthy spinal cord (FIG. 1b, c). Functional enrichment gene ontology (GO) analysis of differentially expressed genes (DEGs) from monocyte/macrophage clusters with ROS producing cells identified “reactive oxygen species metabolic process” as a top biological process term in EAE. This core oxidative stress signature (e.g., Cybb, Ncf2, Ncf4, and Gpx1) was represented in infiltrating monocyte/macrophage clusters and only in the Mg5 microglia cluster (FIG. 1e). Cluster analysis and differential gene expression further showed that ROS⁺ cells were transcriptionally heterogenous (FIG. 1f; FIG. 9F-H). Homeostatic microglia markers (e.g., P2ryl2, Tmem119, Cx3cr1, Hexb, Olfml3) were expressed in all EAE microglia clusters (Mg3, Mg4, and Mg5) at lower levels than in the healthy microglia clusters (Mg1 and Mg2) (FIG. 1f, g). Similar to prior studies indicating that tissue dissociation procedures affect immediate early response gene expression (21,24), in cluster Mg2 differentially expressed immediate early response genes were identified, which were not excluded from the analysis. In addition to microglia and monocyte/macrophages, two subsets of dendritic cells were identified as described (20). These results suggest functional heterogeneity of CNS innate immune populations in relation to oxidative stress.

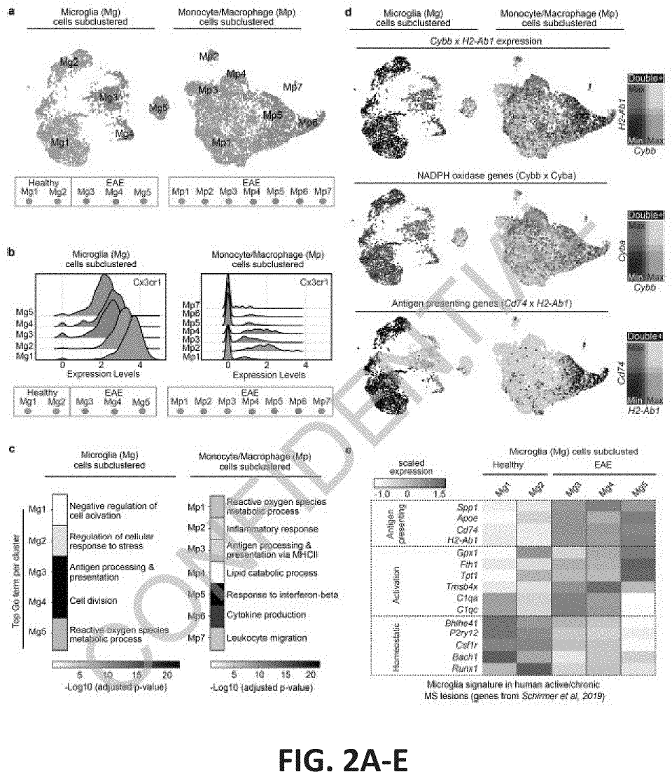

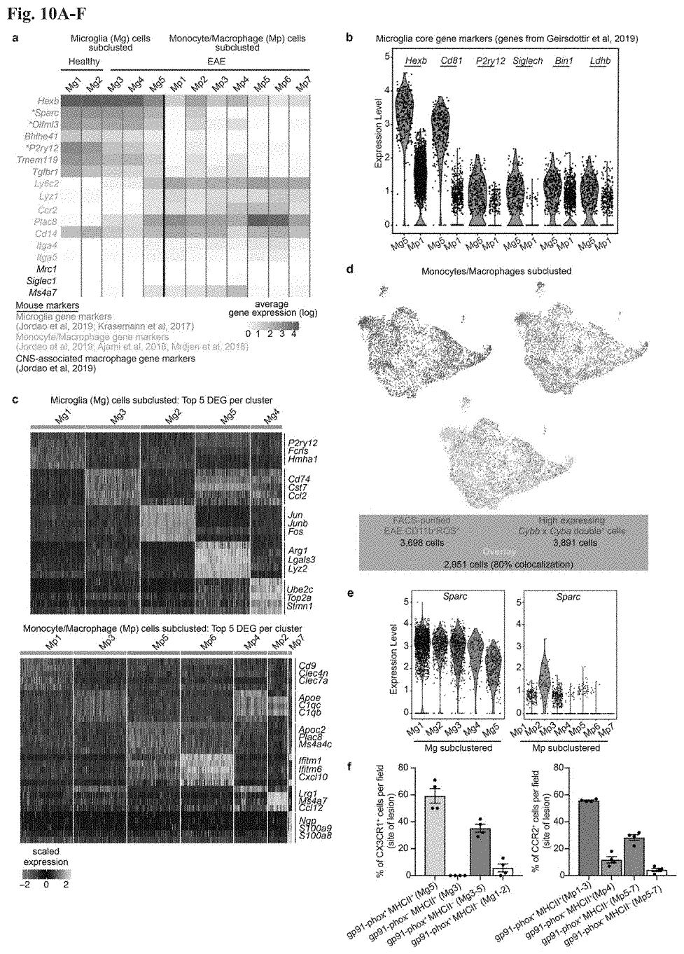

CNS innate immune clusters with oxidative stress and antigen presenting signatures: Single cells were overlaid with gene markers from the core oxidative stress signature and combined with unbiased GO analysis of DEGs from microglia and monocyte/macrophage subclusters (FIG. 2a-d). Subcluster analysis identified five microglia and seven monocyte/macrophage clusters (FIG. 2a). Microglia gene markers, such as Cx3cr1, were highly expressed in all microglia clusters, but not in monocyte/macrophage clusters (FIG. 2b). Cell types of CNS resident and infiltrating immune cells were further validated according to published gene signatures or single cell mass cytometry (CyTOF) (18-20, 25-27), including the highly conserved microglia core genes (FIGS. 10A and B). Clusters Mg5 and Mp1 shared “Reactive oxygen species metabolic process” as top GO term, while clusters Mg3 and Mp3 were enriched for “Antigen Processing and Presentation” (FIG. 2c and FIG. 10C). To identify differentially expressed ROS and antigen presenting genes in single-cell clusters, the expression of the prooxidant gene Cybb, which encodes the nicotinamide adenine dinucleotide phosphate (NADPH) oxidase subunit 2 (gp91-phox) was compared with antigen presenting gene H2-Ab1, which encodes major histocompatibility complex II (MHC II). NADPH oxidase (Cybb) was selected as it is a producer of ROS upregulated in microglia in the cortex of progressive MS, and its genetic depletion is protective in EAE and AD mouse models (3,6,28). Clusters Mg5, Mp1 and Mp2 had increased Cybb and H2-Ab1 expression (FIG. 2d; Cybb/H2-Ab1, yellow), while clusters Mg3,Mg4, Mp3 and Mp4 had only high expression levels of H2-Ab1 (FIG. 2d; Cybb/H2-Ab1, red). Healthy microglia clusters Mg1 and Mg2 did not express Cybb and H2-Ab1 (FIG. 2d; Cybb/H2-Ab1, black). These results were further validated with subcluster overlay of Cd74, which encodes the MHC II invariant chain, and Cyba, which encoded the NADPH oxidase subunit p22-phox. Mg5, Mp3, and Mp1 clusters had high co-expression of NADPH oxidase and antigen presenting genes (FIG. 2d; Cyba/Cybb and Cd74/H2-Ab1, yellow). Cells co-expressing Cyba/Cybb were overlaid with Tox-seq clusters to validate ROS⁺ innate immune cells (FIG. 10D). Mg5 highly expressed the Cyba/Cybb genes, although less than infiltrating monocytes/macrophages (FIGS. 1e, 2d). Microglia activation and homeostatic signatures have been identified in active/chronic MS lesions by single nuclei RNA-seq (16). By comparing the EAE Toxseq dataset in the study with the human MS microglia signatures (16), it was found that among the microglia populations Mg5 exclusively expressed the genes detected in microglia from MS lesions that regulate antigen presentation, glutathione, oxidative stress and iron, such as Cd74, Gpx1, and Fth1 (FIG. 2e). These results suggest differential contribution of CNS innate immune clusters to oxidative stress and antigen presentation.

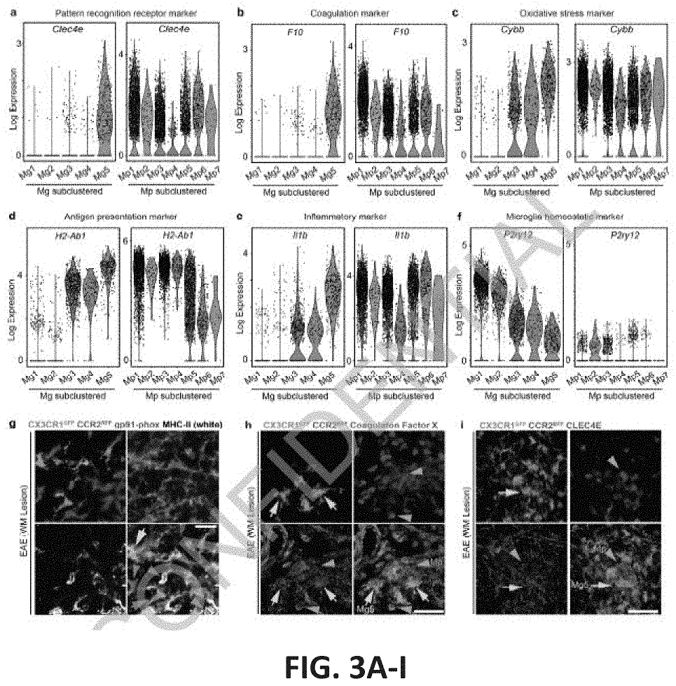

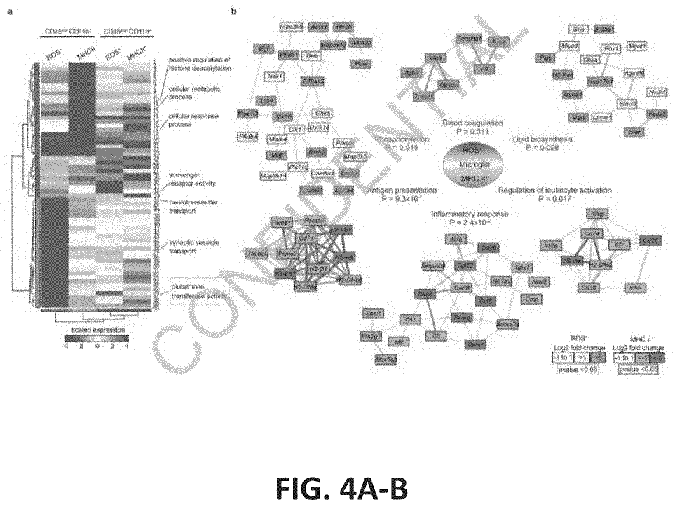

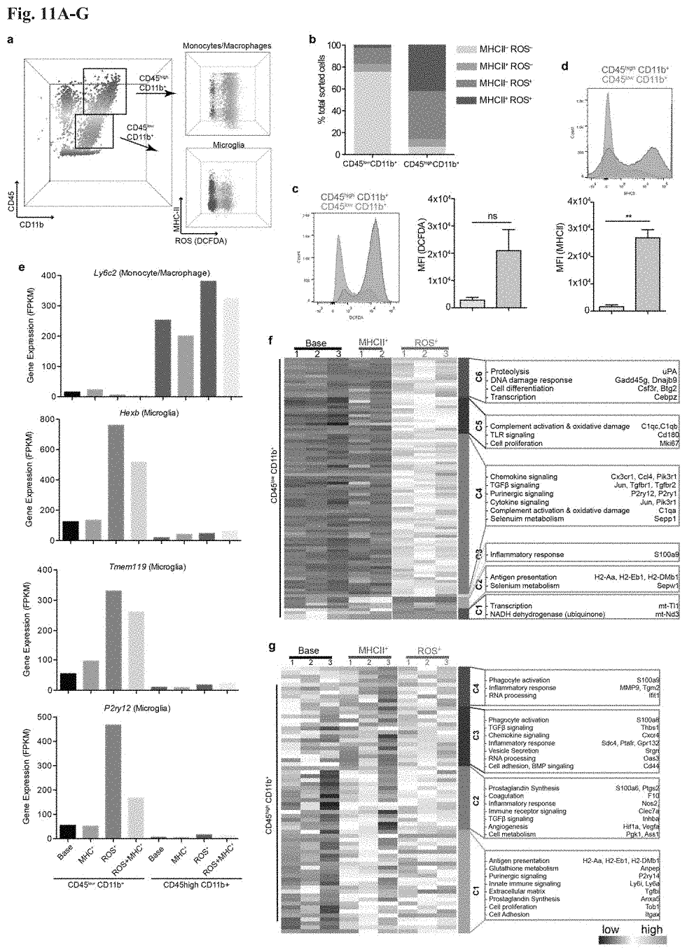

Co-expression of oxidative stress, coagulation, and glutathione pathway genes: Tox-seq identified the Mg5 microglia cluster as a ROS⁺ CNS innate immune population enriched with oxidative stress genes. The transcriptomic signature of Mg5 showed the highest expression for oxidative stress, coagulation, inflammatory, antigen presenting, and pattern recognition receptor markers and the lowest expression of homeostatic markers (FIG. 3). Mg5 exclusively expressed pattern recognition receptor C-type lectin domain family 4 member E (Clec4e) and F10, which encodes coagulation factor X (FIG. 3a, b). Mg5 was also enriched in prooxidant, antigen presenting, and pro-inflammatory markers identified by Cybb, H2-Ab1, and Il1b, respectively (FIG. 3c-e). The homeostatic microglia markers P2ry12, Sparc, Cx3cr1, and Tmem119 were expressed in the Mg5 cluster, but at lower levels than in healthy microglia clusters Mg1 and Mg2 (FIG. 3f and Supp FIGS. 2a and e). Genes specific to the Mg5 cluster were validated in situ by immunostaining for the NADPH oxidase subunit 2 (gp91-phox; encoded by Cybb), MHC II, Coagulation Factor X, and CLEC4E in EAE spinal cords (FIG. 3g-I and FIG. 10F). The transcriptomes of ROS-producing and antigen presenting cells (APCs) were independently validated with bulk RNA-seq of FACS-purified ROS⁺ and MHC II⁺ microglia and monocyte/macrophages from EAE spinal cord (FIG. 11A-E). Gene clustering of DEGs identified distinct upregulated genes in subsets of microglia and monocyte/macrophages that were primarily ROS-producing (FIG. 11F, G). Hierarchical clustering of GO analysis revealed “Scavenger Receptor Activity” and “Glutathione Transferase Activity” upregulated in both ROS⁺ microglia and monocyte/macrophages (FIG. 4a). The ROS⁺ microglia profile included three co-expression networks for high significance GO terms, namely “blood coagulation” (e.g. F9, Itgb3, Serpinc1, Gp9, Gp1ba; P=0.011), “phosphorylation” (e.g. Eif2ak3, Stk30, Pfkfb1, Map3k12, Brsk2) (P=0.015), and “lipid biosynthesis” (e.g. Ggt5, Fads2, Hsd17b1, Srd5a1; P=0.028) (FIG. 4b). These data show that coagulation genes are co-expressed with oxidative stress genes and are in accordance with the role of perivascular microglia as primary sites for increased coagulation activity, fibrin deposition, and ROS release in EAE lesions (29,30).

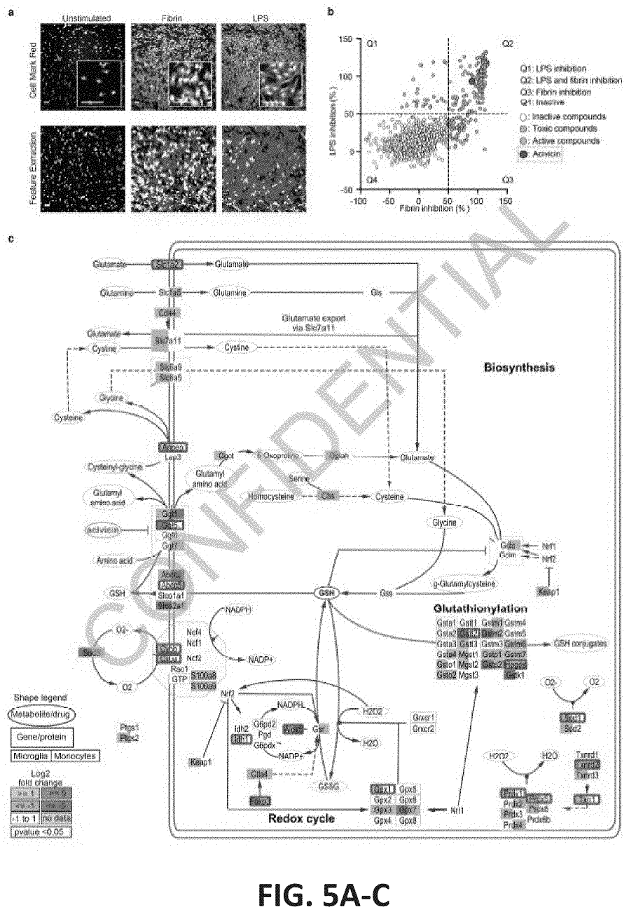

Selection of acivicin by microglia HTS and oxidative stress gene network analysis: Tox-seq identified the coagulation pathway as mechanistically coupled to oxidative stress (FIGS. 3b, 4b). Fibrin, the end product of the coagulation cascade, is deposited in brains from MS and AD and has prooxidant functions in related animal models (9,10,31). A fibrin induced HTS workflow was developed in innate immune cells to discover new small molecule inhibitors to suppress oxidative stress. The HTS workflow consisted of a primary automated microglia HTS of 1,907 compounds that was followed by secondary assays for compound characterization. A high-content HTS of primary microglia stimulated by fibrin or the bacterial innate immune activator lipopolysaccharide (LPS) was first developed (FIG. 5a). 1,907 clinical drugs and bioactive compounds were screened using increased cell size (>800 μm²) as a marker of activation and decreased size (<150 μm²) as a marker of toxicity. 128 compounds were identified that inhibited fibrin-induced microglial activation by >50% without toxicity (<3% cell death) (FIG. 5b and Table 1). Using the Kyoto Encyclopedia of Genes and Genomes (KEGG) database, the molecular targets of 31 compounds were annotated with potential clinical relevance for neurological diseases (Table 2). KEGG analysis identified the glutathione degrading enzyme GGT as the primary target of the HTS hit compound acivicin (FIG. 5b and Tables 1 and 2). Intriguingly, Tox-seq had identified genes regulating glutathione metabolism in ROS⁺ CNS innate immune cells. To examine if acivicin-targeted glutathione and redox pathways were differentially regulated in oxidative stress-producing CNS innate immune cells, a comprehensive oxidative stress gene network was generated and overlaid with the Tox-seq dataset. Using PathVisio and Cytoscape (32,33), an oxidative stress gene network was constructed including the GGT pathway; glutathione metabolism, biosynthesis, uptake and breakdown; the NADPH oxidase complex; the redox cycle and glutathionylation. The oxidative stress gene network was computationally overlaid with genes differentially expressed in ROS⁺ versus MHC subpopulations of microglia and infiltrating monocytes identified by bulk Tox-seq (FIG. 5c). To represent both microglia and infiltrating monocyte gene changes on the same gene network, a split-box view was created with expression levels in microglia and infiltrating monocytes on the left and right sides, respectively (FIG. 5c). This overlay revealed 25 genes that were specifically upregulated in ROS⁺ microglia or monocyte/macrophages throughout the oxidative stress network, including the acivicin target genes Ggt1 and Ggt5, glutathione transferases Gsto2 and Gstt2, and gamma-glutathione peroxidase Gpx7 (FIG. 5c). All 25 genes (Ggt1, Ggt5, Ggt7, Abcc2, Slco2a1, Foxp3, Alox5, Gsr, Gsto1, Gsto2, Gstt2, Gstt3, Mgst2, Gstm1, Gstp2, Gstk1, Gpx7, Slc6a9, Oplah, Cbs, Prdx2, S100a8, S100Aa9, Sod3, Txnrd3) meet the criteria of having log2>1 for either microglia and/or monocytes/macrophages and no downregulation for either subpopulation. Expression of Cyba and Cybb as identified by bulk RNA-seq was lower than Tox-seq single cell analysis potentially due to the low representation of the Cybb/Cyba enriched cluster (FIG. 1e). Based on these findings, the effects of acivicin on innate immune cells and animal models of neuroinflammation was tested.

TABLE 1
IC₅₀ of hits selected from fibrin screen. Dose response inhibition
activity of 128 hits picked from the high-throughput screen
that revealed >50% inhibition of fibrin-induced microglia activation.
IC₅₀ values were calculated for these 128 hits tested in both
fibrin and LPS assay. The list in Table 1 are compounds originated
from Q2 and Q3 in FIG. 5b (Q1 is LPS, Q3 is fibrin, Q2 is both
LPS and fibrin, and Q4 is inactive).

| | | Fibrin | LPS IC₅₀ | Quadrant |
|---|---|---|---|---|
| No. | Name | IC₅₀ (μM) | (μM) | (FIG. 5b) |
| 1 | 2-methoxyestradiol | 3 | 5 | Q2 |
| 2 | Acivicin | 2 | 1 | Q2 |
| 3 | Acrisorcin | 7 | 5 | Q2 |
| 4 | Albendazole | 4 | 6 | Q3 |
| 5 | Alclometazone dipropionate | <0.001 | 2 | Q2 |
| 6 | Algestone acetophenide | 1 | 9 | Q2 |
| 7 | Almotriptan | >20 | >20 | Q2 |
| 8 | Amisulpride | 20 | >20 | Q3 |
| 9 | Amoxapine | 20 | 11 | Q3 |
| 10 | Artesunate | 7 | >20 | Q3 |
| 11 | Atorvastatin calcium | 0.6 | 3 | Q2 |
| 12 | Bacitracin | >20 | >20 | Q3 |
| 13 | Benzoyl peroxide | 20 | >20 | Q2 |
| 14 | Benzydamine hydrochloride | 6 | 3 | Q2 |
| 15 | Benzyl isothiocyanate | 10 | >20 | Q2 |
| 16 | Betamethasone | <0.001 | 0.01 | Q2 |
| 17 | Betamethasone sodium | 0.05 | 0.2 | Q2 |
| | phosphate | | | |
| 18 | Betamethasone valerate | 20 | >20 | Q2 |
| 19 | Betazole hydrochloride | 12 | >20 | Q3 |
| 20 | Chlormadinone acetate | 17 | >20 | Q3 |
| 21 | Chlorthalidone | >20 | >20 | Q3 |
| 22 | Clobetasol propionate | <0.001 | <0.001 | Q2 |
| 23 | Cloperastine hydrochloride | 7 | 9 | Q3 |
| 24 | Colistimethate sodium | >20 | >20 | Q2 |
| 25 | Cyclosporin A | 20 | 19 | Q3 |
| 26 | Cyproterone | 17 | >20 | Q3 |
| 27 | Deflazacort | <0.001 | 0.04 | Q2 |
| 28 | Demeclocycline hydrochloride | 6 | 15 | Q3 |
| 29 | Desoxycorticosterone acetate | 0.5 | 5 | Q3 |
| 30 | Dexamethasone | 15 | >20 | Q3 |
| 31 | Dexamethasone sodium | 0.004 | 2 | Q2 |
| | phosphate | | | |
| 32 | Dichlorisone acetate | 0.03 | 4 | Q2 |
| 33 | Diflorasone diacetate | 0.01 | >20 | Q3 |
| 34 | Docetaxel | 0.002 | >20 | Q3 |
| 35 | Docetaxel | 20 | >20 | Q3 |
| 36 | Eplerenore | 3 | >20 | Q2 |
| 37 | Ethacrynic acid | >20 | >20 | Q3 |
| 38 | Ethinyl estradiol | >20 | >20 | Q3 |
| 39 | Fenbendazole | 3 | 4 | Q3 |
| 40 | Fludrocortisone acetate | <0.001 | 0.4 | Q2 |
| 41 | Flumethasone | <0.001 | <0.001 | Q2 |
| 42 | Flumethazone pivalate | <0.001 | 0.004 | Q2 |
| 43 | Flunisolide | <0.001 | <0.001 | Q2 |
| 44 | Fluocinolone acetonide | <0.001 | <0.001 | Q2 |
| 45 | Fluorouracil | 7 | >20 | Q3 |
| 46 | Ftaxilide | >20 | >20 | Q3 |
| 47 | Fusidic acid | 19 | >20 | Q2 |
| 48 | Gentamicin sulfate | 4 | >20 | Q2 |
| 49 | Hycanthone | 1 | 1 | Q2 |
| 50 | Hydrocortisone | 0.008 | 0.1 | Q2 |
| 51 | Hydrocortisone | 0.03 | 18 | Q2 |
| 52 | Hydrocortisone butyrate | 0.02 | 2 | Q2 |
| 53 | Hydrocortisone hemisuccinate | 0.02 | 7 | Q2 |
| 54 | Hydrocortisone valerate | 0.05 | 1 | Q2 |
| 55 | Hydroxytoluic acid | 20 | >20 | Q3 |
| 56 | Ibudilast | >20 | >20 | Q2 |
| 57 | Idazoxan hydrochloride | 0.4 | 2 | Q3 |
| 58 | Imipenem | >20 | >20 | Q3 |
| 59 | Iopanic acid | 5 | >20 | Q3 |
| 60 | Irbesartan | >20 | >20 | Q3 |
| 61 | Isoxicam | 7 | >20 | Q3 |
| 62 | Levonordefrin | 0.9 | >20 | Q3 |
| 63 | Mebendazole | 0.2 | 6 | Q2 |
| 64 | Mechlorethamine | 7 | 0.1 | Q2 |
| 65 | Medroxyprogesterone | >20 | 8 | Q3 |
| 66 | Medroxyprogesterone acetate | 0.3 | >20 | Q2 |
| 67 | Medroxyprogesterone acetate | 0.5 | >20 | Q2 |
| 68 | Megestrol acetate | 5 | >20 | Q3 |
| 69 | Melengestrol acetate | 0.07 | 13 | Q2 |
| 70 | Mephentermine sulfate | >20 | >20 | Q3 |
| 71 | Mequinol | >20 | >20 | Q2 |
| 72 | Metampicillin sodium | 20 | >20 | Q3 |
| 73 | Methylphenidate | >20 | >20 | Q2 |
| | hydrochloride | | | |
| 74 | Methylprednisolone | <0.001 | 0.02 | Q2 |
| 75 | Miconazole nitrate | 20 | >20 | Q3 |
| 76 | Midodrine hydrochloride | >20 | >20 | Q2 |
| 77 | Milnacipran hydrochloride | 8 | 20 | Q3 |
| 78 | Mirtazapine | >20 | >20 | Q3 |
| 79 | Molsidomine | >20 | >20 | Q2 |
| 80 | Monensin | 0.004 | 0.2 | Q2 |
| 81 | Mycophenolate mofetil | 2 | 1 | Q2 |
| 82 | Mycophenolic acid | 1 | 1 | Q2 |
| 83 | Naftopidil dihydrochloride | 7 | >20 | Q3 |
| 84 | Nortriptyline | 13 | 3 | Q2 |
| | hydrochloride | | | |
| 85 | Oxaliplatin | 5 | 15 | Q3 |
| 86 | Oxiglutatione disodium salt | >20 | >20 | Q3 |
| 87 | Oxybendazole | 7 | 12 | Q3 |
| 88 | Perhexiline maleate | 8 | 3 | Q2 |
| 89 | Phenytoin sodium | >20 | >20 | Q2 |
| 90 | Pimozide | >20 | 18 | Q3 |
| 91 | Pitavastatin calcium | 1 | 5 | Q2 |
| 92 | Prednicarbate | 0.002 | >20 | Q2 |
| 93 | Prednisolone sodium | 0.1 | 4 | Q2 |
| | phosphate | | | |
| 94 | Prednisone | 3 | >20 | Q3 |
| 95 | Prednisone | 12 | >20 | Q3 |
| 96 | R(−)-Apomorphine | >20 | 14 | Q3 |
| | hydrochloride | | | |
| 97 | Ractopamine hydrochloride | 20 | >20 | Q3 |
| 98 | Rapamycin | <0.001 | <0.001 | Q2 |
| 99 | Rasagiline | 20 | >20 | Q3 |
| 100 | Rebamipide | 2 | 11 | Q2 |
| 101 | Ribavirin | 1 | 2 | Q2 |
| 102 | Risedronate sodium | 19 | >20 | Q3 |
| 103 | Ritodrine hydrochloride | 0.6 | >20 | Q3 |
| 104 | Rosuvastatin | 0.3 | 3 | Q2 |
| 105 | Rubitecan | 2 | 9 | Q3 |
| 106 | Salicin | >20 | >20 | Q2 |
| 107 | Salinomycin, sodium | 2 | 2 | Q2 |
| 108 | Salmeterol | 1 | 14 | Q3 |
| 109 | Saquinavir | >20 | >20 | Q2 |
| 110 | Securinine | 2 | 6 | Q2 |
| 111 | Selamectin | 6 | >20 | Q3 |
| 112 | Sulbactam | >20 | >20 | Q3 |
| 113 | Sulfaquinoxaline sodium | >20 | >20 | Q2 |
| 114 | Tegaserod maleate | 1 | 19 | Q2 |
| 115 | Teniposide | 2 | 1 | Q2 |
| 116 | Tepoxalin | 3 | 18 | Q2 |
| 117 | Thiabendazole | >20 | >20 | Q2 |
| 118 | Tolazamide | 12 | >20 | Q3 |
| 119 | Tolnaftate | 2 | 17 | Q2 |
| 120 | Toltrazuril | >20 | >20 | Q2 |
| 121 | Triamcinolone | 0.04 | 0.4 | Q2 |
| 122 | Triamcinolone diacetate | 0.04 | 0.8 | Q2 |
| 123 | Trifluoperazine | 12 | 6 | Q3 |
| | dihydrochloride | | | |
| 124 | Trimipramine maleate | 7 | 5 | Q2 |
| 125 | Tyrothricin | 3 | 0.5 | Q2 |
| 126 | Vinblastine sulfate | 0.02 | 0.2 | Q2 |
| 127 | Xylazine hydrochloride | 2 | 8 | Q2 |
| 128 | Zoledronic acid | 2 | 8 | Q3 |

TABLE 2
Selection of 31 small molecules from the HTS, of which 27 compounds inhibited fibrin- and/or LPS-induced microglia activation
(i.e. compounds originated from Q1, Q2, Q3 in FIG. 5b; Q1 is LPS, Q3 is fibrin, Q2 is both LPS and fibrin, and Q4 is
inactive). Molecular targets and biological functions for each compound were retrieved from the KEGG database.

| | | | | | Quadrant |
|---|---|---|---|---|---|
| # | Name of small molecule | Primary target (gene name) | Biological Function | Reference | (FIG. 5b) |
| 1 | Acivicin | Gamma-glutamyl transferase | Gamma-glutamyl cycle, glutathione | KEGG: D02755; | Q2 |
| | | (GGT) | metabolism | PMID: 7914892 | |
| 2 | Nylidrin hydrochloride | Adrenergic receptor beta | Calcium signaling pathway, cAMP | KEGG: D01543 | Q4 |
| | (C19H26ClNO2) | (ADRB) | signaling pathway | | |
| 3 | Vinblastine sulfate | ATP-binding cassette transporter | Multidrug resistance, Microtuble | KEGG: D01068 | Q2 |
| | | (ABCB1), Tubulin beta chain | dynamics | | |
| | | (TUBB) | | | |
| 4 | Colchicine | Tubulin beta chain (TUBB) | Cell motility, Microtuble dynamics, | KEGG: D00570 | Q2 |
| | | | Mitosis | | |
| 5 | Podofilox | Tubulin beta chain (TUBB) | Cell motility, Microtuble dynamics, | KEGG: D05529 | Q2 |
| | | | Mitosis | | |
| 6 | Fendiline hydrochloride | Calcium channel L type | Calcium signaling | KEGG: D07943 | Q4 |
| | | (CACNA1) | | | |
| 7 | Tannic acid | Chemokine ligand 12 (CXCL12) | Chemokine signaling | PMID: 12912963 | Q4 |
| 8 | Teniposide | DNA Topoisomerase II (Top2) | DNA replication, DNA damage | KEGG: D02698 | Q2 |
| | | | checkpoint | | |
| 9 | Prochlorperazine dimaleate | Dopamine receptor (DRD2) | Dopamine Receptor Signaling, cAMP- | KEGG: D00493 | Q4 |
| | | | mediated signaling, GPCR signaling | | |
| 10 | Thioridazine hydrochloride | Dopamine receptor (DRD2) | Dopamine Receptor Signaling, cAMP- | KEGG: D00798 | Q2 |
| | | | mediated signaling, GPCR signaling | | |
| 11 | Betamethasone | Glucocorticoid receptor (NR3C1) | Glucocorticoid Receptor Signaling | KEGG: D00244 | Q2 |
| 12 | Dexamethasone | Glucocorticoid receptor (NR3C1) | Glucocorticoid Receptor Signaling | KEGG: D00292 | Q3 |
| 13 | Dexamethasone acetate | Glucocorticoid receptor (NR3C1) | Glucocorticoid Receptor Signaling | KEGG: D02174 | Q2 |
| 14 | Dexamethasone sodium phosphate | Glucocorticoid receptor (NR3C1) | Glucocorticoid Receptor Signaling | KEGG: D00975 | Q2 |
| 15 | Fludrocortisone acetate | Glucocorticoid receptor (NR3C1) | Glucocorticoid Receptor Signaling | KEGG: D00986 | Q2 |
| 16 | Fluocinolone acetonide | Glucocorticoid receptor (NR3C1) | Glucocorticoid Receptor Signaling | KEGG: D01825 | Q2 |
| 17 | Fluocinonide | Glucocorticoid receptor (NR3C1) | Glucocorticoid Receptor Signaling | KEGG: D00325 | Q2 |
| 18 | FluoromethoIone | Glucocorticoid receptor (NR3C1) | Glucocorticoid Receptor Signaling | KEGG: D01367 | Q2 |
| 19 | Flurandrenolide | Glucocorticoid receptor (NR3C1) | Glucocorticoid Receptor Signaling | KEGG: D00328 | Q2 |
| 20 | Hydrocortisone | Glucocorticoid receptor (NR3C1) | Glucocorticoid Receptor Signaling | KEGG: D00088 | Q2 |
| 21 | Hydrocortisone acetate | Glucocorticoid receptor (NR3C1) | Glucocorticoid Receptor Signaling | KEGG: D00165 | Q2 |
| 22 | Hydrocortisone butyrate | Glucocorticoid receptor (NR3C1) | Glucocorticoid Receptor Signaling | KEGG: D01619 | Q2 |
| 23 | Hydrocortisone hemisuccinate | Glucocorticoid receptor (NR3C1) | Glucocorticoid Receptor Signaling | KEGG: D04467 | Q2 |
| 24 | Hydrocortisone sodium phosphate | Glucocorticoid receptor (NR3C1) | Glucocorticoid Receptor Signaling | KEGG: D00977 | Q2 |
| 25 | Methylprednisolone | Glucocorticoid receptor (NR3C1) | Glucocorticoid Receptor Signaling | KEGG: D00407 | Q2 |
| 26 | Prednisolone | Glucocorticoid receptor (NR3C1) | Glucocorticoid Receptor Signaling | KEGG: D00472 | Q2 |
| 27 | Prednisolone acetate | Glucocorticoid receptor (NR3C1) | Glucocorticoid Receptor Signaling | KEGG: D00980 | Q2 |
| 28 | Triamcinolone diacetate | Glucocorticoid receptor (NR3C1) | Glucocorticoid Receptor Signaling | KEGG: D00984 | Q2 |
| 29 | Mycophenolic acid | Inosine-5′-monophosphate | Purine Nucleotide Biosynthesis | KEGG: D05096 | Q2 |
| | | dehydrogenase (IMPDH) | | | |
| 30 | Maprotiline hydrochloride | Solute carrier family 6, member 2 | Neurotransmitter transport | KEGG: D00818 | Q1 |
| | | (SLC6A2) | | | |
| 31 | Desipramine hydrochloride | Solute carrier family 6 member 2, | Serotonin Receptor Signaling; | KEGG: D00812 | Q1 |
| | | 4 (SLC6A2; SLC6A4) | neurotransmitter transport | | |

TABLE 3
Top 64 compounds sorted for selectivity to fibrin vs LPS.

| | | | | Fibrin | LPS | | | | Cell | Cell | |
|---|---|---|---|---|---|---|---|---|---|---|---|
| | | | Cherry- | IC50 | IC50 | KEGG | | Fibrin | Toxicity | Toxicity | |
| | | | picked Hit | (uM) | (uM) | Analysis | Fibrin | Selec- | IC50 (uM) | IC50 (uM) | |
| | | Quadrant | (Suppl. | (Suppl. | (Suppl. | (Suppl. | Selec- | tivity | [cFib | [LPS | |
| No. | Name | (FIG. 5b) | Table 7) | Table 7) | Table 7) | Table 8) | tivity | (LOG10) | assay] | assay] | Therapy |
| 1 | Docetaxel | Q3 | HIT | 0.002 | >20 | | 9814 | 3.99 | >20 | >20 | antineoplastic |
| 2 | Prednicarbate | Q2 | HIT | 0.002 | >20 | | 8510 | 3.93 | >20 | >20 | antiinflammatory, |
| | | | | | | | | | | | glucocorticoid |
| 3 | Diflorasone | Q3 | HIT | 0.01 | >20 | | 2188 | 3.34 | >20 | >20 | antiinflammatory, |
| | Diacetate | | | | | | | | | | glucocorticoid |
| 4 | Alclometazone | Q2 | HIT | <0.001 | 2 | | 2153 | 3.33 | >20 | >20 | antiinflammatory, |
| | Dipropion | | | | | | | | | | glucocorticoid |
| 5 | Hydrocortisone | Q2 | HIT | 0.03 | 18 | KEGG | 568 | 2.75 | >20 | >20 | glucocorticoid |
| 6 | Dexamethasone | Q2 | HIT | 0.004 | 2 | KEGG | 383 | 2.58 | >20 | >20 | glucocorticoid, |
| | Sodium | | | | | | | | | | antiinflammatory |
| 7 | Fludrocortisone | Q2 | HIT | <0.001 | 0.4 | KEGG | 362 | 2.56 | >20 | >20 | mineralocorticoid |
| | Acetate | | | | | | | | | | |
| 8 | Hydrocortisone | Q2 | HIT | 0.02 | 7 | KEGG | 350 | 2.54 | >20 | >20 | glucocorticoid |
| | Hemisuc | | | | | | | | | | |
| 9 | Melengestrol | Q2 | HIT | 0.07 | 13 | | 196 | 2.29 | >20 | >20 | antineoplastic, |
| | Acetate | | | | | | | | | | progestin |
| 10 | Dichlorisone | Q2 | HIT | 0.03 | 4 | | 169 | 2.23 | >20 | >20 | antipruretic |
| | Acetate | | | | | | | | | | |
| 11 | Hydrocortisone | Q2 | HIT | 0.02 | 2 | KEGG | 89 | 1.95 | >20 | >20 | glucocorticoid, |
| | Butyrate | | | | | | | | | | antiinflammatory |
| 12 | Medroxypro- | Q2 | HIT | 0.3 | >20 | | 70 | 1.84 | >20 | >20 | progestogen |
| | gesterone Ac | | | | | | | | | | |
| 13 | Monensin | Q2 | HIT | 0.004 | 0.2 | | 49 | 1.69 | >20 | 2 | antibiotic, |
| | Sodium | | | | | | | | | | antibacterial; |
| | | | | | | | | | | | antibacterial |
| 14 | Deflazacort | Q2 | HIT | <0.001 | 0.04 | | 42 | 1.63 | 20 | >20 | antiinflammatory |
| 15 | Medroxy- | Q2 | HIT | 0.5 | >20 | | 41 | 1.61 | >20 | >20 | contraceptive |
| | progesterone Ac | | | | | | | | | | |
| 16 | Ritodrine | Q3 | HIT | 0.6 | >20 | | 33 | 1.51 | >20 | >20 | muscle relaxant |
| | Hydrochloride | | | | | | | | | | (smooth) |
| 17 | Prednisolone | Q2 | HIT | 0.1 | 4 | | 31 | 1.49 | >20 | >20 | antiinflammatory, |
| | Sodium Ph | | | | | | | | | | glucocorticoid |
| 18 | Mebendazole | Q2 | HIT | 0.2 | 6 | | 31 | 1.49 | >20 | >20 | anthelmintic |
| 19 | Hydrocortisone | Q2 | HIT | 0.05 | 1 | | 28 | 1.45 | >20 | >20 | antiinflammatory, |
| | Valerate | | | | | | | | | | glucocorticoid |
| 20 | Levonordefrin | Q3 | HIT | 0.9 | >20 | | 21 | 1.33 | >20 | >20 | vasoconstrictor |
| 21 | Tegaserod | Q2 | HIT | 1 | 19 | | 20 | 1.29 | >20 | >20 | 5HT4 receptor |
| | Maleate | | | | | | | | | | agonist, |
| | | | | | | | | | | | peristaltic stimula |
| 22 | Triamcinolone | Q2 | HIT | 0.04 | 0.8 | KEGG | 17 | 1.23 | >20 | >20 | antiinflammatory |
| | Diacetate | | | | | | | | | | |
| 23 | Hydrocortisone | Q2 | HIT | 0.008 | 0.1 | KEGG | 16 | 1.21 | >20 | >20 | glucocorticoid, |
| | | | | | | | | | | | antiinflammatory |
| 24 | Methylprednisolone | Q2 | HIT | <0.001 | 0.02 | KEGG | 15 | 1.19 | >20 | >20 | glucocorticoid |
| 25 | Rosuvastatin | Q2 | HIT | 0.3 | 3 | | 12 | 1.09 | 20 | >20 | antihyperlipidemic |
| 26 | Vinblastine | Q2 | HIT | 0.02 | 0.2 | KEGG | 11 | 1.05 | >20 | >20 | antineoplastic, |
| | Sulfate | | | | | | | | | | spindle poison |
| 27 | Salmeterol | Q3 | HIT | 1 | 14 | | 10 | 1.02 | >20 | >20 | Bronchodilator |
| 28 | Triamcinolone | Q2 | HIT | 0.04 | 0.4 | | 10 | 1.00 | >20 | >20 | glucocorticoid |
| 29 | Betamethasone | Q2 | HIT | <0.001 | 0.01 | KEGG | 10 | 0.99 | >20 | >20 | glucocorticoid, |
| | | | | | | | | | | | antiinflammatory |
| 30 | Desoxycortico- | Q3 | HIT | 0.5 | 5 | | 10 | 0.98 | >20 | >20 | mineralocorticoid |
| | sterone Ac | | | | | | | | | | |
| 31 | Algestone | Q2 | HIT | 1 | 9 | | 9 | 0.95 | >20 | >20 | antiacne, progestin |
| | Acetophenide | | | | | | | | | | |
| 32 | Eplerenore | Q2 | HIT | 3 | >20 | | 8 | 0.88 | >20 | >20 | antihypertensive |
| 33 | Tolnaftate | Q2 | HIT | 2 | 17 | | 7 | 0.87 | >20 | >20 | antifungal |
| 34 | Rebamipide | Q2 | HIT | 2 | 11 | | 6 | 0.80 | >20 | >20 | antiulcer, |
| | | | | | | | | | | | antioxidant |
| 35 | Prednisone | Q3 | HIT | 3 | >20 | | 6 | 0.78 | >20 | >20 | glucocorticoid |
| 36 | Tepoxalin | Q2 | HIT | 3 | 18 | | 6 | 0.75 | >20 | >20 | antipsoratic |
| 37 | Xylazine | Q2 | HIT | 2 | 8 | | 5 | 0.72 | >20 | >20 | ″alpha2 |
| | Hydrochloride | | | | | | | | | | Adrenoceptor |
| | | | | | | | | | | | agonist; |
| | | | | | | | | | | | anesthetic |
| 38 | Gentamicin | Q2 | HIT | 4 | >20 | | 5 | 0.71 | >20 | >20 | antibacterial |
| | Sulfate | | | | | | | | | | |
| 39 | Idazoxan | Q3 | HIT | 0.4 | 2 | | 5 | 0.69 | >20 | >20 | alpha2- |
| | Hydrochloride | | | | | | | | | | adrenergic blocker |
| 40 | Atorvastatin | Q2 | HIT | 0.6 | 3 | | 5 | 0.67 | >20 | >20 | antihyperlipidemic, |
| | Calcium | | | | | | | | | | HMGCoA |
| | | | | | | | | | | | reductase i |
| 41 | Rubitecan | Q3 | HIT | 2 | 9 | | 4 | 0.63 | >20 | >20 | antineoplastic |
| 42 | Megestrol | Q3 | HIT | 5 | >20 | | 4 | 0.63 | >20 | >20 | progestogen, |
| | Acetate | | | | | | | | | | antineoplastic |
| 43 | Betamethasone | Q2 | HIT | 0.05 | 0.2 | | 4 | 0.62 | >20 | >20 | antiinflammatory, |
| | Sodium | | | | | | | | | | glucocorticoid |
| 44 | Pitavastatin | Q2 | HIT | 1 | 5 | | 4 | 0.61 | >20 | >20 | HMGA reductase |
| | Calcium | | | | | | | | | | inhibitor |
| 45 | Flumethazone | Q2 | HIT | <0.001 | 0.004 | | 4 | 0.58 | >20 | >20 | glucocorticoid, |
| | Pivalate | | | | | | | | | | antiinflammatory |
| 46 | Iopanic Acid | Q3 | HIT | 5 | >20 | | 4 | 0.58 | >20 | >20 | radioopaque |
| | | | | | | | | | | | agent |
| 47 | Zoledronic | Q3 | HIT | 2 | 8 | | 4 | 0.55 | >20 | >20 | Antiosteoporotic |
| | Acid | | | | | | | | | | |
| 48 | Selamectin | Q3 | HIT | 6 | >20 | | 3 | 0.51 | >20 | >20 | anthelmintic, |
| | | | | | | | | | | | antiparasitic, |
| | | | | | | | | | | | antimite |
| 49 | Oxaliplatin | Q3 | HIT | 5 | 15 | | 3 | 0.48 | >20 | >20 | antineoplastic |
| 50 | Artesunate | Q3 | HIT | 7 | >20 | | 3 | 0.45 | >20 | >20 | Antimalarial |
| 51 | Naftopidil | Q3 | HIT | 7 | >20 | | 3 | 0.45 | >20 | >20 | ″alpha1 |
| | Dihydrochlorid | | | | | | | | | | Adrenoceptor |
| | | | | | | | | | | | antagonist; |
| | | | | | | | | | | | antihy |
| 52 | Fluorouracil | Q3 | HIT | 7 | >20 | | 3 | 0.44 | >20 | >20 | antineoplastic, |
| | | | | | | | | | | | pyrimidine |
| | | | | | | | | | | | antimetabolite |
| 53 | Isoxicam | Q3 | HIT | 7 | >20 | | 3 | 0.43 | >20 | >20 | antiinflammatory |
| 54 | Milnacipran | Q3 | HIT | 8 | 20 | | 3 | 0.40 | >20 | >20 | inhibitor of |
| | Hydrochlorid | | | | | | | | | | norepinephrine |
| | | | | | | | | | | | and seritonin |
| 55 | Securinine | Q2 | HIT | 2 | 6 | | 3 | 0.40 | >20 | 12 | GABAA receptor |
| | | | | | | | | | | | blocker, CNS |
| | | | | | | | | | | | stimulant |
| 56 | Demeclocycline | Q3 | HIT | 6 | 15 | | 2 | 0.38 | >20 | >20 | antibacterial |
| | Hydroch | | | | | | | | | | |
| 57 | Benzyl | Q2 | HIT | 10 | >20 | | 2 | 0.29 | >20 | >20 | antineoplastic, |
| | Isothiocyanate | | | | | | | | | | antibacterial, |
| | | | | | | | | | | | antifungal |
| 58 | Ribavirin | Q2 | HIT | 1 | 2 | | 2 | 0.29 | >20 | >20 | antiviral |
| 59 | Tolazamide | Q3 | HIT | 12 | >20 | | 2 | 0.23 | >20 | >20 | ″Oral |
| | | | | | | | | | | | hypoglycemic |
| | | | | | | | | | | | agent; |
| | | | | | | | | | | | stimulates pa |
| 60 | Oxybendazole | Q3 | HIT | 7 | 12 | | 2 | 0.22 | >20 | >20 | anthelmintic |
| 61 | Albendazole | Q3 | HIT | 4 | 6 | | 2 | 0.22 | >20 | >20 | anthelmintic |
| 62 | Betazole | Q3 | HIT | 12 | >20 | | 2 | 0.22 | >20 | >20 | gastric secretion |
| | Hydrochloride | | | | | | | | | | stimulant |
| 63 | Prednisone | Q3 | HIT | 12 | >20 | | 2 | 0.21 | >20 | >20 | antiinflammatory, |
| | | | | | | | | | | | glucocorticoid |
| 64 | 2- | Q2 | HIT | 3 | 5 | | 2 | 0.21 | >20 | >20 | angiogenesis |
| | Methoxyestradiol | | | | | | | | | | inhibitor |

| | | | Fibrin % | | LPS % | | | Fibrin | LPS | |
|---|---|---|---|---|---|---|---|---|---|---|
| | | Fibrin % | cell | LPS % | cell | Quadrant | | IC50 | IC50 | KEGG |
| Name | PAINS | inhibition | toxicity | inhibition | toxicity | (FIG. 5b) | Hit | (uM) | (uM) | Analysis |
| Acivicin | 0 | 90 | 2 | 93 | 4 | Q2 | HIT | 2 | 1 | KEGG |
| Betamethasone | 0 | 103 | 2 | 94 | 5 | Q2 | HIT | <0.001 | 0.01 | KEGG |
| Dexamethasone Sodium Phosphate | 0 | 112 | 2 | 100 | 3 | Q2 | HIT | 0.004 | 2 | KEGG |
| Fludrocortisone Acetate | 0 | 104 | 2 | 101 | 4 | Q2 | HIT | <0.001 | 0.4 | KEGG |
| Fluocinolone Acetonide | 0 | 105 | 2 | 104 | 4 | Q2 | HIT | <0.001 | <0.001 | KEGG |
| Hydrocortisone | 0 | 105 | 2 | 101 | 5 | Q2 | HIT | 0.008 | 0.1 | KEGG |
| Hydrocortisone | 0 | 104 | 2 | 96 | 2 | Q2 | HIT | 0.03 | 18 | KEGG |
| Hydrocortisone Butyrate | 0 | 108 | 2 | 78 | 2 | Q2 | HIT | 0.02 | 2 | KEGG |
| Hydrocortisone Hemisuccinate | 0 | 100 | 1 | 64 | 1 | Q2 | HIT | 0.02 | 7 | KEGG |
| Methylprednisolone | 0 | 110 | 3 | 86 | 2 | Q2 | HIT | <0.001 | 0.02 | KEGG |
| Mycophenolic Acid | 0 | 94 | 1 | 91 | 2 | Q2 | HIT | 1 | 1 | KEGG |
| Teniposide | 0 | 91 | 2 | 100 | 18 | Q2 | HIT | 2 | 1 | KEGG |
| Triamcinolone Diacetate | 0 | 110 | 2 | 100 | 5 | Q2 | HIT | 0.04 | 0.8 | KEGG |
| Vinblastine Sulfate | 0 | 106 | 2 | 108 | 8 | Q2 | HIT | 0.02 | 0.2 | KEGG |
| Dexamethasone | 0 | 57 | 2 | 17 | 0 | Q3 | HIT | 15 | >20 | KEGG |
| 2-Methoxyestradiol | 0 | 63 | 2 | 65 | 2 | Q2 | HIT | 3 | 5 | |
| Acrisorcin | 2 | 87 | 2 | 113 | 52 | Q2 | HIT | 7 | 5 | |
| Alclometazone Dipropionate | 0 | 105 | 2 | 82 | 2 | Q2 | HIT | <0.001 | 2 | |
| Algestone Acetophenide | 0 | 62 | 1 | 61 | 2 | Q2 | HIT | 1 | 9 | |
| Almotriptan | 0 | 101 | 2 | 85 | 1 | Q2 | HIT | >20 | >20 | |
| Atorvastatin Calcium | 0 | 107 | 3 | 88 | 2 | Q2 | HIT | 0.6 | 3 | |
| Benzoyl Peroxide | 0 | 75 | 1 | 82 | 2 | Q2 | HIT | 20 | >20 | |
| Benzydamine Hydrochloride | 0 | 75 | 1 | 102 | 6 | Q2 | HIT | 6 | 3 | |
| Benzyl Isothiocyanate | 0 | 77 | 2 | 68 | 2 | Q2 | HIT | 10 | >20 | |
| Betamethasone Sodium Phosphate | 0 | 107 | 2 | 82 | 3 | Q2 | HIT | 0.05 | 0.2 | |
| Betamethasone Valerate | 0 | 69 | 1 | 55 | 1 | Q2 | HIT | 20 | >20 | |
| Clobetasol Propionate | 0 | 108 | 3 | 105 | 3 | Q2 | HIT | <0.001 | <0.001 | |
| Colistimethate Sodium | 0 | 72 | 1 | 112 | 4 | Q2 | HIT | >20 | >20 | |
| Deflazacort | 0 | 111 | 2 | 105 | 4 | Q2 | HIT | <0.001 | 0.04 | |
| Dichlorisone Acetate | 0 | 108 | 3 | 74 | 1 | Q2 | HIT | 0.03 | 4 | |
| Eplerenore | 0 | 63 | 2 | 69 | 2 | Q2 | HIT | 3 | >20 | |
| Flumethasone | 0 | 109 | 2 | 101 | 6 | Q2 | HIT | <0.001 | <0.001 | |
| Flumethazone Pivalate | 0 | 101 | 1 | 94 | 3 | Q2 | HIT | <0.001 | 0.004 | |
| Flunisolide | 0 | 105 | 2 | 102 | 5 | Q2 | HIT | <0.001 | <0.001 | |
| Fusidic Acid | 0 | 77 | 1 | 74 | 2 | Q2 | HIT | 19 | >20 | |
| Gentamicin Sulfate | 0 | 105 | 1 | 101 | 4 | Q2 | HIT | 4 | >20 | |
| Hycanthone | 2 | 103 | 2 | 71 | 2 | Q2 | HIT | 1 | 1 | |
| Hydrocortisone Valerate | 0 | 95 | 1 | 73 | 1 | Q2 | HIT | 0.05 | 1 | |
| Ibudilast | 0 | 75 | 1 | 57 | 1 | Q2 | HIT | >20 | >20 | |
| Mebendazole | 0 | 57 | 1 | 72 | 2 | Q2 | HIT | 0.2 | 6 | |
| Mechlorethamine | 0 | 77 | 1 | 125 | 68 | Q2 | HIT | 7 | 0.1 | |
| Medroxyprogesterone Acetate | 0 | 72 | 1 | 56 | 1 | Q2 | HIT | 0.5 | >20 | |
| Medroxyprogesterone Acetate | 0 | 57 | 1 | 54 | 1 | Q2 | HIT | 0.3 | >20 | |
| Melengestrol Acetate | 0 | 86 | 2 | 67 | 2 | Q2 | HIT | 0.07 | 13 | |
| Mequinol | 0 | 98 | 2 | 76 | 1 | Q2 | HIT | >20 | >20 | |
| Methylphenidate Hydrochloride | 0 | 81 | 2 | 75 | 1 | Q2 | HIT | >20 | >20 | |
| Midodrine Hydrochloride | 0 | 95 | 3 | 52 | 1 | Q2 | HIT | >20 | >20 | |
| Molsidomine | 0 | 103 | 2 | 124 | 5 | Q2 | HIT | >20 | >20 | |
| Monensin Sodium | 0 | 101 | 3 | 117 | 6 | Q2 | HIT | 0.004 | 0.2 | |
| Mycophenolate Mofetil | 0 | 94 | 2 | 79 | 2 | Q2 | HIT | 2 | 1 | |
| Nortriptyline Hydrochloride | 1 | 98 | 2 | 114 | 81 | Q2 | HIT | 13 | 3 | |
| Perhexiline Maleate | 0 | 99 | 3 | 123 | 69 | Q2 | HIT | 8 | 3 | |
| Phenytoin Sodium | 0 | 82 | 1 | 63 | 1 | Q2 | HIT | >20 | >20 | |
| Pitavastatin Calcium | 0 | 100 | 1 | 66 | 1 | Q2 | HIT | 1 | 5 | |
| Prednicarbate | 0 | 61 | 1 | 73 | 1 | Q2 | HIT | 0.002 | >20 | |
| Prednisolone Sodium Phosphate | 0 | 108 | 3 | 96 | 3 | Q2 | HIT | 0.1 | 4 | |
| Rapamycin | 0 | 86 | 3 | 53 | 2 | Q2 | HIT | <0.001 | <0.001 | |
| Rebamipide | 0 | 65 | 1 | 72 | 2 | Q2 | HIT | 2 | 11 | |
| Ribavirin | 0 | 102 | 2 | 107 | 3 | Q2 | HIT | 1 | 2 | |
| Rosuvastatin | 0 | 93 | 2 | 95 | 4 | Q2 | HIT | 0.3 | 3 | |
| Salicin | 0 | 76 | 1 | 61 | 1 | Q2 | HIT | >20 | >20 | |
| Salinomycin, Sodium | 0 | 100 | 3 | 101 | 1 | Q2 | HIT | 2 | 2 | |
| Saquinavir | 0 | 51 | 1 | 56 | 1 | Q2 | HIT | >20 | >20 | |
| Securinine | 0 | 104 | 2 | 106 | 1 | Q2 | HIT | 2 | 6 | |
| Sulfaquinoxaline Sodium | 0 | 106 | 3 | 104 | 3 | Q2 | HIT | >20 | >20 | |
| Tegaserod Maleate | 0 | 100 | 2 | 86 | 2 | Q2 | HIT | 1 | 19 | |
| Tepoxalin | 0 | 76 | 1 | 66 | 2 | Q2 | HIT | 3 | 18 | |
| Thiabendazole | 0 | 58 | 1 | 62 | 1 | Q2 | HIT | >20 | >20 | |
| Tolnaftate | 0 | 76 | 1 | 60 | 1 | Q2 | HIT | 2 | 17 | |
| Toltrazuril | 0 | 104 | 3 | 90 | 2 | Q2 | HIT | >20 | >20 | |
| Triamcinolone | 0 | 107 | 3 | 99 | 4 | Q2 | HIT | 0.04 | 0.4 | |
| Trimipramine Maleate | 0 | 52 | 1 | 92 | 5 | Q2 | HIT | 7 | 5 | |
| Tyrothricin | 0 | 89 | 2 | 102 | 2 | Q2 | HIT | 3 | 0.5 | |
| Xylazine Hydrochloride | 0 | 102 | 1 | 72 | 2 | Q2 | HIT | 2 | 8 | |
| Albendazole | 0 | 64 | 1 | 16 | 1 | Q3 | HIT | 4 | 6 | |
| Amisulpride | 0 | 54 | 2 | 31 | 1 | Q3 | HIT | 20 | >20 | |
| Amoxapine | 0 | 58 | 0 | 43 | 1 | Q3 | HIT | 20 | 11 | |
| Artesunate | 0 | 63 | 1 | 20 | 0 | Q3 | HIT | 7 | >20 | |
| Bacitracin | 0 | 82 | 1 | 21 | 0 | Q3 | HIT | >20 | >20 | |
| Betazole Hydrochloride | 0 | 59 | 1 | 38 | 1 | Q3 | HIT | 12 | >20 | |
| Chlormadinone Acetate | 0 | 72 | 1 | 43 | 0 | Q3 | HIT | 17 | >20 | |
| Chlorthalidone | 0 | 77 | 2 | 35 | 1 | Q3 | HIT | >20 | >20 | |
| Cloperastine Hydrochloride | 0 | 64 | 2 | 44 | 0 | Q3 | HIT | 7 | 9 | |
| Cyclosporin A | 0 | 62 | 1 | 8 | 0 | Q3 | HIT | 20 | 19 | |
| Cyproterone | 0 | 51 | 1 | 5 | 0 | Q3 | HIT | 17 | >20 | |
| Demeclocycline Hydrochloride | 0 | 75 | 1 | 31 | 1 | Q3 | HIT | 6 | 15 | |
| Desoxycorticosterone Acetate | 0 | 83 | 1 | 47 | 1 | Q3 | HIT | 0.5 | 5 | |
| Diflorasone Diacetate | 0 | 76 | 1 | 39 | 1 | Q3 | HIT | 0.01 | >20 | |
| Docetaxel | 0 | 64 | 2 | 32 | 1 | Q3 | HIT | 20 | >20 | |
| Docetaxel | 0 | 78 | 1 | 32 | 1 | Q3 | HIT | 0.002 | >20 | |
| Ethacrynic Acid | 0 | 57 | 2 | 17 | 1 | Q3 | HIT | >20 | >20 | |
| Ethinyl Estradiol | 0 | 58 | 1 | 37 | 2 | Q3 | HIT | >20 | >20 | |
| Fenbendazole | 0 | 75 | 1 | 10 | 1 | Q3 | HIT | 3 | 4 | |
| Fluorouracil | 0 | 56 | 1 | 40 | 1 | Q3 | HIT | 7 | >20 | |
| Ftaxilide | 0 | 78 | 2 | 42 | 1 | Q3 | HIT | >20 | >20 | |
| Hydroxytoluic Acid | 0 | 69 | 1 | 33 | 0 | Q3 | HIT | 20 | >20 | |
| Idazoxan Hydrochloride | 0 | 70 | 2 | 14 | 0 | Q3 | HIT | 0.4 | 2 | |
| Imipenem | 0 | 53 | 1 | 20 | 0 | Q3 | HIT | >20 | >20 | |
| Iopanic Acid | 0 | 51 | 1 | 35 | 0 | Q3 | HIT | 5 | >20 | |
| Irbesartan | 0 | 66 | 2 | 31 | 1 | Q3 | HIT | >20 | >20 | |
| Isoxicam | 0 | 67 | 1 | 30 | 1 | Q3 | HIT | 7 | >20 | |
| Levonordefrin | 2 | 70 | 1 | 36 | 0 | Q3 | HIT | 0.9 | >20 | |
| Medroxyprogesterone | 0 | 61 | 2 | 6 | 0 | Q3 | HIT | >20 | 8 | |
| Megestrol Acetate | 0 | 66 | 1 | 24 | 0 | Q3 | HIT | 5 | >20 | |
| Mephentermine Sulfate | 0 | 60 | 2 | 31 | 1 | Q3 | HIT | >20 | >20 | |
| Metampicillin Sodium | 0 | 68 | 1 | 35 | 0 | Q3 | HIT | 20 | >20 | |
| Miconazole Nitrate | 0 | 53 | 1 | 23 | 0 | Q3 | HIT | 20 | >20 | |
| Milnacipran Hydrochloride | 0 | 72 | 1 | 31 | 1 | Q3 | HIT | 8 | 20 | |
| Mirtazapine | 0 | 57 | 2 | 36 | 1 | Q3 | HIT | >20 | >20 | |
| Naftopidil Dihydrochloride | 0 | 59 | 1 | 32 | 0 | Q3 | HIT | 7 | >20 | |
| Oxaliplatin | 0 | 96 | 7 | 41 | 1 | Q3 | HIT | 5 | 15 | |
| Oxiglutatione Disodium Salt | 0 | 84 | 1 | 38 | 1 | Q3 | HIT | >20 | >20 | |
| Oxybendazole | 0 | 78 | 2 | 15 | 1 | Q3 | HIT | 7 | 12 | |
| Pimozide | 0 | 70 | 2 | 45 | 0 | Q3 | HIT | >20 | 18 | |
| Prednisone | 0 | 66 | 1 | 45 | 1 | Q3 | HIT | 3 | >20 | |
| Prednisone | 0 | 74 | 2 | 38 | 0 | Q3 | HIT | 12 | >20 | |
| R(−)-Apomorphine Hydrochloride | 2 | 63 | 1 | 18 | 0 | Q3 | HIT | >20 | 14 | |
| Ractopamine Hydrochloride | 0 | 50 | 1 | 39 | 0 | Q3 | HIT | 20 | >20 | |
| Rasagiline | 0 | 76 | 1 | 28 | 1 | Q3 | HIT | 20 | >20 | |
| Risedronate Sodium | 0 | 81 | 2 | 16 | 0 | Q3 | HIT | 19 | >20 | |
| Ritodrine Hydrochloride | 0 | 51 | 1 | 36 | 1 | Q3 | HIT | 0.6 | >20 | |
| Rubitecan | 0 | 73 | 2 | 30 | 0 | Q3 | HIT | 2 | 9 | |
| Salmeterol | 0 | 56 | 1 | 29 | 1 | Q3 | HIT | 1 | 14 | |
| Selamectin | 0 | 51 | 1 | 42 | 1 | Q3 | HIT | 6 | >20 | |
| Sulbactam | 0 | 51 | 1 | 33 | 0 | Q3 | HIT | >20 | >20 | |
| Tolazamide | 0 | 88 | 1 | 42 | 0 | Q3 | HIT | 12 | >20 | |
| Trifluoperazine Dihydrochloride | 1 | 69 | 2 | 26 | 0 | Q3 | HIT | 12 | 6 | |
| Zoledronic Acid | 0 | 83 | 2 | 49 | 1 | Q3 | HIT | 2 | 8 | |
| Desipramine hydrochloride | 0 | −2 | 1 | 83 | 4 | Q1 | | | | KEGG |
| Maprotiline hydrochloride | 0 | 31 | 1 | 67 | 1 | Q1 | | | | KEGG |
| Colchicine | 0 | 114 | 12 | 113 | 10 | Q2 | | | | KEGG |
| Dexamethasone Acetate | 0 | 112 | 4 | 105 | 7 | Q2 | | | | KEGG |
| Fluocinonide | 0 | 112 | 3 | 106 | 6 | Q2 | | | | KEGG |
| Fluorometholone | 0 | 104 | 6 | 84 | 1 | Q2 | | | | KEGG |
| Flurandrenolide | 0 | 110 | 6 | 98 | 5 | Q2 | | | | KEGG |
| Hydrocortisone Acetate | 0 | 112 | 4 | 89 | 4 | Q2 | | | | KEGG |
| Podofilox/Podophyllotoxin | 0 | 110 | 8 | 107 | 9 | Q2 | | | | KEGG |
| Prednisolone | 0 | 114 | 4 | 100 | 4 | Q2 | | | | KEGG |
| Prednisolone Acetate | 0 | 109 | 4 | 111 | 6 | Q2 | | | | KEGG |
| Thioridazine hydrochloride | 1 | 116 | 72 | 118 | 73 | Q2 | | | | KEGG |
| Fendiline hydrochloride | 0 | −33 | 1 | 10 | 0 | Q4 | | | | KEGG |
| Nylidrin hydrochloride | 0 | 6 | 0 | 24 | 1 | Q4 | | | | KEGG |
| Prochlorperazine | 1 | 6 | 0 | 17 | 1 | Q4 | | | | KEGG |
| Tannic Acid | 2 | −60 | 0 | 32 | 1 | Q4 | | | | KEGG |
| Amlodipine | 0 | −29 | 0 | 59 | 2 | Q1 | | | | |
| Amodiaquine Dihydrochloride | 3 | 4 | 1 | 114 | 85 | Q1 | | | | |
| Astermizole | 0 | 10 | 1 | 69 | 2 | Q1 | | | | |
| Bronopol | 0 | 43 | 2 | 119 | 61 | Q1 | | | | |
| Chlorhexidine, Chlorhexidine | 0 | 15 | 1 | 69 | 8 | Q1 | | | | |
| Dihydrochloride | | | | | | | | | | |
| Chlormidazole | 0 | 38 | 1 | 70 | 2 | Q1 | | | | |
| Colistin Sulfate | 0 | 19 | 1 | 58 | 1 | Q1 | | | | |
| Deslanoside | 0 | −3 | 1 | 67 | 1 | Q1 | | | | |
| Fluoxetine Hydrochloride, Fluoxetine | 0 | 46 | 1 | 65 | 2 | Q1 | | | | |
| Imipramine | 0 | −4 | 0 | 60 | 1 | Q1 | | | | |
| Iodamide | 0 | 41 | 0 | 67 | 1 | Q1 | | | | |
| Loperamide | 0 | 12 | 1 | 63 | 2 | Q1 | | | | |
| Maprotiline | 0 | −4 | 1 | 78 | 2 | Q1 | | | | |
| Mesoridazine | 1 | −5 | 0 | 66 | 2 | Q1 | | | | |
| Methylene Blue | 3 | 25 | 1 | 73 | 1 | Q1 | | | | |
| Oxyphenbutazone | 1 | 2 | 1 | 114 | 97 | Q1 | | | | |
| Phenacemide | 0 | −9 | 1 | 66 | 1 | Q1 | | | | |
| Polymyxin B Sulfate | 0 | −25 | 1 | 76 | 2 | Q1 | | | | |
| Polymyxin B1 | 0 | −21 | 1 | 60 | 1 | Q1 | | | | |
| Prazosin | 0 | 43 | 3 | 118 | 77 | Q1 | | | | |
| Promazine | 1 | 46 | 1 | 117 | 64 | Q1 | | | | |
| Quinacrine | 2 | 24 | 1 | 118 | 65 | Q1 | | | | |
| Raloxifene | 0 | 7 | 0 | 60 | 1 | Q1 | | | | |
| Reserpine | 3 | −4 | 3 | 64 | 7 | Q1 | | | | |
| Sanguinarine Chloride, Sanguinarine | 0 | 24 | 0 | 101 | 55 | Q1 | | | | |
| Sulfate | | | | | | | | | | |
| Tamsulosin | 0 | 20 | 1 | 59 | 2 | Q1 | | | | |
| Tilorone | 0 | −21 | 0 | 60 | 1 | Q1 | | | | |
| Trichlormethine, Trichlormethine | 0 | −10 | 1 | 96 | 31 | Q1 | | | | |
| Hydrochloride | | | | | | | | | | |
| Acarbose | 0 | 81 | 11 | 62 | 5 | Q2 | | | | |
| Acriflavinium Hydrochloride | 2 | 110 | 57 | 129 | 63 | Q2 | | | | |
| Alexidine Hydrochloride | 0 | 108 | 5 | 120 | 9 | Q2 | | | | |
| Alprostadil | 0 | 100 | 9 | 87 | 3 | Q2 | | | | |
| Amcinonide | 0 | 112 | 7 | 119 | 6 | Q2 | | | | |
| Aminacrine | 2 | 117 | 72 | 118 | 67 | Q2 | | | | |
| Amsacrine | 0 | 70 | 3 | 120 | 54 | Q2 | | | | |
| Auranofin | 0 | 106 | 94 | 106 | 98 | Q2 | | | | |
| Benzalkonium Chloride | 0 | 112 | 71 | 122 | 31 | Q2 | | | | |
| Benzethonium Chloride | 0 | 111 | 85 | 114 | 86 | Q2 | | | | |
| Benzoylpas | 0 | 107 | 4 | 87 | 2 | Q2 | | | | |
| Betamethasone Acetate | 0 | 107 | 3 | 100 | 4 | Q2 | | | | |
| Bismuth Subsalicylate | 0 | 93 | 8 | 98 | 27 | Q2 | | | | |
| Bopindolol | 0 | 105 | 6 | 81 | 2 | Q2 | | | | |
| Bortezomib | 0 | 106 | 86 | 106 | 56 | Q2 | | | | |
| Budesonide | 0 | 110 | 3 | 108 | 5 | Q2 | | | | |
| Budesonide | 0 | 105 | 6 | 79 | 1 | Q2 | | | | |
| Cepharanthine | 0 | 114 | 85 | 130 | 58 | Q2 | | | | |
| Cerivastatin, Cerivastatin Na | 0 | 103 | 13 | 101 | 5 | Q2 | | | | |
| Cetrimonium | 0 | 114 | 28 | 132 | 12 | Q2 | | | | |
| Cetylpyridinium Bromide | 0 | 112 | 25 | 114 | 39 | Q2 | | | | |
| Cinoctramide | 0 | 90 | 4 | 53 | 1 | Q2 | | | | |
| Cinoxacin | 0 | 117 | 50 | 115 | 12 | Q2 | | | | |
| Compactin, Mevastatin | 0 | 105 | 3 | 89 | 2 | Q2 | | | | |
| Cycloheximide | 0 | 108 | 45 | 123 | 45 | Q2 | | | | |
| Dactinomycin | 3 | 106 | 88 | 106 | 50 | Q2 | | | | |
| Dactinomycin | 3 | 112 | 75 | 114 | 66 | Q2 | | | | |
| Danazol | 0 | 105 | 28 | 99 | 14 | Q2 | | | | |
| Dasatinib | 0 | 109 | 14 | 70 | 10 | Q2 | | | | |
| Dasatinib | 0 | 105 | 65 | 64 | 8 | Q2 | | | | |
| Daunorubicin | 3 | 116 | 77 | 114 | 57 | Q2 | | | | |
| Desonide | 0 | 108 | 17 | 112 | 5 | Q2 | | | | |
| Desoximetasone | 0 | 101 | 5 | 91 | 3 | Q2 | | | | |
| Desoxymetasone | 0 | 108 | 3 | 105 | 3 | Q2 | | | | |
| Dexamethasone | 0 | 112 | 4 | 111 | 6 | Q2 | | | | |
| Dexamethasone Phosphate | 0 | 103 | 5 | 93 | 2 | Q2 | | | | |
| Dinoprostone | 0 | 102 | 9 | 85 | 2 | Q2 | | | | |
| Dipyrone | 0 | 106 | 26 | 101 | 36 | Q2 | | | | |
| Disulfiram | 0 | 114 | 90 | 131 | 75 | Q2 | | | | |
| Doxorubicin | 3 | 106 | 75 | 108 | −1 | Q2 | | | | |
| Doxorubicin | 3 | 113 | 82 | 118 | 32 | Q2 | | | | |
| Emetine, Emetine Dihydrochloride | 0 | 117 | 82 | 115 | 46 | Q2 | | | | |
| Enoxacin | 0 | 113 | 84 | 119 | 12 | Q2 | | | | |
| Epirubicin, Epirubicin Hydrochloride | 3 | 113 | 79 | 123 | 58 | Q2 | | | | |
| Estrone | 0 | 106 | 93 | 106 | 25 | Q2 | | | | |
| Ethylmercurithiosalicylic acid | 0 | 109 | 92 | 127 | 95 | Q2 | | | | |
| Flunisolide | 0 | 104 | 5 | 101 | 4 | Q2 | | | | |
| Fluocinonide | 0 | 103 | 4 | 88 | 2 | Q2 | | | | |
| Fluorometholone | 0 | 112 | 3 | 106 | 7 | Q2 | | | | |
| Flurandrenolide | 0 | 102 | 5 | 103 | 5 | Q2 | | | | |
| Fluticasone Propionate | 0 | 108 | 7 | 117 | 3 | Q2 | | | | |
| Fluticasone Propionate | 0 | 99 | 5 | 96 | 4 | Q2 | | | | |
| Fluvastatin | 0 | 110 | 10 | 101 | 3 | Q2 | | | | |
| Fluvastatin | 0 | 102 | 12 | 102 | 7 | Q2 | | | | |
| Gentian Violet | 3 | 117 | 38 | 118 | 73 | Q2 | | | | |
| Gramicidin | 0 | 114 | 43 | 118 | 64 | Q2 | | | | |
| Halcinonide | 0 | 103 | 8 | 95 | 2 | Q2 | | | | |
| Haloprogin | 0 | 78 | 42 | 106 | 70 | Q2 | | | | |
| Hexamethonium | 0 | 108 | 9 | 100 | 2 | Q2 | | | | |
| Homidium Bromide | 2 | 84 | 17 | 103 | 9 | Q2 | | | | |
| Homoharringtonie | 0 | 106 | 90 | 106 | 67 | Q2 | | | | |
| Hydrocortisone | 0 | 104 | 4 | 57 | 1 | Q2 | | | | |
| Idarubicin, Idarubicin Hydrochloride | 3 | 106 | 90 | 106 | 40 | Q2 | | | | |
| Inosine | 0 | 111 | 7 | 115 | 2 | Q2 | | | | |
| Isoflupredone Acetate | 0 | 106 | 3 | 90 | 2 | Q2 | | | | |
| Isotretinoin | 0 | 112 | 13 | 55 | 1 | Q2 | | | | |
| Lovastatin | 0 | 106 | 3 | 87 | 2 | Q2 | | | | |
| Medrysone | 0 | 111 | 4 | 87 | 4 | Q2 | | | | |
| Menadione | 3 | 113 | 87 | 123 | 62 | Q2 | | | | |
| Mestranol | 0 | 106 | 90 | 90 | 27 | Q2 | | | | |
| Methylbenzethonium Chloride | 0 | 105 | 17 | 108 | 10 | Q2 | | | | |
| Mitomycin C, Mitomycin | 3 | 112 | 74 | 110 | 49 | Q2 | | | | |
| Mitoxantrone | 3 | 113 | 88 | 122 | 40 | Q2 | | | | |
| Mometasone Furoate | 0 | 100 | 3 | 106 | 4 | Q2 | | | | |
| Nadolol | 0 | 107 | 4 | 108 | 3 | Q2 | | | | |
| Naltrexone Hydrochloride | 0 | 107 | 4 | 79 | 2 | Q2 | | | | |
| Nifenazone | 0 | 114 | 85 | 132 | 78 | Q2 | | | | |
| Nifuroxazide | 0 | 112 | 6 | 120 | 14 | Q2 | | | | |
| Ondansetron | 0 | 106 | 20 | 92 | 3 | Q2 | | | | |
| Oxyquinoline | 0 | 117 | 80 | 116 | 57 | Q2 | | | | |
| Phenylmercuric Acetate | 0 | 109 | 82 | 127 | 80 | Q2 | | | | |
| Pimethixene Maleate | 1 | 112 | 6 | 92 | 2 | Q2 | | | | |
| Piperacillin | 0 | 103 | 6 | 80 | 2 | Q2 | | | | |
| Prednisolone | 0 | 97 | 3 | 87 | 3 | Q2 | | | | |
| Prednisolone Hemisuccinate | 0 | 106 | 3 | 96 | 3 | Q2 | | | | |
| Proflavine | 0 | 113 | 82 | 132 | 59 | Q2 | | | | |
| Puromycin Hydrochloride, | 0 | 107 | 66 | 124 | 52 | Q2 | | | | |
| Puromycin Dihydrochloride | | | | | | | | | | |
| Pyrithione Zinc | 0 | 109 | 86 | 127 | 87 | Q2 | | | | |
| Pyrvinium | 2 | 113 | 24 | 115 | 45 | Q2 | | | | |
| Rimantadine | 0 | 106 | 93 | 106 | 56 | Q2 | | | | |
| Simvastatin | 0 | 108 | 7 | 102 | 4 | Q2 | | | | |
| Sirolimus | 0 | 108 | 4 | 81 | 1 | Q2 | | | | |
| Sunitinib | 0 | 106 | 75 | 101 | 27 | Q2 | | | | |
| Tetramizole Hydrochloride | 0 | 107 | 8 | 112 | 5 | Q2 | | | | |
| Thiostrepton | 0 | 109 | 64 | 65 | 1 | Q2 | | | | |
| Thiram | 0 | 109 | 91 | 126 | 64 | Q2 | | | | |
| Topotecan, Topotecan | 3 | 112 | 63 | 112 | 40 | Q2 | | | | |
| Hydrochloride | | | | | | | | | | |
| Triamcinolone Acetonide | 0 | 111 | 4 | 104 | 4 | Q2 | | | | |
| Vincristine | 0 | 105 | 33 | 104 | 10 | Q2 | | | | |
| Vincristine Sulfate | 0 | 108 | 16 | 119 | 7 | Q2 | | | | |
| Vindesine, Vindesine Sulfate | 0 | 106 | 30 | 102 | 9 | Q2 | | | | |
| Vinorelbine | 0 | 108 | 3 | 106 | 9 | Q2 | | | | |
| Vorinostat | 0 | 112 | 89 | 112 | 50 | Q2 | | | | |
| Digoxin | 0 | 99 | 4 | 26 | 1 | Q3 | | | | |
| Oxaliplatin | 0 | 66 | 0 | 21 | 0 | Q3 | | | | |
| Suloctidil | 0 | 112 | 90 | 3 | 0 | Q3 | | | | |
| (−)-ephedrine, N-methyl, N- | 0 | −52 | 0 | 7 | 0 | Q4 | | | | |
| methyl (−)ephedrine [1r,2s] | | | | | | | | | | |
| (¬±)-atenolol, Atenolol | 0 | 6 | 0 | −7 | 0 | Q4 | | | | |
| (¬±)-baclofen, Baclofen | 0 | −8 | 0 | 9 | 0 | Q4 | | | | |
| (¬±)-gamma-vinyl Gaba, Vigabatrin | 0 | 8 | 1 | 12 | 0 | Q4 | | | | |
| (¬±)-ibuprofen, Ibuprofen | 0 | −20 | 0 | 16 | 0 | Q4 | | | | |
| (¬±)-octopamine Hydrochloride, | 0 | −48 | 1 | 16 | 0 | Q4 | | | | |
| Octopamine Hydrochloride | | | | | | | | | | |
| (¬±)-p-aminoglutethimide, | 0 | 3 | 0 | 6 | 0 | Q4 | | | | |
| Aminoglutethimide | | | | | | | | | | |
| (¬±)-p-chlorophenylalanine, | 0 | 16 | 1 | 5 | 0 | Q4 | | | | |
| Fenclonine | | | | | | | | | | |
| (r)-bicalutamide, Bicalutamide | 0 | −1 | 0 | 20 | 0 | Q4 | | | | |
| 1-(Isopropylamino)-3-(1- | 0 | −18 | 1 | −5 | 0 | Q4 | | | | |
| Naphthyloxy)-2-Propanol | | | | | | | | | | |
| 2-Aminoethanesulfonic Acid | 0 | −13 | 0 | 19 | 0 | Q4 | | | | |
| 2-Phenyl-Ethanol | 0 | −6 | 1 | 15 | 0 | Q4 | | | | |
| 2-thiouracil | 0 | −15 | 1 | 7 | 0 | Q4 | | | | |
| 2,6-di-t-butyl-4-methylphenol, | 0 | −4 | 1 | 5 | 0 | Q4 | | | | |
| Butylated Hydroxytoluene | | | | | | | | | | |
| 2H-1-BENZOPYRAN-2-ONE | 0 | −35 | 0 | 13 | 0 | Q4 | | | | |
| 3-acetamidophenol | 0 | 4 | 1 | 5 | 0 | Q4 | | | | |
| 3-Hydroxy-3-Methyl-Glutaric Acid | 0 | 9 | 0 | 8 | 0 | Q4 | | | | |
| 3-methyl-1-phenyl-2-pyrazolin-5-one | 0 | −15 | 1 | 15 | 0 | Q4 | | | | |
| (mci-186), Edaravone | | | | | | | | | | |
| 4-acetamidobenzoic acid | 0 | 32 | 1 | 42 | 0 | Q4 | | | | |
| 4-Aminobenzoic Acid | 0 | −31 | 1 | 13 | 1 | Q4 | | | | |
| 5-azacytidine, Azacitidine | 0 | 9 | 1 | −7 | 0 | Q4 | | | | |
| 5-fluoro-5′-deoxyuridine, | 0 | −27 | 1 | 9 | 0 | Q4 | | | | |
| Doxifluridine | | | | | | | | | | |
| 7-hydroxy-4-methyl-2H-chromen-2- | 0 | 12 | 0 | 4 | 0 | Q4 | | | | |
| one | | | | | | | | | | |
| Abacavir Sulfate | 0 | −2 | 1 | 3 | 0 | Q4 | | | | |
| Acamprosate Calcium | 0 | 12 | 1 | 22 | 0 | Q4 | | | | |
| Acarbose | 0 | −22 | 1 | −5 | 0 | Q4 | | | | |
| Acebutolol | 0 | −11 | 1 | 19 | 0 | Q4 | | | | |
| Aceclidine Hydrochloride, Aceclidine | 0 | 2 | 1 | 0 | 0 | Q4 | | | | |
| Aceclofenac | 0 | −2 | 1 | 8 | 0 | Q4 | | | | |
| Acedapsone | 0 | 0 | 0 | 4 | 0 | Q4 | | | | |
| Aceglutamide | 0 | 36 | 1 | 20 | 0 | Q4 | | | | |
| Acemetacin | 0 | −16 | 1 | 17 | 0 | Q4 | | | | |
| Acenocoumarol, Acenocoumarin | 0 | −38 | 0 | 14 | 0 | Q4 | | | | |
| Acepromazine | 1 | 9 | 1 | 31 | 1 | Q4 | | | | |
| Acesulfame Potassium | 0 | −3 | 0 | 16 | 0 | Q4 | | | | |
| Acetaminophen | 0 | 14 | 0 | 22 | 0 | Q4 | | | | |
| Acetaminosalol | 0 | −15 | 1 | 20 | 1 | Q4 | | | | |
| Acetanilide | 0 | −31 | 0 | 9 | 0 | Q4 | | | | |
| Acetarsol | 0 | −12 | 0 | 13 | 0 | Q4 | | | | |
| Acetazolamide | 0 | −12 | 0 | 4 | 1 | Q4 | | | | |
| Acetohexamide | 0 | 12 | 1 | 14 | 0 | Q4 | | | | |
| Acetohydroxamic Acid | 0 | −3 | 0 | 12 | 0 | Q4 | | | | |
| Acetophenazine Maleate | 0 | 34 | 1 | 15 | 0 | Q4 | | | | |
| Acetrizoic acid | 0 | 14 | 1 | 9 | 0 | Q4 | | | | |
| Acetyl-l-leucine | 0 | −10 | 5 | 45 | 3 | Q4 | | | | |
| Acetylcholine | 0 | −39 | 0 | 9 | 0 | Q4 | | | | |
| Acetylcysteine | 0 | −19 | 0 | 7 | 0 | Q4 | | | | |
| Acetylcysteine | 0 | −30 | 1 | 13 | 0 | Q4 | | | | |
| Acetylsalicylic acid | 0 | −21 | 1 | 17 | 0 | Q4 | | | | |
| Acexamic Acid | 0 | −25 | 1 | 13 | 0 | Q4 | | | | |
| Aciclovir | 0 | −20 | 1 | 3 | 1 | Q4 | | | | |
| Acipimox | 0 | −11 | 1 | 20 | 0 | Q4 | | | | |
| Aconitine | 0 | −23 | 0 | 12 | 0 | Q4 | | | | |
| Actarit | 0 | 11 | 1 | 9 | 0 | Q4 | | | | |
| Adapalene | 0 | −12 | 2 | 45 | 4 | Q4 | | | | |
| Adenine | 0 | −7 | 1 | 22 | 1 | Q4 | | | | |
| Adenosine | 0 | −6 | 1 | 3 | 1 | Q4 | | | | |
| Adenosine monophosphate | 0 | −30 | 1 | 4 | 0 | Q4 | | | | |
| Adenosine Phosphate | 0 | 6 | 1 | 31 | 2 | Q4 | | | | |
| Adiphenine Hydrochloride | 0 | −30 | 1 | 12 | 0 | Q4 | | | | |
| Adipic Acid, Hexanedioic Acid | 0 | −24 | 0 | 8 | 0 | Q4 | | | | |
| Adrenaline Bitartrate, Epinephrine | 2 | 35 | 0 | 19 | 0 | Q4 | | | | |
| Bitartrate | | | | | | | | | | |
| Adrenolone Hydrochloride | 2 | 28 | 0 | 32 | 0 | Q4 | | | | |
| Afalanine | 0 | 0 | 1 | 10 | 0 | Q4 | | | | |
| Ajmaline | 0 | −6 | 1 | 13 | 0 | Q4 | | | | |
| Aklomide | 0 | −18 | 0 | −2 | 0 | Q4 | | | | |
| Alaproclate Hydrochloride, | 0 | −7 | 1 | 15 | 0 | Q4 | | | | |
| Alaproclate | | | | | | | | | | |
| Alendronic acid | 0 | 5 | 1 | 18 | 0 | Q4 | | | | |
| Alfacalcidol | 0 | −26 | 1 | 4 | 0 | Q4 | | | | |
| Alfuzosin Hydrochloride, Alfuzosin | 0 | 1 | 1 | 18 | 0 | Q4 | | | | |
| Alimemazine | 1 | 32 | 1 | 39 | 0 | Q4 | | | | |
| Aliskiren | 0 | 2 | 1 | 21 | 0 | Q4 | | | | |
| Allantoin | 0 | −21 | 1 | 6 | 0 | Q4 | | | | |
| Allopurinol | 0 | −17 | 1 | 16 | 0 | Q4 | | | | |
| Allylisothiocyanate | 0 | 11 | 1 | 18 | 0 | Q4 | | | | |
| Allylthiourea | 0 | −7 | 1 | 11 | 0 | Q4 | | | | |
| Aloin | 0 | −35 | 1 | 14 | 0 | Q4 | | | | |
| Alpha-cypermethrin | 0 | −15 | 1 | 11 | 0 | Q4 | | | | |
| Alpha-tochopherol | 0 | 18 | 0 | 21 | 0 | Q4 | | | | |
| Alpha-tochopheryl Acetate | 0 | 22 | 1 | 8 | 0 | Q4 | | | | |
| Alprazolam | 0 | 33 | 1 | 20 | 0 | Q4 | | | | |
| Alprenolol Hydrochloride, Alprenolol | 0 | 1 | 0 | 13 | 1 | Q4 | | | | |
| Alrestatin | 0 | 11 | 1 | 12 | 0 | Q4 | | | | |
| Althiazide | 0 | −11 | 1 | 12 | 1 | Q4 | | | | |
| Altrenogest | 0 | 6 | 1 | 15 | 0 | Q4 | | | | |
| Altretamine | 0 | −29 | 1 | 16 | 0 | Q4 | | | | |
| Alverine | 0 | −17 | 0 | 6 | 0 | Q4 | | | | |
| Amantadine | 0 | −7 | 1 | 3 | 0 | Q4 | | | | |
| Amantadine | 0 | −32 | 1 | −5 | 0 | Q4 | | | | |
| Ambroxol Hydrochloride | 0 | −19 | 0 | 9 | 0 | Q4 | | | | |
| Amifostine | 0 | −38 | 0 | 15 | 0 | Q4 | | | | |
| Amikacin Sulfate | 0 | 3 | 1 | 1 | 0 | Q4 | | | | |
| Amiloride | 0 | 20 | 1 | 7 | 0 | Q4 | | | | |
| Aminocaproic Acid | 0 | 5 | 1 | 7 | 0 | Q4 | | | | |
| Aminoguanidine | 0 | −25 | 0 | 12 | 0 | Q4 | | | | |
| Aminohippuric Acid | 0 | −7 | 1 | 16 | 1 | Q4 | | | | |
| Aminohydroxybutyric Acid | 0 | −4 | 1 | 17 | 0 | Q4 | | | | |
| Aminolevulinic acid | 0 | −4 | 1 | 10 | 0 | Q4 | | | | |
| Aminopentamide Sulfate | 0 | 3 | 1 | 12 | 0 | Q4 | | | | |
| Aminophenazone | 0 | −41 | 1 | 20 | 1 | Q4 | | | | |
| Aminophylline, Theophylline | 0 | 35 | 1 | 25 | 0 | Q4 | | | | |
| Aminopterin | 0 | −12 | 1 | 17 | 0 | Q4 | | | | |
| Aminosalicylic Acid | 0 | 9 | 0 | 9 | 0 | Q4 | | | | |
| Aminothiazole | 0 | −31 | 1 | 17 | 1 | Q4 | | | | |
| Amiodarone | 0 | −4 | 1 | 13 | 0 | Q4 | | | | |
| Amisulpride | 0 | −5 | 1 | 12 | 0 | Q4 | | | | |
| Amitraz | 0 | −18 | 0 | 9 | 0 | Q4 | | | | |
| Amitriptyline | 1 | 14 | 1 | 30 | 1 | Q4 | | | | |
| Amlexanox | 0 | −17 | 0 | −4 | 0 | Q4 | | | | |
| Amlodipine | 0 | −17 | 0 | 36 | 2 | Q4 | | | | |
| Amorolfine | 0 | 6 | 1 | 22 | 0 | Q4 | | | | |
| Amoxicillin | 0 | −14 | 0 | 12 | 0 | Q4 | | | | |
| Amphotericin B | 0 | −25 | 1 | 29 | 1 | Q4 | | | | |
| Amphotericin B | 0 | −39 | 1 | −6 | 0 | Q4 | | | | |
| Ampicillin | 0 | 1 | 1 | 15 | 0 | Q4 | | | | |
| Ampiroxicam | 0 | −9 | 1 | 14 | 0 | Q4 | | | | |
| Amprenavir | 0 | −10 | 1 | 6 | 0 | Q4 | | | | |
| Amprolium | 0 | −10 | 1 | 8 | 0 | Q4 | | | | |
| Ampyzine Sulfate | 0 | −18 | 0 | 11 | 1 | Q4 | | | | |
| Amrinone, Inamrinone | 0 | 1 | 1 | 18 | 0 | Q4 | | | | |
| Amylene Hydrate | 0 | −18 | 1 | 18 | 1 | Q4 | | | | |
| Anagrelide | 0 | −2 | 1 | 19 | 1 | Q4 | | | | |
| Anagrelide | 0 | −22 | 1 | −2 | 0 | Q4 | | | | |
| Anastrozole | 0 | 31 | 1 | 16 | 0 | Q4 | | | | |
| Ancitabine Hydrochloride | 0 | −4 | 1 | 27 | 1 | Q4 | | | | |
| Anethole | 0 | −37 | 0 | 13 | 0 | Q4 | | | | |
| Aniracetam | 0 | 6 | 1 | 4 | 0 | Q4 | | | | |
| Anisindione | 2 | −15 | 0 | 13 | 0 | Q4 | | | | |
| Anisodamine Hydrobromide | 0 | −41 | 1 | 4 | 0 | Q4 | | | | |
| Antazoline | 0 | −2 | 1 | 17 | 1 | Q4 | | | | |
| Anthralin | 0 | −10 | 1 | 13 | 1 | Q4 | | | | |
| Antipyrine | 0 | −8 | 0 | 13 | 1 | Q4 | | | | |
| Apomorphine Hydrochloride | 2 | 11 | 1 | −4 | 0 | Q4 | | | | |
| Apramycin Sulfate | 0 | 11 | 1 | 25 | 1 | Q4 | | | | |
| Aprepitant | 0 | 20 | 1 | 1 | 0 | Q4 | | | | |
| Arecoline | 0 | 17 | 1 | 14 | 0 | Q4 | | | | |
| Argatroban | 0 | −10 | 1 | 8 | 0 | Q4 | | | | |
| Arginine Hydrochloride | 0 | 9 | 0 | 42 | 2 | Q4 | | | | |
| Aripiprazole | 0 | −21 | 0 | 16 | 0 | Q4 | | | | |
| Aripiprazole | 0 | −48 | 1 | 3 | 0 | Q4 | | | | |
| Armodafinil | 0 | −10 | 0 | 19 | 1 | Q4 | | | | |
| Arsanilic Acid | 0 | −42 | 0 | 12 | 0 | Q4 | | | | |
| Arsenic Trioxide | 0 | 14 | 1 | 14 | 0 | Q4 | | | | |
| Artemether | 0 | −31 | 1 | 0 | 0 | Q4 | | | | |
| Artemether | 0 | −1 | 0 | 4 | 0 | Q4 | | | | |
| Artemisinin | 0 | −25 | 0 | 12 | 0 | Q4 | | | | |
| Artenimol | 0 | −56 | 0 | 11 | 0 | Q4 | | | | |
| Artesunate | 0 | 47 | 1 | 7 | 0 | Q4 | | | | |
| Ascorbic Acid | 0 | −8 | 0 | 12 | 1 | Q4 | | | | |
| Ascorbyl Palmitate | 0 | 38 | 1 | 20 | 0 | Q4 | | | | |
| Aspartame | 0 | −15 | 1 | 7 | 0 | Q4 | | | | |
| Atazanavir | 0 | −17 | 0 | 13 | 1 | Q4 | | | | |
| Atomoxetine Hydrochloride | 0 | 15 | 1 | 7 | 1 | Q4 | | | | |
| Atovaquone | 3 | −15 | 0 | 13 | 0 | Q4 | | | | |
| Atracurium Besylate | 0 | −3 | 0 | 5 | 0 | Q4 | | | | |
| Atracurium Besylate | 0 | 2 | 1 | 28 | 0 | Q4 | | | | |
| Atropine | 0 | −21 | 0 | 4 | 0 | Q4 | | | | |
| Atropine Oxide | 0 | −30 | 1 | 6 | 0 | Q4 | | | | |
| Avermectin B1, Abamectin | 0 | 20 | 1 | 9 | 0 | Q4 | | | | |
| (avermectin B1a Shown) | | | | | | | | | | |
| Avobenzone | 0 | 22 | 1 | 20 | 0 | Q4 | | | | |
| Azaperone | 0 | −4 | 0 | 12 | 0 | Q4 | | | | |
| Azaserine | 3 | 8 | 1 | 2 | 0 | Q4 | | | | |
| Azatadine | 0 | −9 | 1 | 7 | 0 | Q4 | | | | |
| Azathioprine | 0 | −14 | 0 | 16 | 0 | Q4 | | | | |
| Azelaic Acid | 0 | 6 | 0 | 19 | 0 | Q4 | | | | |
| Azelastine, Azelastine Hydrochloride | 0 | 5 | 0 | 11 | 0 | Q4 | | | | |
| Azilsartan medoxomil | 0 | 5 | 0 | 31 | 1 | Q4 | | | | |
| Azithromycin | 0 | −16 | 0 | 11 | 0 | Q4 | | | | |
| Azlocillin | 0 | −14 | 1 | 6 | 1 | Q4 | | | | |
| Azlocillin Sodium | 0 | −5 | 0 | 17 | 0 | Q4 | | | | |
| Aztreonam | 0 | 3 | 1 | 1 | 0 | Q4 | | | | |
| Aztreonam | 0 | 44 | 0 | 34 | 0 | Q4 | | | | |
| Bacampicillin Hydrochloride | 0 | −12 | 1 | 8 | 0 | Q4 | | | | |
| Bacitracin | 0 | −7 | 0 | 6 | 1 | Q4 | | | | |
| Balsalazide | 3 | −2 | 0 | 11 | 0 | Q4 | | | | |
| Bambuterol | 0 | −24 | 1 | 9 | 0 | Q4 | | | | |
| Bambuterol | 0 | −10 | 1 | 13 | 1 | Q4 | | | | |
| Barbital | 0 | −5 | 0 | 11 | 0 | Q4 | | | | |
| Becanamycin Sulfate, Bekanamycin | 0 | −10 | 1 | 16 | 0 | Q4 | | | | |
| Sulfate | | | | | | | | | | |
| Beclamide | 0 | 19 | 1 | 2 | 0 | Q4 | | | | |
| Beclomethasone | 0 | −8 | 1 | −3 | 0 | Q4 | | | | |
| Beclomethasone Dipropionate | 0 | 7 | 1 | 28 | 1 | Q4 | | | | |
| Bemotrizinol | 0 | −23 | 1 | 16 | 0 | Q4 | | | | |
| Benazepril | 0 | 0 | 1 | 26 | 1 | Q4 | | | | |
| Bendrofumethiazide, | 0 | −15 | 1 | 12 | 1 | Q4 | | | | |
| Bendroflumethiazide | | | | | | | | | | |
| Benfluorex | 0 | −26 | 1 | −3 | 0 | Q4 | | | | |
| Benfotiamine | 0 | −47 | 0 | 18 | 0 | Q4 | | | | |
| Benserazide | 3 | −16 | 0 | 8 | 0 | Q4 | | | | |
| Benurestat | 0 | 6 | 1 | 22 | 1 | Q4 | | | | |
| Benzbromarone | 0 | 0 | 0 | 24 | 1 | Q4 | | | | |
| Benzocaine | 0 | −24 | 1 | 7 | 0 | Q4 | | | | |
| Benzoclidine | 0 | −55 | 1 | 5 | 0 | Q4 | | | | |
| Benzoic Acid | 0 | −6 | 0 | 6 | 0 | Q4 | | | | |
| Benzonatate | 0 | 0 | 1 | 12 | 0 | Q4 | | | | |
| Benzoxiquine | 0 | −2 | 1 | 14 | 0 | Q4 | | | | |
| Benzthiazide | 0 | −8 | 1 | 7 | 0 | Q4 | | | | |
| Benztropine | 0 | −7 | 1 | 30 | 0 | Q4 | | | | |
| Benzydamine | 0 | −16 | 1 | 30 | 1 | Q4 | | | | |
| Benzyl Alcohol | 0 | −54 | 0 | 11 | 0 | Q4 | | | | |
| Benzyl Benzoate | 0 | 35 | 1 | 32 | 1 | Q4 | | | | |
| Benzylpenicillin | 0 | 29 | 1 | 42 | 0 | Q4 | | | | |
| Bephenium Hydroxynapthoate | 0 | 25 | 1 | 19 | 0 | Q4 | | | | |
| Bepridil | 0 | 1 | 1 | 15 | 0 | Q4 | | | | |
| Berberine | 0 | −62 | 0 | 4 | 0 | Q4 | | | | |
| Bergapten | 0 | −5 | 1 | 22 | 0 | Q4 | | | | |
| Beta-carotene | 0 | −15 | 0 | −3 | 0 | Q4 | | | | |
| Beta-escin | 0 | −11 | 0 | 11 | 0 | Q4 | | | | |
| Beta-naphthol | 0 | −17 | 0 | 15 | 0 | Q4 | | | | |
| Beta-propiolactone, Propiolactone | 0 | −12 | 1 | 8 | 0 | Q4 | | | | |
| Betahistine | 0 | −29 | 1 | 13 | 0 | Q4 | | | | |
| Betamethasone | 0 | −4 | 1 | −9 | 0 | Q4 | | | | |
| Betamethasone 17,21-dipropionate | 0 | 21 | 1 | 29 | 0 | Q4 | | | | |
| Betamipron | 0 | −6 | 1 | 14 | 0 | Q4 | | | | |
| Betaxolol | 0 | −42 | 0 | 13 | 0 | Q4 | | | | |
| Betaxolol | 0 | 2 | 1 | 1 | 0 | Q4 | | | | |
| Bethanechol | 0 | 14 | 1 | 15 | 0 | Q4 | | | | |
| Bexarotene | 0 | −3 | 1 | 10 | 1 | Q4 | | | | |
| Bezafibrate | 0 | −4 | 0 | 18 | 0 | Q4 | | | | |
| Bifonazole | 0 | −3 | 1 | 14 | 0 | Q4 | | | | |
| Biotin | 0 | 24 | 1 | 12 | 0 | Q4 | | | | |
| Biperiden | 0 | 2 | 1 | 17 | 1 | Q4 | | | | |
| BIPHENYL-4-YL-ACETALDEHYDE | 0 | −26 | 0 | 7 | 0 | Q4 | | | | |
| Bisacodyl | 0 | 10 | 0 | 8 | 0 | Q4 | | | | |
| Bisoctrizole | 0 | 33 | 0 | 20 | 0 | Q4 | | | | |
| Bisoprolol | 0 | 1 | 0 | 13 | 0 | Q4 | | | | |
| Bithionol | 0 | 11 | 1 | 14 | 0 | Q4 | | | | |
| Bitoscanate | 0 | −9 | 1 | 3 | 1 | Q4 | | | | |
| Bleomycin (bleomycin B2 Shown) | 0 | 27 | 0 | 12 | 0 | Q4 | | | | |
| Bornyl Acetate | 0 | −38 | 0 | 2 | 0 | Q4 | | | | |
| Bosentan | 0 | −14 | 1 | 10 | 0 | Q4 | | | | |
| Bretylium | 0 | −26 | 0 | −9 | 0 | Q4 | | | | |
| Brimonidine | 0 | −36 | 1 | −9 | 0 | Q4 | | | | |
| Brinzolamide | 0 | −3 | 0 | 5 | 0 | Q4 | | | | |
| Bromfenac | 2 | −12 | 1 | 0 | 0 | Q4 | | | | |
| Bromhexine | 0 | −16 | 0 | 11 | 0 | Q4 | | | | |
| Bromindione | 2 | 17 | 1 | 27 | 0 | Q4 | | | | |
| Bromocriptine Mesylate | 0 | 26 | 1 | 24 | 0 | Q4 | | | | |
| Bromopride | 0 | −2 | 1 | 4 | 0 | Q4 | | | | |
| Bromperidol | 0 | −6 | 1 | 7 | 0 | Q4 | | | | |
| Brompheniramine | 0 | −20 | 1 | −7 | −1 | Q4 | | | | |
| Broxaldine | 0 | 19 | 1 | 6 | 0 | Q4 | | | | |
| Broxyquinoline | 0 | −25 | 0 | 8 | 0 | Q4 | | | | |
| Brucine | 0 | 6 | 1 | 13 | 0 | Q4 | | | | |
| Bucetin | 0 | −25 | 0 | 12 | 0 | Q4 | | | | |
| Bucladesine | 0 | 10 | 1 | 24 | 0 | Q4 | | | | |
| Bufexamac | 0 | −2 | 1 | 17 | 0 | Q4 | | | | |
| Buflomedil, Buflomedil Hydrochloride | 0 | −37 | 1 | 13 | 1 | Q4 | | | | |
| Bumetanide | 0 | −3 | 1 | 18 | 1 | Q4 | | | | |
| Bupivacaine | 0 | 12 | 1 | 23 | 0 | Q4 | | | | |
| Bupropion Hydrochloride, | 0 | −15 | 1 | 27 | 2 | Q4 | | | | |
| Bupropion, Amfebutamone | | | | | | | | | | |
| Hydrochloride | | | | | | | | | | |
| Buramate | 0 | 9 | 1 | 17 | 0 | Q4 | | | | |
| Buspirone | 0 | −13 | 0 | 8 | 0 | Q4 | | | | |
| Busulfan | 0 | 1 | 0 | 2 | 0 | Q4 | | | | |
| Butacaine | 0 | −18 | 0 | 3 | 0 | Q4 | | | | |
| Butamben | 0 | −25 | 0 | 14 | 1 | Q4 | | | | |
| Butenafine | 0 | −4 | 1 | −8 | 0 | Q4 | | | | |
| Butoconazole | 0 | −51 | 0 | 10 | 0 | Q4 | | | | |
| Butylated Hydroxyanisole | 0 | −17 | 1 | 26 | 0 | Q4 | | | | |
| Butylparaben | 0 | −16 | 1 | 20 | 0 | Q4 | | | | |
| Cabergoline | 0 | 20 | 1 | 33 | 1 | Q4 | | | | |
| Caffeine | 0 | 2 | 0 | 10 | 0 | Q4 | | | | |
| Calcidiol | 0 | 14 | 1 | 8 | 0 | Q4 | | | | |
| Calcipotriol | 0 | 2 | 1 | −8 | 0 | Q4 | | | | |
| Calcitriol | 0 | −46 | 0 | 0 | 1 | Q4 | | | | |
| Calcium Gluceptate | 0 | 8 | 1 | 8 | 0 | Q4 | | | | |
| Camylofine Dihydrochloride | 0 | −27 | 1 | 15 | 1 | Q4 | | | | |
| Candesartan | 0 | 9 | 1 | 11 | 0 | Q4 | | | | |
| Candesartan Cilextil | 0 | −5 | 1 | 10 | 0 | Q4 | | | | |
| Candicidin | 0 | −41 | 1 | 31 | 0 | Q4 | | | | |
| Canrenoic Acid, Potassium Salt | 0 | −8 | 0 | 8 | 0 | Q4 | | | | |
| Canrenone | 0 | −10 | 1 | 19 | 0 | Q4 | | | | |
| Capecitabine | 0 | 43 | 1 | 25 | 1 | Q4 | | | | |
| Capobenic Acid | 0 | 9 | 1 | 16 | 0 | Q4 | | | | |
| Capreomycin Sulfate | 0 | −32 | 1 | 3 | 0 | Q4 | | | | |
| Capsaicin | 0 | −45 | 1 | 9 | 0 | Q4 | | | | |
| Capsaicin, NGX-4010 | 0 | 5 | 1 | 10 | 0 | Q4 | | | | |
| Captamine | 0 | 9 | 1 | 19 | 0 | Q4 | | | | |
| Captopril | 0 | −11 | 1 | 12 | 1 | Q4 | | | | |
| Carbachol | 0 | −18 | 1 | 6 | 0 | Q4 | | | | |
| Carbadox | 2 | −2 | 1 | 11 | 1 | Q4 | | | | |
| Carbamazepine | 0 | −25 | 0 | 4 | 0 | Q4 | | | | |
| Carbarsone | 0 | −4 | 1 | 17 | 0 | Q4 | | | | |
| Carbenicillin | 0 | −4 | 1 | 4 | 0 | Q4 | | | | |
| Carbenoxolone Sodium | 0 | 16 | 0 | 6 | 0 | Q4 | | | | |
| Carbetapentane Citrate | 0 | −13 | 1 | 7 | 0 | Q4 | | | | |
| Carbimazole | 0 | −32 | 0 | 8 | 0 | Q4 | | | | |
| Carbinoxamine | 0 | 6 | 0 | −3 | 0 | Q4 | | | | |
| Carboplatin | 0 | −23 | 1 | 4 | 0 | Q4 | | | | |
| Carboplatin | 0 | −4 | 1 | 7 | 0 | Q4 | | | | |
| Carglumic Acid | 0 | 1 | 1 | 6 | 0 | Q4 | | | | |
| Carisoprodol | 0 | 1 | 0 | −1 | 0 | Q4 | | | | |
| Carmofur | 0 | −13 | 0 | 17 | 0 | Q4 | | | | |
| Carmustine | 0 | 1 | 1 | 9 | 0 | Q4 | | | | |
| Carnitine Hydrochloride, Carnitine | 0 | −23 | 1 | 1 | 0 | Q4 | | | | |
| (dl) Hydrochloride | | | | | | | | | | |
| Carprofen | 0 | −2 | 0 | 15 | 0 | Q4 | | | | |
| Carsalam | 0 | −7 | 0 | 11 | 1 | Q4 | | | | |
| Carteolol | 0 | 12 | 0 | 7 | 0 | Q4 | | | | |
| Carvedilol, Carvedilol Phosphate | 0 | 10 | 1 | 15 | 1 | Q4 | | | | |
| Carvedilol, Carvedilol Phosphate | 0 | −17 | 0 | 19 | 0 | Q4 | | | | |
| Carzenide | 0 | −24 | 1 | 14 | 0 | Q4 | | | | |
| Casanthranol [cascaroside A | 0 | −49 | 0 | 9 | 0 | Q4 | | | | |
| Shown] | | | | | | | | | | |
| Cefaclor | 0 | −14 | 1 | 19 | 0 | Q4 | | | | |
| Cefadroxil | 0 | 1 | 0 | 3 | 0 | Q4 | | | | |
| Cefalonium | 0 | 9 | 1 | 11 | 0 | Q4 | | | | |
| Cefalotin | 0 | 1 | 1 | 1 | 0 | Q4 | | | | |
| Cefamandole Nafate | 0 | −3 | 0 | 12 | 0 | Q4 | | | | |
| Cefamandole Sodium | 0 | −12 | 1 | 16 | 0 | Q4 | | | | |
| Cefapirin | 0 | −26 | 0 | 7 | 0 | Q4 | | | | |
| Cefazolin | 0 | 0 | 1 | 11 | 0 | Q4 | | | | |
| Cefdinir | 0 | −9 | 1 | 16 | 0 | Q4 | | | | |
| Cefditorin Pivoxil | 0 | 2 | 1 | 21 | 1 | Q4 | | | | |
| Cefepime | 0 | −26 | 1 | 4 | 0 | Q4 | | | | |
| Cefepime Hydrochloride | 0 | 7 | 1 | 16 | 0 | Q4 | | | | |
| Cefixime | 0 | 0 | 1 | −8 | 0 | Q4 | | | | |
| Cefmenoxime Hydrochloride | 0 | −15 | 0 | 2 | 0 | Q4 | | | | |
| Cefmetazole | 0 | −50 | 0 | 8 | 0 | Q4 | | | | |
| Cefonicid Sodium | 0 | −11 | 0 | 15 | 0 | Q4 | | | | |
| Ceforanide | 0 | −2 | 1 | 14 | 1 | Q4 | | | | |
| Cefotaxime | 0 | −20 | 1 | −8 | 0 | Q4 | | | | |
| Cefotaxime Sodium | 0 | −1 | 0 | 3 | 0 | Q4 | | | | |
| Cefotetan | 0 | −11 | 0 | −5 | 0 | Q4 | | | | |
| Cefoxitin | 0 | 6 | 0 | 8 | 0 | Q4 | | | | |
| Cefpiramide | 0 | 12 | 1 | 20 | 0 | Q4 | | | | |
| Cefpodoxime Proxetil | 0 | −39 | 0 | 12 | 0 | Q4 | | | | |
| Cefprozil | 0 | 3 | 0 | 14 | 0 | Q4 | | | | |
| Cefsulodin Sodium | 0 | 3 | 0 | 14 | 0 | Q4 | | | | |
| Ceftazidime | 0 | 14 | 1 | 17 | 1 | Q4 | | | | |
| Ceftazidime | 0 | −3 | 1 | 30 | 1 | Q4 | | | | |
| Ceftibuten | 0 | −7 | 1 | 12 | 0 | Q4 | | | | |
| Ceftiofur Hydrochloride | 0 | 4 | 1 | 14 | 0 | Q4 | | | | |
| Ceftriaxone | 0 | −16 | 1 | 12 | 0 | Q4 | | | | |
| Cefuroxime | 0 | 16 | 1 | 2 | 0 | Q4 | | | | |
| Cefuroxime Axetil | 0 | 17 | 1 | 13 | 0 | Q4 | | | | |
| Cefuroxime Sodium | 0 | −12 | 1 | 19 | 1 | Q4 | | | | |
| Celecoxib | 0 | −31 | 0 | 21 | 0 | Q4 | | | | |
| Cephalexin | 0 | −1 | 0 | 17 | 0 | Q4 | | | | |
| Cephalosporin C Sodium | 0 | −34 | 1 | 8 | 0 | Q4 | | | | |
| Cephradine | 0 | −26 | 0 | −1 | 0 | Q4 | | | | |
| Cetirizine | 0 | 5 | 1 | 0 | 0 | Q4 | | | | |
| Chenodiol | 0 | −32 | 0 | 4 | 0 | Q4 | | | | |
| Chiniofon | 0 | −52 | 1 | −1 | 1 | Q4 | | | | |
| Chlomezanone, Chlormezanone | 0 | −52 | 1 | 12 | 0 | Q4 | | | | |
| Chloralose | 0 | −14 | 1 | 9 | 0 | Q4 | | | | |
| Chlorambucil | 3 | 8 | 1 | −7 | 0 | Q4 | | | | |
| Chloramine-t | 0 | 4 | 1 | 11 | 0 | Q4 | | | | |
| Chloramphenicol | 0 | 19 | 1 | 6 | 0 | Q4 | | | | |
| Chloramphenicol Palmitate | 0 | 15 | 1 | 9 | 1 | Q4 | | | | |
| CHLORAMPHENICOL SUCCINATE | 0 | 24 | 2 | 14 | 1 | Q4 | | | | |
| Chlorazanil Hydrochloride | 0 | 0 | 1 | 3 | 0 | Q4 | | | | |
| Chlorcyclizine | 0 | 9 | 1 | 6 | 0 | Q4 | | | | |
| Chlorindanol | 0 | 23 | 1 | 15 | 0 | Q4 | | | | |
| Chlorindione | 2 | −7 | 1 | 10 | 0 | Q4 | | | | |
| Chlorobutanol | 0 | −20 | 1 | 3 | 0 | Q4 | | | | |
| Chlorocresol | 0 | −25 | 1 | 11 | 0 | Q4 | | | | |
| Chlorophyllide Cu Complex Na Salt | 0 | −36 | 0 | 9 | 0 | Q4 | | | | |
| Chloropyramine | 0 | 25 | 1 | 13 | 0 | Q4 | | | | |
| Chloroquine | 0 | 2 | 0 | 7 | 0 | Q4 | | | | |
| Chlorothiazide | 0 | 20 | 0 | 3 | 0 | Q4 | | | | |
| Chloroxine | 0 | −17 | 1 | 8 | 0 | Q4 | | | | |
| Chloroxylenol | 0 | −15 | 1 | 7 | 0 | Q4 | | | | |
| Chlorphenamine | 0 | 0 | 1 | 13 | 0 | Q4 | | | | |
| Chlorphenesin Carbamate | 0 | −41 | 1 | −4 | 0 | Q4 | | | | |
| Chlorpromazine Hydrochloride, | 1 | 9 | 2 | 25 | 1 | Q4 | | | | |
| Chlorpromazine | | | | | | | | | | |
| Chlorpropamide | 0 | 3 | 1 | 17 | 1 | Q4 | | | | |
| Chlorprothixene | 1 | 29 | 1 | 41 | 1 | Q4 | | | | |
| Chlorprothixene | 1 | 11 | 1 | −19 | 0 | Q4 | | | | |
| Chlorpyrifos | 0 | −7 | 1 | 1 | 0 | Q4 | | | | |
| Chlorquinaldol | 0 | −14 | 1 | 15 | 0 | Q4 | | | | |
| Chlortetracycline | 0 | −15 | 1 | −6 | 0 | Q4 | | | | |
| Chlortetracycline, Chlortetracycline | 0 | 27 | 2 | 23 | 0 | Q4 | | | | |
| Hydrochloride | | | | | | | | | | |
| Chlorzoxazone | 0 | 30 | 1 | 17 | 1 | Q4 | | | | |
| Cholecalciferol | 0 | −10 | 1 | 4 | 0 | Q4 | | | | |
| Cholecalciferol | 0 | −17 | 1 | −10 | 0 | Q4 | | | | |
| Cholesterol | 0 | 4 | 0 | 9 | 0 | Q4 | | | | |
| Cholic Acid | 0 | −31 | 0 | −5 | 0 | Q4 | | | | |
| Choline | 0 | 0 | 0 | 12 | 0 | Q4 | | | | |
| Chromocarb | 0 | −7 | 1 | 5 | 0 | Q4 | | | | |
| Cianidanol | 2 | −19 | 0 | 7 | 0 | Q4 | | | | |
| Ciclopirox | 0 | −1 | 1 | 40 | 1 | Q4 | | | | |
| Cilastatin | 0 | −6 | 1 | 2 | 0 | Q4 | | | | |
| Cilostazol | 0 | 5 | 1 | 17 | 1 | Q4 | | | | |
| Cimetidine | 0 | −1 | 0 | 22 | 1 | Q4 | | | | |
| Cinalukast | 0 | −21 | 1 | −8 | 0 | Q4 | | | | |
| Cinchocaine | 0 | −57 | 0 | 7 | 0 | Q4 | | | | |
| Cinchonidine | 0 | 13 | 1 | 4 | 0 | Q4 | | | | |
| Cinchonine | 0 | −6 | 1 | 28 | 0 | Q4 | | | | |
| Cinchophen | 0 | −9 | 1 | 36 | 0 | Q4 | | | | |
| Cinnarazine | 0 | −17 | 1 | 6 | 0 | Q4 | | | | |
| Cinnarizine | 0 | −32 | 1 | −2 | 0 | Q4 | | | | |
| Cinromide | 0 | −10 | 1 | 13 | 0 | Q4 | | | | |
| Cintriamide | 0 | −2 | 1 | 15 | 0 | Q4 | | | | |
| Ciprofibrate | 0 | −16 | 0 | 16 | 1 | Q4 | | | | |
| Ciprofloxacin | 0 | −30 | 0 | 17 | 0 | Q4 | | | | |
| Cisapride | 0 | −38 | 0 | 5 | 0 | Q4 | | | | |
| Cisplatin | 0 | −13 | 0 | 20 | 0 | Q4 | | | | |
| Citalopram | 0 | −12 | 0 | 10 | 0 | Q4 | | | | |
| Citalopram | 0 | −30 | 0 | 8 | 0 | Q4 | | | | |
| Citicoline | 0 | 5 | 1 | 16 | 0 | Q4 | | | | |
| Citiolone | 0 | −32 | 0 | 7 | 0 | Q4 | | | | |
| Citric Acid | 0 | −36 | 1 | −6 | 0 | Q4 | | | | |
| Cladribine | 0 | −23 | 1 | 6 | 0 | Q4 | | | | |
| Clarithromycin | 0 | −7 | 1 | 18 | 0 | Q4 | | | | |
| Clavulanate | 0 | −16 | 0 | 5 | 0 | Q4 | | | | |
| Clemastine, Clemastine Fumarate | 0 | 9 | 1 | 27 | 0 | Q4 | | | | |
| Clemizole Hydrochloride | 0 | −38 | 0 | 21 | 1 | Q4 | | | | |
| Clenbuterol | 0 | 11 | 1 | 25 | 0 | Q4 | | | | |
| Clidinium Bromide | 0 | −20 | 1 | 9 | 0 | Q4 | | | | |
| Climbazole | 0 | −10 | 1 | 1 | 0 | Q4 | | | | |
| Clinafoxacin Hydrochloride | 0 | −22 | 0 | 9 | 0 | Q4 | | | | |
| Clindamycin Hydrochloride | 0 | −44 | 0 | 7 | 0 | Q4 | | | | |
| Clindamycin Palmitate Hydrochloride | 0 | −23 | 0 | 11 | 0 | Q4 | | | | |
| Clioquinol | 0 | −11 | 1 | 22 | 1 | Q4 | | | | |
| Clobetasol Propionate | 0 | −22 | 1 | 30 | 2 | Q4 | | | | |
| Clodronate | 0 | −15 | 1 | 1 | 0 | Q4 | | | | |
| Clofarabine | 0 | −4 | 1 | −2 | 0 | Q4 | | | | |
| Clofarabine | 0 | 26 | 1 | 8 | −1 | Q4 | | | | |
| Clofazimine | 3 | 23 | 1 | 15 | 0 | Q4 | | | | |
| Clofibrate | 0 | −20 | 0 | 18 | 1 | Q4 | | | | |
| Clofibric Acid | 0 | −27 | 1 | 15 | 0 | Q4 | | | | |
| Clofoctol | 0 | −26 | 0 | 2 | 0 | Q4 | | | | |
| Clomiphene | 0 | 11 | 1 | 7 | 0 | Q4 | | | | |
| Clomipramine | 0 | −1 | 1 | 32 | 1 | Q4 | | | | |
| Clonazepam | 0 | −12 | 0 | −9 | 0 | Q4 | | | | |
| Clonidine | 0 | −22 | 1 | 14 | 0 | Q4 | | | | |
| Clopidogrel | 0 | −24 | 1 | 7 | 0 | Q4 | | | | |
| Clopidol | 0 | −41 | 1 | 9 | 0 | Q4 | | | | |
| Clorgyline Hydrochloride, Clorgiline | 0 | −28 | 0 | 6 | 0 | Q4 | | | | |
| Hydrochloride | | | | | | | | | | |
| Clorsulon | 0 | 0 | 0 | 17 | 0 | Q4 | | | | |
| Closantel | 0 | −9 | 0 | 5 | 0 | Q4 | | | | |
| Clotrimazole | 0 | −13 | 1 | 13 | 0 | Q4 | | | | |
| Cloxacillin | 0 | −13 | 1 | 17 | 1 | Q4 | | | | |
| Cloxyquin | 0 | −4 | 0 | 18 | 0 | Q4 | | | | |
| Clozapine | 0 | 3 | 0 | 9 | 0 | Q4 | | | | |
| Colesevalam Hydrochloride (high | 0 | 8 | 1 | 3 | 0 | Q4 | | | | |
| Mol Wt Copolymer @10 mg/ml) | | | | | | | | | | |
| Colforsin | 0 | 46 | 1 | 26 | 0 | Q4 | | | | |
| Cortisone | 1 | 37 | 1 | 44 | 1 | Q4 | | | | |
| Cortisone Acetate | 1 | 0 | 1 | 7 | 0 | Q4 | | | | |
| Cotinine | 0 | −39 | 1 | 6 | 1 | Q4 | | | | |
| Coumophos | 0 | −12 | 1 | 16 | 0 | Q4 | | | | |
| Creatinine | 0 | −3 | 1 | 10 | 0 | Q4 | | | | |
| Cresopyrine, Cresopirine | 0 | 13 | 1 | 15 | 1 | Q4 | | | | |
| Cromoglicic acid | 0 | −28 | 0 | 8 | 0 | Q4 | | | | |
| Crotamiton | 0 | 12 | 0 | 3 | 0 | Q4 | | | | |
| Cryoflurane | 0 | −4 | 1 | 12 | 0 | Q4 | | | | |
| Cyanocobalamin | 0 | −1 | 1 | 11 | 0 | Q4 | | | | |
| Cyclamic Acid | 0 | 4 | 0 | 13 | 0 | Q4 | | | | |
| Cyclandelate | 0 | −57 | 0 | 4 | 0 | Q4 | | | | |
| Cyclizine | 0 | −23 | 0 | 34 | 1 | Q4 | | | | |
| Cyclobenzaprine | 0 | −29 | 0 | 35 | 1 | Q4 | | | | |
| Cyclopentolate | 0 | −8 | 1 | 9 | 0 | Q4 | | | | |
| Cyclophosphamide Hydrate, | 0 | −15 | 0 | 12 | 0 | Q4 | | | | |
| Cyclophosphamide, Cytoxan | | | | | | | | | | |
| Cycloserine | 0 | −4 | 0 | 19 | 0 | Q4 | | | | |
| Cyproheptadine | 0 | 24 | 1 | 43 | 1 | Q4 | | | | |
| Cyproterone Acetate | 0 | −10 | 1 | 12 | 1 | Q4 | | | | |
| Cyproterone Acetate | 0 | −24 | 1 | 3 | 0 | Q4 | | | | |
| Cyromazine | 0 | −9 | 1 | 9 | 0 | Q4 | | | | |
| Cysteamine | 0 | 9 | 0 | 12 | 0 | Q4 | | | | |
| Cytarabine | 0 | −19 | 0 | 14 | 0 | Q4 | | | | |
| Cytisine | 0 | 23 | 1 | 16 | 1 | Q4 | | | | |
| D-(+)-maltose | 0 | −7 | 1 | 14 | 0 | Q4 | | | | |
| D-camphor | 0 | −32 | 0 | 6 | 0 | Q4 | | | | |
| D-lactitol Monohydrate | 0 | 11 | 1 | 16 | 0 | Q4 | | | | |
| D-limonene | 0 | −19 | 1 | 11 | 1 | Q4 | | | | |
| D-phenylalanine | 0 | 1 | 1 | 23 | 0 | Q4 | | | | |
| Dabigatran etexilate | 0 | 3 | 0 | 9 | 0 | Q4 | | | | |
| Dacarbazine | 3 | −13 | 0 | 17 | 0 | Q4 | | | | |
| Dalfampridine | 0 | 8 | 2 | 2 | 0 | Q4 | | | | |
| Danazol | 0 | −14 | 1 | 5 | 0 | Q4 | | | | |
| Dantrolene | 0 | −6 | 1 | 14 | 0 | Q4 | | | | |
| Dantron, Danthron | 3 | −9 | 0 | 13 | 0 | Q4 | | | | |
| Dapiprazole | 0 | −9 | 1 | 14 | 0 | Q4 | | | | |
| Dapsone | 0 | −23 | 1 | 11 | 0 | Q4 | | | | |
| Daptomycin (5 Millimolar/dmso) | 0 | −6 | 1 | 17 | 0 | Q4 | | | | |
| Darifenacin Hydrobromide | 0 | 1 | 1 | 0 | 0 | Q4 | | | | |
| Debrisoquin | 0 | 8 | 1 | 17 | 0 | Q4 | | | | |
| Decamethonium | 0 | 1 | 0 | 17 | 0 | Q4 | | | | |
| Decoquinate | 0 | 7 | 1 | 20 | 1 | Q4 | | | | |
| Deet, Diethyltoluamide | 0 | −38 | 0 | 6 | 0 | Q4 | | | | |
| Deferiprone | 0 | 31 | 1 | 18 | 0 | Q4 | | | | |
| Deferoxamine | 0 | 2 | 1 | 32 | 0 | Q4 | | | | |
| Dehydroacetic Acid | 0 | −36 | 0 | 2 | 0 | Q4 | | | | |
| Dehydrocholic Acid | 0 | −30 | 1 | 7 | 0 | Q4 | | | | |
| Delavirdine | 0 | −11 | 1 | 9 | 1 | Q4 | | | | |
| Denatonium Benzoate | 0 | 2 | 0 | 12 | 0 | Q4 | | | | |
| Dequadin | 0 | −32 | 0 | 2 | 0 | Q4 | | | | |
| Deracoxib | 0 | −34 | 0 | 8 | 0 | Q4 | | | | |
| Desloratadine | 0 | 19 | 1 | 45 | 0 | Q4 | | | | |
| Desvenlafaxine | 0 | 13 | 1 | 3 | 1 | Q4 | | | | |
| Dexbrompheniramine, | 0 | 0 | 0 | 10 | 0 | Q4 | | | | |
| Dexbrompheniramine Maleate | | | | | | | | | | |
| Dexchlorpheniramine | 0 | −8 | 1 | 11 | 0 | Q4 | | | | |
| Dexfenfluramine | 0 | −15 | 1 | 5 | 0 | Q4 | | | | |
| Dexibuprofen | 0 | −3 | 1 | 13 | 0 | Q4 | | | | |
| Dexketoprofen | 0 | −23 | 0 | −8 | 0 | Q4 | | | | |
| Dexlansoprazole | 0 | −23 | 0 | −12 | 0 | Q4 | | | | |
| Dexpanthenol | 0 | −1 | 0 | 11 | 0 | Q4 | | | | |
| Dexpropranolol Hydrochloride | 0 | −21 | 0 | 19 | 0 | Q4 | | | | |
| Dextromethorphan | 0 | −18 | 1 | 4 | 0 | Q4 | | | | |
| Dextromethorphan | 0 | −20 | 1 | −5 | 0 | Q4 | | | | |
| Dextropropoxyphene | 0 | −25 | 1 | 15 | 0 | Q4 | | | | |
| Diacerin | 3 | −30 | 1 | 10 | 0 | Q4 | | | | |
| Diacetamate | 0 | −25 | 1 | 21 | 0 | Q4 | | | | |
| Diatrizoate | 0 | 4 | 0 | 5 | 0 | Q4 | | | | |
| Diaveridine | 0 | −8 | 1 | 13 | 1 | Q4 | | | | |
| Diazepam | 0 | −19 | 1 | 5 | 0 | Q4 | | | | |
| Diazinon, Dimpylate | 0 | −7 | 1 | −8 | 0 | Q4 | | | | |
| Diazoxide | 0 | 3 | 0 | 24 | 0 | Q4 | | | | |
| Dibekacin | 0 | 10 | 1 | 20 | 1 | Q4 | | | | |
| Dibenzothiophene | 0 | −21 | 0 | 14 | 0 | Q4 | | | | |
| Dibutyl Phthalate | 0 | −30 | 1 | 9 | 0 | Q4 | | | | |
| Dichlorophene, Dichlorophen | 0 | −23 | 1 | 15 | 0 | Q4 | | | | |
| Dichlorvos | 0 | −1 | 1 | 19 | 1 | Q4 | | | | |
| Diclazuril | 0 | 48 | 1 | 21 | 0 | Q4 | | | | |
| Diclofenac | 0 | −27 | 0 | 12 | 0 | Q4 | | | | |
| Dicloxacillin | 0 | −23 | 0 | 14 | 0 | Q4 | | | | |
| Dicoumarol | 0 | −24 | 1 | 18 | 0 | Q4 | | | | |
| Dicyclomine | 0 | −36 | 0 | 16 | 0 | Q4 | | | | |
| Didanosine | 0 | −8 | 1 | −2 | 0 | Q4 | | | | |
| Dienestrol | 0 | 17 | 1 | 21 | 0 | Q4 | | | | |
| Diethylcarbamazine | 0 | −26 | 0 | 11 | 0 | Q4 | | | | |
| Diethylstilbestrol | 0 | −26 | 1 | 16 | 0 | Q4 | | | | |
| Diflorasone Diacetate | 0 | 42 | 1 | 31 | 0 | Q4 | | | | |
| Difloxacin Hydrochloride | 0 | 25 | 1 | 33 | 1 | Q4 | | | | |
| Diflunisal | 0 | −62 | 0 | 15 | 0 | Q4 | | | | |
| Digitoxin | 0 | −13 | 1 | 5 | 1 | Q4 | | | | |
| Digitoxin | 0 | −1 | 1 | 20 | 0 | Q4 | | | | |
| Digoxin | 0 | −5 | 1 | 10 | 0 | Q4 | | | | |
| Dihydroergotamine | 0 | 7 | 1 | 39 | 1 | Q4 | | | | |
| Dihydroergotamine | 0 | −3 | 0 | 22 | 0 | Q4 | | | | |
| Dihydrostreptomycin Sulfate | 0 | −7 | 1 | 12 | 0 | Q4 | | | | |
| Diiodohydroxyquinoline | 0 | −6 | 1 | 22 | 0 | Q4 | | | | |
| Diloxanide Furoate | 0 | −3 | 1 | 17 | 0 | Q4 | | | | |
| Diltiazem | 0 | 21 | 1 | 9 | 0 | Q4 | | | | |
| Diltiazem Hydrochloride | 0 | −5 | 0 | 15 | 0 | Q4 | | | | |
| Dimenhydrinate | 0 | −17 | 1 | 11 | 0 | Q4 | | | | |
| Dimercaptopropanol, Dimercaprol | 0 | 49 | 1 | 16 | 0 | Q4 | | | | |
| Dimesna | 0 | −19 | 1 | 13 | 0 | Q4 | | | | |
| Dimethadione | 0 | −13 | 1 | 14 | 0 | Q4 | | | | |
| Dimethyl Fumarate | 0 | 4 | 0 | 16 | 0 | Q4 | | | | |
| Dimetridazole | 0 | −16 | 1 | 15 | 1 | Q4 | | | | |
| Diminazene | 3 | −58 | 0 | 11 | 0 | Q4 | | | | |
| Dinitolmide | 0 | −45 | 1 | 10 | 0 | Q4 | | | | |
| Dinoprost Tromethamine | 0 | 36 | 1 | 15 | 0 | Q4 | | | | |
| Diosmin | 0 | −12 | 1 | 10 | 0 | Q4 | | | | |
| Dioxybenzone | 0 | −4 | 1 | 16 | 0 | Q4 | | | | |
| Diperodon Hydrochloride | 0 | 42 | 1 | 26 | 1 | Q4 | | | | |
| Diphenhydramine | 0 | −47 | 0 | 15 | 0 | Q4 | | | | |
| Diphenidol | 0 | −34 | 1 | −5 | 0 | Q4 | | | | |
| Diphenylpyraline | 0 | −69 | 0 | 6 | 0 | Q4 | | | | |
| Dipyridamole | 0 | 11 | 1 | 6 | 0 | Q4 | | | | |
| Dipyrocetyl | 0 | −1 | 1 | 7 | 0 | Q4 | | | | |
| Dirithromycin | 0 | 7 | 1 | 27 | 1 | Q4 | | | | |
| Disopyramide | 0 | −13 | 1 | 1 | 0 | Q4 | | | | |
| Dixanthogen | 0 | 4 | 1 | 17 | 0 | Q4 | | | | |
| Dobutamine | 2 | −29 | 1 | −7 | 0 | Q4 | | | | |
| Dobutamine Hydrochloride | 2 | 22 | 1 | 27 | 0 | Q4 | | | | |
| Doconexent | 0 | 3 | 1 | 11 | 0 | Q4 | | | | |
| Docosanol | 0 | −10 | 0 | −2 | 0 | Q4 | | | | |
| Docusate Sodium | 0 | −39 | 0 | −4 | 0 | Q4 | | | | |
| Dofetilide | 0 | −26 | 1 | 5 | 0 | Q4 | | | | |
| Dolasetron | 0 | −19 | 1 | −3 | 0 | Q4 | | | | |
| Domperidone | 0 | 20 | 1 | 15 | 0 | Q4 | | | | |
| Donepezil | 0 | 5 | 1 | 17 | 0 | Q4 | | | | |
| Dopamine | 2 | −17 | 1 | 9 | 0 | Q4 | | | | |
| Doramectin | 0 | −9 | 1 | 7 | 0 | Q4 | | | | |
| Dorzolamide | 0 | −40 | 1 | 15 | 1 | Q4 | | | | |
| Doxapram Hydrochloride | 0 | −18 | 0 | 9 | 0 | Q4 | | | | |
| Doxazosin | 0 | −26 | 0 | 6 | 0 | Q4 | | | | |
| Doxepin | 0 | −10 | 1 | 16 | 0 | Q4 | | | | |
| Doxepin | 0 | −36 | 1 | 13 | 0 | Q4 | | | | |
| Doxofylline | 0 | −42 | 0 | 18 | 0 | Q4 | | | | |
| Doxycycline | 0 | −19 | 0 | 18 | 0 | Q4 | | | | |
| Doxylamine | 0 | −45 | 1 | 17 | 0 | Q4 | | | | |
| Drofenine Hydrochloride | 0 | −44 | 1 | 9 | 0 | Q4 | | | | |
| Droperidol | 0 | −3 | 1 | 20 | 1 | Q4 | | | | |
| Dropropizine | 0 | −26 | 1 | 11 | 0 | Q4 | | | | |
| Drospirenone | 0 | 11 | 1 | 6 | 1 | Q4 | | | | |
| Duloxetine | 0 | 22 | 0 | 24 | 0 | Q4 | | | | |
| Dutasteride | 0 | 7 | 1 | 22 | 0 | Q4 | | | | |
| Dyclonine | 0 | 7 | 1 | 12 | 0 | Q4 | | | | |
| Dyclonine | 0 | −8 | 1 | 5 | 0 | Q4 | | | | |
| Dydrogesterone | 0 | −11 | 0 | 4 | 0 | Q4 | | | | |
| Dyphylline | 0 | −7 | 0 | −6 | 0 | Q4 | | | | |
| Ebselen | 0 | −49 | 0 | −1 | 0 | Q4 | | | | |
| Ecamsule | 0 | 20 | 1 | 29 | 0 | Q4 | | | | |
| Econazole | 0 | 0 | 0 | 18 | 0 | Q4 | | | | |
| Edetic Acid | 0 | −54 | 0 | 2 | 0 | Q4 | | | | |
| Edetic Acid | 0 | 0 | 0 | 16 | 1 | Q4 | | | | |
| Editol | 0 | 24 | 1 | 3 | 0 | Q4 | | | | |
| Edoxudine | 0 | −10 | 0 | 1 | 0 | Q4 | | | | |
| Edrophonium | 0 | −9 | 1 | −2 | 0 | Q4 | | | | |
| Efaroxan, Efaroxan Hydrochloride | 0 | 1 | 1 | 5 | 0 | Q4 | | | | |
| Efavirenz | 0 | −7 | 1 | −5 | 0 | Q4 | | | | |
| Efloxate | 0 | −8 | 1 | −1 | 0 | Q4 | | | | |
| Eletriptan Hydrobromide | 0 | −14 | 1 | 12 | 0 | Q4 | | | | |
| Ellagic Acid | 2 | −5 | 1 | 24 | 0 | Q4 | | | | |
| Eltanolone | 0 | −6 | 1 | 4 | 0 | Q4 | | | | |
| Emtricitabine | 0 | −73 | 1 | 11 | 1 | Q4 | | | | |
| Enalapril | 0 | 24 | 1 | 6 | 1 | Q4 | | | | |
| Enalaprilat | 0 | 25 | 1 | 18 | 0 | Q4 | | | | |
| Enilconazole | 0 | 24 | 1 | 15 | 1 | Q4 | | | | |
| Enoxolone | 0 | −40 | 1 | 7 | 0 | Q4 | | | | |
| Enrofloxacin | 0 | 5 | 1 | 23 | 1 | Q4 | | | | |
| Entacapone | 2 | −3 | 1 | 11 | 0 | Q4 | | | | |
| Entacapone | 2 | −19 | 1 | 17 | 1 | Q4 | | | | |
| Ephedrine (1r,2s) Hydrochloride | 0 | −26 | 1 | 10 | 0 | Q4 | | | | |
| Epiestriol | 0 | −25 | 0 | 6 | 0 | Q4 | | | | |
| Epinastine | 0 | −38 | 1 | −15 | 0 | Q4 | | | | |
| Eprinomectin | 0 | −3 | 1 | 8 | 1 | Q4 | | | | |
| Eprobemide | 0 | −24 | 0 | 21 | 0 | Q4 | | | | |
| Eprodisate Disodium | 0 | −3 | 1 | 12 | 0 | Q4 | | | | |
| Eprosartan | 0 | −29 | 1 | −8 | 0 | Q4 | | | | |
| Equilin | 0 | 1 | 0 | 15 | 0 | Q4 | | | | |
| Erdosteine | 0 | 2 | 1 | 13 | 0 | Q4 | | | | |
| Ergocalciferol | 0 | −6 | 1 | 7 | 0 | Q4 | | | | |
| Ergonovine | 0 | −23 | 1 | −10 | 0 | Q4 | | | | |
| Ergonovine Maleate | 0 | 9 | 1 | 7 | 0 | Q4 | | | | |
| Ergotamine | 0 | 6 | 1 | −4 | 0 | Q4 | | | | |
| Ergotamine Tartrate | 0 | 31 | 0 | 22 | 1 | Q4 | | | | |
| Erlotinib | 0 | −52 | 1 | −8 | 0 | Q4 | | | | |
| Erythritol | 0 | −25 | 1 | 10 | 0 | Q4 | | | | |
| Erythromycin | 0 | 9 | 1 | 20 | 0 | Q4 | | | | |
| Erythromycin | 0 | −20 | 1 | 13 | 1 | Q4 | | | | |
| Erythromycin Ethylsuccinate | 0 | −14 | 0 | 14 | 0 | Q4 | | | | |
| Erythromycin Propionate | 0 | 23 | 1 | 27 | 0 | Q4 | | | | |
| Erythrosine Sodium | 2 | 1 | 1 | 0 | 0 | Q4 | | | | |
| Esmolol, Esmolol Hydrochloride | 0 | −15 | 1 | 2 | 0 | Q4 | | | | |
| Esomeprazole; Omeprazole | 0 | −17 | 0 | 24 | 0 | Q4 | | | | |
| Esomeprazole; Omeprazole | 0 | 45 | 1 | 13 | 1 | Q4 | | | | |
| Estradiol | 0 | 3 | 0 | 16 | 0 | Q4 | | | | |
| Estradiol | 0 | −10 | 1 | −5 | 0 | Q4 | | | | |
| Estradiol Benzoate | 0 | −3 | 1 | 8 | 0 | Q4 | | | | |
| Estradiol Cypionate | 0 | −25 | 0 | 8 | 0 | Q4 | | | | |
| Estradiol Dipropionate | 0 | 0 | 1 | 17 | 1 | Q4 | | | | |
| Estradiol Valerate | 0 | −8 | 1 | 16 | 1 | Q4 | | | | |
| Estramustine | 0 | 8 | 1 | 16 | 1 | Q4 | | | | |
| Estriol | 0 | −17 | 1 | 8 | 1 | Q4 | | | | |
| Estriol | 0 | −17 | 1 | 16 | 1 | Q4 | | | | |
| Estrone | 0 | 35 | 2 | 16 | 0 | Q4 | | | | |
| Estropipate | 0 | 10 | 1 | 10 | 0 | Q4 | | | | |
| Eszopiclone | 0 | 1 | 0 | 17 | 0 | Q4 | | | | |
| Etamivan | 0 | −16 | 1 | 16 | 0 | Q4 | | | | |
| Ethacridine Lactate | 2 | −64 | 1 | −8 | 0 | Q4 | | | | |
| Ethambutol | 0 | 12 | 1 | 14 | 1 | Q4 | | | | |
| Ethaverine Hydrochloride | 0 | 41 | 1 | 24 | 1 | Q4 | | | | |
| Ethenzamide | 0 | −15 | 1 | 5 | 0 | Q4 | | | | |
| Ethinyl Estradiol | 0 | 9 | 1 | 5 | 0 | Q4 | | | | |
| Ethionamide | 0 | 17 | 1 | 14 | 0 | Q4 | | | | |
| Ethisterone | 0 | −5 | 0 | 13 | 1 | Q4 | | | | |
| Ethopabate | 0 | −43 | 0 | 6 | 0 | Q4 | | | | |
| Ethopropazine | 1 | 31 | 1 | 30 | 0 | Q4 | | | | |
| Ethosuximide | 0 | −9 | 0 | 19 | 0 | Q4 | | | | |
| Ethotoin | 0 | −36 | 1 | 12 | 0 | Q4 | | | | |
| Ethoxzolamide | 0 | 29 | 1 | 16 | 1 | Q4 | | | | |
| Ethyl carbamate | 0 | 44 | 1 | 27 | 0 | Q4 | | | | |
| Ethyl Vanillin | 0 | −15 | 0 | 18 | 0 | Q4 | | | | |
| Ethylparaben | 0 | −5 | 1 | 8 | 1 | Q4 | | | | |
| Ethynodiol Diacetate | 0 | −29 | 1 | −6 | 0 | Q4 | | | | |
| Ethynodiol Diacetate | 0 | −6 | 1 | 16 | 1 | Q4 | | | | |
| Etidronic acid | 0 | −16 | 1 | 18 | 0 | Q4 | | | | |
| Etodolac | 0 | −87 | 0 | 14 | 1 | Q4 | | | | |
| Etomidate | 0 | −19 | 0 | 12 | 0 | Q4 | | | | |
| Etomidate | 0 | −24 | 0 | −7 | 0 | Q4 | | | | |
| Etoposide | 0 | 30 | 1 | 15 | 0 | Q4 | | | | |
| Etoricoxib | 0 | 15 | 1 | −4 | 0 | Q4 | | | | |
| Etretinate | 0 | −19 | 1 | 9 | 0 | Q4 | | | | |
| Eucalyptol | 0 | −15 | 0 | 9 | 0 | Q4 | | | | |
| Eucatropine Hydrochloride | 0 | 1 | 1 | 24 | 0 | Q4 | | | | |
| Eugenol | 0 | 37 | 0 | 20 | 0 | Q4 | | | | |
| Evans Blue | 3 | −37 | 0 | 7 | 0 | Q4 | | | | |
| Exalamide | 0 | −15 | 1 | 18 | 0 | Q4 | | | | |
| Exemestane | 0 | 22 | 1 | 21 | 1 | Q4 | | | | |
| Exemestane | 0 | 31 | 1 | 34 | 0 | Q4 | | | | |
| Ezetimibe | 0 | 41 | 1 | 27 | 0 | Q4 | | | | |
| Famciclovir | 0 | −22 | 0 | 14 | 1 | Q4 | | | | |
| Famotidine | 0 | −82 | 0 | 9 | 0 | Q4 | | | | |
| Famprofazone | 0 | −3 | 1 | 15 | 0 | Q4 | | | | |
| Fasudil Hydrochloride | 0 | −72 | 1 | −21 | 0 | Q4 | | | | |
| Febuxostat | 0 | 3 | 1 | 10 | 0 | Q4 | | | | |
| Felbamate | 0 | −32 | 1 | 0 | 0 | Q4 | | | | |
| Felodipine | 0 | 2 | 1 | 14 | 0 | Q4 | | | | |
| Fenaclon | 0 | 8 | 1 | 16 | 0 | Q4 | | | | |
| Fenamisal, Phenyl Aminosalicylate | 0 | −8 | 1 | 16 | 0 | Q4 | | | | |
| Fenbufen | 0 | 3 | 0 | 25 | 0 | Q4 | | | | |
| Fenipentol | 0 | −1 | 1 | 25 | 0 | Q4 | | | | |
| Fenofibrate | 0 | −37 | 1 | 0 | 0 | Q4 | | | | |
| Fenofibric Acid | 0 | 6 | 1 | 9 | 0 | Q4 | | | | |
| Fenoldopam | 2 | 6 | 1 | 20 | 0 | Q4 | | | | |
| Fenoldopam | 2 | −23 | 0 | 5 | 0 | Q4 | | | | |
| Fenoprofen | 0 | −7 | 0 | −4 | 0 | Q4 | | | | |
| Fenoterol | 0 | 40 | 0 | 26 | 0 | Q4 | | | | |
| Fenretinide | 0 | −42 | 0 | 35 | 0 | Q4 | | | | |
| Fenspiride | 0 | −43 | 1 | −1 | 0 | Q4 | | | | |
| Fexofenadine | 0 | 13 | 1 | 14 | 0 | Q4 | | | | |
| Finasteride | 0 | −46 | 1 | 42 | 1 | Q4 | | | | |
| Finasteride | 0 | 14 | 0 | −16 | 0 | Q4 | | | | |
| Fipexide Hydrochloride | 0 | −15 | 0 | 11 | 0 | Q4 | | | | |
| Fipronil | 0 | −5 | 1 | 12 | 0 | Q4 | | | | |
| Firocoxib | 0 | −10 | 1 | 11 | 1 | Q4 | | | | |
| Flecainide | 0 | −35 | 0 | 5 | 0 | Q4 | | | | |
| Flopropione | 0 | −35 | 1 | 10 | 0 | Q4 | | | | |
| Florfenicol | 0 | −12 | 1 | 16 | 1 | Q4 | | | | |
| Floxuridine | 0 | −16 | 1 | 5 | 0 | Q4 | | | | |
| Floxuridine | 0 | 7 | 1 | −16 | 0 | Q4 | | | | |
| Fluconazole | 0 | 3 | 0 | 19 | 0 | Q4 | | | | |
| Flucytosine, 5-fluorocytosine | 0 | 13 | 1 | 17 | 1 | Q4 | | | | |
| Fludarabine Phosphate | 0 | 1 | 1 | 11 | 0 | Q4 | | | | |
| Flufenamic Acid | 0 | −20 | 0 | −1 | 0 | Q4 | | | | |
| Flumazenil | 0 | 5 | 1 | 14 | 1 | Q4 | | | | |
| Flumequine | 0 | −1 | 0 | 16 | 1 | Q4 | | | | |
| Flunarizine | 0 | −10 | 1 | 12 | 1 | Q4 | | | | |
| Flunixin Meglumine | 0 | −6 | 1 | 8 | 0 | Q4 | | | | |
| Fluocinolone Acetonide | 0 | −35 | 1 | 10 | 0 | Q4 | | | | |
| Fluorescein | 0 | −3 | 0 | 4 | 0 | Q4 | | | | |
| Fluphenazine | 1 | 7 | 1 | 20 | 0 | Q4 | | | | |
| Flurbiprofen | 0 | 17 | 0 | 33 | 0 | Q4 | | | | |
| Flurofamide | 0 | 3 | 0 | −12 | 0 | Q4 | | | | |
| Flurothyl | 0 | 0 | 1 | 28 | 0 | Q4 | | | | |
| Fluroxene | 0 | −19 | 0 | 11 | 0 | Q4 | | | | |
| Flutamide | 0 | 15 | 1 | 23 | 0 | Q4 | | | | |
| Fluvoxamine | 0 | −8 | 0 | −3 | 0 | Q4 | | | | |
| Folic Acid | 0 | 30 | 1 | 22 | 0 | Q4 | | | | |
| Folic Acid | 0 | 11 | 1 | 17 | 1 | Q4 | | | | |
| Fomepizole Hydrochloride, | 0 | 8 | 1 | 11 | 0 | Q4 | | | | |
| Fomepizole | | | | | | | | | | |
| Fomepizole Hydrochloride, | 0 | −7 | 1 | 15 | 0 | Q4 | | | | |
| Fomepizole | | | | | | | | | | |
| Formestane | 0 | −6 | 1 | 10 | 0 | Q4 | | | | |
| Formoterol | 0 | −6 | 1 | 32 | 1 | Q4 | | | | |
| Foscarnet | 0 | 5 | 1 | 10 | 0 | Q4 | | | | |
| Fosfosal | 0 | −29 | 0 | 16 | 1 | Q4 | | | | |
| Fosinopril | 0 | −52 | 1 | −1 | 0 | Q4 | | | | |
| Fosphomycin | 0 | 4 | 1 | 12 | 0 | Q4 | | | | |
| Fulvestrant | 0 | −27 | 0 | 15 | 0 | Q4 | | | | |
| Fulvestrant | 0 | −11 | 1 | 11 | 1 | Q4 | | | | |
| Fumaric Acid | 0 | 19 | 1 | 17 | 0 | Q4 | | | | |
| Furaltadone | 0 | −60 | 1 | 0 | 0 | Q4 | | | | |
| Furazolidone | 0 | 3 | 0 | 8 | −1 | Q4 | | | | |
| Furosemide | 0 | −5 | 1 | 25 | 1 | Q4 | | | | |
| Gaba, Gamma-aminobutyric Acid | 0 | −14 | 1 | 18 | 0 | Q4 | | | | |
| Gabapentin | 0 | −17 | 1 | 19 | 1 | Q4 | | | | |
| Gabapentin | 0 | −3 | 1 | 16 | 0 | Q4 | | | | |
| Gaboxadol | 0 | −43 | 0 | 3 | 0 | Q4 | | | | |
| Gadoteridol | 0 | −18 | 1 | 15 | 0 | Q4 | | | | |
| Galanthamine Hydrobromide, | 0 | 10 | 0 | 9 | 0 | Q4 | | | | |
| Galantamine | | | | | | | | | | |
| Gallamine Triethiodide | 0 | −11 | 1 | 24 | 1 | Q4 | | | | |
| Gallic Acid | 2 | −32 | 0 | 10 | 0 | Q4 | | | | |
| Gamma-Phenyl-Butyric Acid | 0 | −36 | 0 | 9 | 0 | Q4 | | | | |
| Ganaxolone | 0 | −38 | 1 | 12 | 0 | Q4 | | | | |
| Ganciclovir | 0 | −2 | 0 | 13 | 0 | Q4 | | | | |
| Gatifloxacin | 0 | −19 | 1 | 19 | 0 | Q4 | | | | |
| Gefitinib | 0 | 39 | 1 | 17 | 0 | Q4 | | | | |
| Gefitinib | 0 | −52 | 0 | 32 | 0 | Q4 | | | | |
| Gefitinib | 0 | −10 | 1 | 23 | 0 | Q4 | | | | |
| Gemcitabine | 0 | −31 | 1 | 0 | 0 | Q4 | | | | |
| Gemfibrozil | 0 | −47 | 0 | 10 | 0 | Q4 | | | | |
| Gemifloxacin | 0 | −34 | 0 | 6 | 0 | Q4 | | | | |
| Glafenine | 0 | 8 | 1 | 18 | 0 | Q4 | | | | |
| Glibenclamide, Glyburide | 0 | −23 | 1 | 8 | 0 | Q4 | | | | |
| Gliclazide | 0 | 1 | 1 | 20 | 0 | Q4 | | | | |
| Glimepiride | 0 | 0 | 1 | 21 | 0 | Q4 | | | | |
| Glipizide | 0 | 12 | 1 | 18 | 0 | Q4 | | | | |
| Gluconolactone | 0 | −1 | 0 | 17 | 0 | Q4 | | | | |
| Glucosamine Hydrochloride | 0 | −2 | 1 | 19 | 0 | Q4 | | | | |
| Glutathione | 0 | −5 | 1 | 13 | 0 | Q4 | | | | |
| Glycopyrrolate | 0 | −33 | 0 | 3 | 0 | Q4 | | | | |
| Granisetron | 0 | −38 | 1 | 4 | 0 | Q4 | | | | |
| Granisetron Hydrochloride | 0 | −1 | 0 | 14 | 0 | Q4 | | | | |
| Griseofulvin | 0 | 9 | 1 | 13 | 0 | Q4 | | | | |
| Guaiacol | 0 | −51 | 0 | 11 | 0 | Q4 | | | | |
| Guaifenesin | 0 | −15 | 0 | 11 | 0 | Q4 | | | | |
| Guanabenz | 0 | −46 | 1 | −18 | 0 | Q4 | | | | |
| Guanabenz Acetate | 0 | −46 | 0 | −2 | 0 | Q4 | | | | |
| Guanadrel | 0 | −1 | 1 | 5 | 0 | Q4 | | | | |
| Guanethidine | 0 | −44 | 0 | 12 | 0 | Q4 | | | | |
| Guanfacine | 0 | −5 | 1 | 15 | 0 | Q4 | | | | |
| Guanfacine | 0 | −34 | 1 | −11 | 0 | Q4 | | | | |
| Guanidine | 0 | −32 | 0 | −1 | 0 | Q4 | | | | |
| Halazone | 0 | −13 | 0 | 19 | 0 | Q4 | | | | |
| Haloperidol | 0 | −22 | 1 | 12 | 1 | Q4 | | | | |
| Halothane | 0 | 17 | 1 | 17 | 0 | Q4 | | | | |
| Heptaminol Hydrochloride | 0 | −19 | 1 | 13 | 0 | Q4 | | | | |
| Hetacillin Potassium | 0 | −15 | 0 | 15 | 1 | Q4 | | | | |
| Hexachlorophene | 0 | −19 | 0 | 15 | 0 | Q4 | | | | |
| Hexamethylenetetramine | 0 | −9 | 1 | 20 | 0 | Q4 | | | | |
| Hexestrol | 0 | −29 | 1 | 7 | 0 | Q4 | | | | |
| Hexetidine | 0 | −85 | 1 | 19 | 0 | Q4 | | | | |
| Hexylene Glycol | 0 | −8 | 1 | 12 | 0 | Q4 | | | | |
| Hexylresorcinol | 0 | −6 | 1 | 18 | 0 | Q4 | | | | |
| Histamine | 0 | 21 | 1 | 27 | 0 | Q4 | | | | |
| Homatropine Bromide, Homatropine | 0 | 26 | 1 | 30 | 0 | Q4 | | | | |
| Hydrobromide | | | | | | | | | | |
| Homatropine Methylbromide | 0 | −18 | 1 | 12 | 0 | Q4 | | | | |
| Homosalate | 0 | −3 | 1 | 18 | 1 | Q4 | | | | |
| Hydralazine | 0 | −13 | 1 | 22 | 0 | Q4 | | | | |
| Hydrastine (1r,9s) | 0 | −27 | 0 | 10 | 0 | Q4 | | | | |
| Hydrastinine Hydrochloride | 0 | 0 | 1 | 22 | 0 | Q4 | | | | |
| Hydrochlorothiazide | 0 | −29 | 0 | 21 | 0 | Q4 | | | | |
| Hydroflumethiazide | 0 | −9 | 0 | 11 | 0 | Q4 | | | | |
| Hydroquinidine | 0 | −8 | 1 | 18 | 0 | Q4 | | | | |
| Hydroquinone | 0 | 14 | 1 | 12 | 0 | Q4 | | | | |
| Hydroxyamphetamine | 0 | −12 | 1 | −2 | 0 | Q4 | | | | |
| Hydroxychloroquine | 0 | 35 | 1 | 17 | 0 | Q4 | | | | |
| Hydroxyprogesterone | 0 | 9 | 1 | 33 | 1 | Q4 | | | | |
| Hydroxyprogesterone Caproate | 0 | 22 | 1 | 25 | 0 | Q4 | | | | |
| Hydroxyurea | 0 | 1 | 1 | 25 | 1 | Q4 | | | | |
| Hydroxyzine | 0 | −28 | 0 | 10 | 0 | Q4 | | | | |
| Hydroxyzine | 0 | 22 | 1 | 35 | 0 | Q4 | | | | |
| Hyoscyamine | 0 | −20 | 0 | 13 | 0 | Q4 | | | | |
| Hyoscyamine | 0 | −42 | 1 | 10 | 0 | Q4 | | | | |
| Ibandronate | 0 | 40 | 0 | −12 | 0 | Q4 | | | | |
| Ibandronate | 0 | −7 | 1 | 2 | 0 | Q4 | | | | |
| Icosapent ethyl | 0 | −13 | 0 | 1 | 0 | Q4 | | | | |
| Idebenone | 3 | −24 | 1 | 2 | 0 | Q4 | | | | |
| Idoxuridine | 0 | −8 | 1 | −20 | 0 | Q4 | | | | |
| Idoxuridine | 0 | −12 | 1 | 15 | 1 | Q4 | | | | |
| Idramantone | 0 | −7 | 1 | 8 | 0 | Q4 | | | | |
| Ifosfamide | 0 | −11 | 1 | 10 | 0 | Q4 | | | | |
| Iloprost | 0 | 23 | 1 | −1 | 0 | Q4 | | | | |
| Imatinib, Imatinib Mesylate | 0 | −4 | 1 | 11 | 0 | Q4 | | | | |
| Imexon | 0 | −3 | 1 | 12 | 1 | Q4 | | | | |
| Imipenem | 0 | −39 | 1 | 2 | 0 | Q4 | | | | |
| Imiquimod | 0 | −23 | 0 | 15 | 0 | Q4 | | | | |
| Imiquimod | 0 | −25 | 0 | −5 | 0 | Q4 | | | | |
| Indapamide | 0 | −14 | 0 | 11 | 0 | Q4 | | | | |
| Indinavir | 0 | −22 | 1 | 5 | 0 | Q4 | | | | |
| Indomethacin | 0 | −56 | 0 | 13 | 0 | Q4 | | | | |
| Indoprofen | 0 | −34 | 1 | 18 | 0 | Q4 | | | | |
| Inosine | 0 | −31 | 1 | −11 | 0 | Q4 | | | | |
| Inositol | 0 | −21 | 0 | 11 | 0 | Q4 | | | | |
| Iodixanol | 0 | −15 | 0 | 10 | 0 | Q4 | | | | |
| Iopamidol | 0 | −14 | 1 | −4 | 0 | Q4 | | | | |
| Iothalamic Acid | 0 | 5 | 1 | 7 | 0 | Q4 | | | | |
| Ioversol | 0 | −1 | 1 | 18 | 1 | Q4 | | | | |
| Ioxilan | 0 | 7 | 0 | 8 | 0 | Q4 | | | | |
| Ipratropium Bromide | 0 | 6 | 1 | 8 | 0 | Q4 | | | | |
| Ipriflavone | 0 | −29 | 1 | 7 | 0 | Q4 | | | | |
| Iproheptine | 0 | −34 | 1 | 11 | 0 | Q4 | | | | |
| Iproniazid | 0 | −44 | 1 | 10 | 0 | Q4 | | | | |
| Irinotecan, Irinotecan Hydrochloride | 0 | −6 | 0 | 10 | 1 | Q4 | | | | |
| (trihydrate) | | | | | | | | | | |
| Irsogladine Maleate | 0 | −27 | 1 | 14 | 0 | Q4 | | | | |
| Isaxonine | 0 | −46 | 0 | −1 | 0 | Q4 | | | | |
| Isobutamben | 0 | −5 | 1 | −1 | 0 | Q4 | | | | |
| Isoetarine | 2 | 21 | 1 | 23 | 0 | Q4 | | | | |
| Isoniazid | 0 | −29 | 1 | 19 | 0 | Q4 | | | | |
| Isoprenaline | 2 | 29 | 0 | 37 | 0 | Q4 | | | | |
| Isopropamide | 0 | 19 | 0 | 24 | 0 | Q4 | | | | |
| Isosorbide Dinitrate | 0 | −14 | 0 | 10 | 0 | Q4 | | | | |
| Isosorbide Mononitrate | 0 | −17 | 0 | 15 | 1 | Q4 | | | | |
| Isovaleramide | 0 | −10 | 1 | 1 | 0 | Q4 | | | | |
| Isoxsuprine Hydrochloride | 0 | −13 | 0 | 23 | 0 | Q4 | | | | |
| Isradipine | 0 | 9 | 1 | 2 | 0 | Q4 | | | | |
| Itopride, Itopride Hydrochloride | 0 | −49 | 0 | 25 | 0 | Q4 | | | | |
| Itraconazole | 3 | −27 | 1 | 1 | 0 | Q4 | | | | |
| Itraconazole | 3 | −26 | 1 | 3 | 1 | Q4 | | | | |
| Ivermectin | 0 | −15 | 1 | 10 | 0 | Q4 | | | | |
| Ivermectin | 0 | 6 | 0 | 19 | 0 | Q4 | | | | |
| Kainic Acid | 0 | −77 | 0 | 14 | 0 | Q4 | | | | |
| Kanamycin Sulfate, Kanamycin A | 0 | −13 | 0 | 20 | 1 | Q4 | | | | |
| Sulfate | | | | | | | | | | |
| Ketamine | 0 | −86 | 1 | −2 | 0 | Q4 | | | | |
| Ketanserin Tartrate | 0 | 17 | 1 | 15 | 0 | Q4 | | | | |
| Ketoconazole | 3 | −57 | 0 | −1 | 0 | Q4 | | | | |
| Ketoprofen | 0 | 34 | 0 | 25 | 0 | Q4 | | | | |
| Ketorolac | 0 | 15 | 0 | 26 | 0 | Q4 | | | | |
| Ketotifen | 0 | 1 | 1 | 15 | 0 | Q4 | | | | |
| Khellin | 0 | −16 | 0 | 5 | 0 | Q4 | | | | |
| L-alpha-methyl Dopa, Methyldopa | 2 | 4 | 0 | 15 | 0 | Q4 | | | | |
| L-aspartic Acid, Aspartic Acid (I) | 0 | 5 | 1 | 13 | 1 | Q4 | | | | |
| L-glutamine, Glutamine (I) | 0 | 4 | 0 | 14 | 0 | Q4 | | | | |
| L-Isoleucine | 0 | −35 | 0 | 17 | 0 | Q4 | | | | |
| L(−)-norephedrine, | 0 | −15 | 1 | 7 | 0 | Q4 | | | | |
| Phenylpropanolamine Hydrochloride | | | | | | | | | | |
| Labetalol | 0 | 32 | 1 | 30 | 1 | Q4 | | | | |
| Lacitol | 0 | −24 | 1 | 18 | 1 | Q4 | | | | |
| Lactic Acid | 0 | 0 | 1 | 14 | 1 | Q4 | | | | |
| Lactose Monohydrate | 0 | −22 | 1 | 9 | 0 | Q4 | | | | |
| Lactulose | 0 | −14 | 0 | 4 | 0 | Q4 | | | | |
| Lamivudine | 0 | −14 | 1 | −20 | 0 | Q4 | | | | |
| Lamivudine | 0 | 38 | 1 | 28 | 0 | Q4 | | | | |
| Lamotrigine | 0 | −12 | 1 | 12 | 0 | Q4 | | | | |
| Lanatoside C | 0 | 17 | 1 | 35 | 1 | Q4 | | | | |
| Lansoprazole | 0 | 3 | 0 | 15 | 0 | Q4 | | | | |
| Lapatinib | 0 | −54 | 1 | 10 | 0 | Q4 | | | | |
| Lapatinib | 0 | −20 | 1 | −1 | 0 | Q4 | | | | |
| Latamoxef | 0 | −18 | 1 | 22 | 0 | Q4 | | | | |
| Latanoprost | 0 | 10 | 1 | 4 | 0 | Q4 | | | | |
| Lefunamide, Leflunomide | 0 | −22 | 0 | 10 | 0 | Q4 | | | | |
| Letrozole | 0 | 15 | 1 | 16 | 0 | Q4 | | | | |
| Leucovorin | 0 | −29 | 1 | 8 | 0 | Q4 | | | | |
| Leuprolide | 0 | 5 | 1 | −3 | −1 | Q4 | | | | |
| Levamisole | 0 | −15 | 0 | 12 | 0 | Q4 | | | | |
| Levcycloserine | 0 | 2 | 1 | 12 | 0 | Q4 | | | | |
| Levetiracetam | 0 | −24 | 1 | 7 | 0 | Q4 | | | | |
| Levobunolol | 0 | −28 | 1 | 30 | 0 | Q4 | | | | |
| Levobunolol Hydrochloride | 0 | −19 | 0 | 12 | 0 | Q4 | | | | |
| Levobupivacaine | 0 | 34 | 1 | −10 | 0 | Q4 | | | | |
| Levocabastine | 0 | 7 | 1 | 4 | 0 | Q4 | | | | |
| Levocarnitine | 0 | 8 | 1 | 15 | 0 | Q4 | | | | |
| Levocarnitine Propionate | 0 | −16 | 1 | 12 | 1 | Q4 | | | | |
| Hydrochloride | | | | | | | | | | |
| Levocetirizine Dihydrochloride | 0 | −44 | 0 | −2 | 0 | Q4 | | | | |
| Levodopa | 2 | 17 | 1 | −5 | 0 | Q4 | | | | |
| Levofloxacin | 0 | 12 | 1 | 31 | 0 | Q4 | | | | |
| Levonorgestrel | 0 | 16 | 1 | −13 | 0 | Q4 | | | | |
| Levosalbutamol | 0 | −29 | 0 | 11 | 0 | Q4 | | | | |
| Levosimendan | 2 | 46 | 1 | 42 | 0 | Q4 | | | | |
| Levosulpiride | 0 | −36 | 1 | 18 | 0 | Q4 | | | | |
| Lidocaine | 0 | −23 | 0 | 8 | 0 | Q4 | | | | |
| Linagliptin | 0 | 2 | 1 | 14 | 1 | Q4 | | | | |
| Lincomycin | 0 | −29 | 1 | −5 | 0 | Q4 | | | | |
| Lincomycin Hydrochloride | 0 | −4 | 1 | 14 | 0 | Q4 | | | | |
| Lindane | 0 | 42 | 0 | 11 | 0 | Q4 | | | | |
| Linezolid | 3 | −18 | 1 | 19 | 0 | Q4 | | | | |
| Liothyronine | 0 | 0 | 1 | 21 | 1 | Q4 | | | | |
| Liothyronine | 0 | 29 | 1 | 20 | 1 | Q4 | | | | |
| Lipoamide | 0 | −18 | 0 | 8 | 0 | Q4 | | | | |
| Lipoic Acid | 0 | −38 | 1 | 39 | 0 | Q4 | | | | |
| Lisinopril | 0 | 1 | 1 | 3 | 0 | Q4 | | | | |
| Lisuride | 0 | −27 | 1 | −9 | 0 | Q4 | | | | |
| Lithium hydride | 0 | −6 | 1 | 20 | 0 | Q4 | | | | |
| Lobeline | 0 | −38 | 1 | −5 | 0 | Q4 | | | | |
| Lobendazole | 0 | −4 | 0 | 20 | 0 | Q4 | | | | |
| Lofexidine | 0 | −30 | 0 | 0 | 0 | Q4 | | | | |
| Lofexidine | 0 | −22 | 1 | 10 | 0 | Q4 | | | | |
| Lomefloxacin | 0 | 7 | 1 | 15 | 0 | Q4 | | | | |
| Lomerizine, Lomerizine | 0 | −5 | 1 | 8 | 0 | Q4 | | | | |
| Dihydrochloride | | | | | | | | | | |
| Lomustine | 0 | 0 | 1 | 19 | 1 | Q4 | | | | |
| Lonidamine | 0 | 7 | 1 | 19 | 0 | Q4 | | | | |
| Loratadine | 0 | 7 | 0 | 8 | 0 | Q4 | | | | |
| Lorazepam | 0 | −13 | 1 | −1 | 0 | Q4 | | | | |
| Lorglumide Sodium | 0 | −29 | 1 | 13 | 1 | Q4 | | | | |
| Lornoxicam | 0 | 22 | 0 | 10 | 1 | Q4 | | | | |
| Losartan, Losartan Potassium | 0 | −3 | 1 | 19 | 1 | Q4 | | | | |
| Loxapine | 0 | −41 | 0 | 9 | 0 | Q4 | | | | |
| Loxoprofen | 0 | −19 | 1 | 10 | 0 | Q4 | | | | |
| Lufenuron | 0 | 15 | 1 | 13 | 0 | Q4 | | | | |
| Lumiracoxib | 0 | 14 | 1 | 18 | 0 | Q4 | | | | |
| Mafenide | 0 | 10 | 1 | −3 | 0 | Q4 | | | | |
| Malathion | 0 | −8 | 0 | 8 | 0 | Q4 | | | | |
| Mangafodipir Trisodium | 0 | 1 | 1 | 11 | 0 | Q4 | | | | |
| Manidipine Hydrochloride | 0 | −43 | 1 | 12 | 1 | Q4 | | | | |
| Mannitol | 0 | 5 | 1 | 0 | 0 | Q4 | | | | |
| Mebeverine | 0 | −11 | 0 | 7 | 0 | Q4 | | | | |
| Mebhydrolin Naphthalenesulfonate | 3 | 4 | 1 | 17 | 0 | Q4 | | | | |
| Meclizine | 0 | −33 | 0 | 12 | 0 | Q4 | | | | |
| Meclocycline Sulfosalicylate | 0 | 20 | 1 | 18 | 0 | Q4 | | | | |
| Meclofenamic Acid | 0 | 12 | 1 | 14 | 0 | Q4 | | | | |
| Meclofenoxate | 0 | −23 | 1 | 24 | 0 | Q4 | | | | |
| Mecysteine Hydrochloride | 0 | 20 | 1 | 22 | 0 | Q4 | | | | |
| Mefenamic Acid | 0 | −6 | 1 | 3 | 1 | Q4 | | | | |
| Mefexamide, Mefexamide | 0 | −13 | 0 | −2 | 0 | Q4 | | | | |
| Hydrochloride | | | | | | | | | | |
| Mefloquine | 0 | −18 | 1 | 13 | 0 | Q4 | | | | |
| Megestrol Acetate | 0 | 11 | 1 | 24 | 1 | Q4 | | | | |
| Meglumine | 0 | 25 | 0 | 5 | 0 | Q4 | | | | |
| Meglumine | 0 | −19 | 1 | 11 | 1 | Q4 | | | | |
| Melatonin | 0 | 4 | 1 | −3 | 0 | Q4 | | | | |
| Meloxicam | 0 | −1 | 1 | 20 | 0 | Q4 | | | | |
| Melphalan | 3 | 28 | 0 | 3 | 0 | Q4 | | | | |
| Memantine | 0 | 2 | 0 | 5 | 0 | Q4 | | | | |
| Menthol | 0 | −20 | 1 | 0 | 0 | Q4 | | | | |
| Meparfylon | 0 | −49 | 0 | 3 | 0 | Q4 | | | | |
| Mepartricin | 0 | 6 | 1 | −1 | 0 | Q4 | | | | |
| Mepenzolate | 0 | 45 | 1 | 34 | 1 | Q4 | | | | |
| Mephenesin | 0 | 14 | 0 | 13 | 0 | Q4 | | | | |
| Mepiroxol | 0 | −6 | 1 | 11 | 0 | Q4 | | | | |
| Mepivacaine | 0 | 7 | 0 | 10 | 0 | Q4 | | | | |
| Meprylcaine Hydrochloride | 0 | −10 | 0 | 7 | 0 | Q4 | | | | |
| Mepyramine | 0 | −11 | 0 | 13 | 0 | Q4 | | | | |
| Merbromin | 2 | −6 | 0 | 36 | 0 | Q4 | | | | |
| Mercaptopurine | 0 | 2 | 0 | 9 | 0 | Q4 | | | | |
| Meropenem | 0 | 47 | 1 | 28 | 0 | Q4 | | | | |
| Meropenem | 0 | −1 | 1 | 3 | 0 | Q4 | | | | |
| Mesalazine | 0 | 9 | 1 | 19 | 0 | Q4 | | | | |
| Mesna | 0 | 18 | 1 | 5 | 0 | Q4 | | | | |
| Mesoridazine | 1 | −5 | 1 | 40 | 1 | Q4 | | | | |
| Mestranol | 0 | −6 | 0 | 10 | 1 | Q4 | | | | |
| Meta-cresyl Acetate | 0 | −7 | 1 | −3 | 0 | Q4 | | | | |
| Metaraminol | 0 | −30 | 0 | 6 | 1 | Q4 | | | | |
| Metaraminol | 0 | 39 | 1 | 25 | 1 | Q4 | | | | |
| Metaxalone | 0 | 13 | 1 | 6 | 0 | Q4 | | | | |
| Metergoline | 2 | −12 | 1 | 16 | 0 | Q4 | | | | |
| Metformin | 0 | −15 | 0 | 0 | 0 | Q4 | | | | |
| Methacholine | 0 | 2 | 1 | 9 | 0 | Q4 | | | | |
| Methacycline | 0 | −10 | 0 | 11 | 0 | Q4 | | | | |
| Methapyrilene | 0 | 17 | 1 | 13 | 0 | Q4 | | | | |
| Methazolamide | 0 | 2 | 1 | 10 | 0 | Q4 | | | | |
| Methazolamide | 0 | −32 | 1 | 11 | 0 | Q4 | | | | |
| Methimazole | 0 | −29 | 0 | 12 | 0 | Q4 | | | | |
| Methiothepin Mesylate, Metitepine | 1 | 11 | 1 | −5 | 0 | Q4 | | | | |
| Maleate | | | | | | | | | | |
| Methocarbamol | 0 | 4 | 0 | 10 | 0 | Q4 | | | | |
| Methoprene (s) | 0 | −9 | 1 | 12 | 0 | Q4 | | | | |
| Methotrexate | 0 | −32 | 1 | −4 | 0 | Q4 | | | | |
| Methotrexate(+/−) | 0 | 0 | 0 | 0 | 0 | Q4 | | | | |
| Methoxamine | 0 | 3 | 1 | 1 | 0 | Q4 | | | | |
| Methoxsalen | 0 | −31 | 0 | 14 | 0 | Q4 | | | | |
| Methscopolamine Bromide | 0 | 1 | 1 | 12 | 0 | Q4 | | | | |
| Methscopolamine Bromide | 0 | 14 | 1 | −15 | 0 | Q4 | | | | |
| Methsuximide | 0 | −39 | 0 | 19 | 0 | Q4 | | | | |
| Methyclothiazide | 0 | 25 | 1 | 17 | 0 | Q4 | | | | |
| Methylatropine Nitrate | 0 | −2 | 1 | 14 | 0 | Q4 | | | | |
| Methylergometrine | 0 | −43 | 0 | 7 | 0 | Q4 | | | | |
| Methyltestosterone | 0 | −40 | 1 | −2 | 0 | Q4 | | | | |
| Methylthiouracil | 0 | 31 | 0 | 16 | 0 | Q4 | | | | |
| Methysergide Maleate | 2 | 1 | 0 | 16 | 0 | Q4 | | | | |
| Methysergide Maleate | 2 | 26 | 1 | 7 | 0 | Q4 | | | | |
| Meticillin | 0 | −19 | 0 | 18 | 0 | Q4 | | | | |
| Meticrane | 0 | −33 | 0 | 9 | 0 | Q4 | | | | |
| Metoclopramide | 0 | −2 | 1 | 12 | 0 | Q4 | | | | |
| Metolazone | 0 | −2 | 1 | 19 | 0 | Q4 | | | | |
| Metoprolol | 0 | 4 | 1 | 22 | 0 | Q4 | | | | |
| Metrifonate | 0 | −34 | 0 | 8 | 0 | Q4 | | | | |
| Metronidazole | 0 | −12 | 0 | 15 | 1 | Q4 | | | | |
| Metyrapone | 0 | −60 | 1 | 39 | 0 | Q4 | | | | |
| Mexeneone | 0 | −8 | 1 | 9 | 0 | Q4 | | | | |
| Mexiletine | 0 | 2 | 1 | 13 | 0 | Q4 | | | | |
| Mianserin | 0 | −18 | 0 | 17 | 0 | Q4 | | | | |
| Mifepristone | 3 | 12 | 1 | −3 | 0 | Q4 | | | | |
| Mifepristone | 3 | 8 | 1 | 7 | 0 | Q4 | | | | |
| Miglitol | 0 | −9 | 0 | 13 | 0 | Q4 | | | | |
| Miglustat | 0 | −6 | 1 | 0 | 0 | Q4 | | | | |
| Milrinone | 0 | 4 | 0 | 23 | 0 | Q4 | | | | |
| Miltefosine | 0 | −31 | 1 | 11 | 0 | Q4 | | | | |
| Minaprine | 0 | −22 | 0 | 10 | 0 | Q4 | | | | |
| Minocycline | 0 | −6 | 1 | 16 | 0 | Q4 | | | | |
| Minoxidil | 0 | −5 | 1 | 9 | 1 | Q4 | | | | |
| Misoprostol | 0 | 13 | 1 | 32 | 0 | Q4 | | | | |
| Mitotane | 0 | −19 | 0 | −1 | 0 | Q4 | | | | |
| Moclobemide | 0 | −6 | 1 | 14 | 0 | Q4 | | | | |
| Modafinil | 0 | −5 | 1 | 25 | 0 | Q4 | | | | |
| Modaline Sulfate | 0 | −9 | 1 | 25 | 0 | Q4 | | | | |
| Moexipril | 0 | −29 | 1 | 9 | 1 | Q4 | | | | |
| Moguisteine | 0 | −2 | 1 | 15 | 0 | Q4 | | | | |
| Monobenzone | 0 | 7 | 1 | 16 | 0 | Q4 | | | | |
| Montelukast | 0 | −5 | 1 | 12 | 0 | Q4 | | | | |
| Montelukast Sodium | 0 | 21 | 1 | 16 | 0 | Q4 | | | | |
| Morantel Citrate | 0 | −11 | 1 | 11 | 0 | Q4 | | | | |
| Moroxydine Hydrochloride | 0 | −22 | 1 | 4 | 0 | Q4 | | | | |
| Moxidectin | 0 | 1 | 0 | 14 | 1 | Q4 | | | | |
| Moxifloxacin Hydrochloride | 0 | 7 | 1 | 14 | 0 | Q4 | | | | |
| Moxisylyte | 0 | −22 | 0 | 3 | 0 | Q4 | | | | |
| Mupirocin | 0 | 11 | 1 | 20 | 0 | Q4 | | | | |
| N-acetylneuramic Acid, | 0 | −9 | 1 | 19 | 1 | Q4 | | | | |
| Aceneuramic Acid | | | | | | | | | | |
| N-acetylprocainamide | 0 | −11 | 0 | 9 | 1 | Q4 | | | | |
| Hydrochloride, Acecainide | | | | | | | | | | |
| Hydrochloride | | | | | | | | | | |
| Nabumetone | 0 | −13 | 1 | 15 | 0 | Q4 | | | | |
| Nadide | 0 | −14 | 1 | 5 | 0 | Q4 | | | | |
| Nadifloxacin | 0 | −14 | 1 | 10 | 0 | Q4 | | | | |
| Nafcillin Sodium | 0 | −7 | 0 | 15 | 0 | Q4 | | | | |
| Nafronyl Oxalate | 0 | 15 | 1 | 15 | 0 | Q4 | | | | |
| Naftifine | 0 | −12 | 0 | 3 | 0 | Q4 | | | | |
| Nalbuphine | 0 | −8 | 1 | 16 | 0 | Q4 | | | | |
| Nalbuphine Hydrochloride | 0 | −11 | 1 | 15 | 0 | Q4 | | | | |
| Nalidixic Acid | 0 | −1 | 1 | 13 | 1 | Q4 | | | | |
| Naloxone | 0 | −41 | 1 | 21 | 1 | Q4 | | | | |
| Naloxone Hydrochloride | 0 | −16 | 0 | 11 | 0 | Q4 | | | | |
| Nanofin | 0 | 17 | 1 | 12 | 0 | Q4 | | | | |
| Naphazoline | 0 | −1 | 1 | 13 | 1 | Q4 | | | | |
| Naproxen | 0 | −25 | 0 | 15 | 0 | Q4 | | | | |
| Naproxol | 0 | −10 | 0 | 16 | 0 | Q4 | | | | |
| Natamycin | 0 | −7 | 1 | 35 | 0 | Q4 | | | | |
| Nateglinide | 0 | −20 | 1 | −2 | 1 | Q4 | | | | |
| Nateglinide | 0 | −18 | 1 | 13 | 0 | Q4 | | | | |
| Nebivolol | 0 | −10 | 1 | 6 | 0 | Q4 | | | | |
| Nefazodone | 0 | −37 | 0 | 3 | 0 | Q4 | | | | |
| Nefiracetam | 0 | −21 | 0 | 13 | 0 | Q4 | | | | |
| Nefopam | 0 | −15 | 0 | 3 | 0 | Q4 | | | | |
| Nelarabin | 0 | −10 | 0 | 0 | 0 | Q4 | | | | |
| Nelfinavir | 0 | 16 | 1 | 13 | 0 | Q4 | | | | |
| Neomycin Sulfate | 0 | 15 | 0 | 18 | 0 | Q4 | | | | |
| Neostigmine | 0 | −15 | 0 | 15 | 0 | Q4 | | | | |
| Netilmicin Sulfate | 0 | −6 | 1 | 12 | 0 | Q4 | | | | |
| Nevirapine | 0 | 3 | 0 | 15 | 0 | Q4 | | | | |
| Niacin | 0 | 8 | 1 | 13 | 0 | Q4 | | | | |
| Nialamide | 0 | −6 | 1 | 11 | 0 | Q4 | | | | |
| Nicardipine | 0 | 11 | 0 | 12 | 0 | Q4 | | | | |
| Nicergoline | 2 | 6 | 1 | 7 | 0 | Q4 | | | | |
| Nicolsamide, Niclosamide | 0 | −2 | 1 | 21 | 1 | Q4 | | | | |
| Nicopholine | 0 | 26 | 0 | 21 | 0 | Q4 | | | | |
| Nicotinamide | 0 | −8 | 1 | 16 | 0 | Q4 | | | | |
| Nicotine | 0 | 2 | 1 | 21 | 1 | Q4 | | | | |
| Nicotinyl Alcohol Tartrate | 0 | −5 | 0 | 4 | 0 | Q4 | | | | |
| Nifedipine | 0 | −36 | 0 | 2 | 0 | Q4 | | | | |
| Niflumic Acid | 0 | −14 | 1 | 9 | 0 | Q4 | | | | |
| Nifursol | 0 | 47 | 1 | 20 | 0 | Q4 | | | | |
| Nikethamide | 0 | 12 | 0 | −4 | 0 | Q4 | | | | |
| Nilotinib | 0 | −35 | 1 | 17 | 1 | Q4 | | | | |
| Nilutamide | 0 | 33 | 1 | 15 | 0 | Q4 | | | | |
| Nilutamide | 0 | −30 | 1 | 4 | 0 | Q4 | | | | |
| Nimesulide | 0 | −19 | 1 | 13 | 0 | Q4 | | | | |
| Nimodipine | 0 | −6 | 1 | 2 | 0 | Q4 | | | | |
| Nimustine | 0 | −1 | 1 | 10 | 0 | Q4 | | | | |
| Nisoldipine | 0 | 6 | 1 | 25 | 1 | Q4 | | | | |
| Nitarsone | 0 | −3 | 1 | 19 | 0 | Q4 | | | | |
| Nitazoxanide | 0 | −16 | 1 | 22 | 0 | Q4 | | | | |
| Nithiamide | 0 | 3 | 1 | 21 | 1 | Q4 | | | | |
| Nitrazepam | 0 | −1 | 1 | −4 | 0 | Q4 | | | | |
| Nitrendipine | 0 | −12 | 1 | 18 | 0 | Q4 | | | | |
| Nitrofural | 0 | −14 | 0 | 1 | 0 | Q4 | | | | |
| Nitrofurantoin | 0 | −3 | 1 | 11 | 0 | Q4 | | | | |
| Nitroglycerin | 0 | 2 | 1 | 17 | 1 | Q4 | | | | |
| Nitromide | 0 | −7 | 0 | 11 | 0 | Q4 | | | | |
| Nitroxoline | 0 | −5 | 1 | −6 | 0 | Q4 | | | | |
| Nizatidine | 0 | −18 | 0 | 14 | 0 | Q4 | | | | |
| Nomifensine | 0 | −12 | 0 | 13 | 0 | Q4 | | | | |
| Nonoxynol-9 | 0 | −7 | 0 | 23 | 0 | Q4 | | | | |
| Norepinephrine | 2 | 44 | 1 | 32 | 0 | Q4 | | | | |
| Norepinephrine | 2 | 6 | 1 | −1 | 0 | Q4 | | | | |
| Norethindrone | 0 | 9 | 0 | 15 | 1 | Q4 | | | | |
| Norethindrone Acetate | 0 | −5 | 1 | 18 | 0 | Q4 | | | | |
| Norethisterone | 0 | 18 | 1 | 1 | 0 | Q4 | | | | |
| Norethynodrel | 0 | 1 | 0 | 10 | 0 | Q4 | | | | |
| Norfloxacin | 0 | 0 | 1 | 9 | 0 | Q4 | | | | |
| Norgestimate | 0 | −5 | 0 | 12 | 0 | Q4 | | | | |
| Norgestrel | 0 | 4 | 1 | 22 | 1 | Q4 | | | | |
| Norgestrel, Levonorgestrel | 0 | 13 | 0 | 14 | 1 | Q4 | | | | |
| Noscapine Hydrochloride | 0 | −15 | 1 | 23 | 0 | Q4 | | | | |
| Novobiocin | 0 | −17 | 0 | 8 | 0 | Q4 | | | | |
| Nystatin | 0 | −34 | 0 | 39 | 1 | Q4 | | | | |
| Nystatin | 0 | −76 | 1 | −15 | 1 | Q4 | | | | |
| Octisalate | 0 | −17 | 0 | 5 | 0 | Q4 | | | | |
| Octodrine | 0 | 2 | 0 | −2 | 0 | Q4 | | | | |
| Octreotide | 0 | 3 | 1 | −5 | 1 | Q4 | | | | |
| Ofloxacin | 0 | −13 | 1 | 16 | 0 | Q4 | | | | |
| Olanzapine | 0 | 6 | 1 | 9 | 0 | Q4 | | | | |
| Oleandomycin Phosphate | 0 | −9 | 1 | 16 | 0 | Q4 | | | | |
| Oleic Acid | 0 | 23 | 1 | 25 | 1 | Q4 | | | | |
| Olmesartan | 0 | −13 | 0 | 18 | 0 | Q4 | | | | |
| Olmesartan Medoxomil | 0 | −5 | 1 | 12 | 0 | Q4 | | | | |
| Olopatadine | 0 | −42 | 1 | −9 | 0 | Q4 | | | | |
| Olsalazine | 3 | −36 | 1 | 22 | 1 | Q4 | | | | |
| Oltipraz | 0 | −2 | 1 | −1 | 0 | Q4 | | | | |
| Ondansetron | 0 | −6 | 1 | 14 | 0 | Q4 | | | | |
| Orbifloxacin | 0 | −18 | 0 | 15 | 0 | Q4 | | | | |
| Orciprenaline | 0 | 15 | 0 | 25 | 0 | Q4 | | | | |
| Orlistat | 0 | 17 | 1 | 5 | 1 | Q4 | | | | |
| Orlistat | 0 | 15 | 1 | −1 | 0 | Q4 | | | | |
| Ornidazole | 0 | −22 | 0 | 12 | 0 | Q4 | | | | |
| Ornithine Aketoglutarate, Ornithine | 0 | −36 | 1 | 14 | 0 | Q4 | | | | |
| Hydrochloride | | | | | | | | | | |
| Orotic Acid | 0 | −5 | 0 | 16 | 0 | Q4 | | | | |
| Orphenadrine | 0 | −17 | 0 | 10 | 0 | Q4 | | | | |
| Oseltamivir | 0 | −28 | 1 | 26 | 1 | Q4 | | | | |
| Oseltamivir Phosphate | 0 | −7 | 1 | 13 | 0 | Q4 | | | | |
| Ouabain | 0 | −5 | 0 | 11 | 0 | Q4 | | | | |
| Ouabain | 0 | −45 | 0 | −13 | 0 | Q4 | | | | |
| Oxacillin Sodium | 0 | −20 | 0 | 11 | 0 | Q4 | | | | |
| Oxaprozin | 0 | 0 | 1 | 15 | 1 | Q4 | | | | |
| Oxcarbazepine, Trileptal | 0 | 3 | 1 | 35 | 0 | Q4 | | | | |
| Oxeladin | 0 | −27 | 1 | 6 | 0 | Q4 | | | | |
| Oxethazaine | 0 | 25 | 1 | 42 | 0 | Q4 | | | | |
| Oxfendazole | 0 | 10 | 0 | 20 | 1 | Q4 | | | | |
| Oxiconazole | 0 | 12 | 1 | 15 | 1 | Q4 | | | | |
| Oxidopamine Hydrochloride | 2 | 1 | 0 | 19 | 0 | Q4 | | | | |
| Oxiniacic Acid | 0 | −6 | 1 | 12 | 0 | Q4 | | | | |
| Oxolamine Citrate | 0 | −1 | 1 | 10 | 0 | Q4 | | | | |
| Oxolinic Acid | 0 | 7 | 0 | 13 | 1 | Q4 | | | | |
| Oxybenzone | 0 | −15 | 0 | 12 | 0 | Q4 | | | | |
| Oxybuprocaine | 0 | 7 | 1 | 11 | 0 | Q4 | | | | |
| Oxybutynin | 0 | −9 | 0 | 0 | 1 | Q4 | | | | |
| Oxymetazoline | 0 | −13 | 1 | 20 | 0 | Q4 | | | | |
| Oxymetholone | 0 | −18 | 1 | −7 | 0 | Q4 | | | | |
| Oxyphencyclimine | 0 | 15 | 1 | 11 | 0 | Q4 | | | | |
| Oxyphenonium | 0 | −14 | 1 | 30 | 1 | Q4 | | | | |
| Oxytetracycline | 0 | 0 | 1 | 10 | 0 | Q4 | | | | |
| P-Cresol | 0 | −15 | 0 | 15 | 0 | Q4 | | | | |
| Paliperidone | 0 | −26 | 1 | 9 | 0 | Q4 | | | | |
| Palmidrol | 0 | −40 | 0 | 7 | 0 | Q4 | | | | |
| Pamidronate | 0 | 0 | 1 | −5 | 0 | Q4 | | | | |
| Pancuronium | 0 | −4 | 1 | 1 | 0 | Q4 | | | | |
| Pancuronium Bromide | 0 | 9 | 1 | 11 | 0 | Q4 | | | | |
| Pantethine | 0 | −53 | 1 | 7 | 0 | Q4 | | | | |
| Panthenol | 0 | −32 | 1 | 6 | 0 | Q4 | | | | |
| Pantoprazole, Pantoprazole Sodium | 0 | −8 | 0 | −1 | 0 | Q4 | | | | |
| Salt | | | | | | | | | | |
| Pantothenic Acid | 0 | 7 | 0 | 5 | 1 | Q4 | | | | |
| Papaverine | 0 | 12 | 0 | 27 | 0 | Q4 | | | | |
| Parachlorophenol | 0 | 21 | 0 | 15 | 0 | Q4 | | | | |
| Paramethadione | 0 | −15 | 0 | 3 | 0 | Q4 | | | | |
| Pargyline | 0 | −25 | 1 | 11 | 0 | Q4 | | | | |
| Paromomycin Sulfate | 0 | 9 | 1 | 14 | 0 | Q4 | | | | |
| Paroxetine | 0 | 38 | 1 | 15 | 0 | Q4 | | | | |
| Paroxypropione | 0 | −11 | 1 | 17 | 0 | Q4 | | | | |
| Pasiniazid | 0 | −61 | 1 | 17 | 0 | Q4 | | | | |
| Pazufloxacin Mesylate, Pazufloxacin | 0 | −10 | 1 | 11 | 0 | Q4 | | | | |
| Pefloxacin | 0 | −22 | 0 | 21 | 0 | Q4 | | | | |
| Pefloxacin | 0 | −16 | 0 | 13 | 1 | Q4 | | | | |
| Pemetrexed | 0 | −8 | 0 | 9 | 0 | Q4 | | | | |
| Pempidine Tartrate | 0 | −19 | 1 | 13 | 0 | Q4 | | | | |
| Penbutolol | 0 | −8 | 1 | 17 | 0 | Q4 | | | | |
| Penciclovir | 0 | −44 | 1 | 4 | 0 | Q4 | | | | |
| Penfluridol | 0 | 3 | 1 | 8 | 0 | Q4 | | | | |
| Penicillamine | 0 | −15 | 0 | 16 | 0 | Q4 | | | | |
| Pentagastrin | 0 | −6 | 1 | 14 | 1 | Q4 | | | | |
| Pentamidine | 0 | 6 | 1 | 33 | 1 | Q4 | | | | |
| Pentetic Acid, | 0 | 11 | 0 | −2 | 0 | Q4 | | | | |
| Diethylenetriaminepentaacetic Acid | | | | | | | | | | |
| Pentetrazole, Pentylenetetrazol | 0 | −12 | 1 | 18 | 1 | Q4 | | | | |
| Pentoxifylline | 0 | 4 | 0 | 6 | 0 | Q4 | | | | |
| Perflubron emulsion | 0 | 16 | 1 | −10 | 0 | Q4 | | | | |
| Pergolide | 0 | −18 | 0 | 16 | 0 | Q4 | | | | |
| Perindopril | 0 | −27 | 0 | 18 | 0 | Q4 | | | | |
| Permethrin | 0 | −21 | 0 | 11 | 0 | Q4 | | | | |
| Perphenazine | 1 | 23 | 1 | 23 | 1 | Q4 | | | | |
| Phenacetin | 0 | −5 | 1 | 16 | 0 | Q4 | | | | |
| Phenazopyridine | 3 | 9 | 0 | 6 | 0 | Q4 | | | | |
| Phenelzine | 0 | 2 | 1 | −4 | 0 | Q4 | | | | |
| Phenethicillin Potassium | 0 | −2 | 0 | 3 | 0 | Q4 | | | | |
| Phenformin | 0 | −30 | 1 | 13 | 0 | Q4 | | | | |
| Phenindione | 2 | −10 | 1 | 0 | 0 | Q4 | | | | |
| Pheniramine | 0 | −9 | 1 | 1 | 0 | Q4 | | | | |
| Phenobarbital | 0 | −22 | 0 | −7 | 0 | Q4 | | | | |
| Phenolphthalein | 0 | 10 | 1 | 13 | 0 | Q4 | | | | |
| Phenothiazine | 1 | −84 | 0 | 24 | 1 | Q4 | | | | |
| Phenothrin | 0 | −50 | 0 | 11 | 0 | Q4 | | | | |
| Phenoxybenzamine | 0 | 8 | 1 | −5 | 0 | Q4 | | | | |
| Phenoxymethylpenicillin | 0 | 0 | 1 | 14 | 0 | Q4 | | | | |
| Phensuximide | 0 | −7 | 1 | 4 | 0 | Q4 | | | | |
| Phentolamine | 0 | −21 | 0 | 17 | 0 | Q4 | | | | |
| Phenylacetic acid | 0 | −1 | 1 | 13 | 0 | Q4 | | | | |
| Phenylacetic acid | 0 | −54 | 1 | 3 | 0 | Q4 | | | | |
| Phenylbutazone | 1 | −23 | 0 | 5 | 0 | Q4 | | | | |
| Phenylephrine | 0 | 38 | 1 | 31 | 0 | Q4 | | | | |
| Phenylpropanolamine | 0 | −2 | 1 | −6 | 1 | Q4 | | | | |
| Phosphonoserine | 0 | −6 | 1 | 10 | 0 | Q4 | | | | |
| Phthalylsulfacetamide | 0 | 1 | 1 | −1 | 0 | Q4 | | | | |
| Phthalylsulfathiazole | 0 | 3 | 1 | 13 | 1 | Q4 | | | | |
| Phylloquinone | 3 | 4 | 0 | 31 | 2 | Q4 | | | | |
| Physostigmine | 0 | −73 | 0 | 18 | 0 | Q4 | | | | |
| Picolamine | 0 | −1 | 1 | 4 | 0 | Q4 | | | | |
| Piconol | 0 | −1 | 1 | 26 | 1 | Q4 | | | | |
| Pidolic Acid | 0 | −57 | 1 | 15 | 0 | Q4 | | | | |
| Pidotimod | 0 | −10 | 1 | 14 | 0 | Q4 | | | | |
| Pilocarpine | 0 | −2 | 1 | 14 | 0 | Q4 | | | | |
| Pinacidil | 0 | 15 | 1 | 26 | 1 | Q4 | | | | |
| Pindolol | 0 | −13 | 1 | 24 | 1 | Q4 | | | | |
| Pioglitazone | 0 | 2 | 1 | 33 | 1 | Q4 | | | | |
| Pipamperone | 0 | −4 | 1 | 8 | 0 | Q4 | | | | |
| Pipemidic Acid | 0 | −27 | 1 | 12 | 0 | Q4 | | | | |
| Piperacetazine | 1 | −32 | 0 | 48 | 2 | Q4 | | | | |
| Piperacillin Sodium | 0 | −25 | 0 | 17 | 0 | Q4 | | | | |
| Piperazine | 0 | −3 | 0 | 16 | 0 | Q4 | | | | |
| Piperidolate Hydrochloride | 0 | −21 | 0 | 8 | 0 | Q4 | | | | |
| Piperine | 0 | −13 | 1 | 10 | 0 | Q4 | | | | |
| Piperonyl Butoxide | 0 | 0 | 0 | 12 | 0 | Q4 | | | | |
| Pipobroman | 0 | −28 | 0 | 0 | 0 | Q4 | | | | |
| Piracetam | 0 | −21 | 0 | 9 | 0 | Q4 | | | | |
| Pirenperone | 0 | 2 | 1 | 20 | 0 | Q4 | | | | |
| Pirenzepine | 0 | 16 | 0 | 19 | 0 | Q4 | | | | |
| Piroctone Olamine | 0 | 44 | 1 | 39 | 1 | Q4 | | | | |
| Piromidic Acid | 0 | −30 | 1 | 7 | 0 | Q4 | | | | |
| Piroxicam | 0 | 4 | 1 | 19 | 0 | Q4 | | | | |
| Pizotyline Malate | 0 | 8 | 1 | 37 | 1 | Q4 | | | | |
| Praesterone Acetate, Prasterone | 0 | −3 | 1 | 5 | 0 | Q4 | | | | |
| Acetate | | | | | | | | | | |
| Pralidoxime | 0 | −12 | 1 | 17 | 0 | Q4 | | | | |
| Pramipexole | 0 | −19 | 1 | 1 | 0 | Q4 | | | | |
| Pramocaine | 0 | 14 | 0 | 13 | 0 | Q4 | | | | |
| Pranoprofen | 0 | −14 | 1 | 8 | 0 | Q4 | | | | |
| Prasterone | 0 | 1 | 1 | 15 | 0 | Q4 | | | | |
| Prasugrel | 0 | 15 | 1 | 22 | 1 | Q4 | | | | |
| Pravastatin | 0 | 10 | 1 | 10 | 0 | Q4 | | | | |
| Pravastatin Sodium | 0 | 21 | 1 | 11 | 0 | Q4 | | | | |
| Praziquantel | 0 | −3 | 1 | 21 | 0 | Q4 | | | | |
| Pregabalin | 0 | −5 | 1 | 1 | 0 | Q4 | | | | |
| Pregnenolone | 0 | −11 | 1 | 12 | 0 | Q4 | | | | |
| Pregnenolone Succinate | 0 | 4 | 1 | 21 | 1 | Q4 | | | | |
| Pridinol | 0 | −18 | 1 | 4 | 0 | Q4 | | | | |
| Prilocaine | 0 | −3 | 0 | 12 | 0 | Q4 | | | | |
| Primaquine | 0 | −58 | 0 | 7 | 0 | Q4 | | | | |
| Primidone | 0 | −15 | 1 | 12 | 0 | Q4 | | | | |
| Probenecid | 0 | 14 | 1 | 13 | 0 | Q4 | | | | |
| Probucol | 0 | 12 | 0 | −1 | 0 | Q4 | | | | |
| Procainamide, Pronestyl | 0 | −4 | 0 | 10 | 0 | Q4 | | | | |
| Procaine, Novocain | 0 | −10 | 0 | 25 | 0 | Q4 | | | | |
| Procarbazine | 0 | −21 | 1 | 9 | 0 | Q4 | | | | |
| Procaterol | 0 | 16 | 1 | 21 | 0 | Q4 | | | | |
| Procodazole | 0 | −15 | 1 | 15 | 0 | Q4 | | | | |
| Procyclidine | 0 | 0 | 1 | 26 | 1 | Q4 | | | | |
| Progesterone | 0 | 36 | 1 | 36 | 0 | Q4 | | | | |
| Proglumide | 0 | −4 | 1 | −1 | 0 | Q4 | | | | |
| Proguanil; Chlorguanide | 0 | −7 | 0 | 20 | 1 | Q4 | | | | |
| Hydrochloride | | | | | | | | | | |
| Promethazine | 1 | −30 | 0 | 5 | 0 | Q4 | | | | |
| Pronetalol Hydrochloride | 0 | −12 | 1 | 10 | 0 | Q4 | | | | |
| Propafenone | 0 | 16 | 1 | 21 | 1 | Q4 | | | | |
| Propantheline | 0 | −7 | 1 | 20 | 0 | Q4 | | | | |
| Proparacaine | 0 | −6 | 1 | 13 | 0 | Q4 | | | | |
| Propofol | 0 | −18 | 0 | 11 | 0 | Q4 | | | | |
| Propoxur | 0 | −51 | 1 | 8 | 0 | Q4 | | | | |
| Propoxycaine | 0 | 13 | 0 | 14 | 0 | Q4 | | | | |
| Propranolol | 0 | −30 | 0 | 2 | 0 | Q4 | | | | |
| Propylthiouracil | 0 | −4 | 0 | 8 | 0 | Q4 | | | | |
| Proscillaridin | 0 | 8 | 1 | 16 | 0 | Q4 | | | | |
| Protionamide, Ektebin | 0 | −18 | 1 | 12 | 0 | Q4 | | | | |
| Protirelin | 0 | 2 | 1 | 15 | 1 | Q4 | | | | |
| Protoporphyrin Ix | 0 | −70 | 1 | 2 | 0 | Q4 | | | | |
| Protriptyline | 0 | 21 | 1 | 15 | 0 | Q4 | | | | |
| Proxyphylline | 0 | −21 | 1 | 11 | 0 | Q4 | | | | |
| Prulifloxacin | 0 | 7 | 1 | 16 | 0 | Q4 | | | | |
| Pseudoephedrine | 0 | −13 | 1 | 5 | 0 | Q4 | | | | |
| Pyrantel Pamoate, Oxantel Pamoate | 0 | −4 | 1 | 14 | 0 | Q4 | | | | |
| Pyrantel Pamoate, Oxantel Pamoate | 0 | 5 | 1 | 17 | 1 | Q4 | | | | |
| Pyrazinamide | 0 | −20 | 0 | 13 | 0 | Q4 | | | | |
| Pyrethrins | 0 | 4 | 0 | 11 | 0 | Q4 | | | | |
| Pyridostigmine | 0 | 4 | 1 | 9 | 0 | Q4 | | | | |
| Pyridoxine | 0 | 1 | 1 | 21 | 0 | Q4 | | | | |
| Pyrimethamine | 0 | −11 | 1 | 12 | 1 | Q4 | | | | |
| Pyrithyldione | 0 | −24 | 1 | 9 | 0 | Q4 | | | | |
| Pyritinol | 0 | −15 | 1 | 4 | 0 | Q4 | | | | |
| Pyronaridine Tetraphosphate | 3 | 16 | 1 | 9 | 0 | Q4 | | | | |
| Quetiapine | 0 | −2 | 1 | 13 | 0 | Q4 | | | | |
| Quinapril | 0 | −24 | 1 | 13 | 0 | Q4 | | | | |
| Quinaprilat | 0 | −5 | 1 | 5 | 0 | Q4 | | | | |
| Quinestrol | 0 | 15 | 0 | 24 | 0 | Q4 | | | | |
| Quinethazone | 0 | 15 | 1 | 17 | 1 | Q4 | | | | |
| Quinidine | 0 | −6 | 1 | 20 | 0 | Q4 | | | | |
| Quinine | 0 | −32 | 1 | −7 | 0 | Q4 | | | | |
| Quinine Ethyl Carbonate | 0 | −21 | 0 | −11 | 0 | Q4 | | | | |
| Quinine Sulfate | 0 | −30 | 1 | 14 | 1 | Q4 | | | | |
| Quipazine Dimaleate, Quipazine | 0 | −18 | 1 | 9 | 0 | Q4 | | | | |
| Maleate | | | | | | | | | | |
| Rabeprazole | 0 | −46 | 0 | 3 | 0 | Q4 | | | | |
| Racecadotril | 0 | −51 | 1 | 18 | 0 | Q4 | | | | |
| Racephedrine Hydrochloride | 0 | −26 | 1 | 17 | 0 | Q4 | | | | |
| Racepinephrine | 2 | 27 | 1 | 29 | 0 | Q4 | | | | |
| Ramelteon | 0 | 25 | 0 | 20 | 0 | Q4 | | | | |
| Ramifenazone | 0 | −11 | 0 | 9 | 0 | Q4 | | | | |
| Ramipril | 0 | 8 | 0 | 16 | 0 | Q4 | | | | |
| Ramoplanin [a2 Shown; 2 mm] | 0 | −3 | 1 | 12 | 0 | Q4 | | | | |
| Ranitidine | 0 | 35 | 1 | 15 | 0 | Q4 | | | | |
| Ranitidine | 0 | 23 | 1 | 16 | 0 | Q4 | | | | |
| Ranolazine, Ranolazine | 0 | −53 | 0 | 12 | 0 | Q4 | | | | |
| Dihydrochloride | | | | | | | | | | |
| Ranolazine, Ranolazine | 0 | −18 | 1 | 2 | 0 | Q4 | | | | |
| Dihydrochloride | | | | | | | | | | |
| Reboxetine Mesylate | 0 | −33 | 1 | 8 | 0 | Q4 | | | | |
| Remacemide Hydrochloride | 0 | −21 | 1 | 13 | 0 | Q4 | | | | |
| Remoxipride | 0 | −14 | 1 | 3 | 0 | Q4 | | | | |
| Repaglinide | 0 | −14 | 1 | 18 | 0 | Q4 | | | | |
| Resorcinol | 0 | −4 | 0 | 8 | 0 | Q4 | | | | |
| Resorcinol Monoacetate | 0 | 12 | 1 | 26 | 1 | Q4 | | | | |
| Retinyl Acetate | 0 | 3 | 1 | 15 | 0 | Q4 | | | | |
| Retinyl Palmitate | 0 | −15 | 0 | 11 | 0 | Q4 | | | | |
| Riboflavin | 0 | −23 | 1 | −8 | 0 | Q4 | | | | |
| Riboflavin | 0 | 5 | 0 | 13 | 0 | Q4 | | | | |
| Riboflavin 5-phosphate Sodium | 0 | 11 | 0 | 24 | 0 | Q4 | | | | |
| Ribostamycin Sulfate | 0 | −16 | 0 | 13 | 0 | Q4 | | | | |
| Rifabutin | 1 | −51 | 0 | 2 | 0 | Q4 | | | | |
| Rifampicin | 3 | −21 | 1 | 7 | 0 | Q4 | | | | |
| Rifampin | 3 | 8 | 1 | 20 | 0 | Q4 | | | | |
| Rifapentine | 3 | −23 | 1 | −7 | 0 | Q4 | | | | |
| Rifaximin | 0 | 6 | 0 | 22 | 2 | Q4 | | | | |
| Riluzole | 0 | 0 | 1 | 7 | 0 | Q4 | | | | |
| Rimantadine | 0 | −19 | 0 | 2 | 0 | Q4 | | | | |
| Risedronate | 0 | 47 | 1 | 15 | 0 | Q4 | | | | |
| Risperidone | 0 | −20 | 0 | 18 | 0 | Q4 | | | | |
| Risperidone | 0 | −40 | 1 | −13 | 0 | Q4 | | | | |
| Ritonavir | 0 | −8 | 0 | 11 | 0 | Q4 | | | | |
| Rivastigmine | 0 | −35 | 1 | 5 | 0 | Q4 | | | | |
| Rivastigmine | 0 | −37 | 1 | 10 | 0 | Q4 | | | | |
| Rizatriptan | 0 | −9 | 1 | 10 | 0 | Q4 | | | | |
| Rocuronium | 0 | −22 | 1 | 5 | 0 | Q4 | | | | |
| Rofecoxib | 0 | 15 | 0 | 14 | 0 | Q4 | | | | |
| Rolipram | 0 | 11 | 0 | 11 | 0 | Q4 | | | | |
| Rolitetracycline | 0 | 4 | 1 | 9 | 0 | Q4 | | | | |
| Ronidazole | 0 | 31 | 1 | 20 | 0 | Q4 | | | | |
| Ropinirole | 0 | −26 | 1 | 15 | 0 | Q4 | | | | |
| Ropivacaine, Ropivacaine | 0 | 18 | 1 | 5 | 0 | Q4 | | | | |
| Hydrochloride | | | | | | | | | | |
| Rosiglitazone, Rosiglitazone | 0 | 5 | 1 | 20 | 1 | Q4 | | | | |
| Maleate | | | | | | | | | | |
| Roxarsone | 0 | −18 | 1 | 17 | 1 | Q4 | | | | |
| Roxatidine acetate | 0 | 22 | 1 | 6 | 0 | Q4 | | | | |
| Roxithromycin | 0 | −4 | 0 | 17 | 1 | Q4 | | | | |
| Rufloxacin Hydrochloride | 0 | −3 | 1 | 21 | 0 | Q4 | | | | |
| Rutin | 2 | 1 | 1 | 18 | 0 | Q4 | | | | |
| S-(−)-carbidopa, Carbidopa | 2 | −91 | 0 | 16 | 0 | Q4 | | | | |
| Saccharin | 0 | −8 | 0 | 11 | 0 | Q4 | | | | |
| Salbutamol | 0 | 25 | 1 | 25 | 0 | Q4 | | | | |
| Salicyl Alcohol | 0 | −5 | 1 | 19 | 0 | Q4 | | | | |
| Salicylamide | 0 | −20 | 1 | 16 | 1 | Q4 | | | | |
| Salicylanilide | 0 | 1 | 1 | 13 | 0 | Q4 | | | | |
| Salicylic Acid | 0 | −32 | 0 | 14 | 0 | Q4 | | | | |
| Salsalate | 0 | −22 | 1 | 14 | 1 | Q4 | | | | |
| Sarafloxacin | 0 | 2 | 0 | 4 | 0 | Q4 | | | | |
| Saxagliptin | 0 | 34 | 1 | 38 | 0 | Q4 | | | | |
| Scopolamine | 0 | −26 | 1 | −6 | 1 | Q4 | | | | |
| Scopolamine Hydrobromide | 0 | 5 | 1 | 3 | 1 | Q4 | | | | |
| Secnidazole | 0 | −24 | 1 | 22 | 2 | Q4 | | | | |
| Selegiline | 0 | −36 | 0 | −5 | 0 | Q4 | | | | |
| Selegiline | 0 | 9 | 1 | 19 | 0 | Q4 | | | | |
| Selenomethionine | 0 | −5 | 1 | 10 | 0 | Q4 | | | | |
| Semustine | 0 | −7 | 1 | 18 | 0 | Q4 | | | | |
| Sennoside A | 0 | −10 | 1 | 14 | 0 | Q4 | | | | |
| Seratrodast | 3 | 20 | 1 | 14 | 0 | Q4 | | | | |
| Sertaconazole | 0 | −25 | 1 | 11 | 0 | Q4 | | | | |
| Sertraline | 0 | 16 | 1 | 22 | 0 | Q4 | | | | |
| Sevoflurane | 0 | −1 | 1 | 17 | 1 | Q4 | | | | |
| Sibutramine | 0 | −12 | 1 | 8 | 0 | Q4 | | | | |
| Sildenafil, Sildenafil Citrate | 0 | 22 | 0 | 20 | 0 | Q4 | | | | |
| Sisomicin Sulfate | 0 | −41 | 0 | 10 | 0 | Q4 | | | | |
| Sitagliptin | 0 | −16 | 0 | 4 | 0 | Q4 | | | | |
| Skf-525a Hydrochloride, Proadifen | 0 | −10 | 0 | 5 | 0 | Q4 | | | | |
| Hydrochloride | | | | | | | | | | |
| Sodium Dehydrocholate, | 0 | 21 | 1 | 28 | 1 | Q4 | | | | |
| Dehydrocholate Sodium | | | | | | | | | | |
| Sodium Gluconate | 0 | 1 | 1 | 11 | 0 | Q4 | | | | |
| Sodium Monofluorophosphate | 0 | −10 | 0 | 0 | 0 | Q4 | | | | |
| Sodium Nitroprusside | 0 | 27 | 3 | 12 | 1 | Q4 | | | | |
| Sodium Oxybate | 0 | −5 | 1 | 34 | 0 | Q4 | | | | |
| Sodium Tetradecyl Sulfate | 0 | −5 | 1 | 13 | 0 | Q4 | | | | |
| Solifenacin Succinate | 0 | −2 | 0 | −4 | 0 | Q4 | | | | |
| Sorafenib | 0 | −67 | 1 | 35 | 1 | Q4 | | | | |
| Sorbitol | 0 | −9 | 1 | 14 | 0 | Q4 | | | | |
| Sorbitol | 0 | −65 | 0 | 5 | 0 | Q4 | | | | |
| Sotalol | 0 | 6 | 1 | 6 | 0 | Q4 | | | | |
| Spaglumic Acid | 0 | −15 | 1 | 15 | 0 | Q4 | | | | |
| Sparfloxacin | 0 | −12 | 0 | 11 | 0 | Q4 | | | | |
| Sparfloxacin | 0 | −14 | 1 | 14 | 1 | Q4 | | | | |
| Sparteine Sulfate | 0 | 7 | 1 | 20 | 0 | Q4 | | | | |
| Spectinomycin | 0 | 14 | 0 | 19 | 0 | Q4 | | | | |
| Spiperone Hydrochloride, Spiperone | 0 | 2 | 2 | 12 | 0 | Q4 | | | | |
| Spiramycin | 0 | 17 | 1 | 14 | 0 | Q4 | | | | |
| Spironolactone | 0 | −9 | 0 | 20 | 0 | Q4 | | | | |
| Spironolactone | 0 | −15 | 1 | 12 | 1 | Q4 | | | | |
| Stanozolol | 0 | −5 | 1 | 33 | 1 | Q4 | | | | |
| Stavudine | 0 | −17 | 1 | 19 | 0 | Q4 | | | | |
| Streptomycin Sulfate | 0 | −46 | 0 | 13 | 0 | Q4 | | | | |
| Streptozocin | 0 | −30 | 0 | 19 | 1 | Q4 | | | | |
| Strychnine | 0 | 1 | 1 | 18 | 0 | Q4 | | | | |
| Succimer | 0 | −37 | 0 | −5 | 0 | Q4 | | | | |
| Succinylcholine | 0 | −54 | 1 | −5 | 0 | Q4 | | | | |
| Succinylsulfathiazole | 0 | 15 | 1 | 16 | 1 | Q4 | | | | |
| Sucralfate | 0 | −35 | 1 | 22 | 0 | Q4 | | | | |
| Sucralose | 0 | 0 | 1 | 16 | 0 | Q4 | | | | |
| Sulbentine | 0 | 48 | 1 | 13 | 0 | Q4 | | | | |
| Sulconazole | 0 | 10 | 1 | 13 | 1 | Q4 | | | | |
| Sulfabenzamide | 0 | −15 | 1 | 19 | 1 | Q4 | | | | |
| Sulfacarbamide | 0 | 45 | 1 | 46 | 0 | Q4 | | | | |
| Sulfacetamide | 0 | −2 | 1 | 10 | 0 | Q4 | | | | |
| Sulfachlorpyridazine | 0 | 18 | 1 | 14 | 0 | Q4 | | | | |
| Sulfadiazine | 0 | −19 | 0 | 13 | 0 | Q4 | | | | |
| Sulfadimethoxine | 0 | 42 | 1 | 13 | 0 | Q4 | | | | |
| Sulfadoxine | 0 | −12 | 1 | 3 | 0 | Q4 | | | | |
| Sulfaguanidine | 0 | −23 | 1 | 12 | 0 | Q4 | | | | |
| Sulfamerazine | 0 | −2 | 1 | 12 | 0 | Q4 | | | | |
| Sulfameter | 0 | 4 | 1 | 11 | 0 | Q4 | | | | |
| Sulfamethazine | 0 | −9 | 0 | 14 | 0 | Q4 | | | | |
| Sulfamethizole | 0 | −3 | 1 | 16 | 0 | Q4 | | | | |
| Sulfamethoxazole | 0 | −17 | 0 | 17 | 0 | Q4 | | | | |
| Sulfamethoxypyridazine | 0 | 21 | 1 | 15 | 0 | Q4 | | | | |
| Sulfamonomethoxine | 0 | 38 | 1 | 19 | 1 | Q4 | | | | |
| Sulfanilamide | 0 | −14 | 0 | 11 | 0 | Q4 | | | | |
| Sulfanilate Zinc | 0 | −6 | 0 | 15 | 0 | Q4 | | | | |
| Sulfanitran | 0 | 10 | 1 | 18 | 0 | Q4 | | | | |
| Sulfaphenazole | 0 | −4 | 1 | 14 | 0 | Q4 | | | | |
| Sulfapyridine | 0 | −37 | 1 | 20 | 0 | Q4 | | | | |
| Sulfasalazine | 3 | −29 | 0 | 13 | 0 | Q4 | | | | |
| Sulfathiazole | 0 | −17 | 0 | 9 | 0 | Q4 | | | | |
| Sulfinpyrazone | 1 | −6 | 1 | 13 | 1 | Q4 | | | | |
| Sulfisoxazole | 0 | −11 | 0 | 6 | 0 | Q4 | | | | |
| Sulfisoxazole Acetyl | 0 | −3 | 1 | 17 | 1 | Q4 | | | | |
| Sulindac | 0 | 21 | 1 | 6 | 0 | Q4 | | | | |
| Sulindac Sulfone | 0 | −27 | 1 | 3 | 0 | Q4 | | | | |
| Sulisobenzone | 0 | −19 | 0 | −2 | 0 | Q4 | | | | |
| Sulpiride | 0 | −7 | 1 | 11 | 0 | Q4 | | | | |
| Sulpiride | 0 | −52 | 1 | 7 | 0 | Q4 | | | | |
| Sumatriptan | 0 | −7 | 1 | 8 | 0 | Q4 | | | | |
| Suramin | 0 | −5 | 0 | 18 | 0 | Q4 | | | | |
| Suxibuzone | 1 | 25 | 1 | 23 | 0 | Q4 | | | | |
| Symclosene | 0 | −13 | 0 | 11 | 0 | Q4 | | | | |
| Synephrine | 0 | −16 | 0 | 12 | 0 | Q4 | | | | |
| Tacrine | 0 | −2 | 1 | 5 | 0 | Q4 | | | | |
| Tacrolimus | 0 | 27 | 1 | 14 | 0 | Q4 | | | | |
| Tadalafil | 3 | −1 | 1 | 16 | 0 | Q4 | | | | |
| Tamoxifen | 0 | 14 | 1 | 5 | 1 | Q4 | | | | |
| Tamsulosin Hydrchloride | 0 | −31 | 0 | 12 | 0 | Q4 | | | | |
| Tapentadol Hydrochloride | 0 | −12 | 1 | 12 | 0 | Q4 | | | | |
| Taxol, Paclitaxel | 0 | 45 | 1 | 19 | 1 | Q4 | | | | |
| Tazobactam | 0 | 2 | 1 | −9 | 0 | Q4 | | | | |
| Tazobactam | 0 | −11 | 0 | 11 | 0 | Q4 | | | | |
| Teicoplanin [a(2-1) Shown] | 0 | 43 | 1 | 20 | 0 | Q4 | | | | |
| Telenzepine Dihydrochloride, | 0 | 3 | 1 | 14 | 0 | Q4 | | | | |
| Telenzepine Hydrochloride | | | | | | | | | | |
| Telithromycin | 0 | 0 | 1 | 17 | 0 | Q4 | | | | |
| Telmisartan | 0 | −24 | 0 | −2 | 0 | Q4 | | | | |
| Temozolomide | 0 | −31 | 0 | 7 | 0 | Q4 | | | | |
| Temozolomide | 0 | −26 | 1 | −12 | 0 | Q4 | | | | |
| Tenatoprazole | 0 | −17 | 1 | 9 | 0 | Q4 | | | | |
| Tenofovir | 0 | −10 | 0 | 17 | 0 | Q4 | | | | |
| Tenoxicam | 0 | 11 | 1 | 15 | 0 | Q4 | | | | |
| Tenylidone | 2 | −9 | 1 | 15 | 0 | Q4 | | | | |
| Terazosin | 0 | −1 | 1 | 9 | 0 | Q4 | | | | |
| Terbinafine, Terbinafine | 0 | −23 | 0 | 6 | 0 | Q4 | | | | |
| Hydrochloride | | | | | | | | | | |
| Terbutaline | 0 | 39 | 1 | 28 | 0 | Q4 | | | | |
| Terconazole | 3 | −1 | 1 | −10 | 0 | Q4 | | | | |
| Terconazole | 3 | 27 | 1 | 35 | 0 | Q4 | | | | |
| Terfenadine | 0 | 19 | 11 | 7 | 0 | Q4 | | | | |
| Terpene Hydrate | 0 | −6 | 1 | 8 | 0 | Q4 | | | | |
| Testosterone | 0 | −1 | 0 | 5 | 0 | Q4 | | | | |
| Testosterone Propionate | 0 | 21 | 1 | 8 | 0 | Q4 | | | | |
| Tetracaine | 0 | 4 | 1 | 9 | 1 | Q4 | | | | |
| Tetracycline | 0 | 7 | 0 | 19 | 0 | Q4 | | | | |
| Tetraquinone | 3 | −10 | 0 | 15 | 0 | Q4 | | | | |
| Tetryzoline | 0 | −29 | 0 | 16 | 0 | Q4 | | | | |
| Thalidomide | 0 | 8 | 0 | 23 | 1 | Q4 | | | | |
| Theobromine | 0 | 11 | 0 | 18 | 1 | Q4 | | | | |
| Thiamine | 0 | 27 | 2 | −5 | 0 | Q4 | | | | |
| Thiamine | 0 | −10 | 0 | 30 | 0 | Q4 | | | | |
| Thiamphenicol | 0 | −19 | 0 | 12 | 0 | Q4 | | | | |
| Thiamylal | 0 | −1 | 0 | 26 | 0 | Q4 | | | | |
| Thiodiglycol | 0 | −20 | 0 | 9 | 0 | Q4 | | | | |
| Thiopental | 0 | 3 | 0 | 13 | 0 | Q4 | | | | |
| Thiotepa | 0 | −15 | 1 | 15 | 1 | Q4 | | | | |
| Thiothixene | 1 | −4 | 0 | 37 | 0 | Q4 | | | | |
| Thioxolone, Tioxolone | 0 | −18 | 0 | 12 | 0 | Q4 | | | | |
| Thonzonium Bromide | 0 | −1 | 0 | 19 | 1 | Q4 | | | | |
| Thonzylamine Hydrochloride | 0 | −21 | 0 | 6 | 0 | Q4 | | | | |
| Thymol | 0 | 15 | 1 | 20 | 0 | Q4 | | | | |
| Thymopentin | 0 | −20 | 1 | 20 | 0 | Q4 | | | | |
| Thyroxine, Levothyroxine | 0 | 5 | 1 | 25 | 1 | Q4 | | | | |
| Tiapride | 0 | −18 | 0 | 6 | 0 | Q4 | | | | |
| Tibolone | 0 | −8 | 0 | 4 | 0 | Q4 | | | | |
| Ticarcillin Disodium | 0 | −22 | 0 | 7 | 0 | Q4 | | | | |
| Ticlopidine | 0 | −13 | 1 | 22 | 0 | Q4 | | | | |
| Tigecycline | 0 | 7 | 0 | 13 | 0 | Q4 | | | | |
| Tiletamine Hydrochloride | 0 | 8 | 0 | 22 | 0 | Q4 | | | | |
| Timolol | 0 | 11 | 1 | −7 | 0 | Q4 | | | | |
| Timolol Maleate | 0 | −4 | 0 | 19 | 0 | Q4 | | | | |
| Timonacic | 0 | −38 | 0 | 19 | 0 | Q4 | | | | |
| Tinidazole | 0 | 2 | 0 | 14 | 1 | Q4 | | | | |
| Tioconazole | 0 | 4 | 1 | 12 | 0 | Q4 | | | | |
| Tioconazole | 0 | −16 | 1 | 8 | 1 | Q4 | | | | |
| Tioguanine | 0 | 12 | 1 | 18 | 0 | Q4 | | | | |
| Tiopronin | 0 | −8 | 1 | 6 | 0 | Q4 | | | | |
| Tiotropium | 0 | −32 | 0 | −1 | 0 | Q4 | | | | |
| Tiratricol | 0 | −51 | 1 | 13 | 0 | Q4 | | | | |
| Tizanidine | 0 | 2 | 1 | 2 | 1 | Q4 | | | | |
| Tobramycin | 0 | 1 | 1 | 24 | 0 | Q4 | | | | |
| Tocainide | 0 | −25 | 1 | 0 | 0 | Q4 | | | | |
| Todralazine Hydrochloride | 0 | 0 | 1 | 19 | 0 | Q4 | | | | |
| Tolazoline, Priscoline | 0 | −90 | 0 | 17 | 0 | Q4 | | | | |
| Tolbutamide | 0 | 31 | 1 | 24 | 0 | Q4 | | | | |
| Tolcapone | 2 | −43 | 1 | 9 | 0 | Q4 | | | | |
| Tolfenamic Acid | 0 | 3 | 1 | 36 | 1 | Q4 | | | | |
| Tolmetin | 0 | 48 | 0 | 28 | 0 | Q4 | | | | |
| Tolonium Chloride | 0 | −4 | 1 | 43 | 1 | Q4 | | | | |
| Tolperisone | 0 | −7 | 1 | 11 | 0 | Q4 | | | | |
| Tolterodine | 0 | −36 | 1 | 15 | 0 | Q4 | | | | |
| Topiramate | 0 | 4 | 1 | 5 | 0 | Q4 | | | | |
| Torasemide | 0 | −29 | 1 | 7 | 0 | Q4 | | | | |
| Toremifene | 0 | 14 | 1 | 19 | 0 | Q4 | | | | |
| Tramadol | 0 | 26 | 1 | 12 | 0 | Q4 | | | | |
| Tramadol Hydrochloride | 0 | 35 | 0 | 21 | 0 | Q4 | | | | |
| Trandolapril | 0 | −5 | 1 | 15 | 0 | Q4 | | | | |
| Tranexamic Acid | 0 | −4 | 1 | 13 | 0 | Q4 | | | | |
| Tranilast | 0 | 35 | 0 | 22 | 0 | Q4 | | | | |
| Tranylcypromine | 0 | 5 | 1 | −5 | 0 | Q4 | | | | |
| Tranylcypromine Sulfate | 0 | 3 | 0 | 17 | 0 | Q4 | | | | |
| Travoprost | 0 | 4 | 1 | 15 | 0 | Q4 | | | | |
| Trazodone | 0 | −10 | 0 | −2 | 0 | Q4 | | | | |
| Tretinon, Tretinoin | 0 | 27 | 1 | 34 | 1 | Q4 | | | | |
| Triacetin | 0 | −32 | 1 | 15 | 0 | Q4 | | | | |
| Triamcinolone | 0 | −2 | 1 | −14 | 0 | Q4 | | | | |
| Triamterene | 0 | 40 | 1 | 24 | 0 | Q4 | | | | |
| Trichlormethiazide | 0 | 17 | 1 | 13 | 0 | Q4 | | | | |
| Triclosan | 0 | −6 | 1 | 16 | 1 | Q4 | | | | |
| Triethylenetetramine | 0 | 3 | 1 | 10 | 0 | Q4 | | | | |
| Triflupromazine | 1 | 49 | 1 | 37 | 1 | Q4 | | | | |
| Trifluridine | 0 | 10 | 1 | 16 | 1 | Q4 | | | | |
| Trihexyphenidyl | 0 | 5 | 1 | 14 | 1 | Q4 | | | | |
| Trilostane | 0 | −24 | 0 | 11 | 0 | Q4 | | | | |
| Trimebutine | 0 | −11 | 1 | 13 | 0 | Q4 | | | | |
| Trimetazidine | 0 | −18 | 1 | 1 | 0 | Q4 | | | | |
| Trimethadione | 0 | −5 | 1 | 14 | 0 | Q4 | | | | |
| Trimethobenzamide | 0 | 16 | 1 | 17 | 0 | Q4 | | | | |
| Trimethoprim | 0 | −26 | 0 | 17 | 0 | Q4 | | | | |
| Trimethyl Glycine | 0 | −14 | 0 | 22 | 0 | Q4 | | | | |
| Trimetozine | 0 | 2 | 1 | 12 | 0 | Q4 | | | | |
| Trioxsalen | 0 | −18 | 1 | 18 | 0 | Q4 | | | | |
| Tripelennamine | 0 | 14 | 1 | 25 | 0 | Q4 | | | | |
| Triprolidine | 0 | −24 | 1 | 23 | 0 | Q4 | | | | |
| Triptorelin | 0 | −15 | 1 | 4 | 0 | Q4 | | | | |
| Tris | 0 | −18 | 1 | 41 | 1 | Q4 | | | | |
| Troclosene Potassium | 0 | −8 | 1 | 1 | 0 | Q4 | | | | |
| Troglitazone | 0 | −22 | 1 | 4 | 0 | Q4 | | | | |
| Tropicamide | 0 | −4 | 1 | 17 | 0 | Q4 | | | | |
| Tropisetron | 0 | −28 | 0 | 14 | 0 | Q4 | | | | |
| Trospium Chloride | 0 | −39 | 0 | 9 | 1 | Q4 | | | | |
| Troxerutin | 0 | −10 | 0 | 8 | 0 | Q4 | | | | |
| Tryptophan | 0 | −6 | 1 | 16 | 0 | Q4 | | | | |
| Tuaminoheptane Sulfate | 0 | −7 | 1 | 19 | 0 | Q4 | | | | |
| Tubocurarine | 0 | −1 | 0 | 19 | 0 | Q4 | | | | |
| Tulobuterol, Tulobuterol | 0 | 21 | 0 | 36 | 0 | Q4 | | | | |
| Hydrochloride | | | | | | | | | | |
| Tylosin Tartrate | 0 | 9 | 1 | 11 | 0 | Q4 | | | | |
| Tyloxapol | 0 | −1 | 1 | 17 | 0 | Q4 | | | | |
| Tyrosine | 0 | −7 | 1 | 21 | 1 | Q4 | | | | |
| Undecylenic Acid, Zinc | 0 | −13 | 1 | 12 | 0 | Q4 | | | | |
| Undecylenate | | | | | | | | | | |
| Undecylenic Acid, Zinc | 0 | −6 | 1 | 14 | 0 | Q4 | | | | |
| Undecylenate | | | | | | | | | | |
| Uracil | 0 | 3 | 0 | 11 | 0 | Q4 | | | | |
| Urapidil Hydrochloride | 0 | −6 | 1 | 20 | 0 | Q4 | | | | |
| Urea | 0 | −14 | 1 | 20 | 0 | Q4 | | | | |
| Ursodeoxycholic Acid | 0 | −18 | 1 | 1 | 0 | Q4 | | | | |
| Ursodiol | 0 | −21 | 0 | 18 | 0 | Q4 | | | | |
| Valaciclovir | 0 | 12 | 1 | 15 | 0 | Q4 | | | | |
| Valdecoxib | 0 | −39 | 1 | −4 | 0 | Q4 | | | | |
| Valganciclovir | 0 | −12 | 0 | 14 | 1 | Q4 | | | | |
| Valproic Acid | 0 | −28 | 0 | 27 | 0 | Q4 | | | | |
| Valsartan | 0 | −17 | 0 | 12 | 0 | Q4 | | | | |
| Vancomycin | 0 | −1 | 1 | −2 | 0 | Q4 | | | | |
| Vancomycin Hydrochloride | 0 | −20 | 1 | 9 | 0 | Q4 | | | | |
| Vardenafil | 0 | −21 | 0 | 12 | 0 | Q4 | | | | |
| Vardenafil | 0 | 10 | 1 | 13 | 0 | Q4 | | | | |
| Vatalanib | 0 | −26 | 1 | −3 | 0 | Q4 | | | | |
| Vecuronium | 0 | −24 | 1 | 0 | 1 | Q4 | | | | |
| Vecuronium Bromide | 0 | 7 | 1 | 23 | 0 | Q4 | | | | |
| Venlafaxine, Venlafaxine | 0 | 3 | 1 | 20 | 0 | Q4 | | | | |
| Hydrochloride | | | | | | | | | | |
| Verapamil | 0 | 5 | 0 | 19 | 0 | Q4 | | | | |
| Vidarabine | 0 | −3 | 1 | 14 | 0 | Q4 | | | | |
| Vilazodone | 0 | 1 | 1 | 27 | 1 | Q4 | | | | |
| Vincamine | 0 | 1 | 0 | 20 | 0 | Q4 | | | | |
| Vinorelbine | 0 | −12 | 1 | −11 | 0 | Q4 | | | | |
| Vinpocetine | 0 | −19 | 0 | 0 | 0 | Q4 | | | | |
| Viomycin Sulfate | 0 | −7 | 0 | 19 | 0 | Q4 | | | | |
| Vitamin A | 0 | 15 | 1 | 17 | 0 | Q4 | | | | |
| Vitamin C | 0 | −28 | 1 | 18 | 0 | Q4 | | | | |
| Voriconazole | 0 | −42 | 0 | 5 | 0 | Q4 | | | | |
| Warfarin, Warfarin Sodium | 0 | −4 | 1 | 16 | 0 | Q4 | | | | |
| Xylometazoline | 0 | −5 | 0 | 19 | 1 | Q4 | | | | |
| Yohimbine | 3 | 4 | 1 | −17 | 0 | Q4 | | | | |
| Yohimbine Hydrochloride | 3 | −8 | 1 | 19 | 1 | Q4 | | | | |
| Zafirlukast | 0 | −21 | 0 | 3 | 1 | Q4 | | | | |
| Zalcitabine | 0 | −10 | 0 | 11 | 0 | Q4 | | | | |
| Zaleplon | 0 | 16 | 1 | 13 | 0 | Q4 | | | | |
| Zaprinast | 0 | −34 | 1 | 13 | 0 | Q4 | | | | |
| Zidovudine | 3 | −20 | 1 | 9 | 0 | Q4 | | | | |
| Zidovudine | 3 | −7 | 0 | 13 | 0 | Q4 | | | | |
| Zileuton | 0 | −12 | 0 | 21 | 0 | Q4 | | | | |
| Ziprasidone Mesylate, Ziprasidone | 0 | 14 | 1 | 35 | 1 | Q4 | | | | |
| Zolmitriptan | 0 | 9 | 1 | 18 | 0 | Q4 | | | | |
| Zolpidem, Zolpidem Tartrate | 0 | −12 | 0 | 4 | 0 | Q4 | | | | |
| Zomepirac | 0 | −4 | 1 | 10 | 0 | Q4 | | | | |
| Zonisamide | 0 | −37 | 0 | −12 | 0 | Q4 | | | | |
| Zopiclone | 0 | 11 | 1 | 5 | 0 | Q4 | | | | |
| Zoxazolamine | 0 | −14 | 0 | 11 | 0 | Q4 | | | | |
| | 0 | 14 | 1 | 6 | 0 | Q4 | | | | |
| | 0 | −3 | 2 | −4 | 1 | Q4 | | | | |
| | 0 | 20 | 1 | 7 | 0 | Q4 | | | | |
| | 0 | 0 | 1 | −2 | 0 | Q4 | | | | |

Table 4 provides a list of all 1907 compounds with labeling Q1, Q2, Q3 and Q4quadrants in FIG. 5b.

TABLE 5
provides which compounds scored for their selectivity for fibrin. This criterion
further differentiates the compounds and demonstrates those that are selective.

| | | | | Fibrin | LPS | | | | Cell | Cell | |
|---|---|---|---|---|---|---|---|---|---|---|---|
| | | | Cherry- | IC50 | IC50 | | | | Toxicity | Toxicity | |
| | | | picked | (uM) | (uM) | KEGG | | | IC50 | IC50 | |
| | | | Hit | (Suppl. | (Suppl. | Analysis | | Fibrin | (uM) | (uM) | |
| | | Quadrant | (Suppl. | Table | Table | (Suppl. | Fibrin | Selectivity | [cFib | [LPS | |
| No. | Name | (FIG. 5b) | Table 7) | 7) | 7) | Table 8) | Selectivity | (LOG10) | assay] | assay] | Therapy |
| 1 | Docetaxel | Q3 | HIT | 0.002 | >20 | | 9814 | 3.99 | >20 | >20 | antineoplastic |
| 2 | Prednicarbate | Q2 | HIT | 0.002 | >20 | | 8510 | 3.93 | >20 | >20 | antiinflammatory, |
| | | | | | | | | | | | glucocorticoid |
| 3 | Diflorasone | Q3 | HIT | 0.01 | >20 | | 2188 | 3.34 | >20 | >20 | antiinflammatory, |
| | Diacetate | | | | | | | | | | glucocorticoid |
| 4 | Alclometazone | Q2 | HIT | <0.001 | 2 | | 2153 | 3.33 | >20 | >20 | antiinflammatory, |
| | Dipropionate | | | | | | | | | | glucocorticoid |
| 5 | Hydrocortisone | Q2 | HIT | 0.03 | 18 | KEGG | 568 | 2.75 | >20 | >20 | glucocorticoid |
| 6 | Dexamethasone | Q2 | HIT | 0.004 | 2 | KEGG | 383 | 2.58 | >20 | >20 | glucocorticoid, |
| | Sodium Phosphate | | | | | | | | | | antiinflammatory |
| 7 | Fludrocortisone | Q2 | HIT | <0.001 | 0.4 | KEGG | 362 | 2.56 | >20 | >20 | mineralocorticoid |
| | Acetate | | | | | | | | | | |
| 8 | Hydrocortisone | Q2 | HIT | 0.02 | 7 | KEGG | 350 | 2.54 | >20 | >20 | glucocorticoid |
| | Hemisuccinate | | | | | | | | | | |
| 9 | Melengestrol | Q2 | HIT | 0.07 | 13 | | 196 | 2.29 | >20 | >20 | antineoplastic, |
| | Acetate | | | | | | | | | | progestin |
| 10 | Dichlorisone | Q2 | HIT | 0.03 | 4 | | 169 | 2.23 | >20 | >20 | antipruretic |
| | Acetate | | | | | | | | | | |
| 11 | Hydrocortisone | Q2 | HIT | 0.02 | 2 | KEGG | 89 | 1.95 | >20 | >20 | glucocorticoid, |
| | Butyrate | | | | | | | | | | antiinflammatory |
| 12 | Medroxyprogesterone | Q2 | HIT | 0.3 | >20 | | 70 | 1.84 | >20 | >20 | progestogen |
| | Acetate | | | | | | | | | | |
| 13 | Monensin Sodium | Q2 | HIT | 0.004 | 0.2 | | 49 | 1.69 | >20 | 2 | antibiotic, |
| | | | | | | | | | | | antibacterial; |
| | | | | | | | | | | | antibacterial |
| 14 | Deflazacort | Q2 | HIT | <0.001 | 0.04 | | 42 | 1.63 | 20 | >20 | antiinflammatory |
| 15 | Medroxyprogesterone | Q2 | HIT | 0.5 | >20 | | 41 | 1.61 | >20 | >20 | contraceptive |
| | Acetate | | | | | | | | | | |
| 16 | Ritodrine | Q3 | HIT | 0.6 | >20 | | 33 | 1.51 | >20 | >20 | muscle relaxant |
| | Hydrochloride | | | | | | | | | | (smooth) |
| 17 | Prednisolone | Q2 | HIT | 0.1 | 4 | | 31 | 1.49 | >20 | >20 | antiinflammatory, |
| | Sodium Phosphate | | | | | | | | | | glucocorticoid |
| 18 | Mebendazole | Q2 | HIT | 0.2 | 6 | | 31 | 1.49 | >20 | >20 | anthelmintic |
| 19 | Hydrocortisone | Q2 | HIT | 0.05 | 1 | | 28 | 1.45 | >20 | >20 | antiinflammatory, |
| | Valerate | | | | | | | | | | glucocorticoid |
| 20 | Levonordefrin | Q3 | HIT | 0.9 | >20 | | 21 | 1.33 | >20 | >20 | vasoconstrictor |
| 21 | Tegaserod Maleate | Q2 | HIT | 1 | 19 | | 20 | 1.29 | >20 | >20 | 5HT4 receptor |
| | | | | | | | | | | | agonist, peristaltic |
| | | | | | | | | | | | stimulant |
| 22 | Triamcinolone | Q2 | HIT | 0.04 | 0.8 | KEGG | 17 | 1.23 | >20 | >20 | antiinflammatory |
| | Diacetate | | | | | | | | | | |
| 23 | Hydrocortisone | Q2 | HIT | 0.008 | 0.1 | KEGG | 16 | 1.21 | >20 | >20 | glucocorticoid, |
| | | | | | | | | | | | antiinflammatory |
| 24 | Methylprednisolone | Q2 | HIT | <0.001 | 0.02 | KEGG | 15 | 1.19 | >20 | >20 | glucocorticoid |
| 25 | Rosuvastatin | Q2 | HIT | 0.3 | 3 | | 12 | 1.09 | 20 | >20 | antihyperlipidemic |
| 26 | Vinblastine Sulfate | Q2 | HIT | 0.02 | 0.2 | KEGG | 11 | 1.05 | >20 | >20 | antineoplastic, |
| | | | | | | | | | | | spindle poison |
| 27 | Salmeterol | Q3 | HIT | 1 | 14 | | 10 | 1.02 | >20 | >20 | Bronchodilator |
| 28 | Triamcinolone | Q2 | HIT | 0.04 | 0.4 | | 10 | 1.00 | >20 | >20 | glucocorticoid |
| 29 | Betamethasone | Q2 | HIT | <0.001 | 0.01 | KEGG | 10 | 0.99 | >20 | >20 | glucocorticoid, |
| | | | | | | | | | | | antiinflammatory |
| 30 | Desoxycorticosterone | Q3 | HIT | 0.5 | 5 | | 10 | 0.98 | >20 | >20 | mineralocorticoid |
| | Acetate | | | | | | | | | | |
| 31 | Algestone | Q2 | HIT | 1 | 9 | | 9 | 0.95 | >20 | >20 | antiacne, progestin |
| | Acetophenide | | | | | | | | | | |
| 32 | Eplerenore | Q2 | HIT | 3 | >20 | | 8 | 0.88 | >20 | >20 | antihypertensive |
| 33 | Tolnaftate | Q2 | HIT | 2 | 17 | | 7 | 0.87 | >20 | >20 | antifungal |
| 34 | Rebamipide | Q2 | HIT | 2 | 11 | | 6 | 0.80 | >20 | >20 | antiulcer, |
| | | | | | | | | | | | antioxidant |
| 35 | Prednisone | Q3 | HIT | 3 | >20 | | 6 | 0.78 | >20 | >20 | glucocorticoid |
| 36 | Tepoxalin | Q2 | HIT | 3 | 18 | | 6 | 0.75 | >20 | >20 | antipsoratic |
| 37 | Xylazine | Q2 | HIT | 2 | 8 | | 5 | 0.72 | >20 | >20 | “alpha2 |
| | Hydrochloride | | | | | | | | | | Adrenoceptor |
| | | | | | | | | | | | agonist; anesthetic, |
| | | | | | | | | | | | analgesic”; |
| | | | | | | | | | | | analgesic |
| 38 | Gentamicin Sulfate | Q2 | HIT | 4 | >20 | | 5 | 0.71 | >20 | >20 | antibacterial |
| 39 | Idazoxan | Q3 | HIT | 0.4 | 2 | | 5 | 0.69 | >20 | >20 | alpha2-adrenergic |
| | Hydrochloride | | | | | | | | | | blocker |
| 40 | Atorvastatin | Q2 | HIT | 0.6 | 3 | | 5 | 0.67 | >20 | >20 | antihyperlipidemic, |
| | Calcium | | | | | | | | | | HMGCoA reductase |
| | | | | | | | | | | | inhibitor |
| 41 | Rubitecan | Q3 | HIT | 2 | 9 | | 4 | 0.63 | >20 | >20 | antineoplastic |
| 42 | Megestrol Acetate | Q3 | HIT | 5 | >20 | | 4 | 0.63 | >20 | >20 | progestogen, |
| | | | | | | | | | | | antineoplastic |
| 43 | Betamethasone | Q2 | HIT | 0.05 | 0.2 | | 4 | 0.62 | >20 | >20 | antiinflammatory, |
| | Sodium Phosphate | | | | | | | | | | glucocorticoid |
| 44 | Pitavastatin | Q2 | HIT | 1 | 5 | | 4 | 0.61 | >20 | >20 | HMGA reductase |
| | Calcium | | | | | | | | | | inhibitor |
| 45 | Flumethazone | Q2 | HIT | <0.001 | 0.004 | | 4 | 0.58 | >20 | >20 | glucocorticoid, |
| | Pivalate | | | | | | | | | | antiinflammatory |
| 46 | lopanic Acid | Q3 | HIT | 5 | >20 | | 4 | 0.58 | >20 | >20 | radioopaque agent |
| 47 | Zoledronic Acid | Q3 | HIT | 2 | 8 | | 4 | 0.55 | >20 | >20 | Antiosteoporotic |
| 48 | Selamectin | Q3 | HIT | 6 | >20 | | 3 | 0.51 | >20 | >20 | anthelmintic, |
| | | | | | | | | | | | antiparasitic, |
| | | | | | | | | | | | antimite |
| 49 | Oxaliplatin | Q3 | HIT | 5 | 15 | | 3 | 0.48 | >20 | >20 | antineoplastic |
| 50 | Artesunate | Q3 | HIT | 7 | >20 | | 3 | 0.45 | >20 | >20 | Antimalarial |
| 51 | Naftopidil | Q3 | HIT | 7 | >20 | | 3 | 0.45 | >20 | >20 | “alpha1 |
| | Dihydrochloride | | | | | | | | | | Adrenoceptor |
| | | | | | | | | | | | antagonist; |
| | | | | | | | | | | | antihypertensive, |
| | | | | | | | | | | | alpha-blocker, |
| | | | | | | | | | | | 5HT1a |
| | | | | | | | | | | | agonist”; |
| | | | | | | | | | | | antihypertensive, |
| | | | | | | | | | | | alpha- |
| | | | | | | | | | | | blocker, 5HT1a |
| | | | | | | | | | | | agonist |
| 52 | Fluorouracil | Q3 | HIT | 7 | >20 | | 3 | 0.44 | >20 | >20 | antineoplastic, |
| | | | | | | | | | | | pyrimidine |
| | | | | | | | | | | | antimetabolite |
| 53 | Isoxicam | Q3 | HIT | 7 | >20 | | 3 | 0.43 | >20 | >20 | antiinflammatory |
| 54 | Milnacipran | Q3 | HIT | 8 | 20 | | 3 | 0.40 | >20 | >20 | inhibitor of |
| | Hydrochloride | | | | | | | | | | norepinephrine and |
| | | | | | | | | | | | seritonin uptake, |
| | | | | | | | | | | | treatment of |
| | | | | | | | | | | | fibromyalgia |
| 55 | Securinine | Q2 | HIT | 2 | 6 | | 3 | 0.40 | >20 | 12 | GABAA receptor |
| | | | | | | | | | | | blocker, CNS |
| | | | | | | | | | | | stimulant |
| 56 | Demeclocycline | Q3 | HIT | 6 | 15 | | 2 | 0.38 | >20 | >20 | antibacterial |
| | Hydrochloride | | | | | | | | | | |
| 57 | Benzyl | Q2 | HIT | 10 | >20 | | 2 | 0.29 | >20 | >20 | antineoplastic, |
| | Isothiocyanate | | | | | | | | | | antibacterial, |
| | | | | | | | | | | | antifungal |
| 58 | Ribavirin | Q2 | HIT | 1 | 2 | | 2 | 0.29 | >20 | >20 | antiviral |
| 59 | Tolazamide | Q3 | HIT | 12 | >20 | | 2 | 0.23 | >20 | >20 | “Oral hypoglycemic |
| | | | | | | | | | | | agent; stimulates |
| | | | | | | | | | | | pancreatic islet cells |
| | | | | | | | | | | | to secrete insulin, |
| | | | | | | | | | | | antidiabetic”; |
| | | | | | | | | | | | antidiabetic |
| 60 | Oxybendazole | Q3 | HIT | 7 | 12 | | 2 | 0.22 | >20 | >20 | anthelmintic |
| 61 | Albendazole | Q3 | HIT | 4 | 6 | | 2 | 0.22 | >20 | >20 | anthelmintic |
| 62 | Betazole | Q3 | HIT | 12 | >20 | | 2 | 0.22 | >20 | >20 | gastric secretion |
| | Hydrochloride | | | | | | | | | | stimulant |
| 63 | Prednisone | Q3 | HIT | 12 | >20 | | 2 | 0.21 | >20 | >20 | antiinflammatory, |
| | | | | | | | | | | | glucocorticoid |
| 64 | 2- | Q2 | HIT | 3 | 5 | | 2 | 0.21 | >20 | >20 | angiogenesis |
| | Methoxyestradiol | | | | | | | | | | inhibitor |
| 65 | Dexamethasone | Q3 | HIT | 15 | >20 | KEGG | 1 | 0.13 | >20 | >20 | Nuclear receptor |
| | | | | | | | | | | | ligands: |
| | | | | | | | | | | | corticosteroid |
| 66 | Fenbendazole | Q3 | HIT | 3 | 4 | | 1 | 0.12 | >20 | >20 | anthelmintic |
| 67 | Cloperastine | Q3 | HIT | 7 | 9 | | 1 | 0.09 | >20 | >20 | antitussive |
| | Hydrochloride | | | | | | | | | | |
| 68 | Cyproterone | Q3 | HIT | 17 | >20 | | 1 | 0.08 | >20 | >20 | antiandrogen |
| 69 | Chlormadinone | Q3 | HIT | 17 | >20 | | 1 | 0.07 | >20 | >20 | progestin, |
| | Acetate | | | | | | | | | | antiandrogen |
| 70 | Fusidic Acid | Q2 | HIT | 19 | >20 | | 1 | 0.03 | >20 | >20 | antibacterial |
| 71 | Risedronate | Q3 | HIT | 19 | >20 | | 1 | 0.02 | >20 | >20 | calcium regulator, |
| | Sodium | | | | | | | | | | Antibone resorptive |
| 72 | Salinomycin, | Q2 | HIT | 2 | 2 | | 1 | 0.02 | >20 | 17 | antibiotic, |
| | Sodium | | | | | | | | | | antibacterial; |
| | | | | | | | | | | | antibacterial |
| 73 | Mycophenolic Acid | Q2 | HIT | 1 | 1 | KEGG | 1 | 0.00 | >20 | >20 | immune |
| | | | | | | | | | | | suppressant, |
| | | | | | | | | | | | antineoplastic, |
| | | | | | | | | | | | antiviral |
| 74 | Almotriptan | Q2 | HIT | >20 | >20 | | 1 | 0.00 | >20 | >20 | 5HT 1B/2D receptor |
| | | | | | | | | | | | agonist |
| 75 | Amisulpride | Q3 | HIT | 20 | >20 | | 1 | 0.00 | >20 | >20 | antipsychotic |
| 76 | Bacitracin | Q3 | HIT | >20 | >20 | | 1 | 0.00 | >20 | >20 | antibacterial |
| 77 | Benzoyl Peroxide | Q2 | HIT | 20 | >20 | | 1 | 0.00 | >20 | >20 | keratolytic |
| 78 | Betamethasone | Q2 | HIT | 20 | >20 | | 1 | 0.00 | >20 | >20 | glucocorticoid |
| | Valerate | | | | | | | | | | |
| 79 | Chlorthalidone | Q3 | HIT | >20 | >20 | | 1 | 0.00 | >20 | >20 | diuretic, |
| | | | | | | | | | | | antihypertensive |
| 80 | Clobetasol | Q2 | HIT | <0.001 | <0.001 | | 1 | 0.00 | >20 | >20 | glucocorticoid, |
| | Propionate | | | | | | | | | | antiinflammatory |
| 81 | Colistimethate | Q2 | HIT | >20 | >20 | | 1 | 0.00 | >20 | >20 | antibacterial |
| | Sodium | | | | | | | | | | |
| 82 | Docetaxel | Q3 | HIT | 20 | >20 | | 1 | 0.00 | >20 | >20 | antineoplastic |
| 83 | Ethacrynic Acid | Q3 | HIT | >20 | >20 | | 1 | 0.00 | >20 | >20 | diuretic |
| 84 | Ethinyl Estradiol | Q3 | HIT | >20 | >20 | | 1 | 0.00 | >20 | >20 | estrogen, plus |
| | | | | | | | | | | | progestogen as oral |
| | | | | | | | | | | | contraceptive |
| 85 | Flumethasone | Q2 | HIT | <0.001 | <0.001 | | 1 | 0.00 | >20 | >20 | antiinflammatory |
| 86 | Flunisolide | Q2 | HIT | <0.001 | <0.001 | | 1 | 0.00 | >20 | >20 | antiinflammatory |
| 87 | Fluocinolone | Q2 | HIT | <0.001 | <0.001 | KEGG | 1 | 0.00 | >20 | >20 | glucocorticoid, |
| | Acetonide | | | | | | | | | | antiinflammatory |
| 88 | Ftaxilide | Q3 | HIT | >20 | >20 | | 1 | 0.00 | >20 | >20 | antiulcer |
| 89 | Hydroxytoluic Acid | Q3 | HIT | 20 | >20 | | 1 | 0.00 | >20 | >20 | analgesic, antiseptic |
| 90 | Ibudilast | Q2 | HIT | >20 | >20 | | 1 | 0.00 | >20 | >20 | antiinflammatory |
| 91 | Imipenem | Q3 | HIT | >20 | >20 | | 1 | 0.00 | >20 | >20 | antibacterial, |
| | | | | | | | | | | | Antibiotic; |
| | | | | | | | | | | | antibacterial |
| 92 | Irbesartan | Q3 | HIT | >20 | >20 | | 1 | 0.00 | >20 | >20 | angiotensin 2 |
| | | | | | | | | | | | receptor antagonist |
| 93 | Mephentermine | Q3 | HIT | >20 | >20 | | 1 | 0.00 | >20 | >20 | vasoconstrictor |
| | Sulfate | | | | | | | | | | |
| 94 | Mequinol | Q2 | HIT | >20 | >20 | | 1 | 0.00 | >20 | >20 | skin depigmentor |
| 95 | Metampicillin | Q3 | HIT | 20 | >20 | | 1 | 0.00 | >20 | >20 | antimicrobial; |
| | Sodium | | | | | | | | | | antibacterial |
| 96 | Methylphenidate | Q2 | HIT | >20 | >20 | | 1 | 0.00 | >20 | >20 | CNS stimulant |
| | Hydrochloride | | | | | | | | | | |
| 97 | Miconazole Nitrate | Q3 | HIT | 20 | >20 | | 1 | 0.00 | >20 | >20 | antifungal (topical) |
| 98 | Midodrine | Q2 | HIT | >20 | >20 | | 1 | 0.00 | >20 | >20 | antihypertensive, |
| | Hydrochloride | | | | | | | | | | vasoconstrictor |
| 99 | Mirtazapine | Q3 | HIT | >20 | >20 | | 1 | 0.00 | >20 | >20 | antidepressant |
| 100 | Molsidomine | Q2 | HIT | >20 | >20 | | 1 | 0.00 | >20 | >20 | antianginal |
| 101 | Oxiglutatione | Q3 | HIT | >20 | >20 | | 1 | 0.00 | >20 | >20 | antioxidant |
| | Disodium Salt | | | | | | | | | | |
| 102 | Phenytoin Sodium | Q2 | HIT | >20 | >20 | | 1 | 0.00 | >20 | >20 | “Anticonvulsant; |
| | | | | | | | | | | | anti- |
| | | | | | | | | | | | epileptic”; |
| | | | | | | | | | | | anticonvulsant, |
| | | | | | | | | | | | antieleptic |
| 103 | Ractopamine | Q3 | HIT | 20 | >20 | | 1 | 0.00 | >20 | >20 | beta-adrenergic |
| | Hydrochloride | | | | | | | | | | agonist, growth |
| | | | | | | | | | | | stimulant |
| 104 | Rapamycin | Q2 | HIT | <0.001 | <0.001 | | 1 | 0.00 | >20 | >20 | Inhibitors: FRAP |
| | | | | | | | | | | | inhibitor |
| 105 | Rasagiline | Q3 | HIT | 20 | >20 | | 1 | 0.00 | >20 | >20 | antiparkinsonian |
| 106 | Salicin | Q2 | HIT | >20 | >20 | | 1 | 0.00 | >20 | >20 | analgesic, |
| | | | | | | | | | | | antipyretic |
| 107 | Saquinavir | Q2 | HIT | >20 | >20 | | 1 | 0.00 | >20 | >20 | antiviral |
| 108 | Sulbactam | Q3 | HIT | >20 | >20 | | 1 | 0.00 | >20 | >20 | b-lactamase |
| | | | | | | | | | | | inhibitor |
| 109 | Sulfaquinoxaline | Q2 | HIT | >20 | >20 | | 1 | 0.00 | >20 | >20 | antibacterial, |
| | Sodium | | | | | | | | | | coccidiostat |
| 110 | Thiabendazole | Q2 | HIT | >20 | >20 | | 1 | 0.00 | >20 | >20 | anthelmintic |
| 111 | Toltrazuril | Q2 | HIT | >20 | >20 | | 1 | 0.00 | >20 | >20 | coccidiostat |
| 112 | Cyclosporin A | Q3 | HIT | 20 | 19 | | 1 | −0.01 | >20 | >20 | “Calcineurin |
| | | | | | | | | | | | phosphatase |
| | | | | | | | | | | | inhibitor; |
| | | | | | | | | | | | immunosuppressant”; |
| | | | | | | | | | | | immunosuppressant |
| 113 | Hycanthone | Q2 | HIT | 1 | 1 | | 1 | −0.04 | >20 | 11 | anthelmintic, |
| | | | | | | | | | | | hepatotoxic |
| 114 | Pimozide | Q3 | HIT | >20 | 18 | | 1 | −0.06 | >20 | >20 | “Ca2+ channel |
| | | | | | | | | | | | antagonist; |
| | | | | | | | | | | | antipsychotic; D2 |
| | | | | | | | | | | | dopamine receptor |
| | | | | | | | | | | | antagonist”; |
| | | | | | | | | | | | antipsychotic |
| 115 | Acrisorcin | Q2 | HIT | 7 | 5 | | 1 | −0.13 | 20 | 6 | antifungal |
| 116 | Trimipramine | Q2 | HIT | 7 | 5 | | 1 | −0.15 | >20 | 12 | antidepressant |
| | Maleate | | | | | | | | | | |
| 117 | R(−)-Apomorphine | Q3 | HIT | >20 | 14 | | 1 | −0.16 | >20 | >20 | Dopamine receptor |
| | Hydrochloride | | | | | | | | | | agonist |
| 118 | Mycophenolate | Q2 | HIT | 2 | 1 | | 1 | −0.23 | >20 | >20 | immunesuppressant, |
| | Mofetil | | | | | | | | | | antineoplastic, |
| | | | | | | | | | | | antiviral |
| 119 | Amoxapine | Q3 | HIT | 20 | 11 | | 1 | −0.24 | >20 | >20 | “Tricyclic |
| | | | | | | | | | | | antidepressant; |
| | | | | | | | | | | | inhibits neuronal |
| | | | | | | | | | | | uptake of |
| | | | | | | | | | | | norepinephrine”; |
| | | | | | | | | | | | antidepressant, |
| | | | | | | | | | | | inhibits |
| | | | | | | | | | | | norepinephrine |
| | | | | | | | | | | | uptake |
| 120 | Acivicin | Q2 | HIT | 2 | 1 | KEGG | 1 | −0.27 | >20 | >20 | antineoplastic |
| 121 | Benzydamine | Q2 | HIT | 6 | 3 | | 1 | −0.29 | 20 | 8 | analgesic, |
| | Hydrochloride | | | | | | | | | | antipyretic, |
| | | | | | | | | | | | antiinflammatory |
| 122 | Trifluoperazine | Q3 | HIT | 12 | 6 | | 0 | −0.31 | >20 | 12 | “Calmodulin |
| | Dihydrochloride | | | | | | | | | | antagonist; |
| | | | | | | | | | | | dopamine receptor |
| | | | | | | | | | | | antagonist; |
| | | | | | | | | | | | antipsychotic; |
| | | | | | | | | | | | sedative”; |
| | | | | | | | | | | | antipsychotic |
| 123 | Teniposide | Q2 | HIT | 2 | 1 | KEGG | 0 | −0.32 | 20 | 4 | antineoplastic |
| 124 | Perhexiline | Q2 | HIT | 8 | 3 | | 0 | −0.35 | 20 | 11 | coronary |
| | Maleate | | | | | | | | | | vasodilator |
| 125 | Medroxyprogesterone | Q3 | HIT | >20 | 8 | | 0 | −0.42 | >20 | 17 | progestogen |
| 126 | Nortriptyline | Q2 | HIT | 13 | 3 | | 0 | −0.61 | 20 | 10 | Tricyclic |
| | Hydrochloride | | | | | | | | | | antidepressant; |
| | | | | | | | | | | | antidepressant |
| 127 | Tyrothricin | Q2 | HIT | 3 | 0.5 | | 0 | −0.80 | >20 | 20 | topical antibacterial |
| | | | | | | | | | | | (topical) |
| 128 | Mechlorethamine | Q2 | HIT | 7 | 0.1 | | 0 | −1.68 | 20 | 0.5 | antineoplastic, |
| | | | | | | | | | | | alkylating agent |

TABLE 6
provides compounds listing those compounds in Table 5 with a cut-off of 1.5 log selectivity for fibrin (the top 16 compounds in Table 5).

| | | | | Fibrin | LPS | | | | Cell | Cell | |
|---|---|---|---|---|---|---|---|---|---|---|---|
| | | | Cherry- | IC50 | IC50 | | | | Toxicity | Toxicity | |
| | | | picked | (uM) | (uM) | KEGG | | | IC50 | IC50 | |
| | | | Hit | (Suppl. | (Suppl. | Analysis | | Fibrin | (uM) | (uM) | |
| | | Quadrant | (Suppl. | Table | Table | (Suppl. | Fibrin | Selectivity | [cFib | [LPS | |
| No. | Name | (FIG. 5b) | Table 7) | 7) | 7) | Table 8) | Selectivity | (LOG10) | assay] | assay] | Therapy |
| 1 | Docetaxel | Q3 | HIT | 0.002 | >20 | | 9814 | 3.99 | >20 | >20 | antineoplastic |
| 2 | Prednicarbate | Q2 | HIT | 0.002 | >20 | | 8510 | 3.93 | >20 | >20 | antiinflammatory, |
| | | | | | | | | | | | glucocorticoid |
| 3 | Diflorasone | Q3 | HIT | 0.01 | >20 | | 2188 | 3.34 | >20 | >20 | antiinflammatory, |
| | Diacetate | | | | | | | | | | glucocorticoid |
| 4 | Alclometazone | Q2 | HIT | <0.001 | 2 | | 2153 | 3.33 | >20 | >20 | antiinflammatory, |
| | Dipropionate | | | | | | | | | | glucocorticoid |
| 5 | Hydrocortisone | Q2 | HIT | 0.03 | 18 | KEGG | 568 | 2.75 | >20 | >20 | glucocorticoid |
| 6 | Dexamethasone | Q2 | HIT | 0.004 | 2 | KEGG | 383 | 2.58 | >20 | >20 | glucocorticoid, |
| | Sodium Phosphate | | | | | | | | | | antiinflammatory |
| 7 | Fludrocortisone | Q2 | HIT | <0.001 | 0.4 | KEGG | 362 | 2.56 | >20 | >20 | mineralocorticoid |
| | Acetate | | | | | | | | | | |
| 8 | Hydrocortisone | Q2 | HIT | 0.02 | 7 | KEGG | 350 | 2.54 | >20 | >20 | glucocorticoid |
| | Hemisuccinate | | | | | | | | | | |
| 9 | Melengestrol | Q2 | HIT | 0.07 | 13 | | 196 | 2.29 | >20 | >20 | antineoplastic, |
| | Acetate | | | | | | | | | | progestin |
| 10 | Dichlorisone | Q2 | HIT | 0.03 | 4 | | 169 | 2.23 | >20 | >20 | antipruretic |
| | Acetate | | | | | | | | | | |
| 11 | Hydrocortisone | Q2 | HIT | 0.02 | 2 | KEGG | 89 | 1.95 | >20 | >20 | glucocorticoid, |
| | Butyrate | | | | | | | | | | antiinflammatory |
| 12 | Medroxyprogesterone | Q2 | HIT | 0.3 | >20 | | 70 | 1.84 | >20 | >20 | progestogen |
| | Acetate | | | | | | | | | | |
| 13 | Monensin Sodium | Q2 | HIT | 0.004 | 0.2 | | 49 | 1.69 | >20 | 2 | antibiotic, |
| | | | | | | | | | | | antibacterial; |
| | | | | | | | | | | | antibacterial |
| 14 | Deflazacort | Q2 | HIT | <0.001 | 0.04 | | 42 | 1.63 | 20 | >20 | antiinflammatory |
| 15 | Medroxyprogesterone | Q2 | HIT | 0.5 | >20 | | 41 | 1.61 | >20 | >20 | contraceptive |
| | Acetate | | | | | | | | | | |
| 16 | Ritodrine | Q3 | HIT | 0.6 | >20 | | 33 | 1.51 | >20 | >20 | muscle relaxant |
| | Hydrochloride | | | | | | | | | | (smooth) |

TABLE 7
provides compounds and noting those that have been indicated for MS or EAE.

| | | | | | | | | | | | log | ratio | | | Reported effects |
|---|---|---|---|---|---|---|---|---|---|---|---|---|---|---|---|
| | dead_IC50 | fib_IC50 | | | LPS_IC50 | | | | fib | LPS | brain/ | brain/ | brain/ | | in MS or EAE or |
| Name | (uM) | (uM) | fib_HS | fib_R-sq | (uM) | LPS_HS | LPS_R-sq | Activity | specificity | specificity | blood | blood | blood_scale | Therapy | neuroinflammation |
| flurandrenolide | | 0.002 | 0.00 | | 0.002 | 0.00 | | fib/LPS | 1.0 | 1.0 | −1.37 | 0.04 | 3 | antiinflammatory | none |
| flumethazone | | 0.002 | −0.23 | 0.12 | 0.002 | 0.00 | | fib/LPS | 1.0 | 1.0 | | | 4 | glucocorticoid, | none |
| pivalate | | | | | | | | | | | | | | antiinflammatory | |
| fluocinolone | | 0.002 | 0.94 | 0.71 | 0.002 | 0.53 | 0.33 | fib/LPS | 1.0 | 1.0 | −1.34 | 0.05 | 3 | glucocorticoid, | Protection in |
| acetonide | | | | | | | | | | | | | | antiinflammatory | retinal |
| | | | | | | | | | | | | | | | neuroinflammation |
| | | | | | | | | | | | | | | | (microglia!) |
| triamcinolone | | 0.002 | ~−12 | 0.96 | 0.002 | −0.42 | 0.94 | fib/LPS | 1.0 | 1.0 | | | 4 | antiinflammatory | none |
| diacetate | | | | | | | | | | | | | | | |
| dexamethasone | | 0.002 | 0.00 | | 0.002 | 0.14 | 0.07 | fib/LPS | 1.0 | 1.0 | −1.16 | 0.07 | 3 | glucocorticoid | Protection in |
| | | | | | | | | | | | | | | | several rodent EAE |
| | | | | | | | | | | | | | | | models, optical |
| | | | | | | | | | | | | | | | neuritis, |
| | | | | | | | | | | | | | | | EAN, EAU, |
| | | | | | | | | | | | | | | | BBB disruption, |
| | | | | | | | | | | | | | | | reactive oxygen |
| | | | | | | | | | | | | | | | species generation |
| | | | | | | | | | | | | | | | in microglia |
| dexamethasone | | 0.002 | 0.00 | | 0.002 | 0.00 | | fib/LPS | 1.0 | 1.0 | −1.13 | 0.07 | 3 | glucocorticoid, | see |
| acetate | | | | | | | | | | | | | | antiinflammatory | dexamethasone |
| dexamethasone | | 0.002 | −0.49 | 0.68 | 0.005 | −0.60 | 0.97 | fib/LPS | 2.3 | 0.4 | | | 4 | glucocorticoid, | see |
| sodium | | | | | | | | | | | | | | antiinflammatory | dexamethasone |
| phosphate | | | | | | | | | | | | | | | |
| Nylidrin | | 0.002 | −0.24 | 0.48 | 0.007 | −0.51 | 0.86 | fib/LPS | 3.3 | 0.3 | 0.14 | 1.37 | 1 | beta | none |
| hydrochloride | | | | | | | | | | | | | | Adrenoceptor | |
| | | | | | | | | | | | | | | agonist; | |
| | | | | | | | | | | | | | | peripheral | |
| | | | | | | | | | | | | | | vasodilator | |
| fludrocortisone | | 0.002 | −1.10 | 0.76 | 0.002 | −0.29 | 0.83 | fib/LPS | 1.0 | 1.0 | −1.22 | 0.06 | 3 | mineralocorticoid | none |
| acetate | | | | | | | | | | | | | | | |
| fluocinonide | | 0.002 | −0.30 | 0.77 | 0.002 | −0.41 | 0.37 | fib/LPS | 1.0 | 1.0 | −1.30 | 0.05 | 3 | antiinflammatory, | none |
| | | | | | | | | | | | | | | glucocorticoid | |
| prednisolone | | 0.002 | −0.78 | 0.94 | 0.002 | −0.80 | 0.94 | fib/LPS | 1.0 | 1.0 | −1.30 | 0.05 | 3 | glucocorticoid | see |
| | | | | | | | | | | | | | | | methylprednisolone |
| 6alpha- | | 0.002 | −0.26 | 0.36 | 0.002 | −0.40 | 0.84 | fib/LPS | 1.0 | 1.0 | −1.19 | 0.06 | 3 | glucocorticoid | see |
| methylprednisolone | | | | | | | | | | | | | | | methylprednisolone |
| acetate | | | | | | | | | | | | | | | |
| fluoromethoIone | | 0.002 | −0.27 | 0.65 | 0.002 | 0.00 | | fib/LPS | 1.0 | 1.0 | −0.65 | 0.23 | 3 | glucocorticoid, | none |
| | | | | | | | | | | | | | | antiinflammatory | |
| prednisolone | | 0.003 | −0.66 | 0.89 | 0.003 | −0.53 | 0.95 | fib/LPS | 1.2 | 0.9 | −1.27 | 0.05 | 3 | glucocorticoid | Protection in EAE |
| acetate | | | | | | | | | | | | | | | and collagen- |
| | | | | | | | | | | | | | | | induced arthritis + |
| | | | | | | | | | | | | | | | see |
| | | | | | | | | | | | | | | | methylprednisolone |
| clopamide | | 0.003 | −0.88 | 0.88 | 0.002 | −1.30 | 0.91 | fib/LPS | 0.7 | 1.5 | −0.98 | 0.11 | 3 | diuretic | none |
| methylprednisolone | | 0.003 | −0.68 | 0.87 | 0.002 | −0.45 | 0.82 | fib/LPS | 0.6 | 1.6 | −1.22 | 0.06 | 3 | glucocorticoid | Treatment for MS; |
| | | | | | | | | | | | | | | | Protection in EAE |
| hydrocortisone | | 0.004 | −0.39 | 0.84 | 0.036 | −1.10 | 0.83 | fib/LPS | 8.4 | 0.1 | | | 4 | glucocorticoid | Beneficial in |
| hemisuccinate | | | | | | | | | | | | | | | combination |
| | | | | | | | | | | | | | | | therapy in MS |
| | | | | | | | | | | | | | | | (1970's) |
| hydrocortisone | | 0.010 | −0.67 | 0.97 | 0.017 | −0.95 | 0.98 | fib/LPS | 1.6 | 0.6 | −1.26 | 0.05 | 3 | glucocorticoid, | Protection in EAE |
| acetate | | | | | | | | | | | | | | antiinflammatory | (1970's) |
| podofilox | | 0.014 | −1.20 | 0.98 | 0.002 | −0.50 | 0.96 | fib/LPS | 0.2 | 5.8 | −0.95 | 0.11 | 3 | antineoplastic, | none |
| | | | | | | | | | | | | | | inhibits | |
| | | | | | | | | | | | | | | microtubule | |
| | | | | | | | | | | | | | | assembly, and | |
| | | | | | | | | | | | | | | human DNA | |
| | | | | | | | | | | | | | | topoisomerasell; | |
| | | | | | | | | | | | | | | antimitotic | |
| | | | | | | | | | | | | | | agent | |
| colchicine | | 0.015 | −3.20 | 0.94 | 0.009 | −1.10 | 0.97 | fib/LPS | 0.6 | 1.8 | −0.84 | 0.15 | 3 | antimitotic, | Beneficial with |
| | | | | | | | | | | | | | | antigout agent | polyunsaturated |
| | | | | | | | | | | | | | | | FA in MS (1970's) |
| hydrocortisone | | 0.018 | −0.65 | 0.90 | 0.002 | −0.58 | 0.97 | fib/LPS | 0.1 | 9.1 | −1.29 | 0.05 | 3 | glucocorticoid, | see hydrocortisone |
| | | | | | | | | | | | | | | antiinflammatory | hemisuccinate/ |
| | | | | | | | | | | | | | | | acetate |
| podophyllotoxin | | 0.022 | −1.10 | 0.99 | 0.012 | −0.87 | 0.93 | fib/LPS | 0.5 | 1.8 | −0.92 | 0.12 | 3 | | none |
| acetate | | | | | | | | | | | | | | | |
| dihydromundulone | | 0.022 | −0.67 | 0.96 | 0.006 | −0.56 | 0.99 | fib/LPS | 0.3 | 3.6 | −0.03 | 0.94 | 2 | | none |
| cefamandole | | 0.052 | −0.66 | 0.91 | 0.002 | −0.35 | 0.83 | fib/LPS | 0.0 | 33.2 | | | 4 | antimicrobial | none |
| sodium | | | | | | | | | | | | | | | |
| hydrocortisone | | 0.071 | −0.60 | 0.95 | 0.005 | −0.45 | 0.93 | fib/LPS | 0.1 | 15.4 | −0.91 | 0.12 | 3 | glucocorticoid, | see hydrocortisone |
| butyrate | | | | | | | | | | | | | | antiinflammatory | hemisuccinate/ |
| | | | | | | | | | | | | | | | acetate |
| hydrocortisone | | 0.128 | −0.52 | 0.73 | 0.161 | −0.69 | 0.95 | fib/LPS | 1.3 | 0.8 | | | 4 | glucocorticoid | see hydrocortisone |
| sodium | | | | | | | | | | | | | | | hemisuccinate/ |
| phosphate | | | | | | | | | | | | | | | acetate |
| 5,7- | | 0.172 | −0.56 | 0.90 | 0.021 | −0.59 | 0.75 | fib/LPS | 0.1 | 8.0 | −0.49 | 0.32 | 2 | | none |
| dihydroxyisoflavone | | | | | | | | | | | | | | | |
| nigericin | | 0.206 | −2.30 | 0.91 | 0.065 | −0.35 | 0.54 | fib/LPS | 0.3 | 3.2 | | | 4 | antibiotic | none |
| sodium | | | | | | | | | | | | | | | |
| mycophenolic | | 0.289 | −1.70 | 0.98 | 0.193 | −1.50 | 0.96 | fib/LPS | 0.7 | 1.5 | −0.67 | 0.22 | 3 | antineoplastic | Promising in co- |
| acid | | | | | | | | | | | | | | | therapy with |
| | | | | | | | | | | | | | | | IFNbeta1a in MS; |
| | | | | | | | | | | | | | | | Improvement in |
| | | | | | | | | | | | | | | | neuromyelitis |
| | | | | | | | | | | | | | | | optica |
| beta-peltatin | | 0.505 | −2.10 | 0.97 | 0.252 | −1.50 | 0.92 | fib/LPS | 0.5 | 2.0 | −0.69 | 0.21 | 3 | antineoplastic, | none |
| | | | | | | | | | | | | | | cytotoxic | |
| acivicin | | 0.570 | −0.83 | 0.93 | 1.662 | ~−19 | 0.76 | fib/LPS | 2.9 | 0.3 | −1.68 | 0.02 | 3 | antineoplastic | none |
| mycophenolic | | 0.606 | −0.83 | 0.92 | 0.771 | −1.60 | 0.79 | fib/LPS | 1.3 | 0.8 | −0.67 | 0.22 | 3 | antineoplastic | Promising in co- |
| acid | | | | | | | | | | | | | | | therapy with |
| | | | | | | | | | | | | | | | IFNbeta1a in MS; |
| | | | | | | | | | | | | | | | Improvement in |
| | | | | | | | | | | | | | | | neuromyelitis |
| | | | | | | | | | | | | | | | optica |
| hycanthone | | 0.619 | −0.84 | 0.94 | 0.354 | −0.67 | 0.88 | fib/LPS | 0.6 | 1.7 | 0.09 | 1.22 | 1 | anthelmintic, | none |
| | | | | | | | | | | | | | | hepatotoxic | |
| colchiceine | | 0.625 | −2.00 | 0.96 | 0.820 | −3.00 | 0.99 | fib/LPS | 1.3 | 0.8 | −1.10 | 0.08 | 3 | antimitotic | Patent on use of |
| | | | | | | | | | | | | | | | colchic(e)ine |
| | | | | | | | | | | | | | | | in MS + |
| | | | | | | | | | | | | | | | see clochicine |
| teniposide | | 0.821 | −1.10 | 0.91 | 0.240 | −0.66 | 0.94 | fib/LPS | 0.3 | 3.4 | | | 4 | antineoplastic | none |
| 2,4- | | 0.906 | −1.00 | 0.84 | 1.006 | −0.63 | 0.84 | fib/LPS | 1.1 | 0.9 | 1.12 | 13.21 | 0 | herbicide | none |
| dichlorophenoxyacetic | | | | | | | | | | | | | | | |
| acid, isooctyl ester | | | | | | | | | | | | | | | |
| Thioridazine | | 1.012 | −1.30 | 0.83 | 0.677 | −0.38 | 0.53 | fib/LPS | 0.7 | 1.5 | 1.46 | 28.77 | 0 | Dopamine | none |
| hydrochloride | | | | | | | | | | | | | | receptor | |
| | | | | | | | | | | | | | | antagonist; | |
| | | | | | | | | | | | | | | Ca2+ | |
| | | | | | | | | | | | | | | channel | |
| | | | | | | | | | | | | | | antagonist; | |
| | | | | | | | | | | | | | | antipsychotic | |
| monensin | | 1.143 | −3.80 | 0.97 | 0.212 | −0.48 | 0.76 | fib/LPS | 0.2 | 5.4 | | | 4 | antibiotic | none |
| sodium | | | | | | | | | | | | | | | |
| (monensin a is | | | | | | | | | | | | | | | |
| shown) | | | | | | | | | | | | | | | |
| dehydrodeoxysappanone | | 1.237 | −0.96 | 0.91 | 0.657 | −0.89 | 0.97 | fib/LPS | 0.5 | 1.9 | −0.22 | 0.61 | 2 | | none |
| b dimethyl ether | | | | | | | | | | | | | | | |
| vinblastine | | 1.394 | −0.80 | 0.85 | 0.628 | −0.57 | 0.84 | fib/LPS | 0.5 | 2.2 | | | 4 | antineoplastic, | none |
| sulfate | | | | | | | | | | | | | | spindle poison | |
| maprotiline | | 1.399 | −0.63 | 0.50 | 0.289 | −0.39 | 0.68 | fib/LPS | 0.2 | 4.8 | 0.92 | 8.22 | 0 | antidepressant | none |
| hydrochloride | | | | | | | | | | | | | | | |
| brazilein | | 1.466 | −0.89 | 0.95 | 2.174 | −1.70 | 0.92 | fib/LPS | 1.5 | 0.7 | −1.22 | 0.06 | 3 | | none |
| Desipramine | | 1.616 | −0.36 | 0.65 | 0.002 | 0.00 | | fib/LPS | 0.0 | 1037.2 | 0.78 | 6.04 | 0 | Antidepressant | none |
| hydrochloride | | | | | | | | | | | | | | | |
| fendiline | | 1.747 | −0.72 | 0.80 | 0.900 | −0.51 | 0.25 | fib/LPS | 0.5 | 1.9 | 1.31 | 20.37 | 0 | coronary | none |
| hydrochloride | | | | | | | | | | | | | | vasodilator | |
| colforsin | | 2.083 | −0.94 | 0.97 | 1.346 | −0.65 | 0.90 | fib/LPS | 0.6 | 1.5 | | | 4 | adenylate | none |
| | | | | | | | | | | | | | | cyclase | |
| | | | | | | | | | | | | | | activator, | |
| | | | | | | | | | | | | | | antiglaucoma | |
| neomycin | | 2.297 | −0.75 | 0.93 | 5.000 | | | fib/LPS | 2.2 | 0.5 | | | 4 | antibacterial | Reduction EAE via |
| sulfate | | | | | | | | | | | | | | | alteration gut |
| | | | | | | | | | | | | | | | microflora |
| 6,7-dichloro-3- | | 2.648 | −1.30 | 0.85 | 5.000 | | | fib/LPS | 1.9 | 0.5 | −0.52 | 0.30 | 3 | NMDA | none |
| hydroxy-2- | | | | | | | | | | | | | | and | |
| quinoxalinecarboxylic | | | | | | | | | | | | | | kainate | |
| acid | | | | | | | | | | | | | | receptor | |
| | | | | | | | | | | | | | | antagonist | |
| salinomycin, | | 2.970 | −4.80 | 0.77 | 4.575 | ~−11 | 0.76 | fib/LPS | 1.5 | 0.6 | | | 4 | antibiotic | none |
| sodium | | | | | | | | | | | | | | | |
| Prochlorperazine | | 4.381 | ~−11 | 0.80 | 2.014 | −2.30 | 0.83 | fib/LPS | 0.5 | 2.2 | 1.14 | 13.77 | 0 | Antipsychotic | none |
| dimaleate | | | | | | | | | | | | | | agent; | |
| | | | | | | | | | | | | | | used in | |
| | | | | | | | | | | | | | | the | |
| | | | | | | | | | | | | | | treatment of | |
| | | | | | | | | | | | | | | spastic | |
| | | | | | | | | | | | | | | gastrointestinal | |
| | | | | | | | | | | | | | | disorders | |
| tannic acid | | 4.955 | ~−21 | 0.54 | 4.379 | −3.70 | 0.91 | fib/LPS | 0.9 | 1.1 | | | 4 | nonspecific | none |
| | | | | | | | | | | | | | | enzyme/ | |
| | | | | | | | | | | | | | | receptor | |
| | | | | | | | | | | | | | | blocker | |
| salinomycin, | 3.862 | 0.061 | −0.53 | 0.89 | 0.056 | −0.31 | 0.62 | tox/fib/ | 0.9 | 1.1 | | | 4 | antibiotic | none |
| sodium | | | | | | | | LPS | | | | | | | |
| pomiferin | 3.445 | 0.251 | −0.43 | 0.50 | 0.123 | −0.29 | 0.51 | tox/fib/ | 0.5 | 2.0 | | | 4 | antioxidant | none |
| | | | | | | | | LPS | | | | | | | |
| betamethasone | 2.085 | 0.002 | 0.00 | | 0.002 | 0.00 | | tox/fib/ | 1.0 | 1.0 | −1.16 | 0.07 | 3 | glucocorticoid, | none |
| | | | | | | | | LPS | | | | | | antiinflammatory | |
| colchicine | 0.520 | 0.022 | ~−20 | 0.94 | 0.054 | ~−21 | 0.31 | tox/fib/ | 2.5 | 0.4 | −0.84 | 0.15 | 3 | antimitotic, | Beneficial with |
| | | | | | | | | LPS | | | | | | antigout agent | polyunsaturated |
| | | | | | | | | | | | | | | | FA in MS (1970's) |

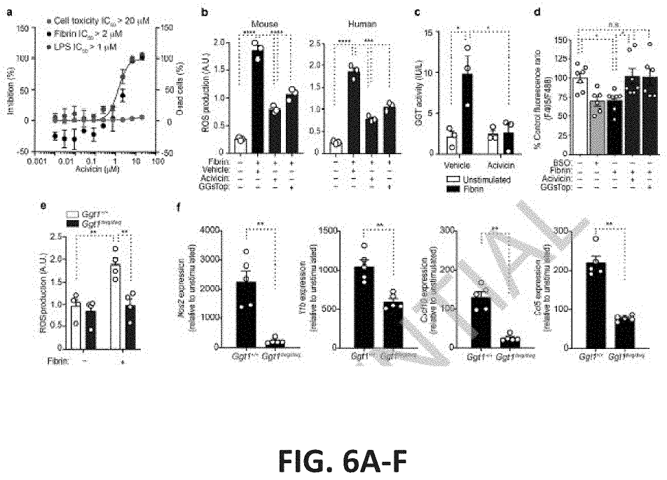

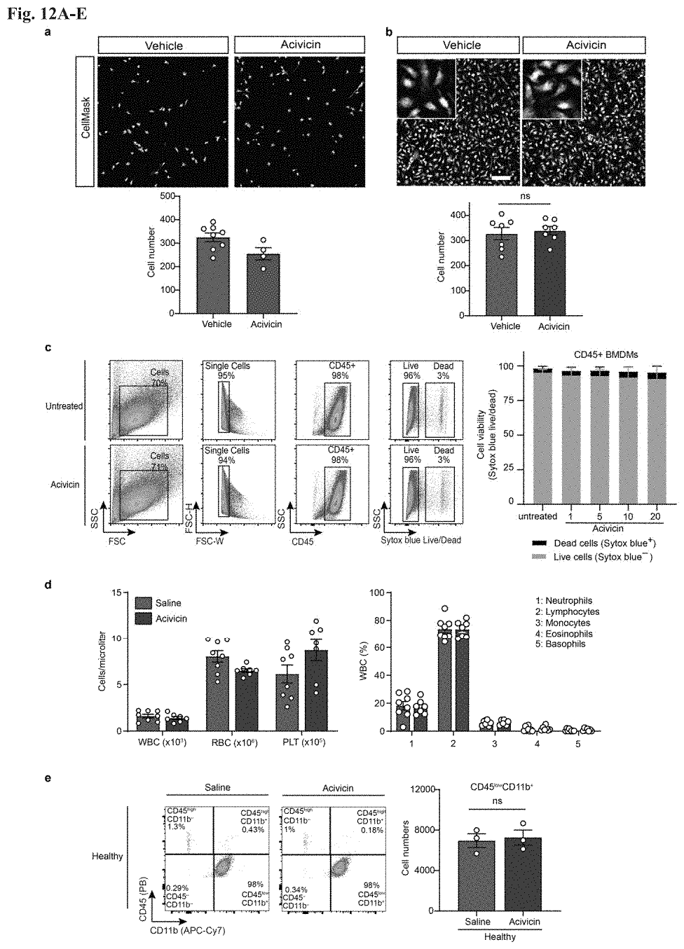

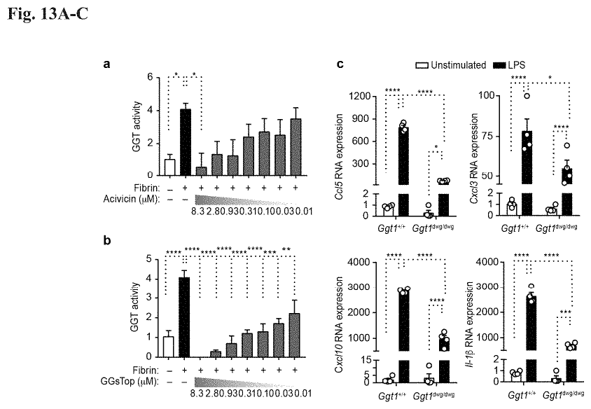

Acivicin suppresses oxidative stress in innate immune cells: Although acivicin has been studied primarily in cancer cells (34), its functions in inflammation and neurological diseases are poorly understood. The effects of acivicin were tested in innate immune cell activation using a series of secondary assays, such as ROS generation, glutathione regulation, gene expression of prooxidant and inflammatory genes, GGT activity, and antigen presentation. Acivicin inhibited fibrin- and LPS-induced microglial activation in a dose-dependent manner, and decreased ROS generation in mouse and human macrophages (FIG. 6a, b). Acivicin did not affect microglia cell number or macrophage cell number in vitro, or the total cell blood count or microglia numbers in the spinal cord in vivo (FIG. 12). This is in accordance with prior studies showing that acivicin at a similar concentration range does not affect neuronal viability (35). Similar to acivicin, the GGT inhibitor GGsTop also inhibited ROS generation (FIG. 6b). GGT degrades the antioxidant glutathione, resulting in increased oxidative stress (36). As shown by GGT activity assay and quantitative real-time cell imaging of intracellular glutathione (37), acivicin and GGsTop inhibited fibrin-induced degradation of glutathione via GGT activation (FIG. 6c, d and FIG. 513A, B). ROS generation and expression of oxidative injury and pro-inflammatory genes were reduced in fibrin- or LPS-stimulated macrophages isolated from Ggt1^{dwg/dwg} mice (38) compared to Ggt1^{+/+} controls (FIG. 6e, f and FIG. 13C). Collectively, these findings show that inhibition of GGT suppresses oxidative and inflammatory functions in innate immune cells.

Therapeutic effects of acivicin in neuroinflammation: In patients with MS and neuromyelitis optica, serum GGT levels correlate with clinical disability, BBB disruption, and inflammatory markers (39). GGTLC1, which encodes the GGT light chain 1, is detected in the cortex of MS patients and is associated with oxidative damage and neuronal injury (40). Serum GGT activity positively correlates with dementia risk and glutathione S-transferase alpha in the plasma of AD patients correlates with late-onset AD progression (41,42). The effects of acivicin treatment were tested in autoimmune acute and chronic progressive models of neuroinflammation, as well as in models of microglia-mediated neurodegeneration. Using immunohistochemistry and FACS, the expression of GGT in EAE spinal cord was first tested.

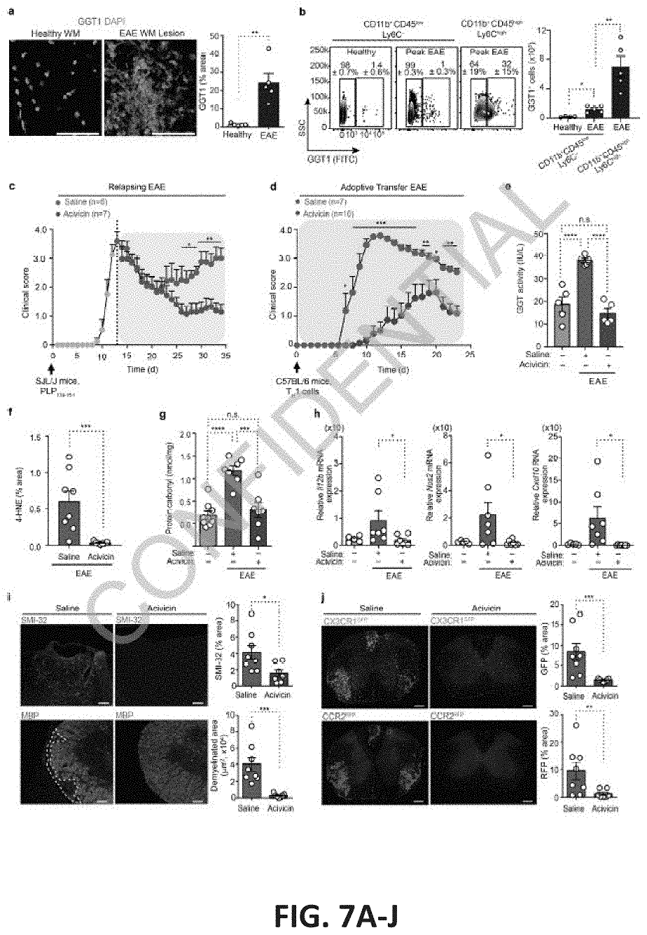

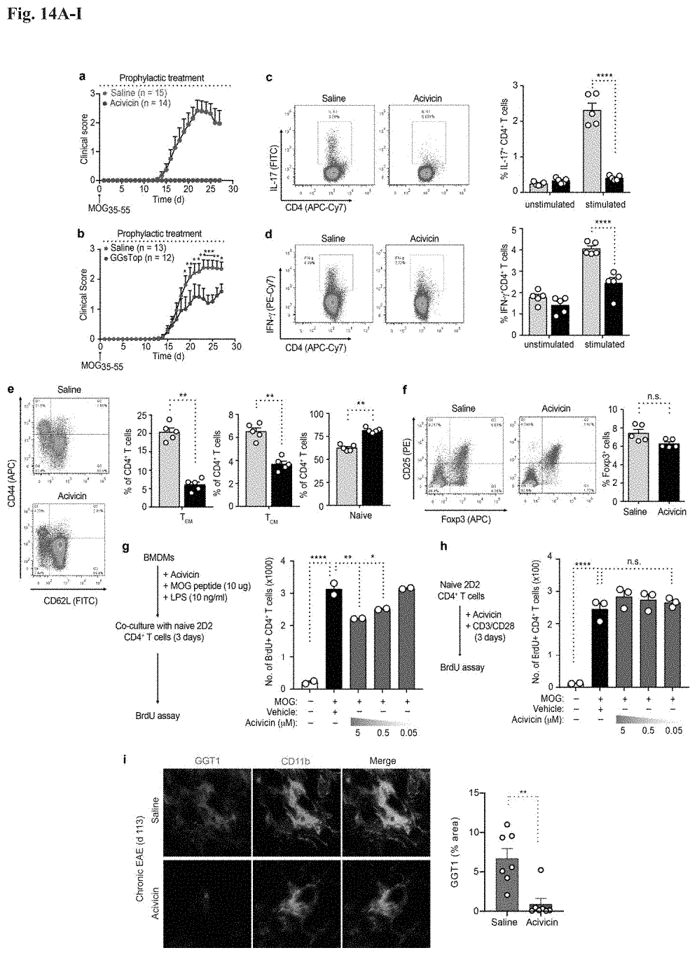

GGT was not detected in healthy spinal cord but increased in microglia and infiltrating monocyte/macrophages in EAE lesions (FIG. 7a, b). Administration of acivicin suppressed the clinical severity during relapses in PLP₁₃₉₋₁₅₁ EAE in SJL/J mice (FIG. 7c). Given prophylactically, acivicin delayed disease onset and severity in adoptive transfer EAE induced by PLP₁₃₉₋₁₅₁-specific T cells and in active MOG35-55 EAE and decreased GGT activity in the EAE spinal cord (FIG. 7d, e and FIG. 14A). Similarly, prophylactic administration of GGsTop reduced neurologic signs in MOG₃₅₋₅₅ EAE (FIG. 14B). In MOG₃₅₋₅₅ EAE mice, acivicin selectively decreased IFN-γ- and IL-17-producing T cells and suppressed encephalitogenic T cell proliferation only in the presence of APCs (FIG. 14C-H), suggesting that in autoimmune models acivicin may indirectly affect T cell responses by suppressing APC function. Acivicin markedly reduced oxidative stress markers 4-hydroxynonenal (4-HNE) and serum protein carbonyl content, proinflammatory gene expression, demyelination, neuronal damage, microglia activation and monocyte/macrophage infiltration in spinal cord lesions in MOG₃₅₋₅₅ EAE mice (FIG. 7f-j).

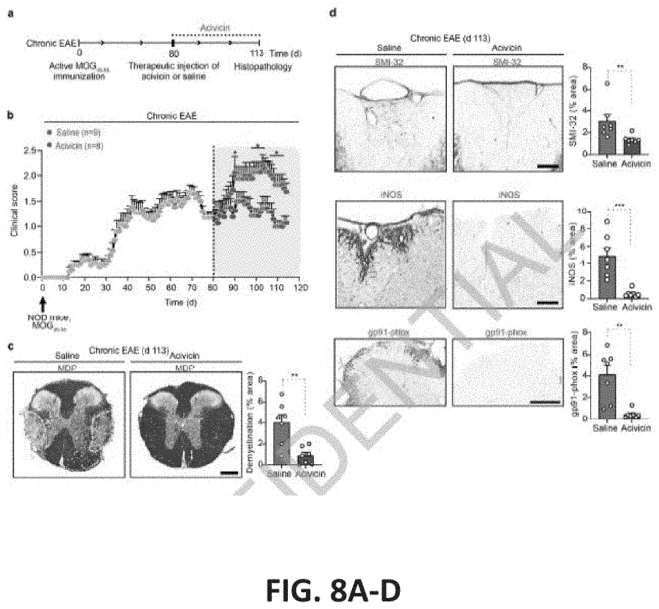

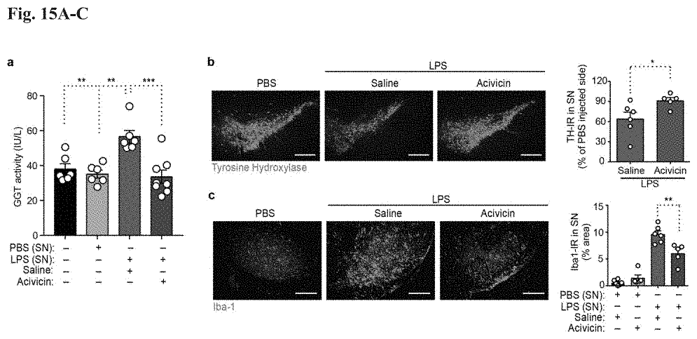

Immunization of non-obese diabetic (NOD) mice with MOG₃₅₋₅₅ results in an acute neurological impairment followed by a chronic phase of progressive accumulation of disability (43). In chronic NOD MOG₃₅₋₅₅ EAE, therapeutic administration of acivicin during the chronic phase even eighty days after EAE induction suppressed progression as indicated by decreased clinical signs (FIG. 8a, b). Acivicin reduced demyelination, axonal damage, expression of GGT1, as well as the oxidative stress markers inducible nitric oxide synthase (iNOS) and NADPH oxidase subunit gp91-phox during the chronic phase of disease (FIG. 8c, d and FIG. 14I). In addition to autoimmune models, acivicin was also tested in a microglial-driven model of neurodegeneration induced by LPS injection in the substantia nigra (SN) (44). Acivicin blocked GGT activity in SN after LPS injection and decreased microglial activation and improved dopaminergic neuronal survival (FIG. 15). These results suggest that acivicin exerts potent anti-inflammatory and neuroprotective effects in neuroinflammatory disease.

### DISCUSSION

The study revealed the oxidative stress transcriptome of CNS innate immune cells in neuroinflammation and identified druggable pathways to suppress neurotoxic innate immunity. By developing a functional transcriptomic and drug discovery pipeline consisting of deep sequencing, small molecule screening, and pathway analysis, novel innate immune cell populations involved in oxidative stress were identified and discovered upstream targeting of glutathione metabolism and redox homeostasis as a therapeutic strategy in neuroinflammation. Using Tox-seq, it was discovered that innate immune cells share a core oxidative stress gene signature mechanistically coupled to coagulation, antigen presentation, and glutathione pathways. The findings introduce the concept of distinct molecular circuits governing oxidative stress and immune-mediated neurodegeneration and reveal their molecular signatures. Given that oxidative stress producing resident and infiltrating innate immune cells are players in MS progression (3,12,45), the oxidative stress signature could enable the identification of specific cell subpopulations contributing to neurotoxicity that could be further characterized with cell-fate mapping studies. Molecular convergence of innate immune cells to an oxidative stress core signature is a springboard for the development of therapies to selectively target CNS innate immune populations that promote oxidative cell injury. Given the broad range of diseases with oxidative stress, the findings have implications for a wide range of diseases including MS, AD, and traumatic brain injury.

The study revealed previously unknown molecular links between coagulation and oxidative stress. In neuroinflammatory lesions, in situ expression of coagulation genes promoting fibrin formation were identified, such as genes encoding coagulation factors IX and X, Vitamin K Dependent Plasma Glycoprotein, and the von Willebrand factor receptors glycoproteins IX and Ib. Intriguingly, coagulation gene expression was differentially increased in ROS⁺ CNS innate immune cells that co-expressed genes regulating oxidative stress, such as the NADPH oxidase subunit gp91-phox and GGT, and iron metabolism. Dysregulation of the coagulation pathway and fibrin deposition correlates with cortical damage, microglia activation, and neuronal loss in MS and EAE (29,30,46-48). Administration of anti-coagulants or inhibition of the interaction of fibrin with the CD11b-CD18 integrin receptor (also known as Mac-1, complement receptor 3, αMβ2) reduces clinical signs, oxidative stress, and neurodegeneration in MS animal models (9,10,31). Fibrin signaling via the CD11b-CD18 receptor may potentiate the crosstalk of NADPH oxidase with the GGT pathway (49) leading to redox regulation. Indeed, fibrin activates NADPH oxidase (9) and GGT (herein) to promote degradation of glutathione and oxidative stress in innate immune cells. Furthermore, inhibition of fibrin interaction with CD11b-CD18 or inhibition of NADPH oxidase (9,10), or GGT (herein) suppresses fibrin-induced ROS generation. NADPH oxidase activation and degradation of glutathione by fibrin can be a prooxidant mechanisms in diseases with blood-brain barrier disruption and vascular pathology. Thus, it is possible that there is a positive-feedback loop between coagulation, oxidative stress, and the pro-inflammatory response, whereby subpopulations of innate immune cells promote the local synthesis of coagulation factors to increase fibrin deposition and promote oxidative injury. Local increases in coagulation activity by innate immune cell subpopulations as an oxidative stress mechanism could be relevant for other diseases in the brain and periphery with vascular damage associated with fibrin deposition and oxidative injury (5,7,10,31,50,51).

Acivicin was selected as an upstream regulator of glutathione and redox homeostasis. GGT mediated cleavage of glutathione causes iron redox cycling, which stimulates the release of hydroxyl radicals (34). Thus, redox restoration by acivicin may protect against EAE progression by homeostatic regulation of glutathione in oxidative stress-producing innate immune cells. In accordance, pharmacologic inhibition of GGT by GGsTop reduced oxidative stress markers and protected from renal reperfusion injury (52). In EAE, acivicin suppressed inflammatory and prooxidant pathways and decreased axonal damage, demyelination, and peripheral cell recruitment into the CNS. Suppression of oxidative stress and reduction of chemokines that facilitate cell recruitment by acivicin might reduce myeloid cell numbers and decrease lesion size. Acivicin also regulates glutamate metabolism and leukotriene responses with potential effects on immune cell recruitment, neuronal and T cell functions (34,53,54). Cell sorting and scRNA-seq studies may be used to determine the drug selectivity of acivicin at the single-cell level and its effects on oxidative stress resistance and additional prooxidant and inflammatory markers. These studies could decipher mechanisms linking oxidative stress and peripheral cell recruitment into the CNS in EAE and other models of neurologic disease. Glutamine analogues like acivicin exhibit dose limiting toxicity in anti-cancer trials potentially due to interference with recycling of glutamine (34). Given the toxicity of high doses of acivicin in the clinic or the consequences of global depletion of GGT1 in mice (34), identification of safe drugs modulating glutathione metabolism might facilitate the restoration of redox hemostasis in neuroinflammatory disease.

In summary, by generating the first oxidative stress cell atlas of innate immunity, cell populations and molecular mechanisms involved in oxidative injury and neurotoxicity in neuroinflammatory disease were identified. Transcriptional signatures of oxidative stress genes that can be used as a resource and follow-up studies to validate additional gene targets, cell populations, and drugs from the HTS screen were defined. Furthermore, a method, Tox-seq, was advanced, which can be used to determine the functional role of oxidative stress producing cells in a wide range of disease states. Given the multiple roles of ROS in oxidative damage and redox regulation (55), Tox-seq could reveal molecular pathways governing ROS-mediated functions in physiology and pathology. Integration of functional transcriptomics and HTS may prioritize druggable pathways that could enhance cherry-picking or in silico screens to identify compounds of interest for preclinical testing. Thus, oxidative stress transcriptomics and drug discovery approaches could identify and target neurotoxic CNS innate immune populations and lead to the development of selective neuroprotective strategies.

### BIBLIOGRAPHY

1. Nikic, I. et al. A reversible form of axon damage in experimental autoimmune encephalomyelitis and multiple sclerosis. Nat Med 17, 495-499 (2011).
2. Locatelli, G. et al. Mononuclear phagocytes locally specify and adapt their phenotype in a multiple sclerosis model. Nat Neurosci 21, 1196-1208 (2018).
3. Fischer, M. T. et al. NADPH oxidase expression in active multiple sclerosis lesions in relation to oxidative tissue damage and mitochondrial injury. Brain 135, 886-899 (2012).
4. Heppner, F. L., Ransohoff, R. M. & Becher, B. Immune attack: the role of inflammation in Alzheimer disease. Nat Rev Neurosci 16, 358-372 (2015).
5. Nortley, R. et al. Amyloid beta oligomers constrict human capillaries in Alzheimer's disease via signaling to pericytes. Science 365 (2019).
6. Park, L. et al. NADPH-oxidase-derived reactive oxygen species mediate the cerebrovascular dysfunction induced by the amyloid beta peptide. J Neurosci 25, 1769-1777 (2005).
7. Park, L. et al. NADPH-oxidase-derived reactive oxygen species mediate the cerebrovascular dysfunction induced by the amyloid beta peptide. J Neurosci 25, 1769-1777 (2005).
8. Back, S. A., Gan, X., Li, Y., Rosenberg, P. A. & Volpe, J. J. Maturation-dependent vulnerability of oligodendrocytes to oxidative stress-induced death caused by glutathione depletion. J Neurosci 18, 6241-6253 (1998).
9. Ryu, J. K. et al. Fibrin-targeting immunotherapy protects against neuroinflammation and neurodegeneration. Nat Immunol 19, 1212-1223 (2018).
10. Merlini, M. et al. Fibrinogen induces microglia-mediated spine elimination and cognitive impairment in Alzheimer's Disease. Neuron 101, 1099-1108 (2019).
11. Weiner, H. L. A shift from adaptive to innate immunity: a potential mechanism of disease progression in multiple sclerosis. J Neurol 255 Suppl 1, 3-11 (2008).
12. Lassmann, H., van Horssen, J. & Mahad, D. Progressive multiple sclerosis: pathology and pathogenesis. Nat. Rev. Neurol. 8, 647-656 (2012).
13. Mahad, D. H., Trapp, B. D. & Lassmann, H. Pathological mechanisms in progressive multiple sclerosis. Lancet Neurol 14, 183-193 (2015).
14. Keren-Shaul, H. et al. A Unique Microglia Type Associated with Restricting Development of Alzheimer's Disease. Cell 169, 1276-1290 e1217 (2017).
15. Hammond, T. R. et al. Single-Cell RNA Sequencing of Microglia throughout the Mouse Lifespan and in the Injured Brain Reveals Complex Cell-State Changes. Immunity 50, 253-271 e256 (2019).
16. Schirmer, L. et al. Neuronal vulnerability and multilineage diversity in multiple sclerosis. Nature (2019).
17. Mathys, H. et al. Single-cell transcriptomic analysis of Alzheimer's disease. Nature 570, 332-337 (2019).
18. Gosselin, D. et al. An environment-dependent transcriptional network specifies human microglia identity. Science 356 (2017).
19. Krasemann, S. et al. The TREM2-APOE Pathway Drives the Transcriptional Phenotype of Dysfunctional Microglia in Neurodegenerative Diseases. Immunity 47, 566-581.e569 (2017).
20. Jordão, M. J. C. et al. Single-cell profiling identifies myeloid cell subsets with distinct fates during neuroinflammation. Science 363, eaat7554 (2019).
21. Van Hove, H. et al. A single-cell atlas of mouse brain macrophages reveals unique transcriptional identities shaped by ontogeny and tissue environment. Nat Neurosci 22, 1021-1035 (2019).
22. Diehn, M. et al. Association of reactive oxygen species levels and radioresistance in cancer stem cells. Nature 458, 780-783 (2009).
23. Butler, A., Hoffman, P., Smibert, P., Papalexi, E. & Satija, R. Integrating single-cell transcriptomic data across different conditions, technologies, and species. Nat Biotechnol 36, 411-420 (2018).
24. Li, Q. et al. Developmental Heterogeneity of Microglia and Brain Myeloid Cells Revealed by Deep Single-Cell RNA Sequencing. Neuron 101, 207-223 e210 (2019).
25. Ajami, B. et al. Single-cell mass cytometry reveals distinct populations of brain myeloid cells in mouse neuroinflammation and neurodegeneration models. Nat Neurosci 21, 541-551 (2018).
26. Mrdjen, D. et al. High-Dimensional Single-Cell Mapping of Central Nervous System Immune Cells Reveals Distinct Myeloid Subsets in Health, Aging, and Disease. Immunity 48, 380-395.e386 (2018).
27. Geirsdottir, L. et al. Cross-Species Single-Cell Analysis Reveals Divergence of the Primate Microglia Program. Cell 179, 1609-1622 e1616 (2019).
28. Choi, B. Y. et al. Inhibition of NADPH oxidase activation reduces EAE-induced white matter damage in mice. J Neuroinflammation 12, 104 (2015).
29. Davalos, D. et al. Early detection of thrombin activity in neuroinflammatory disease. Ann Neurol 75, 303-308 (2014).
30. Davalos, D. et al. Fibrinogen-induced perivascular microglial clustering is required for the development of axonal damage in neuroinflammation. Nat. Commun. 3, 1227 (2012).
31. Petersen, M. A., Ryu, J. K. & Akassoglou, K. Fibrinogen in neurological diseases: mechanisms, imaging and therapeutics. Nat Rev Neurosci 19, 283-301 (2018).
32. Shannon, P. et al. Cytoscape: a software environment for integrated models of biomolecular interaction networks. Genome Res 13, 2498-2504 (2003).
33. Kutmon, M. et al. PathVisio 3: an extendable pathway analysis toolbox. PLoS Comput. Biol. 11, e1004085 (2015).
34. Corti, A., Franzini, M., Paolicchi, A. & Pompella, A. Gamma-glutamyltransferase of cancer cells at the crossroads of tumor progression, drug resistance and drug targeting. Anticancer Res 30, 1169-1181 (2010).
35. Koga, M. et al. Glutathione is a physiologic reservoir of neuronal glutamate. Biochem Biophys Res Commun 409, 596-602 (2011).
36. Drozdz, R. et al. gamma-Glutamyltransferase dependent generation of reactive oxygen species from a glutathione/transferrin system. Free Radic Biol Med 25, 786-792 (1998).
37. Jiang, X. et al. Quantitative real-time imaging of glutathione. Nat. Commun. 8, 16087 (2017).
38. Tsuji, T., Yamada, K. & Kunieda, T. Characterization of the dwg mutations: dwg and dwg(Bayer) are new mutant alleles of the Ggt1 gene. Mamm Genome 20, 711-719 (2009).
39. Shu, Y. et al. Association of serum gamma-glutamyltransferase and C-reactive proteins with neuromyelitis optica and multiple sclerosis. Mult. Scler. Relat. Disord. 18, 65-70 (2017).
40. Fischer, M. T. et al. Disease-specific molecular events in cortical multiple sclerosis lesions. Brain 136, 1799-1815 (2013).
41. Iturria-Medina, Y. et al. Early role of vascular dysregulation on late-onset Alzheimer's disease based on multifactorial data-driven analysis. Nat. Commun. 7, 11934 (2016).
42. Kunutsor, S. K. & Laukkanen, J. A. Gamma glutamyltransferase and risk of future dementia in middle-aged to older Finnish men: A new prospective cohort study. Alzheimers Dement 12, 931-941 (2016).
43. Mayo, L. et al. Regulation of astrocyte activation by glycolipids drives chronic CNS inflammation. Nat Med 20, 1147-1156 (2014).
44. Herrera, A. J., Castano, A., Venero, J. L., Cano, J. & Machado, A. The single intranigral injection of LPS as a new model for studying the selective effects of inflammatory reactions on dopaminergic system. Neurobiol. Dis. 7, 429-447 (2000).
45. International Multiple Sclerosis Genetics Consortium. Multiple sclerosis genomic map implicates peripheral immune cells and microglia in susceptibility. Science 365, eaav7188 (2019).
46. Magliozzi, R. et al. Iron homeostasis, complement, and coagulation cascade as CSF signature of corical lesions in early multiple sclerosis. Ann. Clin. Transl. Neurol. 6, 2150-2163 (2019).
47. Yates, R. L. et al. Fibrin(ogen) and neurodegeneration in the progressive multiple sclerosis cortex. Ann Neurol 82, 259-270 (2017).
48. Han, M. H. et al. Proteomic analysis of active multiple sclerosis lesions reveals therapeutic targets. Nature 451, 1076-1081 (2008).
49. Ravuri, C., Svineng, G., Pankiv, S. & Huseby, N. E. Endogenous production of reactive oxygen species by the NADPH oxidase complexes is a determinant of gammaglutamyltransferase expression. Free Radic. Res. 45, 600-610 (2011).
50. Rodriguez-Rodriguez, A., Egea-Guerrero, J. J., Murillo-Cabezas, F. & Carrillo-Vico, A. Oxidative stress in traumatic brain injury. Curr Med Chem 21, 1201-1211 (2014).
51. Davalos, D. & Akassoglou, K. Fibrinogen as a key regulator of inflammation in disease. Semin Immunopathol 34, 43-62 (2012).
52. Yamamoto, S. et al. Preventive effect of GGsTop, a novel and selective gamma-glutamyl transpeptidase inhibitor, on ischemia/reperfusion-induced renal injury in rats. J Pharmacol Exp Ther 339, 945-951 (2011).
53. Birkner, K. et al. beta1-Integrin- and KV1.3 channel-dependent signaling stimulates glutamate release from Th17 cells. J Clin Invest (2020).
54. Sedlak, T. W. et al. The glutathione cycle shapes synaptic glutamate activity. Proc Natl Acad Sci USA 116, 2701-2706 (2019).
55. Schieber, M. & Chandel, N. S. ROS function in redox signaling and oxidative stress. Curr Biol 24, R453-462 (2014).
56. Saederup, N. et al. Selective chemokine receptor usage by central nervous system myeloid cells in CCR2-red fluorescent protein knock-in mice. PLoS One 5, e13693 (2010).
57. Jung, S. et al. Analysis of fractalkine receptor CX(3)CR1 function by targeted deletion and green fluorescent protein reporter gene insertion. Mol Cell Biol 20, 4106-4114 (2000).
58. Huang da, W., Sherman, B. T. & Lempicki, R. A. Systematic and integrative analysis of large gene lists using DAVID bioinformatics resources. Nat. Protoc. 4, 44-57 (2009).
59. Aronesty, E. ea-utils: “Command-line tools for processing biological sequencing data”. (2011).
60. Robinson, M. D., McCarthy, D. J. & Smyth, G. K. edgeR: a Bioconductor package for differential expression analysis of digital gene expression data. Bioinformatics 26, 139-140 (2010).
61. Robinson, M. D. & Oshlack, A. A scaling normalization method for differential expression analysis of RNA-seq data. Genome Biol. 11, R25 (2010).
62. Zambon, A. C. et al. GO-Elite: a flexible solution for pathway and ontology overrepresentation. Bioinformatics 28, 2209-2210 (2012).
63. Van der Laan, M. J. & Pollard, K. S. A new algorithm for hybrid clustering of gene expression data with visualization and the bootstrap. J. Stat. Plan. Interference 117, 275-303 (2003).
64. Zuberi, K. et al. GeneMANIA prediction server 2013 update. Nucleic Acids Res 41, W115-122 (2013).
65. Butovsky, O. et al. Identification of a unique TGF-beta-dependent molecular and functional signature in microglia. Nat Neurosci 17, 131-143 (2014).
66. Kutmon, M. et al. WikiPathways: capturing the full diversity of pathway knowledge. Nucleic Acids Res 44, D488-494 (2016).
67. Ryu, J. K. et al. Blood coagulation protein fibrinogen promotes autoimmunity and demyelination via chemokine release and antigen presentation. Nat. Commun. 6, 8164 (2015).
68. Adams, R. A. et al. The fibrin-derived gamma377-395 peptide inhibits microglia activation and suppresses relapsing paralysis in central nervous system autoimmune disease. J Exp Med 204, 571-582 (2007).
69. Akassoglou, K. et al. Oligodendrocyte apoptosis and primary demyelination induced by local TNF/p55TNF receptor signaling in the central nervous system of transgenic mice: models for multiple sclerosis with primary oligodendrogliopathy. Am J Pathol 153, 801-813 (1998).
70. Akassoglou, K. et al. Fibrin depletion decreases inflammation and delays the onset of demyelination in a tumor necrosis factor transgenic mouse model for multiple sclerosis. Proc Natl Acad Sci USA 101, 6698-6703 (2004).

PCT/US2018/052694

All publications, nucleotide and amino acid sequence identified by their accession nos., patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

What is claimed is:

1. A method to treat or prevent neurodegeneration in a mammal comparing administering to said mammal an effect amount of a composition comprising a modulator of calcium signaling, a modulator of microtubule dynamics, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof or one or more of the small molecules provided in Table 1 and/or Table 2, provided that the small molecule is not acivicin.

2. A method for inhibiting microglial activation in the CNS of a mammal and/or promoting survival of CNS neurons in a mammal, comprising administering to a mammal in need thereof an effective amount of a composition comprising a modulator of calcium signaling, a modulator of microtubule dynamics, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

3. A method for preventing or reducing the rate of demyelination and/or neuronal injury in a mammal and/or promoting remyelination in a mammal and/or treating or preventing or decreasing oxidative stress in a mammal in need thereof comprising administering to the mammal an effective amount of a modulator of calcium signaling, a modulator of microtubule dynamics, a modulator of chemokine signaling, a modulator of DNA replication, a modulator of dopamine receptor signaling, a modulator of cAMP signaling, a modulator of glucocorticoid-receptor signaling, a modulator of purine nucleotide biosynthesis, a modulator of neurotransmitter transport or a combination thereof.

4. The method of claim 1, wherein the modulator of calcium signaling is a calcium channel blocker, a vasodilator or an adrenoreceptor agonist.

5. The method of claim 4, wherein the calcium channel blocker comprises fendiline.

6. The method of claim 4, wherein the vasodilator comprises nylidrin.

7. The method of claim 1, wherein the modulator of microtubule dynamics is an inhibitor of microtubule assembly.

8. The method of claim 7, wherein the inhibitor of microtubule assembly comprises vinblastine, colchicine and/or podofilox.

9. The method of claim 1, wherein the modulator of chemokine signaling comprises tannic acid.

10. The method of claim 1, wherein the modulator of DNA replication is an inhibitor of DNA topoisomerase II.

11. The method of claim 10, wherein the inhibitor of DNA topoisomerase II comprises teniposide.

12. The method of claim 1, wherein the modulator of dopamine receptor signaling is blocker of dopamine receptors.

13. The method of claim 12, wherein the blocker of dopamine receptors comprises prochlorperazine or thioridazine.

14. The method of claim 1, wherein the modulator of cAMP signaling comprises nylidrin, prochlorperazine or thioridazine.

15. The method of claim 1, wherein the modulator of glucocorticoid-receptor signaling comprises betamethasone, dexamethasone, dexamethasone acetate, dexamethasone sodium phosphate, fludrocortisone acetate, fluocinolone acetonide, fluocinonide, fluorometholone, flurandrenolide, hydrocortisone, hydrocortisone acetate, hydrocortisone butyrate, hydrocortisone hemisuccinate, hydrocortisone sodium phosphate, methylprednisolone, prednisolone or triamcinolone diacetate.

16. The method of claim 1, wherein the modulator of purine nucleotide biosynthesis is an inhibitor of inosine-5′-monophosphate dehydrogenase (IMPDH).

17. The method of claim 16, wherein the inhibitor of inosine-5′-monophosphate dehydrogenase (IMPDH) comprises mycophenolic acid (MPA).

18. The method of claim 1, wherein the modulator of neurotransmitter transport is an inhibitor of norepinephrine reuptake.

19. The method of claim 18, wherein the inhibitor of norepinephrine reuptake is maprotiline.

20. The method of claim 1, wherein said mammal has been diagnosed with a disease, disorder, or injury involving demyelination, dysinyelination, or neurodegeneration.
